(19) United States
(12) Patent Application Publication
(10) Pub. No.: US 2022/0048990 A1
(43) Pub. Date: Feb. 17, 2022

(54) IMMUNOGLOBULIN A ANTIBODIES AND METHODS OF PRODUCTION AND USE

(71) Applicant: GENENTECH, INC., South San Francisco, CA (US)

(72) Inventors: Twyla Noelle Lombana, South San Francisco, CA (US); Julie A. Zorn, South San Francisco, CA (US); Marissa L. Matsumoto, South San Francisco, CA (US); Christoph Spiess, South San Francisco, CA (US); Claudio Ciferri, South San Francisco, CA (US); Alberto Estevez, South San Francisco, CA (US)

(73) Assignee: GENENTECH, INC., South San Francisco, CA (US)

(21) Appl. No.: 17/381,145

(22) Filed: Jul. 20, 2021

Related U.S. Application Data
(63) Continuation of application No. PCT/US2020/014617, filed on Jan. 22, 2020.
(60) Provisional application No. 62/838,071, filed on Apr. 24, 2019.
(60) Provisional application No. 62/795,367, filed on Jan. 22, 2019.

(51) Int. Cl. C07K 16/28; C07K 1/36; C07K 16/46

(52) U.S. Cl. CPC C07K 16/2803; C07K 1/36; C07K 16/283; C07K 2317/92; C07K 2319/00; C07K 2317/52; C07K 2317/41; C07K 16/462

(57) ABSTRACT

The presently disclosed subject matter provides antibodies, e.g., IgA antibodies and IgG-IgA fusion molecules, and compositions comprising such antibodies, as well as methods of making and using such antibodies and compositions.

### CROSS-REFERENCE TO RELATED APPLICATIONS

This application is a continuation of International Patent Application No. PCT/US2020/014617, filed Jan. 22, 2020, which claims priority to U.S. Provisional Application No. 62/795,367, filed on Jan. 22, 2019, and U.S. Provisional Application No. 62/838,071, filed on Apr. 24, 2019, the contents of each of which are incorporated by reference in their entireties, and to each of which priority is claimed.

### SEQUENCE LISTING

The instant application contains a Sequence Listing which has been submitted electronically in ASCII format and is hereby incorporated by reference in its entirety. Said ASCII copy, created on Jun. 22, 2021, is named 00B206_1107_SL.txt and is 35,068 bytes in size. The Sequence Listing does not extend beyond the scope of the specification and thus does not contain new matter.

### FIELD OF THE INVENTION

The present disclosure relates to antibodies, e.g., IgA antibodies and IgG-IgA fusion molecules, and compositions comprising such antibodies, as well as methods of making and using such antibodies and compositions.

### BACKGROUND

Immunoglobulin A (IgA) is a major class of antibody present in the mucosal secretions of most mammals and represents a first line of defense against invasion by inhaled and ingested pathogens at the vulnerable mucosal surfaces. In humans, there are two IgA isotypes, IgA1 and IgA2, distinguished by a 13-residue extension in the hinge region of the IgA1 heavy chain (HC) that is absent in IgA2 molecules (Leusen, Mol. Immunol. 68:35-9 (2015)). Both isotypes are abundant in all organs and tissues, except in the intestines where IgA2 is predominant and in the serum where IgA1 monomer is found almost exclusively (Kerr, Biochem. J. 271:285-96 (1990)). There are three allotypes of IgA2: m1 (Tsuzukida et al., Proc Natl Acad Sci USA 76:1104-8 (1979)), m2 (Toraño et al., Proc Natl Acad Sci USA 75:966-9 (1978)) and mn (Chintalacharuvu et al., J Immunol 152:5299-304 (1994)). The m2 and mn allotypes form canonical light chain (LC)-HC disulfides, whereas the presence of a proline at position 221 of the HC in IgA2m1 results in LC-LC disulfide bond formation (Chintalacharuvu et al., J Immunol 157:3443-9 (1996)). Mutation of proline 221 in the IgA2m1 allotype to arginine (P221R), which is found in the m2 and mn allotypes, restores the canonical LC-HC linkage (Lohse et al., Cancer Res 76:403-17 (2016) and Chintalacharuvu et al. (1996)). Sequence identity between the IgA1 and IgA2 isotypes is quite high at ˜90% and even higher amongst the IgA2 allotypes, with only six residue differences between m1 and m2 and two residue differences between either m1 or m2 with mn (Chintalacharuvu et al. (1994)).

Contrary to other human immunoglobulin classes, IgA has the unique ability to naturally exist as both monomeric and polymeric soluble species, whereas only polymeric IgA (plgA) can bind to pIgR for subsequent transcytosis (Yoo et al., Clin. Immunol. 116:3-10 (2005)). Oligomerization of IgA is facilitated by an 18 residue C-terminal extension of the HC called the tailpiece and the 137 amino acid joining chain (JC). The penultimate residue of the IgA tailpiece, Cys471, of the first IgA monomer mediates disulfide bond formation with Cys15 of the JC, while Cys471 of the second IgA monomer mediates disulfide bond formation with Cys69 of the JC to form a covalent IgA dimer that is held together by a single JC (Zikan et al., Mol Immunol 23:541-4 (1986) and Halpern et al., J Immunol 111:1653-60 (1973)). As each IgA monomer is composed of two HCs, each with a tailpiece, the IgA dimer has two unpaired Cys471 residues through which additional IgA monomers could be linked. Higher order IgA oligomers such as trimers, tetramers and pentamers have been reported (Suzuki et al., Proc Natl Acad Sci USA 112:7809-14 (2015)). Whereas serum IgA is predominantly monomeric, polymeric IgAs are produced by plasma cells in the lamina propria. The presence of the JC in polymeric IgA is required for binding pIgR on the basolateral side of the epithelium and for active transport to the apical side of mucosal tissues (Wu et al., Clin Dev Immunol 11:205-13 (2004)). Upon transcytosis, the extracellular domain of pIgR is proteolytically cleaved creating what is known as the secretory component (SC), which remains covalently attached to the polymeric IgA heavy chain through a disulfide bond between Cys467 in pIgR and Cys311 in one HC (Fallgreen-Gebauer et al., Biol Chem Hoppe-Seyler 374:1023-8 (1993) and Bastian et al., Adv Exp Med Biol 371A:581-3 (1995)). This complex is deemed secretory IgA (slgA), the main determinant of mucosal immunity (Mantis et al., Mucosal Immunol 4:603-11 (2011) and Johansen et al. Mucosal Immunol 4:598-602 (2011)).

Immunoglobulin A (IgA) research has highlighted multiple potential therapeutic applications and unique mechanisms of action for both monomeric and polymeric immunoglobulin A (IgA) antibodies compared to traditional IgG-based therapeutics (Yoo et al. (2005), Bakema et al., MAbs 3:352-61 (2011) and Leusen (2015)). In oncology, monomeric and polymeric anti-EGFR and anti-CD20 IgAs have demonstrated superior tumor cell killing compared to IgG, driven by FcαRI-mediated cytotoxicity or more effective receptor binding and downmodulation (Pascal et al., Haematologica 97:1686-94 (2012), Boross et al., EMBO Molecular Medicine 5:1213-26 (2013) and Lohse et al. (2016)). The cytotoxic activity of IgA could be further increased via dual engagement of both FcγR and FcαRI by IgG/A fusion or hybrid molecules (Li et al., Oncotarget (2017) and Kelton et al., Chem Biol 21:1603-9 (2015)). For infectious disease, IgA multivalent target engagement enabled superior antigen binding and neutralization in influenza infection models (Suzuki et al. (2015)). Additionally, human IgA dimer (dIgA) could be effectively delivered to the kidney lumen in a polycystic kidney disease mouse model via binding to the polymeric immunoglobulin receptor (pIgR), whereas IgG molecules could not (Olsan et al., Journal of Biological Chemistry 290:15679-86 (2015)). Harnessing the specific transcytosis activity of IgA could potentially allow access to therapeutic targets within the luminal side of mucosal tissues that are inefficiently targeted by current IgG therapeutics (Bakema et al. (2011), Olsan et al. (2015) and Borrok et al., JCI Insight 3 (2018)).

Production of recombinant monomeric IgA is more challenging than that of the well-established IgG molecule. IgA antibodies typically suffer from poor expression and heterogenous glycosylation. Whereas human IgG1 typically has only two N-linked glycosylation sites, one in each C_{H}2 domain, human IgA contains multiple glycosylation sites that can be susceptible to glycan heterogeneity (Leusen (2015)). IgA1 has multiple O-linked glycosylation sites in the hinge region and also two N-linked glycosylation sites in the HC constant domain. While IgA2 molecules are not modified by O-linked glycans, they do contain either four (IgA2m1) or five (IgA2m2 and IgA2mn) N-linked glycosylation sites (Yoo et al. (2005) and Bakema et al. (2011)). The JC also contains one N-linked glycosylation site. Assembly of the three polypeptide chains (LC, HC and JC) leads to multiple oligomeric states and further contributes to the overall complexity of recombinant polymeric IgA (Rouwendal et al., MAbs 8:74-86 (2016) and Brunke et al., MAbs 5:936-45 (2013)). With increasing size of an IgA oligomer comes not only an increased number of glycosylation sites, but also the potential for more glycan heterogeneity.

IgA has previously been shown to have a short circulating half-life (<1 day to ˜4 days) in multiple species (Challacombe et al., Immunology 36:331-8 (1979) and Leusen (2015)). Unlike IgG, IgA does not bind the neonatal receptor, FcRn, and therefore, cannot undergo endosomal recycling and escape from lysosomal degradation (Roopenian et al., Nat Rev Immunol 7:715-25 (2007)). In addition to the lack of FcRn binding, immature N-linked glycans can also contribute to shorter serum half-lives of recombinant IgA by making them susceptible targets of carbohydrate-specific, endocytic receptors such as the asialoglycoprotein receptor (ASGPR) (Boross et al. (2013) and (Rifai et al., J Exp Med 191:2171-82 (2000)) and mannose receptor (Lee et al., Science 295:1898-901 (2002) and Heystek et al., J Immunol 168:102-7 (2002)). These scavenging receptors, which are highly concentrated in the liver, recognize glycoproteins bearing incompletely sialylated N-linked glycans and remove them from circulation (Tomana et al., Gastroenterology 94:762-70 (1988) and Daniels et al., Hepatology 9:229-34 (1989)).

Accordingly, there is a need in the art for IgA antibodies that have a longer half-life and for production methods to improve expression levels and polymeric IgA generation.

### SUMMARY

The present disclosure relates to IgA antibodies and compositions comprising such antibodies, as well as methods of making and using such antibodies and compositions.

In certain embodiments, the present disclosure is directed to isolated IgA antibodies. For example, but not by way of limitation, an isolated IgA antibody, or a fragment thereof, of the present disclosure comprises a substitution at amino acid V458. In certain embodiments, amino acid V458 is substituted with an isoleucine (i.e., V4581). In certain embodiments, the isolated IgA antibody is an IgA1, IgA2mn or IgA2m1 antibody.

In certain embodiments, an isolated IgA antibody, or a fragment thereof, of the present disclosure comprises a substitution at amino acid I458. In certain embodiments, amino acid I458 is substituted with a valine (i.e., I458V). In certain embodiments, the isolated IgA antibody is an IgA2m2 antibody.

The present disclosure further provides an isolated IgA antibody that comprises a substitution at amino acid N459 and/or S461. In certain embodiments, amino acid N459 is substituted with a glutamine (i.e., N459Q). In certain embodiments, amino acid S461 is substituted with an alanine (i.e., S461A). In certain embodiments, IgA antibody is an IgA1 or IgA2m1 antibody.

The present disclosure further provides an isolated IgA antibody that comprises one or more substitutions at an amino acid selected from the group consisting of N166, T168, N211, S212, S213, N263, T265, N337, I338, T339, N459, S461 and a combination thereof. In certain embodiments, the IgA antibody has a substitution at amino acid N459 and is an IgA1, IgA2m1 or an IgA2m2 antibody. In certain embodiments, the IgA antibody has a substitution at amino acid N166 and is an IgA2m1 or an IgA2m2 antibody. In certain embodiments, the IgA antibody has a substitution at amino acid S212 and is an IgA2m2 antibody. In certain embodiments, the IgA antibody has a substitution at amino acid N263 and is an IgA1, IgA2m1 or an IgA2m2 antibody. In certain embodiments, the IgA antibody has substitutions at amino acids N337, I338, T339 and is an IgA2m1 or an IgA2m2 antibody. In certain embodiments, the IgA antibody has substitutions at amino acids N337, I338, T339 and one or more substitutions at T168, N211, S212, S213, N263, T265, N459, S461 and a combination thereof. In certain embodiments, the IgA antibody is an IgA2m2 antibody and comprises substitutions at amino acids N166, S212, N263, N337, I338, T339 and N459. For example, but not by way of limitation, the substitutions at amino acids N166, S212, N263, N337, I338, T339 and N459 can be N166A, S212P, N263Q, N337T, I338L, T339S and N459Q.

The present disclosure further provides isolated IgG-IgA fusion molecules. In certain embodiments, an isolated IgG-IgA fusion molecule can comprise a full-length IgG antibody fused at its C-terminus to an Fc region of an IgA antibody, wherein the Fc region of the IgA antibody comprises a sequence comprising P221 or R221 through the C-terminus of the heavy chain of the IgA antibody and where IgG antibody further comprises a deletion of amino acid K447. The present disclosure provides an isolated IgG-IgA fusion molecule comprising a full-length IgG antibody fused at its C-terminus to an Fc region of an IgA antibody, wherein the Fc region of the IgA antibody comprises a sequence comprising C242 through the C-terminus of the heavy chain of the IgA antibody. In certain embodiments, the IgG antibody includes a deletion of amino acid K447. In certain embodiments, the IgG antibody is selected from the group consisting of an IgG1 antibody, an IgG2 antibody, an IgG3 antibody and an IgG4 antibody. For example, but not by way of limitation, the IgG antibody can be an IgG1 antibody. In certain embodiments, the IgA antibody is selected from the group consisting of an IgA1 antibody, an IgA2m1 antibody, an IgA2m2 antibody and an IgA2mn antibody. For example, but not by way of limitation, the IgA antibody is an IgA2m1 antibody.

The present disclosure further provides an isolated nucleic acid that encodes an IgA antibody or IgG-IgA fusion molecule disclosed herein and host cells that include such nucleic acids. The present disclosure further provides methods for producing an antibody that includes culturing a host cell disclosed herein so that the IgA antibody or IgG-IgA fusion molecule is produced. The method can further include recovering the IgA antibody or IgG-IgA fusion molecule from the host cell.

The present disclosure provides pharmaceutical compositions that include an IgA antibody or IgG-IgA fusion molecule disclosed herein and a pharmaceutically acceptable carrier. In certain embodiments, the pharmaceutical composition can further include additional therapeutic agent.

The present disclosure further provides methods of treating an individual having a disease, where the method includes administering to the individual an effective amount of an IgA antibody or IgG-IgA fusion molecule disclosed herein. In certain embodiments, the disease is an inflammatory disease, an autoimmune disease or cancer.

The present disclosure provides methods of increasing the expression of IgA dimers. In certain embodiments, the method includes increasing the amount of DNA encoding a joining chain (JC) that is introduced into a first cell relative to the amount of DNA that encodes the light chain (LC) and the heavy chain (HC), wherein increased expression is relative to the amount of IgA dimers produced in a second cell introduced with equal amounts of JC, LC and HC DNA. For example, but not by way of limitation, the ratio of the amount of DNA encoding the HC to the amount of DNA encoding the LC to the amount of DNA encoding the JC (HC:LC:JC) that is introduced into the first cell is about from about 1:1:2 to about 1:1:5.

In certain embodiments, the present disclosure provides methods of increasing the expression of IgA dimers, trimers or tetramers. In certain embodiments, the method includes decreasing the amount of DNA encoding a joining chain (JC) introduced into a first cell relative to the amount of DNA that encodes the light chain (LC) and the heavy chain (HC), wherein increased expression is relative to the amount of IgA trimers or tetramers produced in a second cell introduced with greater amounts of HC and LC DNA relative to the amount of JC DNA. In certain embodiments, the ratio of the amount of DNA encoding the HC to the amount of DNA encoding the LC to the amount of DNA encoding the JC (HC:LC:JC) that is introduced into the first cell is from about 1:1:0.25 to about 1:1:0.5.

The present disclosure provides methods of increasing the production of an IgA1 or IgA2m1 polymer. In certain embodiments, the method comprises expressing, in a first cell, an IgA1 or IgA2m1 antibody having a substitution at amino acid V458, e.g., V4581, wherein increased production is relative to the amount of IgA1 or IgA2m1 polymers produced in a second cell expressing an IgA1 or IgA2m1 antibody that does not have a substitution at amino acid V458. The present disclosure further provides methods of increasing the production of IgA2m2 dimers that comprise expressing, in a first cell, an IgA2m2 antibody having a substitution at amino acid I458, e.g., I458V, wherein increased production is relative to the amount of IgA2m2 dimers produced in a second cell expressing an IgA2m2 antibody that does not have a substitution at amino acid I458. In certain embodiments, methods for increasing the production of an IgA1 or IgA2m1 polymer includes expressing, in a first cell, an IgA1 or IgA2m1 antibody having a substitution at amino acid N459 and/or S461, e.g., N459Q and/or S461A, wherein increased production is relative to the amount of IgA1 or IgA2m1 polymers produced in a second cell expressing an IgA1 or IgA2m1 antibody that does not have a substitution at amino acid N459 or S461. In certain embodiments, methods of decreasing the production of IgA2m2 polymers includes expressing, in a first cell, an IgA2m2 antibody with a substitution at amino acid C471, e.g., C471S, wherein decreased production is relative to the amount of IgA2m2 polymers produced in a second cell expressing an IgA2m2 antibody that does not have a substitution at amino acid C471. In certain embodiments, IgA antibodies that include a substitution at amino acid C471, e.g., C471S, can further include a substitution at P221, e.g., P221R,

The present disclosure provides methods of increasing transient expression of an IgA2m2 antibody comprising expressing, in a first cell, an IgA2m2 antibody that comprises a substitution at an amino acid selected from the group consisting of N166, S212, N263, N337, I338, T339, N459 and a combination thereof, wherein increased transient expression is relative to the amount of transient expression produced in a second cell expressing an IgA2m2 antibody that does not have a substitution at an amino acid selected from the group consisting of N166, S212, N263, N337, I338, T339, N459 and a combination thereof.

The present disclosure further provides methods of expressing dimers of IgG-IgA fusion molecules that include expressing an IgG-IgA fusion molecule comprising a full-length IgG antibody fused at its C-terminus to an Fc region of an IgA antibody. In certain embodiments, the Fc region of the IgA antibody comprises a sequence comprising P221 or R221 through the C-terminus of the heavy chain of the IgA antibody, wherein the IgG antibody comprises a deletion of amino acid K447. In certain embodiments, the present disclosure provides methods of expressing dimers, trimers or tetramers of IgG-IgA fusion molecules that include expressing an IgG-IgA fusion molecule comprising a full-length IgG antibody fused at its C-terminus to an Fc region of an IgA antibody, wherein the Fc region of the IgA antibody comprises a sequence comprising C242 through the C-terminus of the heavy chain of the IgA antibody. In certain embodiments, the IgG antibody comprises a deletion of amino acid K447.

The present disclosure provides methods for purifying the IgA and IgG-IgA fusion molecules disclosed herein and/or for purifying a specific oligomeric state, e.g., dimer, trimer or tetramer, of the IgA and IgG-IgA fusion molecules disclosed herein. In certain embodiments, a method for purifying an IgA antibody from a mixture comprising an IgA antibody and at least one host cell protein includes applying the mixture to a column comprising Protein L to bind the IgA antibody, washing the Protein L column with a wash buffer comprising PBS and eluting the IgA antibody from the Protein L column by an elution buffer comprising phosphoric acid. In certain embodiments, a method for purifying an oligomeric state of an IgA antibody or an IgG-IgA fusion molecule from a mixture comprising an IgA antibody or an IgG-IgA fusion molecule and at least one host cell protein can include applying the mixture to an affinity purification column comprising Protein L or Protein A to bind the IgA antibody or IgG-IgA fusion molecule, washing the affinity purification column with a wash buffer, eluting the IgA antibody or IgG-IgA fusion molecule from the affinity purification column by an elution buffer to form a first eluate and applying the first eluate to a size exclusion chromatography column to separate different IgA oligomeric states and to obtain a flowthrough comprising an oligomeric state of the IgA antibody or IgG-IgA fusion molecule.

### BRIEF DESCRIPTION OF THE FIGURES

FIG. 1A-1D. Protein sequences of human IgA heavy chain constant domains and J chain. (A) Alignment of protein sequences for the human heavy chain constant domains C_{H}1, C_{H}2, C_{H}3, hinge (Brerski et al. Curr Opin Immunol 40:62-9 (2016)) and tailpiece of IgA1, IgA2m1 and IgA2m2 (Toraño et al. 75:966-9 (1978)). Mismatches relative to the IgA1 sequence are highlighted in gray, N-linked glycosylation motifs are boxed and asterisks indicate amino acid differences in IgA2m2 from IgA1 and IgA2m1 in the tailpiece. (B) Protein sequence of the human J chain with the N-linked glycosylation motif boxed. (C) Protein sequence of the human heavy constant chain domains C_{H}1, C_{H}2, C_{H}3, hinge and tailpiece of IgA2mn. N-linked glycosylation sites are boxed. (D) Schematic of IgA oligomeric states with light chain (LC), heavy chain (HC) and joining chain (JC). IgA polymers represent trimer, tetramer and pentamer species.

FIG. 2A-2F. The oligomeric state of recombinantly produced IgA is affected by the amount of J chain DNA used in transfection and the heavy chain tailpiece sequence. (A-C) Overlay of normalized analytical size-exclusion chromatograms of affinity-purified IgA from small-scale transient transfections performed with varying ratios of light chain (LC), heavy chain (HC) and joining chain (JC) DNA for the following isotypes/allotypes: (A) IgA1, (B) IgA2m1 or (C) IgA2m2. Monomer (M), dimer (D) and polymer (P) peaks are indicated. Values were normalized based on the highest signal of each chromatogram. (D-F) Relative amounts of monomer, dimer, and trimer/tetramer species produced for IgA variants, quantified by analytical SEC. (D) The effect of mutations in the IgA tailpiece of IgA1, IgA2m1 and IgA2m2 at positions 458 and 467 on trimer/tetramer formation. The effect of mutations which remove N-linked glycosylation sites in (E) IgA1 or (F) IgA2m2 on trimer/tetramer formation.

FIG. 3A-3D. Biophysical and structural characterization of recombinant IgA oligomers. (A) Overlay of analytical size-exclusion chromatograms of purified IgA1, IgA2m1, IgA2m1 P221R, and IgA2m2 monomers, dimers and tetramer. (B) SDS-PAGE analysis of non-reduced (DTT) and reduced (+DTT) IgA1, IgA2m1, IgA2m1 P221R, and IgA2m2 monomers (M), dimers (D) and tetramer (T). Heavy chain (HC), light chain (LC) and joining chain (*) are indicated in reduced samples and the LC-LC dimer of IgA2m1 is indicated with an arrowhead. (C-D, upper panels) Reference free 2D classes from negative stain electron microscopy for (C) IgA2m2 dimer or (D) IgA2m2 tetramer. (C-D, lower panels) A raw image particle compared to its assigned 2D class is presented next to a model of IgA superimposed on the 2D class with the Fc domains and Fab fragments highlighted.

FIG. 4A-4B. Recombinantly produced IgA oligomers are stable and functional in vitro. (A) In vitro transcytosis of anti-mIL-13 hIgA monomers, dimers and tetramer in MDCK cells transfected with human pIgR. IgA polymers transcytose, while monomers do not. (B) Thermostability of anti-mIL-13 IgAs, IgG1 and IgG1 Fab fragment are measured by differential scanning fluorimetry (DSF). Only one melting transition was observed for all samples.

FIG. 5A-5C. Recombinant IgA oligomers demonstrate rapid serum clearance in vivo. (A) Serum-time concentration profiles of IgA or IgG in mice. The overall serum exposures of Balb/c mice administered with a single 5 mg/kg intravenous (IV) dose of IgA or IgG molecules at 5 min, 15 min, 30 min, 1 hr, 1 day, 3 days, 7 days and 14 days post dose. All mice were bled retro-orbitally under isoflurane to evaluate serum concentration profile. Human serum IgA monomer was administered at 10 mg/kg and is shown as a dashed line. (B-C) Tissue distribution of IgA or IgG in mice at 1 hr post injection. All graphs are means±SEM for each group with n=4. (B) Concentrations of intact antibodies were subtractive blood normalized per tissue, except blood, as ¹²⁵I (% ID/g tissue). (C) Concentrations of catabolized antibody values were determined by subtracting the ¹²⁵I (% ID/g tissue) from the ¹¹¹In (% ID/g tissue).

FIG. 6A-6C. Incomplete glycosylation of recombinant IgA molecules. (A) Schematic of N-linked glycan processing. (B) Global N-linked glycan analysis of recombinant IgA and IgA purified from human serum. Glycan analysis was done by mass spectrometric analysis after antibody deglycosylation and subsequent glycan enrichment. While human serum IgA shows greater than 90% sialylation, all recombinantly expressed IgA molecules have less than 60% sialylation. (C) Site-specific N-linked glycan analysis of the IgA2m1 dimer reveals heterogenous glycan composition between the different N-linked glycosylation sites on the IgA2m1 heavy chain (HC) and joining chain (JC).

FIG. 7A-7E. IgG1-IgA2m1 Fc fusions and aglycosylated IgA2m2 show increased serum exposures compared to wild-type IgA in vivo and demonstrate ability to transcytose in vitro. (A) Schematic of IgA2m2 tetramer with light chain (LC, black), heavy chain (HC, white) and joining chain with 41 N-linked glycosylation sites (diamond) (left) or aglycosylated (right). (B) Schematic of IgG1, IgA2m1 dimer or IgG1-IgA2m1 Fc dimer formats with LC (black), IgG1 HC (dotted), IgA2m1 HC (white), JC and N-linked glycosylation (diamond). (C) Analytical SEC of iodinated IgG1-IgA2m1 Fc dimers or tetramer after 0 hours (black), 24 hours (orange) or 96 hours (blue) incubation in mouse plasma. The initial IgG1-L-P221R IgA2m1 Fc tetramer and dimer show degradation similar to the peak of anti-HER2 IgG1 (Trastuzumab) control, whereas the reengineered IgG1ΔK-P221 IgA2m1 Fc or IgG1ΔK-C242 IgA2m1 Fc dimers are stable. (D) Serum-time concentration profiles of IgA or IgG in mice. The overall serum exposures of Balb/c mice administered with a single 30 mg/kg IV dose of IgA molecules. The in-house concentration data of a typical human IgG1 (anti-gD) previously dosed as a single intravenous (IV) injection at 30 mg/kg is shown as a dashed line. All mice were bled retro-orbitally or via cardiac puncture under isoflurane to evaluate serum concentration profile. (E) In vitro transcytosis of hIgA in MDCK cells transfected with human pIgR.

FIG. 8A-8B. Raw negative stain EM images of IgA2m2 dimer and tetramer purifications. (A) A raw image by negative stain electron microscopy (EM) of the purified IgA2m2 dimer shows good monodispersed particles. (B) A raw image by negative stain EM of the purified IgA2m2 tetramer shows good monodispersed radial particles.

FIG. 9. Intact antibody distribution normalized to plasma concentrations. Tissue distribution of IgA or IgG in mice at 1 hour post injection. All values represent the % ID/g of tissue after blood correction, normalized to the % ID/mL of plasma. All graphs are means±SD for each group with n=4.

FIG. 10. Tissue distribution of intact antibody after 1 day. Tissue concentrations of intact antibodies were subtractive blood normalized per tissue expressed as ¹²⁵I (% ID/g tissue) and calculated at one day post injection. All graphs are means±SEM for each group with n=4.

FIG. 11. Tissue distribution of degraded antibody after 1 day. Catabolized antibody values were determined by subtracting the ¹²⁵I (% ID/g tissue) from the ¹¹¹In (% ID/g tissue) and calculated at 1 day post injection. All graphs are means±SEM for each group with n=4.

FIG. 12. IgG1-IgA2m1 Fc fusion oligomer schematic. IgG1-L-P221R IgA2m1 Fc fusion (Borrok et al. MAbs: 7:743-51 (2015)) was made as a dimer and tetramer, but shown to have poor stability in mouse plasma (FIG. 7C). To eliminate a potential furin cleavage site, the C-terminal lysine (K) from IgG1 and the intervening leucine residue (L) were deleted. Additionally, the IgA2m1 wild-type (WT) sequence was restored with a proline at position 221 to make the IgG1ΔK-P221 IgA2m1 Fc fusion. The IgG1ΔK-C242 IgA2m1 Fc fusion design is similar, but the IgA2m1 Fc starts at residue C242, thereby deleting the IgA2m1 hinge (Δhinge).

FIG. 13. Global glycan analysis of engineered IgA oligomers. Global N-linked glycan analysis of CHO recombinantly produced dimers of anti-mIL-13 IgG1ΔK fused to P221 or C242 IgA2m1 Fc and the aglycosylated anti-HER2 IgA2m2 tetramer. The dimers of anti-mIL-13 IgG1ΔK fused to P221 or C242 IgA2m1 Fc both have ˜20% sialylation and as expected, no glycosylation is detected for the aglycosylated anti-HER2 IgA2m2 tetramer.

FIG. 14. Protein sequences of IgA heavy chain constant domains from human and other species. Alignment of protein sequences for the human heavy chain constant domains C_{H}1, C_{H}2, C_{H}3, hinge (Brerski et al. (2016)) and tailpiece (Toraño et al. (1978)). Conservation of the protein sequence between species is highlighted gray, while N-linked glycosylation motifs are boxed.

FIG. 15A-15C. (A) Analytical size-exclusion chromatograms of affinity-purified xmuIL13.huIgA1 from small-scale transient transfections performed in Expi293 cells with varying ratios of light chain (LC), heavy chain (HC) and joining chain (JC) DNA between 1:1:0.25 to 1:1:2. (B) Analytical size-exclusion chromatograms of affinity-purified xmuIL13.huIgA1 from small-scale transient transfections performed in Expi293 cells with varying ratios of light chain (LC), heavy chain (HC) and joining chain (JC) DNA between 1:1:1 to 1:1:5. (C) Amounts of dimer and tetramer species produced for the IgA antibody.

FIG. 16A-16B. (A) Analytical size-exclusion chromatograms of affinity-purified xmuIL13. IgA2m1 from small-scale transient transfections performed in Expi293 cells with varying ratios of light chain (LC), heavy chain (HC) and joining chain (JC) DNA. (B) Amounts of dimer and tetramer species produced for the IgA antibody.

FIG. 17A-17B. (A) Analytical size-exclusion chromatograms of affinity-purified xmuIL13. IgA2m1.P221R from small-scale transient transfections performed in Expi293 cells with varying ratios of light chain (LC), heavy chain (HC) and joining chain (JC) DNA. (B) Amounts of dimer and tetramer species produced for the IgA antibody.

FIG. 18A-18E. (A) Analytical size-exclusion chromatograms of affinity-purified xmuIL13.huIgA2m2 from small-scale transient transfections performed in Expi293 cells with varying ratios of light chain (LC), heavy chain (HC) and joining chain (JC) DNA. (B) Analytical size-exclusion chromatograms of affinity-purified xmuIL13.huIgA2m2 from small-scale transient transfections performed in Expi293 cells with varying ratios of light chain (LC), heavy chain (HC) and joining chain (JC) DNA between 1:1:1 to 1:1:5. (C) Amounts of dimer and tetramer species produced for the IgA antibody. (D) Analytical size-exclusion chromatograms of affinity-purified xmuIL13.huIgA2m2 from small-scale transient transfections performed in Expi293 cells with varying ratios of light chain (LC), heavy chain (HC), joining chain (JC) and secretory component (SC) DNA. (E) Confirmation of heavy chain, light chain and J chain of xmuIL13.huIgA2m2 by mass spectrometry.

FIG. 19 depicts the Biacore analysis of the following anti-IL-13 antibodies of the following isotypes/allotypes: IgA1 dimer, IgA2m1 dimer, IgA2m2 dimer and IgA2m2 tetramer.

FIG. 20A-20B. (A) Analytical size-exclusion chromatograms of affinity-purified xmuGP120.3E5.huIgA1 from small-scale transient transfections performed in Expi293 cells with varying ratios of light chain (LC), heavy chain (HC) and joining chain (JC) DNA. (B) Analytical size-exclusion chromatograms of affinity-purified xmuGP120.3E5.IgA2m1.P221R from small-scale transient transfections performed in Expi293 cells with varying ratios of light chain (LC), heavy chain (HC) and joining chain (JC) DNA.

FIG. 21 depicts analytical size-exclusion chromatograms of affinity-purified xmuGP120.3E5.huIgA2m2 from small-scale transient transfections performed in Expi293 cells with varying ratios of light chain (LC), heavy chain (HC) and joining chain (JC) DNA.

FIG. 22 depicts analytical size-exclusion chromatograms of affinity-purified xmuGP120.3E5.huIgA2m2 from small-scale transient transfections performed in Expi293 cells with varying ratios of light chain (LC), heavy chain (HC) and joining chain (JC) DNA.

FIG. 23 depicts analytical size-exclusion chromatograms of affinity-purified mouse xgD.5B6.hIgA2m2 from small-scale transient transfections performed in Expi293 cells with varying ratios of light chain (LC), heavy chain (HC) and joining chain (JC) DNA.

FIG. 24A-24C depicts the modification of the glycosylation sites of IgA2m2 and the J chain. (A) Summary of the modifications made to the heavy chain of IgA2m2 and the J chain and their expression in vitro as compared to wild type. (B) Summary of the transient expression of IgA2m2 single glycosylation variants. (C) Summary of the transient expression of IgA2m2 glycosylation variants with multiple mutations.

FIG. 25 depicts the analysis of the receptor binding properties of IgA monomer from human serum, wild-type IgA2m2 tetramer and IgA2m2 tetramer (aglycosylated) and J-chain (glycosylated).

FIG. 26 depicts the analysis of the glycan properties of each IgA molecule.

FIG. 27. Concentration time profile of IgA molecule after single 10 mg/kg IV injection in female Balb/C mice.

FIG. 28A-28B. (A) Analysis of cysteine mutations to prevent disulfide bonds with the secretory component or the J chain. (B) C471 but not C311 is required for IgA2m2 dimer and higher order oligomer formation when adding joining chain to the light chain and heavy chain.

FIG. 29 depicts the analysis of the co-transfection of the secretory component, joining chain, light chain and heavy chain.

FIG. 30A-30E. (A) Expression levels of xmuIL13.IgA2m2 variants generated to abolish plgR binding. (B) Analytical size-exclusion chromatograms of xmuIL13.IgA2m2 variants from small-scale transient transfections performed in Expi293 cells. (C) Biacore analysis of the xmuIL13. IgA2m2 variants binding to mouse plgR. (D) Biacore analysis of the xmuIL13. IgA2m2 variants binding to human plgR. (E) Biacore analysis of the xmuIL13.IgA2m2 variants binding to human FcαRI.

FIG. 31A-31B depicts the analysis of cell culture conditions to increase sialylation of anti-Jag1 IgA2m2. (A) Matrix of the cell culture conditions for a xJAG1.2B3.hIgA2m2 stable cell line. (B) Analysis of the effect cell culture conditions has on the glycosylation of xJAG1.2B3.hIgA2m2.

FIG. 32 depicts the stability of IgA variants by differential scanning fluorimetry (DSF).

FIG. 33A-33D depicts the characterization and engineering of a full length anti-murine IL-13 IgG1.Leu-P221R.IgA2m1 Fc fusion molecule to increase oligomer stability. (A) Analytical size-exclusion chromatograms of affinity-purified glycosylated Full length anti-murine IL-13 IgG1.Leu-P221R.IgA2m1 Fc fusion molecules from small-scale transient transfections performed in Expi293 cells with varying ratios of light chain (LC), heavy chain (HC) and joining chain (JC) DNA. (B) Biacore analysis of the IgA oligomers binding to mouse plgR. (C) Summary of the binding of the IgA oligomers to mouse plgR and human plgR. (D) Stability of the IgA oligomers by DSF.

FIG. 34A-34C. (A) IgG1 full length-IgA Fc construct design to eliminate furin site and instability. (B) Full length anti-murine IL-13 IgG1-IgA Fc transient expression data of engineered constructs. (C) Mouse plasma stability data for engineered anti-murine IL-13 IgA molecules.

FIG. 35A-35B. (A) Wasatch analysis of IgA oligomer binding to human FcαRI. (B) Summary of the binding of IgA oligomer to FcαRI as determined by Wasatch Surface Plasmon Resonance (SPR).

FIG. 36A-36D. (A) Wasatch analysis of the binding of IgA2m2 dimers and tetramers produced by transient expression in CHO cells and Expi293 cells to mouse and human pIgR. (B) Wasatch analysis of the binding of IgA2m2 glycosylation variants to mouse pIgR. (C) Wasatch analysis of the binding of IgA2m2 glycosylation variants to human pIgR. (D) Summary of the binding of IgA oligomer to mouse and human pIgR as determined by Wasatch SPR.

FIG. 37A-37C. (A) Expression profiles of hIgG1-hIgA1 fusion molecules. (B) Analytical size-exclusion chromatograms of hIgG1-hIgA1 fusion molecules. (C) Biacore analysis of the binding of hIgG1-hIgA1 fusion molecules to mouse and human pIgR and human FcαRI.

FIG. 38 depicts the analysis of the removal of N-linked glycosylation of various IgA1 antibodies.

FIG. 39. Recombinantly expressed human anti-mIL-13 IgA2m2 was affinity purified over a Capto L column. The Capto L eluate was then analyzed by size-exclusion chromatography (SEC) using a 3.5 μm, 7.8 mm×300 mm Water's XBridge Protein BEH SEC 200 Å column on an HPLC. Three main peaks were observed in the analytical SEC elution profile corresponding to higher order polymers (peak 1, including trimer, tetramer, and pentamer), dimer (peak 2) and monomer (peak 3) as determined by multi-angle light scattering (MALS) and negative stain electron microscopy.

FIG. 40. Separation of the mixture of recombinant human anti-mIL-13 IgA2m2 oligomeric species seen in the Capto L affinity column eluate was attempted by size-exclusion chromatography (SEC) using a HiLoad 16/600 Superose 6 prep grade (pg) column. Four main peaks were observed in the Superose 6 elution profile corresponding to high molecular weight aggregates (peak 1, eluting in the void volume of the column), higher order polymers (peak 2, likely including trimer, tetramer, and pentamer), dimer (peak 3) and monomer (peak 4) as determined by multi-angle light scattering (MALS) coupled to analytical SEC and negative stain electron microscopy. The molar mass (MW) and polydispersity index (PDI) of proteins measured from fractions taken from peaks 2 and 3 are indicated.

FIG. 41. Separation of recombinant human anti-mIL-13 IgA2m2 dimers from higher order polymers was achieved by size-exclusion chromatography (SEC) using a 3.5 μm, 7.8 mm×300 mm Water's XBridge Protein BEH SEC 450 Å column on an HPLC. Three main peaks were observed in the analytical SEC elution profile, corresponding to higher order polymers (peak 1, including trimer, tetramer, and pentamer), dimer (peak 2), and monomer (peak 3) as determined by multi-angle light scattering (MALS) coupled to analytical SEC and negative stain electron microscopy.

FIG. 42A-42D. (A) The Capto L affinity column elution of human anti-mIL-13 IgA2m2 was analyzed by size-exclusion chromatography (SEC) using a 3.5 μm, 7.8 mm×300 mm Water's XBridge Protein BEH SEC 200 Å column on an HPLC. Three main peaks were observed corresponding to higher order polymers (peak 1, including trimer, tetramer, and pentamer), dimer (peak 2), and monomer (peak 3) as determined by multi-angle light scattering (MALS) coupled to analytical SEC and negative stain electron microscopy. (B) Peak 1 from panel (A) was isolated by purification over a 3.5 μm, 7.8 mm×300 mm Water's XBridge Protein BEH SEC 450 Å column as in FIG. 41. Peak 1 post purification analysis on a 3.5 μm, 7.8 mm×300 mm Water's XBridge Protein BEH SEC 200 Å column coupled to a MALS detector is shown here. The molar mass (MW) and polydispersity index (PDI) determined by MALS is consistent with the expected mass of predominantly tetrameric IgA2m2. (C) Peak 2 from panel (A) was isolated by purification over a 3.5 μm, 7.8 mm×300 mm Water's XBridge Protein BEH SEC 450 Å column as in FIG. 41. Peak 2 post purification analysis on a 3.5 μm, 7.8 mm×300 mm Water's XBridge Protein BEH SEC 200 Å column coupled to a MALS detector is shown here. The MW and PDI determined by MALS is consistent with the expected mass of predominantly dimeric IgA2m2. (D) Purified protein from peaks 1 and 2 from panels (B) and (C) was analyzed by SDS-PAGE under either non-reducing (−DTT) or reducing (+DTT) conditions. In the reduced samples bands migrating at the expected masses for the heavy chain (HC), light chain (LC) and J chain (JC) are observed.

FIG. 43A-43B. (A) Representative raw image from negative stain electron microscopy (EM) of human anti-mIL-13 IgA2m2 particles from peak 1 in FIG. 42B. (B) Reference free 2D classes from negative stain EM of particles from peak 1 in FIG. 42B indicating the sample is predominantly tetramer.

FIG. 44A-44B. (A) Representative raw image from negative stain electron microscopy (EM) of human anti-mIL-13 IgA2m2 particles from peak 2 in FIG. 42C. (B) Reference free 2D classes from negative stain EM of particles from peak 2 in FIG. 42C indicating the sample is predominantly dimer.

FIG. 45. Mass spectrometry analysis of the human anti-mIL-13 IgA2m2 dimer purified from peak 2 in FIG. 42C. Mass spectrometric analysis performed after heat denaturation, reduction with dithiothreitol, and deglycosylation with PNGaseF confirms the presence of the correct joining chain (JC), light chain (LC) and heavy chain (HC).

FIG. 46A-46C. (A) The Capto L affinity column elution of human anti-mIL-13 IgA1 was analyzed by size-exclusion chromatography (SEC) using a 3.5 μm, 7.8 mm×300 mm Water's XBridge Protein BEH SEC 200 Å column on an HPLC. Prior to separation of oligomers, three main peaks were observed corresponding to higher order polymers (peak 1, including trimer, tetramer, and pentamer), dimer (peak 2), and monomer (peak 3). (B) Peak 2 from panel (A) was isolated by purification over a 3.5 μm, 7.8 mm×300 mm Water's XBridge Protein BEH SEC 450 Å column on an HPLC as in FIG. 41. Peak 2 post purification analysis on a 3.5 μm, 7.8 mm×300 mm Water's XBridge Protein BEH SEC 200 Å column coupled to a multi-angle light scattering (MALS) detector is shown here. The molar mass (MW) and polydispersity index (PDI) determined by MALS is consistent with the expected mass of predominantly dimeric IgA1. (C) Purified protein from peak 2 from panel (B) was analyzed by SDS-PAGE under either non-reducing (−DTT) or reducing (+DTT) conditions. In the reduced samples bands migrating at the expected masses for the heavy chain (HC), light chain (LC), and J chain (JC) are observed.

FIG. 47A-47C. (A) The Capto L affinity column elution of human anti-mIL-13 IgA2m1 was analyzed by size-exclusion chromatography (SEC) using a 3.5 μm, 7.8 mm×300 mm Water's XBridge Protein BEH SEC 200 Å column on an HPLC. Prior to separation of oligomers, three main peaks were observed corresponding to higher order polymers (peak 1, including trimer, tetramer, and pentamer), dimer (peak 2), and monomer (peak 3). (B) Peak 2 from panel (A) was isolated by purification over a 3.5 μm, 7.8 mm×300 mm Water's XBridge Protein BEH SEC 450 Å column on an HPLC as in FIG. 41. Peak 2 post purification analysis on a 3.5 μm, 7.8 mm×300 mm Water's XBridge Protein BEH SEC 200 Å column coupled to a multi-angle light scattering (MALS) detector is shown here. The molar mass (MW) and polydispersity index (PDI) determined by MALS is consistent with the expected mass of predominantly dimeric IgA2m1. (C) Purified protein from peak 2 from panel (B) was analyzed by SDS-PAGE under either non-reducing (−DTT) or reducing (+DTT) conditions. In the reduced samples bands migrating at the expected masses for the heavy chain (HC), light chain (LC), and J chain (JC) are observed.

FIG. 48A-48C. (A) The Capto L affinity column elution of human anti-mIL-13 IgA2m1 containing the P221R mutation in the heavy chain was analyzed by size-exclusion chromatography (SEC) using a 3.5 μm, 7.8 mm×300 mm Water's XBridge Protein BEH SEC 200 Å column on an HPLC. Prior to separation of oligomers, three main peaks were observed corresponding to higher order polymers (peak 1, including trimer, tetramer, and pentamer), dimer (peak 2), and monomer (peak 3). (B) Peak 2 from panel (A) was isolated by purification over a 3.5 μm, 7.8 mm×300 mm Water's XBridge Protein BEH SEC 450 A column on an HPLC as in FIG. 41. Peak 2 post purification analysis on a 3.5 μm, 7.8 mm×300 mm Water's XBridge Protein BEH SEC 200 Å column is shown. (C) Purified protein from peak 2 from panel (B) was analyzed by SDS-PAGE under either non-reducing (−DTT) or reducing (+DTT) conditions. In the reduced samples bands migrating at the expected masses for the heavy chain (HC), light chain (LC), and J chain (JC) are ob served.

FIG. 49 depicts the ability of monomeric and polymeric anti-HER2 IgA antibodies to result in the death of the HER2+ breast cancer cell lines KPL-4, BT474-M1 and SKBR3.

FIG. 50 depicts the ability of monomeric and polymeric anti-HER2 IgA antibodies to result in the death of SKBR3 breast cancer cells in the presence of neutrophils from different donors.

FIG. 51 depicts the ability of glycosylated and aglycosylated IgA polymers and monomer to result in the death of SKBR3 breast cancer cells.

FIG. 52A-B. (A) Biacore analysis of IgA oligomers and tetramers binding to human FcαRI. (B) Summary of the binding of IgA oligomers and tetramers to FcαRI as determined by Biacore SPR.

### DETAILED DESCRIPTION

For clarity and not by way of limitation the detailed description of the presently disclosed subject matter is divided into the following subsections:

I. Definitions;

II. Antibodies;

III. Methods of Antibody Production and Purification;

IV. Methods of Treatment;

V. Pharmaceutical Compositions;

VI. Articles of Manufacture; and

VII. Exemplary Embodiments.

I. Definitions

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “a,” “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone) and “B” (alone). Likewise, the term “and/or” as used in a phrase such as “A, B and/or C” is intended to encompass each of the following aspects: A, B and C; A, B or C; A or C; A or B; B or C; A and C; A and B; B and C; A (alone); B (alone); and C (alone).

The term “antibody” herein is used in the broadest sense and encompasses various antibody structures, including but not limited to monoclonal antibodies, polyclonal antibodies, multispecific antibodies, antibody fragments and antibody fusion molecules so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab', Fab'-SH, F(ab')₂; diabodies; linear antibodies; single-chain antibody molecules (e.g., scFv); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG and IgM, and several of these may be further divided into subclasses (isotypes), e.g., IgG₁, IgG₂, IgG₃, IgG₄, IgA₁ and IgA₂. The heavy chain constant domains that correspond to the different classes of immunoglobulins are called α, δ, ε, γ and μ, respectively.

The term “IgA antibodies” refer to antibodies of the IgA class of antibodies and include the IgA isotypes, IgA1 and IgA2, and the three allotypes of IgA2, m1, m2 and mn.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, R¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result. For example, and not by way of limitation, an “effective amount” can refer to an amount of an antibody, disclosed herein, that is able to alleviate, minimize and/or prevent the symptoms of the disease and/or disorder, prolong survival and/or prolong the period until relapse of the disease and/or disorder.

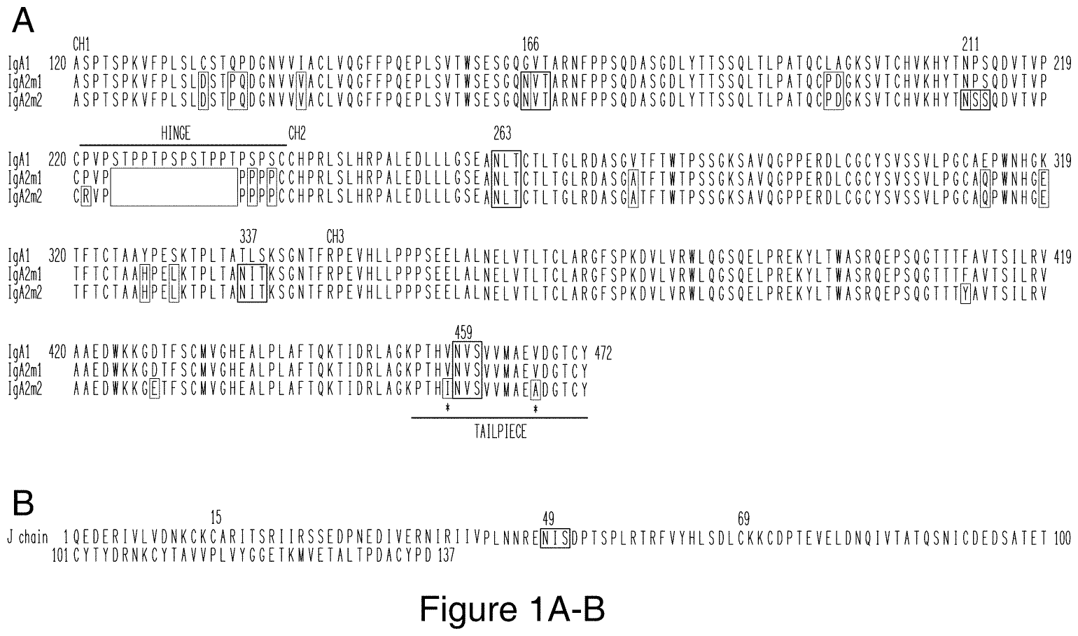

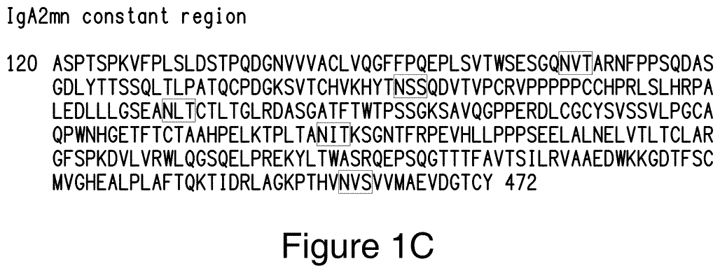

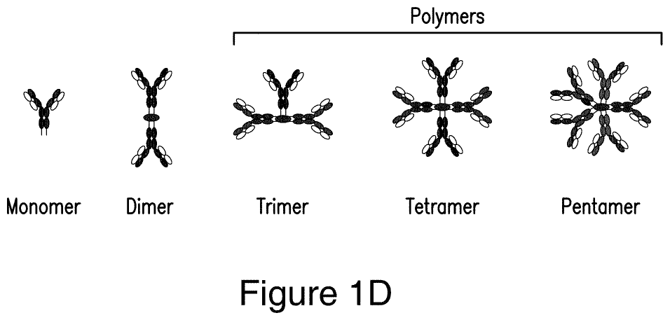

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) or the C-terminal glycine (Gly446) of the Fc region may or may not be present. In certain embodiments, a human IgA heavy chain Fc region extends from Pro221 (P221), Arg221 (R221), Val222 (V222), Pro223 (P223) or from Cys242 (C242) to the carboxyl-terminus of the heavy chain (see FIGS. 1A and C). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Fe receptor” or “FcR” describes a receptor that binds to the Fc region of an antibody. Fc receptors include, but are not limited to, FcαRI (recognizing the Fc region of an IgA antibody) and FcγRII (recognizing the Fc region of an IgG antibody). FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgG antibodies to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.). Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/042072 (Presta) describes antibody variants with improved or diminished binding to FcRs. See also, e.g., Shields et al., J. Biol. Chem. 9(2):6591-6604 (2001).

“Framework” or “FR” refers to variable domain residues other than complementary determining regions (CDRs). The FR of a variable domain generally consists of four FR domains: FR1, FR2, FR3 and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody,” “intact antibody” and “whole antibody” are used herein interchangeably to refer to an antibody having a structure substantially similar to a native antibody structure or having heavy chains that contain an Fc region as defined herein.

The terms “host cell,” “host cell line” and “host cell culture” as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence (also referred to herein as “complementarity determining regions” or “CDRs”) and/or form structurally defined loops (“hypervariable loops”) and/or contain the antigen-contacting residues (“antigen contacts”). Unless otherwise indicated, HVR residues and other residues in the variable domain (e.g., FR residues) are numbered herein according to Kabat et al., supra. Generally, antibodies comprise six HVRs: three in the VH (H1, H2, H3), and three in the VL (L1, L2, L3).

An “immunoconjugate” refers to an antibody conjugated to one or more heterologous molecule(s), including but not limited to a cytotoxic agent.

An “individual” or “subject,” as used interchangeably herein, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al., Nature Medicine 2017, published online 12 Jun. 2017, doi:10.1038/nm.4356 or EP 2101823 B1).

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (C_{H}1, C_{H}2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert,” as used herein, refers to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227- 258; and Pearson et. al. (1997) Genomics 46:24-36 and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier,” as used herein, refers to an ingredient in a pharmaceutical composition, other than an active ingredient, which is nontoxic to a subject. A pharmaceutically acceptable carrier includes, but is not limited to, a buffer, excipient, stabilizer or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the present disclosure can be used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

II. Antibodies

In certain embodiments, the present disclosure is based, in part, on methods of engineering antibodies, e.g., IgA antibodies and IgG-IgA fusion molecules, to exhibit improved serum retention and to increase polymeric antibody production. In certain embodiments, the antibodies of the present disclosure exhibit binding to FcRn. In certain embodiments, the antibodies of the present disclosure exhibit increased IgR-mediated transcytosis. In certain embodiments, the antibodies of the present disclosure exhibit reduced and/or no binding to FcαRI. In certain embodiments, antibodies of the present disclosure can provide superior safety in a therapeutic setting by minimizing pro-inflammatory response following administration.

In certain embodiments, the present disclosure provides antibodies, e.g., IgA antibodies and IgG-IgA fusion molecules, that exhibit improved serum retention. For example, by not by way of limitation, antibodies of the present disclosure, e.g., IgA antibodies and IgG-IgA Fc fusion molecules, are stable in plasma for up to about 1 day, up to about 2 days, up to about 3 days, up to about 4 days or up to about 5 days. In certain embodiments, antibodies of the present disclosure, e.g., IgA antibodies and IgG-IgA fusion molecules, are stable in plasma for up to about 4 days.

In certain embodiments, the present disclosure provides antibodies, e.g., IgA antibodies and IgG-IgA fusion molecules, that have reduced glycosylation or no glycosylation. For example, by not by way of limitation, antibodies of the present disclosure exhibit at least about 10%, at least about 20%, at least about 30%, at least about 40%, at least about 50%, at least about 60%, at least about 70%, at least about 80%, at least about 90% or at least about 95% reduction in glycosylation as compared to unmodified IgA or unmodified IgG-IgA fusion molecules. In certain embodiments, antibodies of the present disclosure are less than about 0.5%, less than about 1%, less than about 2%, less than about 5% glycosylated, less than about 10% glycosylated, less than about 20% glycosylated, less than about 30% glycosylated or less than about 40% glycosylated. In certain embodiments, antibodies of the present disclosure have 0% glycosylation, i.e., are aglycosylated.

A. Exemplary Antibodies

1. IgA Antibody Variants

The present disclosure provides IgA antibodies, e.g., IgA1, IgA2m1, IgA2m2 and IgA2mn antibodies, that have been modified to decrease the extent to which the antibody is glycosylated. Deletion of glycosylation sites of an antibody can be accomplished by altering the amino acid sequence of the antibody such that one or more glycosylation sites are removed. In certain embodiments, an antibody of the present disclosure can be modified to remove one or more, two or more, three or more, four or more, five or more or six or more glycosylation sites, e.g., N-linked glycosylation sites and/or O-linked glycosylation sites.

In certain embodiments, an antibody of the present disclosure can be modified to remove one or more of N-linked glycosylation motifs N-X-S/T, where X is any amino acid. In certain embodiments, the removal of an N-linked glycosylation site can include the modification, e.g., mutation, of one or more amino acids present in the motif of the glycosylation site. For example, but not by way of limitation, the N, X and/or S/T amino acid can be modified, e.g., mutated, in the motif of the glycosylation site. In certain embodiments, all three amino acids of the motif can be mutated.

In certain embodiments, an antibody of the present disclosure can be modified to remove one or more, two or more, three or more, four or more or five or more glycosylation sites from the heavy chain constant domain. For example, but not by way of limitation, an antibody of the present disclosure can be modified to remove one or more, two or more, three or more or all 4 N-linked glycosylation sites at amino acids 166, 211, 263 and/or 337 of the heavy chain constant domain. In certain embodiments, an antibody of the present disclosure can be modified to remove one or more glycosylation sites in the tailpiece of the heavy chain (see FIG. 1A). For example, but not by way of limitation, an antibody of the present disclosure can be modified to remove the N-linked glycosylation site at amino acid 459 of the tailpiece of the heavy chain. In certain embodiments, an IgA1 antibody of the present disclosure can be modified to remove one or more N-linked glycosylation sites at amino acids 263 and/or 449. In certain embodiments, an IgA2m1 antibody of the present disclosure can be modified to remove one or more N-linked glycosylation sites at amino acids 166, 263, 337 and/or 449. In certain embodiments, an IgA2m2 or IgA2mn antibody of the present disclosure can be modified to remove one or more N-linked glycosylation sites at amino acids 166, 211, 263, 337 and/or 449. In certain embodiments, an antibody can be modified to remove all the N-linked glycosylation sites from the heavy chain of the antibody, including the heavy chain constant domain and the tailpiece.

In certain embodiments, an antibody of the present disclosure can be aglycosylated. For example, but not by way of limitation, an aglycosylated antibody of the present disclosure is an antibody that has no glycosylation on the heavy chain of the antibody including the heavy chain constant region and the tailpiece. In certain embodiments, an aglycosylated antibody of the present disclosure is an antibody that has no glycosylation on the heavy chain, including the heavy chain constant region and the tailpiece, and no glycosylation on the J chain.

In certain embodiments, the present disclosure provides an IgA antibody that has one or more, two or more, three or more, four or more, five or more, six or more, seven or more, eight or more, nine or more, ten or more, eleven or more or twelve modifications, e.g., substitutions, at amino acids 166, 168, 211, 212, 213, 263, 265, 337, 338, 339, 459 and/or 461 to reduce the glycosylation of the IgA antibody. For example, but not by way of limitation, the present disclosure provides an IgA antibody that has one or more, two or more, three or more, four or more, five or more, six or more, seven or more, eight or more, nine or more, ten or more, eleven or more or twelve modifications, e.g., substitutions, at amino acids N166, T168, N211, S212, S213, N263, T265, N337, I338, T339, N459 and/or S461 to reduce the glycosylation of the IgA antibody.

In certain embodiments, an IgA1 antibody of the present disclosure has one or more, two or more, three or more or four modifications at amino acids 263, 265, 459 and/or 461, e.g., at amino acids N263, T265, N459 and/or S461. In certain embodiments, an IgA2m1 antibody of the present disclosure has one or more, two or more, three or more, four or more, five or more, six or more, seven or more or eight modifications at amino acids 166, 168, 263, 265, 337, 338, 339, 459 and/or 461, e.g., at amino acids N166, T168, N263, T265, N337, I338, T339, N459 and/or S461. In certain embodiments, an IgA2m2 or IgA2mn antibody of the present disclosure has one or more, two or more, three or more, four or more, five or more, six or more, seven or more, eight or more, nine or more, ten or more, eleven or more or twelve modifications at amino acids 166, 168, 211, 212, 213, 263, 265, 337, 338, 339, 459 and/or 461, e.g., at amino acids N166, T168, N211, S212, S213, N263, T265, N337, I338, T339, N459 and/or S461. In certain embodiments, an IgA2m1, IgA2m2 or IgA2mn antibody of the present disclosure are modified at all three amino acids 337, 338 and 339, e.g., at amino acids N337, 1338 and T339.

In certain embodiments, an IgA antibody of the present disclosure, e.g., an IgA2m1, IgA2m2 or IgA2mn antibody, has a modification at amino acid N166. In certain embodiments, an IgA antibody of the present disclosure, e.g., an IgA2m1, IgA2m2 or IgA2mn antibody, has a modification at amino acid N168. In certain embodiments, an IgA antibody of the present disclosure, e.g., an IgA2m2 or IgA2mn antibody, has a modification at amino acid S211. In certain embodiments, an IgA antibody of the present disclosure, e.g., an IgA2m2 or IgA2mn antibody, has a modification at amino acid S212. In certain embodiments, an IgA antibody of the present disclosure, e.g., an IgA2m2 or IgA2mn antibody, has a modification at amino acid S213. In certain embodiments, an IgA antibody of the present disclosure, e.g., an IgA1, IgA2m1, IgA2m2 or IgA2mn antibody, has a modification at amino acid N263. In certain embodiments, an IgA antibody of the present disclosure, e.g., an IgA1, IgA2m1, IgA2m2 or IgA2mn antibody, has a modification at amino acid N265. In certain embodiments, an IgA antibody of the present disclosure, e.g., an IgA2m1, IgA2m2 or IgA2mn antibody, has modifications at the three amino acids N337, 1338 and T339. In certain embodiments, an IgA antibody of the present disclosure, e.g., an IgA1, IgA2m1, IgA2m2 or IgA2mn antibody, has a modification at amino acid N459. In certain embodiments, an IgA antibody of the present disclosure, e.g., an IgA1, IgA2m1, IgA2m2 or IgA2mn antibody, has a modification at amino acid S461.

In certain embodiments, the amino acid N can be mutated to an A, G or Q amino acid. In certain embodiments, the amino acid S can be mutated to an A amino acid. In certain embodiments, the amino acid T can be mutated to an A amino acid. In certain embodiments, an IgA antibody of the present disclosure, e.g., IgA2m2 or IgA2mn antibody, of the present disclosure can be modified to comprise one or more, two or more, three or more, four or more, five or more, six or more or all seven of the following mutations N166A, S212P, N263Q, N337T, I338L, T339S and N459Q. For example, but not by way of limitation, an IgA1 antibody of the present disclosure can be modified to comprise one or more or all two of the following mutations N263Q and N459Q. In certain embodiments, an IgA2m1 antibody of the present disclosure can be modified to comprise one or more, two or more, three or more, four or more, five or more or all six of the following mutations N166A, N263Q, N337T, I338L, T339S and N459Q. In certain embodiments, an IgA2m2 or IgA2mn antibody of the present disclosure can be modified to comprise one or more, two or more, three or more, four or more, five or more, six or more or all seven of the following mutations N166A, S212P, N263Q, N337T, I338L, T339S and N459Q.

In certain embodiments, a J chain of an antibody of the present disclosure can be modified to remove one or more glycosylation sites. In certain embodiments, an antibody of the present disclosure can be modified to remove the N-linked glycosylation site at amino acid 49 of the J chain, e.g., by modifying one or more amino acids of the glycosylation site motif, which encompasses amino acids 49, 50 and 51. For example, by not by way of limitation, amino acid N49 and/or amino acid S51 of the J chain can be modified. In certain embodiments, amino acid N can be mutated to an A, G or Q amino acid and/or amino acid S can be mutated to an A amino acid. For example, by not by way of limitation, a J chain of an antibody of the present disclosure can comprise a N49A/G/Q mutation and/or a S51A mutation. In certain embodiments, a J chain of an antibody of the present disclosure can comprise an N49Q mutation.

In certain embodiments, an antibody of the present disclosure can be modified to remove one or more, two or more, three or more, four or more or five or more glycosylation sites from the heavy chain and modified to remove one glycosylation site from the J chain. In certain embodiments, an antibody of the present disclosure has one or more, two or more, three or more, four or more, five or more, six or more, seven or more, eight or more, nine or more, ten or more, eleven or more or twelve modifications, e.g., substitutions, at amino acids N166, T168, N211, S212, S213, N263, T265, N337, I338, T339, N459 and/or S461 of the heavy chain and one or two modifications, e.g., substitutions, at amino acids N49 and/or S51 of the J chain. In certain embodiments, an antibody of the present disclosure has one or more, two or more, three or more, four or more, five or more, six or more or all seven modifications, e.g., substitutions, at amino acids N166, S212, N263, N337, I338, T339 and/or N459 of the heavy chain and one or two modifications, e.g., substitutions, at amino acids N49 and/or S51 of the J chain.

In certain embodiments, an IgA antibody, e.g., an IgA1, IgA2m1, IgA2m2 or IgA2mn antibody, of the present disclosure can have one or more modifications at amino acids N459 or S461 to reduce the glycosylation of the IgA antibody. In certain embodiments, a modification of amino acid N459 and/or S461 results in an antibody having an increased ability to generate polymers, e.g., dimers, trimers, tetramers and pentamers.

In certain embodiments, antibodies, e.g., IgA antibodies, of the present disclosure can have a modification, e.g., substitution, at amino acid 458. In certain embodiments, the present disclosure provides IgA1, IgA2m1 and IgA2mn antibodies that have a substitution at amino acid V458. In certain embodiments, the amino acid V458 can be mutated to an isoleucine (i.e., V4581). In certain embodiments, the present disclosure provides IgA2m2 antibodies that have a substitution at amino acid I458. In certain embodiments, the amino acid I458 can be mutated to a valine (i.e., I458V). In certain embodiments, one or more of these modifications can be present in an antibody that has reduced or no glycosylation, as described herein.

In certain embodiments, antibodies, e.g., IgA antibodies, of the present disclosure can have a modification, e.g., substitution, at amino acid C471 and/or C311. In certain embodiments, an IgA antibody can have a mutation at amino acid C471, e.g., C471S. In certain embodiments, an IgA antibody can have a mutation at amino acid C311, e.g., C311S.

In certain embodiments, modifications of an antibody of the present disclosure can be made in order to create antibody variants with certain improved properties. For example, but not by way of limitation, an antibody of the present disclosure that has reduced glycosylation can exhibit improved serum retention. In certain embodiments, an antibody of the present disclosure that has reduced glycosylation can have an increased ability to generate polymers, e.g., dimers, trimers, tetramers and pentamers. In certain embodiments, an antibody of the present disclosure that has reduced glycosylation can exhibit reduced binding to the IgA-specific hFc receptor, FcαRI, e.g., no binding to FcαRI. In certain embodiments, an antibody of the present disclosure that has a modification at amino acid 458, 459 and/or S461 has an increased ability to generate polymers, e.g., dimers, trimers, tetramers and pentamers, as compared to an antibody that does not have one of the modifications. In certain embodiments, an antibody disclosed herein that has a modification at amino acid C471 has a decreased ability to generate polymers, e.g., dimers, trimers, tetramers and pentamers.

2. IgG-IgA Fusion Molecules

The present disclosure further provides antibodies that comprise at least a portion of an IgG antibody and at least a portion of an IgA antibody, referred to herein as IgG-IgA fusion molecules. In certain embodiments, the IgG-IgA fusion molecules of the present disclosure have increased resistance to protease, e.g., furin, activity and/or an increased serum half-life (see Table 9). In certain embodiments, the IgG-IgA fusion molecules of the present disclosure bind to FcRn.

In certain embodiments, the IgG antibody of an IgG-IgA fusion molecule of the present disclosure can be a full-length IgG antibody. In certain embodiments, the IgG antibody can be any IgG antibody that binds to the neonatal Fc receptor (FcRn). For example, but not by way of limitation, the IgG antibody can be IgG1, IgG2, IgG3 or IgG4. In certain embodiments, the IgG antibody is an IgG1 antibody. In certain embodiments, the IgG antibody is an IgG2 antibody. In certain embodiments, the IgG antibody is an IgG3 antibody. In certain embodiments, the IgG antibody is an IgG4 antibody.

In certain embodiments, an IgG-IgA fusion molecule of the present disclosure can include an IgG antibody fused to a fragment of an IgA antibody. In certain embodiments, the IgA antibody can be an IgA1, IgA2m1, IgA2mn or IgA2m2 antibody. In certain embodiments, the IgA fragment can be about 300 amino acids in length, about 250 amino acids in length, about 200 amino acids in length, about 150 amino acids in length, about 100 amino acids in length, about 80 amino acids in length, about 60 amino acids in length, about 40 amino acids in length or about 20 amino acids in length. In certain embodiments, the IgA fragment is about 250 amino acids in length. In certain embodiments, the IgA fragment is about 20 amino acids, e.g., about 18 amino acids, in length. For example, but not by way of limitation, the IgA fragment can include the Fc region of the IgA antibody. In certain embodiments, the IgA fragment can include the CH2 and CH3 domains of the IgA antibody. In certain embodiments, the IgA fragment can further include the hinge region of an IgA antibody. In certain embodiments, the IgA fragment can further include the tailpiece of an IgA antibody.

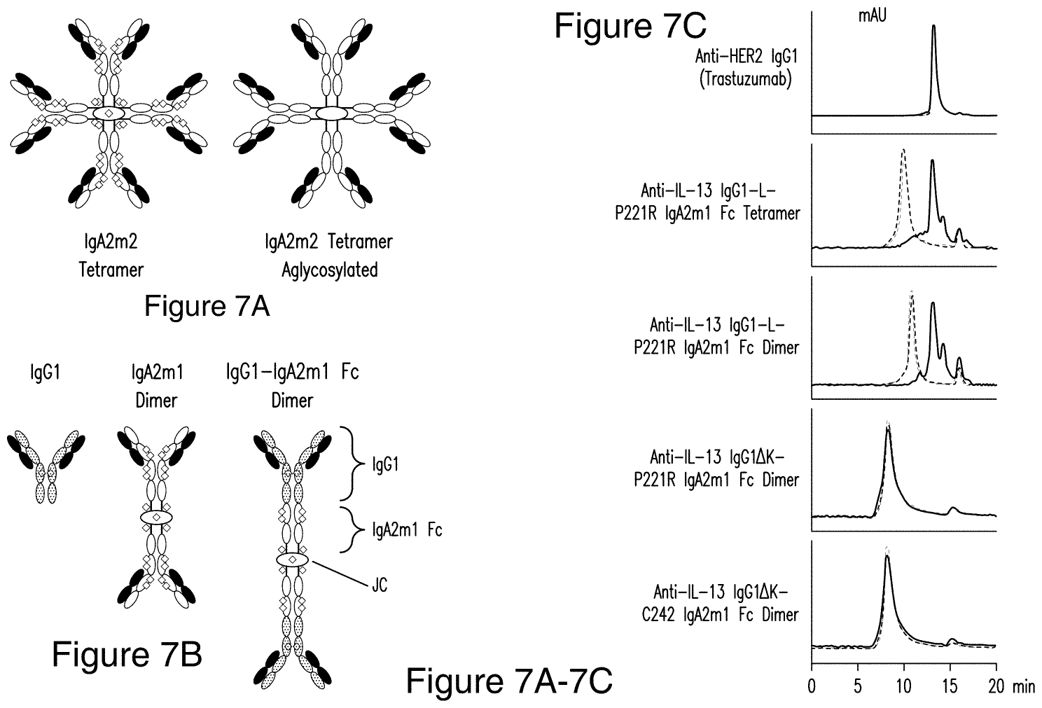

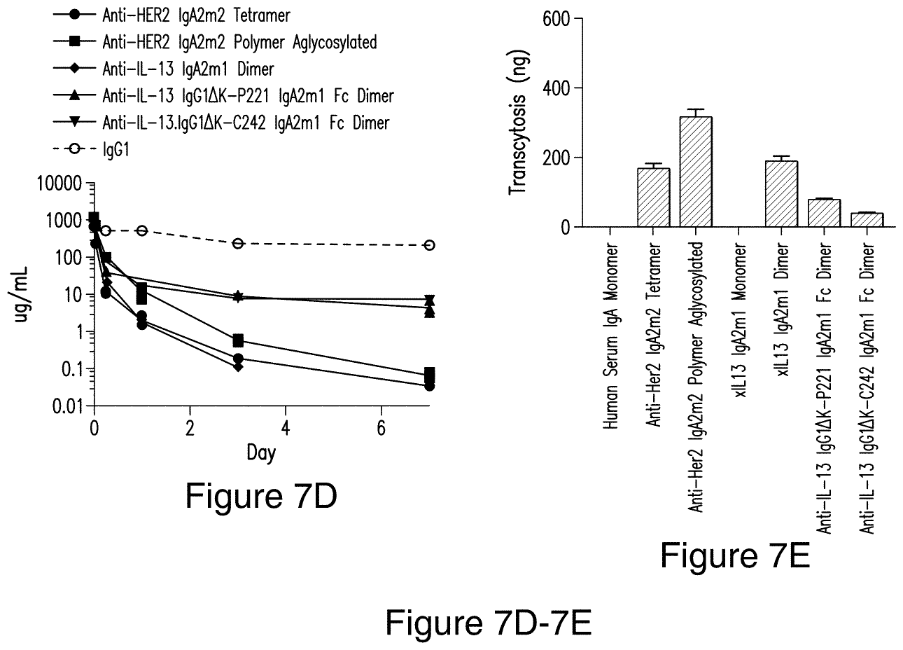

In certain embodiments, an IgG-IgA fusion molecule of the present disclosure can include an IgG antibody and an Fc region of an IgA antibody. In certain embodiments, an IgG-IgA fusion molecule can include an IgG antibody fused at its C-terminus to an Fc region of an IgA antibody, disclosed herein. For example, but not by way of limitation, an IgG-IgA fusion molecule can include full length IgG heavy chain sequences fused at their C-terminus to an Fc region of an IgA heavy chain (see FIGS. 7B, 12 and 34A).

In certain embodiments, the IgA portion, e.g., the Fc region of an IgA antibody, of the fusion molecule can comprise the sequence of P221 through the C-terminus of the heavy chain. For example, but not by way of limitation, the IgA antibody portion can include amino acids P221-Y472 of an IgA antibody. In certain embodiments, the Fc region of the IgA antibody, e.g., an IgA1 or IgA2m1 antibody, can comprise the sequence of P221 through the C-terminus of the heavy chain. In certain embodiments, the P221 amino acid can be mutated to an arginine (R), i.e., P221R. In certain embodiments, the Fc region of the IgA antibody, e.g., an IgA2m2 or IgA2mn antibody, can comprise the sequence of R221 through the C-terminus of the heavy chain, e.g., can include amino acids R221-Y472 of an IgA antibody. Alternatively, the Fc region of the IgA antibody can comprise the sequence of C242 through the C-terminus of the heavy chain, which deletes the hinge region of the IgA antibody. For example, but not by way of limitation, the IgA portion of the fusion molecule can include amino acids C242-Y472 of an IgA antibody. In certain embodiments, the IgA portion, e.g., the Fc region of an IgA antibody, of the fusion molecule can comprise the sequence of V222 through the C-terminus of the heavy chain. For example, but not by way of limitation, the IgA antibody portion can include amino acids V222-Y472 of an IgA antibody, e.g., an IgA1, IgA2m1, IgA2mn or IgA2m2 antibody. In certain embodiments, the IgA portion, e.g., the Fc region of an IgA antibody, of the fusion molecule can comprise the sequence of P223 through the C-terminus of the heavy chain. For example, but not by way of limitation, the IgA antibody portion can include amino acids P223-Y472 of an IgA antibody, e.g., an IgA1, IgA2m1, IgA2mn or IgA2m2 antibody. In certain embodiments, the IgA portion, e.g., the Fc region of an IgA antibody, of the fusion molecule can comprise the sequence of C241 through the C-terminus of the heavy chain. For example, but not by way of limitation, the IgA antibody portion can include amino acids C241-Y472 of an IgA antibody, e.g., an IgA1, IgA2m1, IgA2m2 or IgA2mn antibody.

In certain embodiments, the IgA portion, e.g., the Fc region of an IgA antibody, of the fusion molecule can comprise the sequence of P237 through the C-terminus of the heavy chain. For example, but not by way of limitation, the IgA antibody portion can include amino acids P237-Y472 of an IgA antibody, e.g., an IgA1, IgA2m1, IgA2mn or IgA2m2 antibody. In certain embodiments, the IgA portion, e.g., the Fc region of an IgA antibody, of the fusion molecule can comprise the sequence of P238 through the C-terminus of the heavy chain. For example, but not by way of limitation, the IgA antibody portion can include amino acids P238-Y472 of an IgA antibody, e.g., an IgA2m1, IgA2m2 or IgA2mn antibody. In certain embodiments, the IgA portion, e.g., the Fc region of an IgA antibody, of the fusion molecule can comprise the sequence of S238 through the C-terminus of the heavy chain. For example, but not by way of limitation, the IgA antibody portion can include amino acids S238-Y472 of an IgA antibody, e.g., an IgA1 antibody. In certain embodiments, the IgA portion, e.g., the Fc region of an IgA antibody, of the fusion molecule can comprise the sequence of P239 through the C-terminus of the heavy chain. For example, but not by way of limitation, the IgA antibody portion can include amino acids P239-Y472 of an IgA antibody, e.g., an IgA1, an IgA2m1, IgA2m2 or IgA2mn antibody. In certain embodiments, the IgA portion, e.g., the Fc region of an IgA antibody, of the fusion molecule can comprise the sequence of P240 through the C-terminus of the heavy chain. For example, but not by way of limitation, the IgA antibody portion can include amino acids P240-Y472 of an IgA antibody, e.g., an IgA2m1, IgA2m2 or IgA2mn antibody. In certain embodiments, the IgA portion, e.g., the Fc region of an IgA antibody, of the fusion molecule can comprise the sequence of S240 through the C-terminus of the heavy chain. For example, but not by way of limitation, the IgA antibody portion can include amino acids S240 of an IgA antibody, e.g., an IgA1 antibody. In certain embodiments, the IgA portion of the fusion molecule does not include the tailpiece of an IgA antibody, e.g., amino acids 454-472.

In certain embodiments, an IgG-IgA fusion molecule of the present disclosure can include an Fc region from one IgA isotype and a hinge region from a second isotype. For example, but not by way of limitation, an IgG-IgA fusion molecule of the present disclosure can include a hinge region from an IgA2, e.g., IgA2m1, IgA2m2 or IgA2mn, antibody and include an Fc region from an IgA1 antibody.

In certain embodiments, the heavy chain of the IgG antibody has been modified to remove the C-terminal lysine amino acid, e.g., amino acid K447 of an IgG antibody (e.g., IgG1, IgG2, IgG3 and IgG4). For example, but not by way of limitation, the present disclosure provides an IgG-IgA fusion molecule that includes an IgG antibody that lacks the amino acid K447 and an IgA portion that includes amino acids P221-Y472 or R221-Y472 of an IgA antibody.

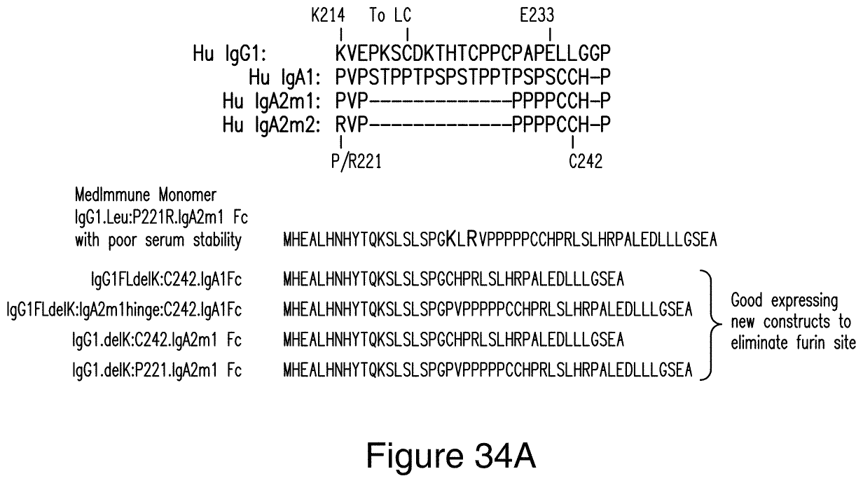

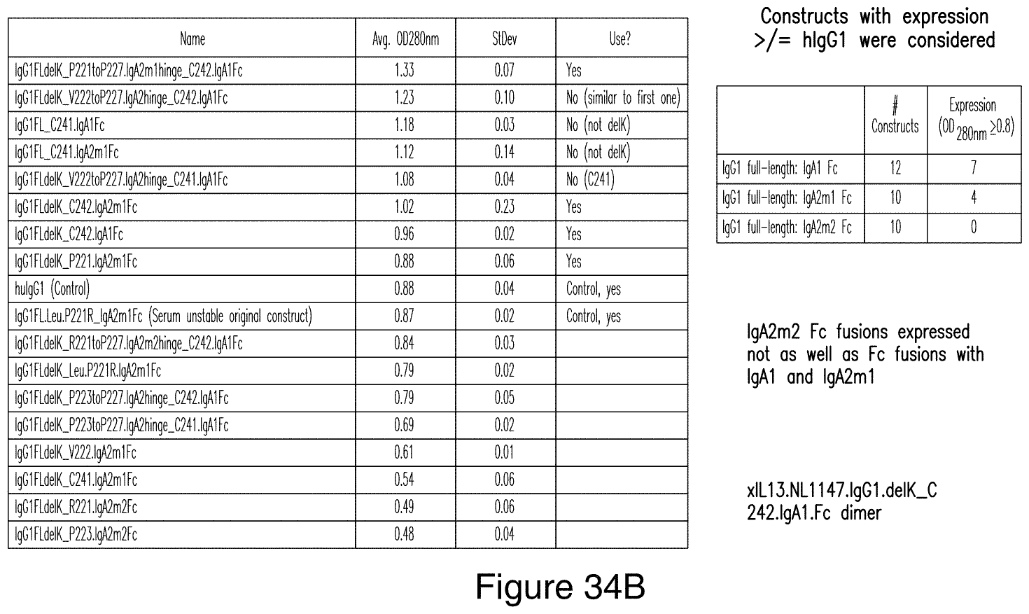

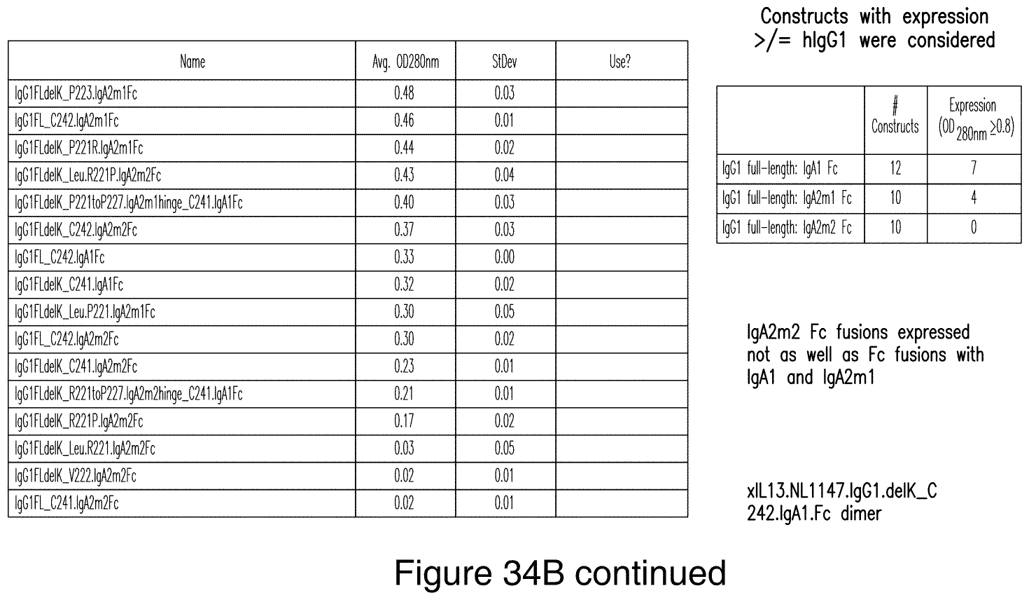

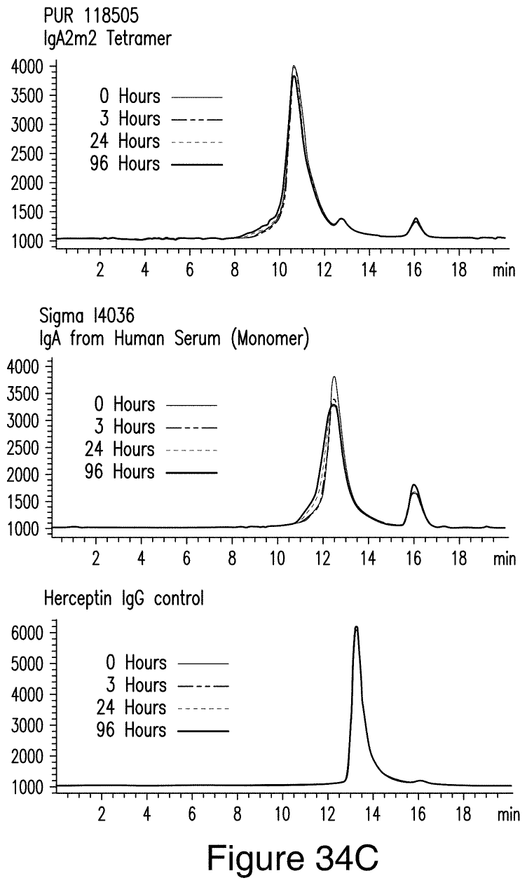

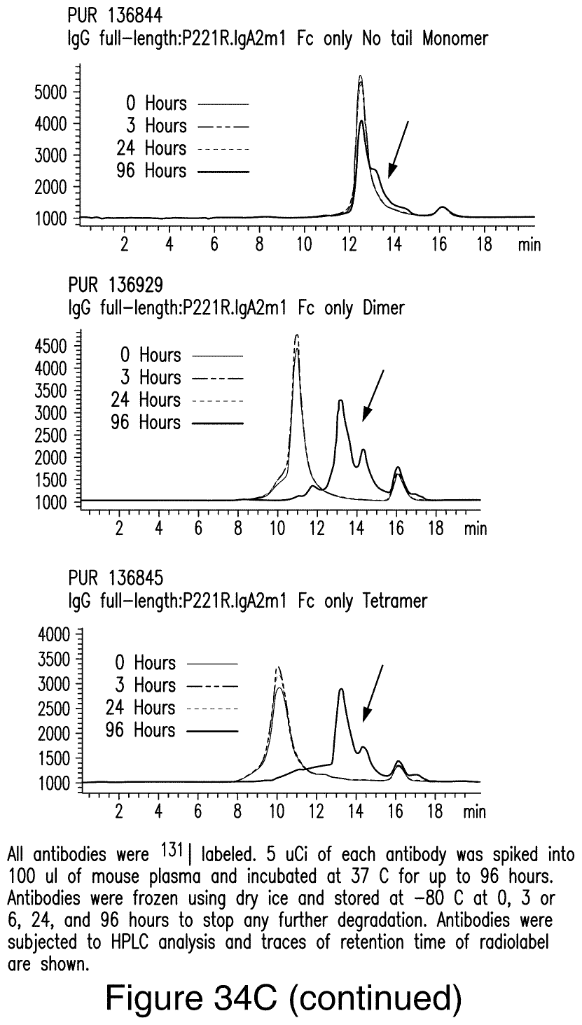

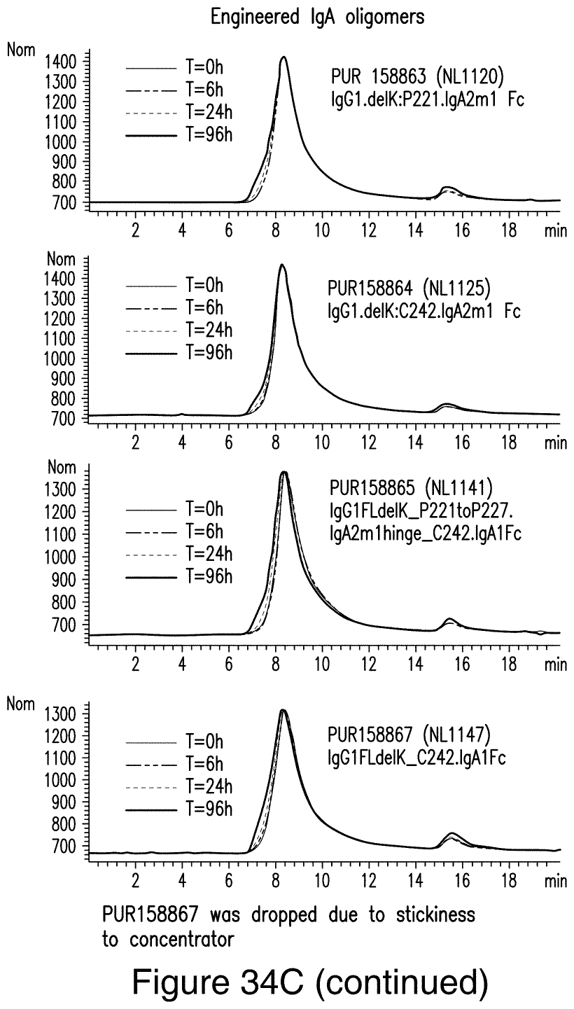

In certain embodiments, the junction between the IgG antibody and the Fc region of the IgA antibody can comprise the amino acid sequence TQKSLSLSPGPVPPPPPCC (SEQ ID NO: 1) or a fragment thereof or conservative substitutions thereof. In certain embodiments, the junction between the IgG antibody and the Fc region of the IgA antibody can comprise the amino acid sequence TQKSLSLSPGC (SEQ ID NO: 2) or a fragment thereof or conservative substitutions thereof. Non-limiting examples of conservative substitutions are provided in Table 1. In certain embodiments, the junction between the IgG antibody and the Fc region of the IgA antibody can comprise an amino acid sequence as disclosed in FIG. 34A.

In certain embodiments, the IgG-IgA Fc fusions of the present disclosure are stable in plasma for up to about 1 day, up to about 2 days, about to about 3 days, up to about 4 days or up to about 5 days. For example, but not by way of limitation, IgG1-IgA Fc fusions of the present disclosure are stable in the plasma for up to about 4 days.

In certain embodiments, an IgG-IgA fusion molecule of the present disclosure can further include one or more amino acid substitutions, as described above, to reduce glycosylation. For example, but not by way of limitation, an IgG-IgA fusion molecule of the present disclosure can be modified to remove glycosylation of the heavy chain of the IgA antibody and/or the J chain of the IgG-IgA fusion molecule. In certain embodiments, the IgA antibody of the IgG-IgA fusion molecule is aglycosylated. In certain embodiments, the IgG-IgA fusion molecules of the present disclosure bind to FcRn but do not bind to FcαRI.

In certain embodiments, an IgG-IgA fusion molecule of the present disclosure can further include a substitution of one or more of Fc region residues 238, 265, 269, 270, 297, 327 and 329. For example, but not by way of limitation, an IgG-IgA fusion molecule of the present disclosure can further include a substitution at amino acid 297, e.g., N297G.

In certain embodiments, an IgG-IgA fusion molecule of the present disclosure can further include a substitution at amino acid C471 and/or C311. In certain embodiments, an IgG-IgA fusion molecule of the present disclosure can have a mutation at amino acid C471, e.g., C471S. In certain embodiments, an IgG-IgA Fc fusion molecule of the present disclosure can have a mutation at amino acid C311, e.g., C311S.

B. Chimeric and Humanized Antibodies

In certain embodiments, an antibody of the present disclosure is a chimeric antibody. Certain chimeric antibodies are described, e.g., in U.S. Pat. No. 4,816,567; and Morrison et al., Proc. Natl. Acad. Sci. USA, 81:6851-6855 (1984)). In certain embodiments, a chimeric antibody of the present disclosure comprises a non-human variable region (e.g., a variable region derived from a mouse, rat, hamster, rabbit or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In certain embodiments, a chimeric antibody of the present disclosure can be a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA, 89:4285 (1992); and Presta et al., J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J Biol. Chem. 272:10678-10684 (1997) and Rosok et al., J. Biol. Chem. 271:22611-22618 (1996)).

C. Human Antibodies

In certain embodiments, an antibody of the present disclosure can be a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

D. Antibody Variants

The presently disclosed subject matter further provides amino acid sequence variants of the disclosed antibodies. For example, it may be desirable to improve the binding affinity and/or other biological properties of the antibody. Amino acid sequence variants of an antibody can be prepared by introducing appropriate modifications into the nucleotide sequence encoding the antibody, or by peptide synthesis. Such modifications include, but are not limited to, deletions from, and/or insertions into and/or substitutions of residues within the amino acid sequences of the antibody. Any combination of deletion, insertion, and substitution can be made to arrive at the final construct, provided that the final antibody, i.e., modified, possesses the desired characteristics, e.g., antigen-binding.

1. Substitution, Insertion and Deletion Variants

In certain embodiments, antibody variants can have one or more amino acid substitutions. Sites of interest for substitutional mutagenesis include the HVRs and FRs. Non-limiting examples of conservative substitutions are shown in Table 1 under the heading of “preferred substitutions.” Non-limiting examples of more substantial changes are provided in Table 1 under the heading of “exemplary substitutions,” and as further described below in reference to amino acid side chain classes. Amino acid substitutions can be introduced into an antibody of interest and the products screened for a desired activity, e.g., retained/improved antigen binding, decreased immunogenicity or improved complement dependent cytotoxicity (CDC) or antibody-dependent cell-mediated cytotoxicity (ADCC).

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

In certain embodiments, non-conservative substitutions will entail exchanging a member of one of these classes for another class.

In certain embodiments, a type of substitutional variant involves substituting one or more hypervariable region residues of a parent antibody, e.g., a humanized or human antibody. Generally, the resulting variant(s) selected for further study will have modifications, e.g., improvements, in certain biological properties such as, but not limited to, increased affinity, reduced immunogenicity, relative to the parent antibody and/or will have substantially retained certain biological properties of the parent antibody. A non-limiting example of a substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

In certain embodiments, alterations (e.g., substitutions) can be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O′Brien et al., ed., Human Press, Totowa, N.J., (2001)). In certain embodiments of affinity maturation, diversity can be introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding can be specifically identified, e.g., using alanine scanning mutagenesis or modeling. CDR-H3 and CDR-L3 in particular are often targeted.

In certain embodiments, substitutions, insertions, or deletions can occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for Antibody-directed enzyme prodrug therapy (ADEPT)) or a polypeptide which increases the serum half-life of the antibody.

2. Fc Region Variants

In certain embodiments, one or more amino acid modifications can be introduced into the Fc region of an antibody provided herein, thereby generating an Fc region variant. The Fc region variant may comprise a human Fc region sequence (e.g., a human IgA Fc region or a human IgG1, IgG2, IgG3 or IgG4 Fc region) comprising an amino acid modification (e.g., a substitution) at one or more amino acid positions.

In certain embodiments, the present disclosure provides an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks IgA-specific hFc receptor, i.e., FcαRI, binding but retains FcRn binding ability. FcR expression on hematopoietic cells is summarized in Table 3 on page 464 of Ravetch and Kinet, Annu. Rev. Immunol. 9:457-492 (1991). For example, the primary cells for mediating ADCC, NK cells, express FcγRIII only, whereas monocytes express FcγRI, FcγRII and FcγRIII.Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (Cell Technology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M.S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)). In certain embodiments, alterations can be made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. See, e.g., U.S. Pat. No. 6,737,056; WO 2004/056312, and Shields et al., J. Biol. Chem. 9(2): 6591-6604 (2001).

In certain embodiments, an antibody variant of the present disclosure comprises an Fc region with one or more amino acid substitutions which improve ADCC, e.g., substitutions at positions 298, 333, and/or 334 of the Fc region (EU numbering of residues).

In certain embodiments, alteration made in the Fc region of an antibody, e.g., a bispecific antibody, disclosed herein, can produce a variant antibody with an increased half-life and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein, which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

3. Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies can be generated as described, e.g., in U.S. Pat. No. 7,521,541.

4. Antibody Derivatives

In certain embodiments, an antibody of the present disclosure can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). In certain embodiments, the radiation can be of any wavelength, and includes, but is not limited to, wavelengths that do not harm ordinary cells, but which heat the nonproteinaceous moiety to a temperature at which cells proximal to the antibody-nonproteinaceous moiety are killed.

5. Immunoconjugates

The presently disclosed subject matter also provides immunoconjugates, which include an antibody, disclosed herein, conjugated to one or more cytotoxic agents, such as chemotherapeutic agents or drugs, growth inhibitory agents, proteins, peptides, toxins (e.g., protein toxins, enzymatically active toxins of bacterial, fungal, plant, or animal origin, or fragments thereof), or radioactive isotopes. For example, an antibody of the disclosed subject matter can be functionally linked (e.g., by chemical coupling, genetic fusion, noncovalent association or otherwise) to one or more other binding molecules, such as another antibody, antibody fragment, peptide or binding mimetic.

In certain embodiments, an immunoconjugate is an antibody-drug conjugate (ADC) in which an antibody of the present disclosure is conjugated to one or more drugs, including but not limited to, a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, an immunoconjugate includes an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In certain embodiments, an immunoconjugate includes an antibody, as described herein, conjugated to a radioactive atom to form a radioconjugate. A variety of radioactive isotopes are available for the production of radioconjugates. Non-limiting examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When a radioconjugate is used for detection, it can include a radioactive atom for scintigraphic studies, for example tc99m or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

Conjugates of an antibody fragment and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used. Non-limiting examples of linkers are disclosed above.

The immunuoconjugates disclosed herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

III. Methods of Antibody Production and Purification

A. Methods of Antibody Production

The antibodies disclosed herein can be produced using any available or known technique in the art. For example, but not by way of limitation, antibodies can be produced using recombinant methods and compositions, e.g., as described in U.S. Pat. No. 4,816,567. Detailed procedures to generate the antibodies of the present disclosure, e.g., IgA antibodies and IgG-IgA fusion molecules, are described in the Examples below.

The presently disclosed subject matter further provides an isolated nucleic acid encoding an antibody disclosed herein. For example, the isolated nucleic acid can encode an amino acid sequence that encodes an aglycosylated antibody of the present disclosure. In certain embodiments, an isolated nucleic acid of the present disclosure can encode an amino acid sequence that encodes an IgA antibody that has been modified to remove one or more, two or more, three or more, four or more, five or more or six or more glycosylation sites, e.g., N-linked glycosylation sites and/or O-linked glycosylation sites. In certain embodiments, an isolated nucleic acid of the present disclosure can encode an amino acid sequence that encodes an IgA antibody that has one or more, two or more, three or more, four or more, five or more, six or more, seven or more, eight or more, nine or more, ten or more, eleven or more or twelve modifications, e.g., substitutions, at amino acids N166, T168, N211, S212, S213, N263, T265, N337, I338, T339, N459 and/or S461. In certain embodiments, an isolated nucleic acid of the present disclosure can encode an amino acid sequence that encodes an IgG-IgA fusion molecule, e.g., IgG-IgA Fc fusion molecule, disclosed herein.

In certain embodiments, the nucleic acid can be present in one or more vectors, e.g., expression vectors. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the disclosed subject matter is intended to include such other forms of expression vectors, such as viral vectors (e.g., replication defective retroviruses, adenoviruses and adeno-associated viruses) that serve equivalent functions.

In certain embodiments, the nucleic acid encoding an antibody of the present disclosure and/or the one or more vectors including the nucleic acid can be introduced into a host cell. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, a host cell can include, e.g., has been transformed with, (1) a vector comprising a nucleic acid that encodes an amino acid sequence comprising the light chain of the antibody, an amino acid sequence comprising the heavy chain of the antibody and an amino acid sequence comprising the J chain of the antibody; (2) (a) a first vector comprising a nucleic acid that encodes an amino acid sequence comprising the light chain of the antibody and an amino acid sequence comprising the heavy chain of the antibody and (b) a second vector comprising a nucleic acid that encodes an amino acid sequence comprising the J chain of the antibody; or (3) (a) a first vector comprising a nucleic acid that encodes an amino acid sequence comprising the light chain of the antibody, (b) a second vector comprising a nucleic acid that encodes an amino acid sequence comprising the heavy chain of the antibody and (c) a third vector comprising a nucleic acid that encodes an amino acid sequence comprising the J chain of the antibody. In certain embodiments, a host cell can include, e.g., has been transformed with, (a) a first vector comprising a nucleic acid that encodes an amino acid sequence comprising the light chain of the antibody, (b) a second vector comprising a nucleic acid that encodes an amino acid sequence comprising the heavy chain of the antibody, (c) a third vector comprising a nucleic acid that encodes an amino acid sequence comprising the J chain of the antibody and (d) a fourth vector comprising a nucleic acid that encodes an amino acid comprising the secretory component of the antibody.

In certain embodiments, the host cell is eukaryotic, e.g., a Chinese Hamster Ovary (CHO) cell. In certain embodiments, the host cell is a 293 cell, e.g., Expi293 cell.

In certain embodiments, the methods of making an antibody of the present disclosure include culturing a host cell, in which one or more nucleic acids encoding the antibody have been introduced, under conditions suitable for expression of the antibody, and optionally recovering the antibody from the host cell and/or host cell culture medium. In certain embodiments, the antibody is recovered from the host cell through chromatography techniques.

For recombinant production of an antibody of the present disclosure, a nucleic acid encoding an antibody, e.g., as described above, can be isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies can be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006). Suitable host cells for the expression of glycosylated antibody can also derived from multicellular organisms (invertebrates and vertebrates). Examples of invertebrate cells include plant and insect cells. Numerous baculoviral strains have been identified which may be used in conjunction with insect cells, particularly for transfection of Spodoptera frupperda cells.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frupperda cells.

In certain embodiments, plant cell cultures can be utilized as host cells. See, e.g., U.S. Pat. Nos. 5,959,177, 6,040,498, 6,420,548, 7,125,978, and 6,417,429 (describing PLANTIBODIES™ technology for producing antibodies in transgenic plants).

In certain embodiments, vertebrate cells can also be used as hosts. For example, and not by way of limitation, mammalian cell lines that are adapted to grow in suspension can be useful. Non-limiting examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

In certain embodiments, an animal system can be used to produce an antibody of the present disclosure. One animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known (see, e.g., Harlow and Lane (1988), Antibodies, A Laboratory Manual, Cold Spring Harbor Laboratory Press, Cold Spring Harbor New York).

1. Methods of Polymeric IgA Production

The present disclosure provides methods for producing polymeric IgA. In certain embodiments, methods of the present disclosure can be used to produce IgA polymers that contain two or more IgA monomers, e.g., from about two to about five IgA monomers. For example, but not by way of limitation, methods of the present disclosure can be used to produce IgA dimers, trimers, tetramers and/or pentamers. In certain embodiments, such methods include altering the ratio of the amount of DNA encoding the J chain to the amount of DNA encoding the light chain (LC) and/or heavy chain (HC) that is introduced, e.g., transfected, into a cell. In certain embodiments, such methods include altering the ratio of the amount of DNA encoding the J chain to the amount of DNA encoding the LC, HC and secretory component (SC) that is introduced, e.g., transfected, into a cell.

The present disclosure provides methods for increasing the production of IgA dimers. In certain embodiments, the method for increasing production of IgA dimers includes increasing the amount of DNA encoding the J chain that is introduced, e.g., transfected, into a cell relative to the amount of DNA encoding the light chain and heavy chain. In certain embodiments, increased expression is relative to the amount of IgA dimers produced in a cell introduced, e.g., transfected, with equal amounts of J chain, heavy chain and light chain DNA. For example, but not by way of limitation, the methods can be used to produce IgA1, IgA2m1, IgA2m1.P221R dimers, IgA2m2 and IgA2mn dimers. In certain embodiments, the method can include introducing into, e.g., transfecting, a host cell with a ratio of the amount of DNA encoding the heavy chain to the amount of DNA encoding the light chain to the amount of DNA encoding the J chain (HC:LC:JC) that is about 1:1:2, about 1:1:3, about 1:1:4 or about 1:1:5 to increase production of IgA dimers, e.g., a ratio from about 1:1:2 to about 1:1:5. In certain embodiments, the method can include transfecting a cell with an amount of DNA encoding the J chain that is about 2 fold greater, about 3 fold greater, about 4 fold greater or about 5 fold greater than the amount of DNA encoding the light chain and/or the amount of DNA encoding the heavy chain.

The present disclosure provides methods for increasing the production of IgA polymers. For example, but not by way of limitation, the present disclosure provides methods for increasing the production of IgA dimers, trimers, tetramers and/or pentamers. In certain embodiments, the method for increasing production of IgA polymers, e.g., dimers, trimers, tetramers and/or pentamers, includes decreasing the amount of DNA encoding the J chain that is introduced into a cell relative to the amount of DNA encoding the light chain and heavy chain. In certain embodiments, increased production is relative to the amount of IgA polymers, e.g., dimers, trimers, tetramers and/or pentamers produced in a cell introduced, e.g., transfected, with equal amounts of heavy chain and light chain DNA relative to the amount of J chain DNA. For example, but not by way of limitation, the methods can be used to produce IgA1, IgA2m1, IgA2m1 P221R, IgA2m2 or IgA2mn polymers, e.g., dimers, trimers, tetramers and/or pentamers. In certain embodiments, the method can include transfecting a host cell with a ratio of the amount of DNA encoding the heavy chain to the amount of DNA encoding the light chain to the amount of DNA encoding the J chain (HC:LC:JC) that is about 1:1:0.25 or about 1:1:0.5, e.g., a ratio from about 1:1:0.25 to about 1:1:0.5, to increase production of IgA trimers, tetramers and/or pentamers. In certain embodiments, the amount of DNA encoding the J chain can be less than about 3 fold greater, less than about 2 fold greater or less than about 1 fold greater than the amount of DNA encoding the light chain and/or the amount of DNA encoding the heavy chain. In certain embodiments, the amount of DNA encoding the J chain can be less than about 0.5 fold or less than about 0.25 fold of the amount of DNA encoding the light chain and/or the amount of DNA encoding the heavy chain.

In certain embodiments, the methods for increasing the production of IgA1, IgA2m1 and/or IgA2mn trimers, tetramers and pentamers can include expressing, in a cell, an IgA1 antibody, an IgA2m1 antibody and/or IgA2mn antibody that has a substitution at amino acid V458. In certain embodiments, the amino acid V458 can be mutated to an isoleucine (i.e., V4581). In certain embodiments, the increase in the production of IgA1, IgA2m1 and/or IgA2mn trimers, tetramers and pentamers is relative to the production of IgA1, IgA2m1 and/or IgA2mn trimers, tetramers and pentamers resulting from the expression of an IgA1 antibody, an IgA2m1 antibody and/or IgA2mn antibody, in a cell, that does not have a substitution at amino acid V458.

In certain embodiments, the methods for increasing the production of IgA2m2 dimers can include expressing an IgA2m2 antibody that has a substitution at amino acid 1458. In certain embodiments, the amino acid I458 can be mutated to a valine (i.e., I458V). In certain embodiments, the increase in the production of IgA2m2 dimers is relative to the production of IgA2m2 dimers resulting from the expression of an IgA2m2 antibody that does not have a substitution at amino acid 1458.

In certain embodiments, the method for increasing the production of IgA polymers can include removing one or more glycosylation sites from the IgA antibody, e.g., by amino acid substitution (as described above), e.g., relative to the production of IgA polymers by an IgA antibody that has not been modified to remove a glycosylation site. In certain embodiments, the method for increasing production of IgA polymers can include one or more substitutions at amino acids N459 and/or S461. For example, but not by way of limitation, the IgA antibody can have a substitution at amino acid N459. In certain embodiments, the IgA antibody can have a substitution at amino acid S461. In certain embodiments, the IgA antibody can have substitutions at amino acids N459 and/or S461. Non-limiting examples of such substitutions include the mutation of N459 to A, G or Q. In certain embodiments, amino acid S461 can be mutated to A. In certain embodiments, a method for increasing the production of IgA1 polymers includes expressing an IgAl antibody with a substitution at amino acids N459 and/or S461, e.g., a substitution at amino acids N459 and S461, e.g., wherein increased expression is relative to the amount of IgA1 polymers produced by expression of an IgA1 antibody that does not have a substitution at amino acids N459 and/or S461. In certain embodiments, a method for increasing the production of IgA2 polymers, e.g., IgA2m1, IgA2m2 and IgA2mn polymers, includes expressing an IgA2 antibody with a substitution at amino acids N459 and/or S461, e.g., a substitution at amino acids N459 and S461. In certain embodiments, the increase in the production of IgA2 polymers is relative to the production of IgA2 polymers resulting from the expression of an IgA2 antibody that does not have a substitution at amino acids N459 and/or S461.

In certain embodiments, a method for reducing the production of IgA polymers (e.g., increasing the production of IgA monomers) includes expressing an IgA antibody, e.g., an IgA1, IgA2m1, IgA2m2 or IgA2mn antibody, with a substitution at amino acid C471, e.g., a C471S mutation. For example, but not by way of limitation, a method for reducing the production of IgA2m2 polymers includes expressing an IgA2m2 antibody with a substitution at amino acid C471, e.g., a C471S mutation. In certain embodiments, the decrease in the production of IgA polymers, e.g., IgA2m2 polymers, is relative to the production of IgA polymers, e.g., IgA2m2 polymers, resulting from the expression of an IgA antibody, e.g., IgA2m2 antibody, that does not have a substitution at amino acid C471.

2. Methods of Polymeric IgG-IgA Fusion Molecule Production

The present disclosure further provides methods for producing IgG-IgA fusion molecules of the present disclosure. In certain embodiments, the present disclosure provides methods for generating dimers of IgG-IgA fusion molecules disclosed herein. In certain embodiments, the present disclosure provides methods for producing polymers, e.g., dimers, trimers and/or tetramers, of IgG-IgA fusion molecules disclosed herein.

In certain embodiments, the methods are directed to the production of dimers of an IgG-IgA fusion molecule. For example, but not by way of limitation, a method of expressing dimers of IgG-IgA fusion molecules can include expressing an IgG-IgA fusion molecule comprising a full-length IgG antibody fused at its C-terminus to an Fc region of an IgA antibody, where the Fc region of the IgA antibody comprises a sequence comprising P221 or R221 through the C-terminus of the heavy chain of the IgA antibody and the IgG antibody comprises a deletion of amino acid K447.

In certain embodiments, the methods are directed to the production of polymers of an IgG-IgA fusion molecule disclosed herein. For example, but not by way of limitation, a method of expressing polymers of IgG-IgA fusion molecules comprises expressing an IgG-IgA fusion molecule comprising a full-length IgG antibody fused at its C-terminus to an Fc region of an IgA antibody, where the Fc region of the IgA antibody comprises a sequence comprising C242 through the C-terminus of the heavy chain of the IgA antibody. In certain embodiments, the IgG antibody includes a deletion of amino acid K447. In certain embodiments, the polymers of the IgG-IgA fusion molecules produced by the method include dimers, trimers and/or tetramers of the IgG-IgA fusion molecule.

B. Methods of Antibody Purification

The present disclosure further provides methods for purifying the antibodies disclosed herein. For example, but not by way of limitation, the present disclosure provides methods for separating the oligomeric states of the antibodies disclosed herein, e.g., separating the dimeric state from the tetrameric state of the antibody.

In certain embodiments, methods for purifying the antibodies of the present disclosure can include purifying the antibodies using a protein affinity column. In certain embodiments, the methods can further include performing size exclusion chromatography (SEC). For example, but not by way of limitation, SEC can be performed to purify and/or isolate specific oligomeric states of an antibody disclosed herein, e.g., an IgA antibody and/or an IgG-IgA fusion molecule. In certain embodiments, SEC can be performed to purify and/or isolate one oligomeric state, e.g., a dimeric state, a trimeric state, a tetrameric state and/or a pentameric state, of an antibody disclosed herein.

In certain embodiments, the protein affinity column can be a Mab Select Sure (GE Healthcare) column. In certain embodiments, antibodies of the present disclosure, e.g., IgA samples that primarily contain one oligomeric state, can be affinity purified using Mab Select Sure (GE Healthcare) followed by SEC with a HiLoad Superdex column (GE Healthcare).

In certain embodiments, antibodies of the present disclosure, e.g., the IgA antibodies disclosed herein, can be purified with Protein L (GE Healthcare) followed by SEC. In certain embodiments, the Protein L column can be washed with a first wash buffer that comprises Tris buffer (25 mM Tris, pH 7.5, 150 mM NaCl, 5 mM EDTA, 2 mM NaN₃). In certain embodiments, the Protein L column can be further washed with a second wash buffer comprising Triton X-114 buffer (25 mM Tris, pH 7.5, 150 mM NaCl, 5 mM EDTA, 0.1% Triton X-114, 2 mM NaN₃) to remove endotoxin. In certain embodiments, the Protein L column can be washed with a third wash buffer that includes Tris buffer, washed with a fourth wash buffer that includes KP buffer (0.4 M potassium phosphate, pH 7.0, 5 mM EDTA, 0.02% Tween20, 2 mM NaN3) and/or washed with a fifth wash buffer that comprises Tris buffer. Alternatively or additionally, the Protein L column can be washed one or more times with a wash buffer comprising PBS.

In certain embodiments, the antibodies can be eluted from the Protein L column using a buffer that comprises 150 mM acetic acid, pH 2.7, which can be neutralized with 1 M arginine, 0.4 M succinate, pH 9.0. Alternatively or additionally, the antibodies can be eluted from the Protein L column using a buffer that comprises 50 mM phosphoric acid at pH 3.0. In certain embodiments, the eluted antibodies can be neutralized with 20× PBS at pH 11.

In certain embodiments, IgA samples that comprise complex oligomers, the Protein L eluate can be further purified using a 3.5 μm, 7.8 mm×300 mm)(Bridge Protein BEH 450 Å SEC column (Waters), e.g., to isolate a particular oligomeric state (e.g., dimeric, trimeric and/or tetrameric state) of the antibody. In certain embodiments, less than 1 mg of total protein in an injection volume no larger than 100 μL was run over the column at 1 mL/min using an Agilent 1260 Infinity HPLC with 0.2 M arginine, 0.137 M succinate, pH 5.0 as the mobile phase and 200 μL fractions were collected. In certain embodiments, fractions from the SEC column can be selectively pooled to isolate predominantly one oligomeric state. One or more runs can be performed, and the fractions of a given oligomer from each run can be pooled together.

IV. Methods of Treatment

The presently disclosed subject matter further provides methods for using the disclosed antibodies, e.g., the IgA and the IgG-IgA fusion molecules. In certain embodiments, the methods are directed to therapeutic uses of the presently disclosed antibodies.

In certain embodiments, one or more antibodies of the presently disclosed subject matter can be used for treating a disease and/or disorder in a subject. For example, but not by way of limitation, an antibody of the present disclosure can be used to treat an inflammatory disease, an autoimmune disease and cancer. In certain embodiments, antibodies of the present disclosure can be used to treat cancer. In certain embodiments, antibodies of the present disclosure that lack binding to FcαRI and cannot activate FcαRI can be used to treat an inflammatory disease, an autoimmune disease and cancer. In certain embodiments, antibodies of the present disclosure can be used to treat diseases and/or disorders that require transcytosis of the antibody for therapeutic effect and/or to access a therapeutic target. For example, but not by way of limitation, an antibody of the present disclosure can be used to treat diseases and/or disorders that require the transcytosis of the antibody across a mucosal membrane.

In certain embodiments, the present disclosure provides an antibody for use in a method of treating an individual having a specific disease and/or disorder comprising administering to the individual an effective amount of the antibody or compositions comprising the same. In certain embodiments, the method further comprises administering to the individual an effective amount of at least one additional therapeutic agent. In certain embodiments, the present disclosure provides an antibody for use in inhibiting a particular molecular pathway and/or mechanism. In certain embodiments, the present disclosure provides an antibody for use in a method of inhibiting a particular molecular pathway and/or mechanism in an individual that comprises administering to the individual an effective of the antibody to inhibit the particular molecular pathway and/or mechanism.

In certain embodiments, the present disclosure provides an antibody for use in activating a particular molecular pathway and/or mechanism. In certain embodiments, the present disclosure provides an antibody for use in a method of activating a particular molecular pathway and/or mechanism in an individual that comprises administering to the individual an effective of the antibody to inhibit the particular molecular pathway and/or mechanism.

An “individual,” “patient” or “subject,” as used interchangeably herein, refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The present disclosure further provides for the use of an antibody in the manufacture or preparation of a medicament for the treatment of a disease and/or disorder in a subject. In certain embodiments, the medicament is for treatment of a particular disease and/or disorder. In certain embodiments, the medicament is for use in a method of treating a particular disease and/or disorder comprising administering to an individual having the disease an effective amount of the medicament. In certain embodiments, the method further comprises administering to the individual an effective amount of at least one additional therapeutic agent. In certain embodiments, the medicament is for inhibiting or activating a particular molecular pathway and/or mechanism. In certain embodiments, the medicament is for use in a method of inhibiting or activating a particular molecular pathway and/or mechanism in an individual comprising administering to the individual an amount effective of the medicament to inhibit a particular molecular pathway and/or mechanism.

In certain embodiments, an antibody for use in the disclosed therapeutic methods can be present in a pharmaceutical composition, as described herein. In certain embodiments, the pharmaceutical composition can include a pharmaceutically acceptable carrier, as described herein. In certain embodiments, the pharmaceutical composition can include one or more of the antibodies of the present disclosure.

Additionally or alternatively, the pharmaceutical composition can include a second therapeutic agent. When one or more of the disclosed antibodies are administered with another therapeutic agent, the one or more antibodies and the other therapeutic agent can be administered in either order or simultaneously. Such combination therapies noted above encompass combined administration (where two or more therapeutic agents are included in the same or separate formulations), and separate administration, in which case, administration of the antibody of the present disclosure can occur prior to, simultaneously, and/or following, administration of the additional therapeutic agent or agents. In certain embodiments, administration of an antibody of the present disclosure and administration of an additional therapeutic agent occur within about one month, or within about one, two or three weeks, or within about one, two, three, four, five or six days, of each other.

An antibody of the present disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the present disclosure would be formulated, dosed and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the present disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. In certain embodiments, an antibody of the present disclosure can be administered on an as needed basis. In certain embodiments, the antibody can be administered to the patient one time or over a series of treatments. For example, but not by way of limitation, the antibody and/or pharmaceutical composition contains an antibody, as disclosed herein, can be administered to a subject twice every day, once every day, once every two days, once every three days, once every four days, once every five days, once every six days, once a week, once every two weeks, once every three weeks, once every month, once every two months, once every three months, once every six months or once every year.

In certain embodiments, depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. In certain embodiments, the daily dosage can be greater than about 100 mg/kg. In certain embodiments, dosage can be adjusted to achieve a plasma antibody concentration of 1-1000 μg/ml and in some methods 25-300 μg/ml.

For repeated administrations over several days or longer, depending on the condition, the treatment could generally be sustained until a desired suppression of disease symptoms occurs. One exemplary dosage of the antibody would be in the range from about 0.05 mg/kg to about 10 mg/kg. In certain embodiments, one or more doses of about 0.5 mg/kg, 2.0 mg/kg, 4.0 mg/kg or 10 mg/kg (or any combination thereof) can be administered to the patient. Alternatively, antibody can be administered as a sustained release formulation, in which case less frequent administration is required. Dosage and frequency can vary based on the half-life of the antibody in the patient. In certain embodiments, such doses may be administered intermittently, e.g., every week or every three weeks (e.g., such that the patient receives from about two to about twenty, or, e.g., about six doses of the antibody). An initial higher loading dose, followed by one or more lower doses may be administered.

In certain embodiments, the method can further include monitoring the subject and determining the effectiveness of the treatment. For example, the progress of this therapy can be easily monitored by conventional techniques and assays.

V. Pharmaceutical Compositions

The presently disclosed subject matter further provides pharmaceutical compositions containing one or more of the presently disclosed antibodies, e.g., the IgA and the IgG-IgA Fc fusion proteins, with a pharmaceutically acceptable carrier. In certain embodiments, the pharmaceutical compositions can include a combination of multiple (e.g., two or more) antibodies and/or antigen-binding portions thereof of the presently disclosed subject matter.

In certain embodiments, the disclosed pharmaceutical compositions can be prepared by combining an antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. For example, but not by way of limitation, lyophilized antibody formulations are described in U.S. Pat. No. 6,267,958. In certain embodiments, aqueous antibody formulations can include those described in U.S. Pat. No. 6,171,586 and WO2006/044908, the latter formulations including a histidine-acetate buffer. In certain embodiments, the antibody can be of a purity greater than about 80%, greater than about 90%, greater than about 91%, greater than about 92%, greater than about 93%, greater than about 94%, greater than about 95%, greater than about 96%, greater than about 97%, greater than about 98%, greater than about 99%, greater than about 99.1%, greater than about 99.2%, greater than about 99.3%, greater than about 99.4%, greater than about 99.5%, greater than about 99.6%, greater than about 99.7%, greater than about 99.8% or greater than about 99.9%.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The carrier can be suitable for intravenous, intramuscular, subcutaneous, parenteral, spinal or epidermal administration (e.g., by injection or infusion). Depending on the route of administration, the antibody, can be coated in a material to protect the compound from the action of acids and other natural conditions that may inactivate the compound.

Pharmaceutical compositions of the present disclosure also can be administered in combination therapy, i.e., combined with other agents. In certain embodiments, pharmaceutical compositions disclosed herein can also contain more than one active ingredient as necessary for the particular indication being treated, for example, those with complementary activities that do not adversely affect each other. In certain embodiments, the pharmaceutical composition can include a second active ingredient for treating the same disease treated by the first therapeutic. Such active ingredients are suitably present in combination in amounts that are effective for the purpose intended. For example, and not by way of limitation, the formulation of the present disclosure can also contain more than one active ingredient as necessary for the particular indication being treated, preferably those with complementary activities that do not adversely affect each other. For example, it may be desirable to further provide a second therapeutic useful for treatment of the same disease. Such active ingredients are suitably present in combination in amounts that are effective for the purpose intended.

A composition of the present disclosure can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J.R. Robinson, ed., Marcel Dekker, Inc., New York, 1978. In certain embodiments, the pharmaceutical compositions are manufactured under Good Manufacturing Practice (GMP) conditions of the U.S. Food and Drug Administration.

Sustained-release preparations containing a disclosed antibody can also be prepared. Suitable examples of sustained-release preparations include semipermeable matrices of solid hydrophobic polymers containing the antibody, which matrices are in the form of shaped articles, e.g. films, or microcapsules. In certain embodiments, active ingredients can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

To administer an antibody of the present disclosure by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7:27 (1984)).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present disclosure is contemplated. Supplementary active compounds can also be incorporated into the compositions.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating one or more disclosed antibodies in the required amount in an appropriate solvent with one or a combination of ingredients enumerated above, as required, followed by sterilization microfiltration, e.g., by filtration through sterile filtration membranes. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Therapeutic compositions can also be administered with medical devices known in the art. For example, a therapeutic composition of the present disclosure can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,223, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

For the therapeutic compositions, formulations of the present disclosure include those suitable for oral, nasal, topical (including buccal and sublingual), rectal, vaginal and/or parenteral administration. The formulations can conveniently be presented in unit dosage form and may be prepared by any methods known in the art of pharmacy. The amount of antibody, which can be combined with a carrier material to produce a single dosage form, vary depending upon the subject being treated, and the particular mode of administration. The amount of the antibody which can be combined with a carrier material to produce a single dosage form generally be that amount of the composition which produces a therapeutic effect. Generally, out of one hundred percent, this amount range from about 0.01 percent to about ninety-nine percent of active ingredient, from about 0.1 percent to about 70 percent, or from about 1 percent to about 30 per cent.

Dosage forms for the topical or transdermal administration of compositions of the present disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

These pharmaceutical compositions can also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In certain embodiments, when the antibodies of the present disclosure are administered as pharmaceuticals, to humans and animals, they can be given alone or as a pharmaceutical composition containing, for example, from about 0.01% to about 99.5% (or about 0.1 to about 90%) of an antibody, described herein, in combination with a pharmaceutically acceptable carrier.

VI. Articles of Manufacture

The presently disclosed subject matter further relates to articles of manufacture materials, e.g., containing one or more of the presently disclosed antibodies, useful for the treatment and/or prevention of the disease and/or disorders described above.

In certain embodiments, the article of manufacture includes a container and a label or package insert on or associated with the container. Non-limiting examples of suitable containers include bottles, vials, syringes, IV solution bags, etc. The containers can be formed from a variety of materials such as glass or plastic. The container can hold a composition which is by itself or combined with another composition effective for treating, preventing and/or diagnosing the condition and may have a sterile access port (for example, the container may be an intravenous solution bag or a vial having a stopper pierceable by a hypodermic injection needle).

In certain embodiments, at least one active agent in the composition is an antibody of the presently disclosed subject matter. The label or package insert can indicate that the composition is used for treating the condition of choice.

In certain embodiments, the article of manufacture can comprise (a) a first container with a composition contained therein, wherein the composition comprises an antibody of the present disclosure; and (b) a second container with a composition contained therein, wherein the composition comprises a further cytotoxic or otherwise therapeutic agent. In certain embodiments, the article of manufacture can further comprise a package insert indicating that the compositions can be used to treat a particular condition.

Alternatively, or additionally, the article of manufacture can further an additional container, e.g., a second or third container, including a pharmaceutically-acceptable buffer, such as, but not limited to, bacteriostatic water for injection (BWFI), phosphate-buffered saline, Ringer's solution and dextrose solution. The article of manufacture can include other materials desirable from a commercial and user standpoint, including other buffers, diluents, filters, needles, and syringes.

VII. Exemplary Embodiments

A. In certain non-limiting embodiments, the presently disclosed subject matter provides for an isolated IgA antibody, or a fragment thereof, wherein the IgA antibody comprises a substitution at amino acid V458, N459 and/or S461.

A1. The foregoing isolated IgA antibody of A, wherein amino acid V458 is substituted with an isoleucine (V4581), amino acid N459 is substituted with a glutamine (N459Q), a glycine (N459G) or an alanine (N459A), and/or amino acid S461 is substituted with an alanine (S461A).

B. In certain non-limiting embodiments, the presently disclosed subject matter provides for an isolated IgA antibody, or a fragment thereof, wherein the IgA antibody comprises a substitution at amino acid I458.

B1. The foregoing isolated IgA antibody of B, wherein amino acid I458 is substituted with a valine (I458V).

C. In certain non-limiting embodiments, the presently disclosed subject matter provides for an isolated IgA antibody, or a fragment thereof, wherein the IgA antibody comprises one or more substitutions at an amino acid selected from the group consisting of N166, T168, N211, S212, S213, N263, T265, N337, I338, T339, N459, S461 and a combination thereof.

C1. The foregoing isolated IgA antibody of C, wherein the substitutions at amino acids N166, S212, N263, N337,1338, T339 and N459 are N166A, S212P, N263Q, N337T, I338L, T339S and N459Q.

C2. The foregoing isolated IgA antibody of any one of A-C1, wherein the IgA antibody is an IgA1, IgA2m1, IgA2m2 or IgA2mn antibody.

C3. The foregoing isolated IgA antibody of any one of C and C1, wherein the IgA antibody has substitutions at amino acids N337, 1338 and T339 and one or more substitutions at T168, N211, S212, S213, N263, T265, N459, S461 and a combination thereof.

C4. The foregoing isolated IgA antibody of C3, wherein the IgA antibody is an IgA2m1, IgA2m2 or IgA2mn antibody.

C5. The foregoing isolated IgA antibody of any one of A-C4, wherein the IgA antibody is humanized, a chimeric antibody or human antibody.

D. In certain non-limiting embodiments, the presently disclosed subject matter provides for an isolated IgG-IgA fusion molecule comprising a full-length IgG antibody fused at its C-terminus to an Fc region of an IgA antibody, wherein the Fc region of the IgA antibody comprises a sequence comprising P221 or R221 through the C-terminus of the heavy chain of the IgA antibody, and wherein the IgG antibody further comprises a deletion of amino acid K447.

E. In certain non-limiting embodiments, the presently disclosed subject matter provides for an isolated IgG-IgA fusion molecule comprising a full-length IgG antibody fused at its C-terminus to an Fc region of an IgA antibody, wherein the Fc region of the IgA antibody comprises a sequence comprising C242 through the C-terminus of the heavy chain of the IgA antibody.

E1. The foregoing isolated IgG-IgA fusion molecule of E, wherein the IgG antibody further comprises a deletion of amino acid K447.

E2. The foregoing isolated IgG-IgA fusion molecule of any one of D-E1, wherein the IgG antibody is selected from the group consisting of an IgG1 antibody, an IgG2 antibody, an IgG3 antibody and an IgG4 antibody.

E3. The foregoing isolated IgG-IgA fusion molecule of any one of D-E2, wherein the IgG antibody is an IgG1 antibody.

E4. The foregoing isolated IgG-IgA fusion molecule of any one of D-E3, wherein the IgA antibody is selected from the group consisting of an IgA1 antibody, an IgA2m1 antibody, an IgA2m2 antibody and an IgA2mn antibody.

E5. The foregoing isolated IgG-IgA fusion molecule of any one of D-E4, wherein the IgA antibody is an IgA2m1 antibody.

F. In certain non-limiting embodiments, the presently disclosed subject matter provides for an isolated nucleic acid encoding the IgA antibody of any one of A-C4 or the IgG-IgA fusion molecule of any one of D-E5.

G. In certain non-limiting embodiments, the presently disclosed subject matter provides for a host cell comprising the nucleic acid of F.

H. In certain non-limiting embodiments, the presently disclosed subject matter provides for a method of producing an IgA antibody or IgG-IgA comprising culturing the host cell of G so that the IgA antibody or IgG-IgA fusion molecule is produced.

H1. The foregoing method of H, further comprising recovering the IgA antibody or IgG-IgA fusion molecule from the host cell.

H2. The foregoing IgA antibody or IgG-IgA fusion molecule produced from H or recovered from H1.

I. In certain non-limiting embodiments, the presently disclosed subject matter provides for A pharmaceutical composition comprising one or more IgA antibodies of any one of A-C4 and H2, or one or more IgG-IgA fusion molecules of any one of D-E5 and H2 and a pharmaceutically acceptable carrier.

. The foregoing pharmaceutical composition of I, further comprising an additional therapeutic agent.

J. In certain non-limiting embodiments, the presently disclosed subject matter provides for a method of treating an individual having a disease, wherein the method comprises administering to the individual an effective amount of one or more IgA antibodies of any one of A-C4 and H2, or one or more IgG-IgA fusion molecules of any one of D-E5 and H2.

J1. The foregoing method of J, wherein the disease is an inflammatory disease, an autoimmune disease or cancer.

K. The foregoing IgA antibody of any one of A-C4 and H2 or the IgG-IgA fusion molecule of any one of D-E5 and H2for use as a medicament.

L. The foregoing IgA antibody of any one of A-C4 and H2 or the IgG-IgA fusion molecule of any one of D-E5 and H2 for use in treating a disease.

M. The foregoing IgA antibody or IgG-IgA fusion molecule of L, wherein the disease is an inflammatory disease, an autoimmune disease or cancer.

N. In certain non-limiting embodiments, the presently disclosed subject matter provides for a use of the IgA antibody of any one of A-C4 and H2 or the IgG-IgA fusion molecule of any one of D-E5 and H2 in the manufacture of a medicament for treatment of a disease.

N1. The foregoing use of N, wherein the disease is an inflammatory disease, an autoimmune disease or cancer.

O. In certain non-limiting embodiments, the presently disclosed subject matter provides for a method of increasing the expression of IgA dimers comprising increasing the amount of DNA encoding a joining chain (JC) that is introduced into a first cell relative to the amount of DNA that encodes the light chain (LC) and the heavy chain (HC), wherein increased expression is relative to the amount of IgA dimers produced in a second cell introduced with equal amounts of JC, LC and HC DNA.

O1. The foregoing method of O, wherein the ratio of the amount of DNA encoding the HC to the amount of DNA encoding the LC to the amount of DNA encoding the JC (HC:LC:JC) that is introduced into the first cell is from about 1:1:2 to about 1:1:5.

P. In certain non-limiting embodiments, the presently disclosed subject matter provides for a method of increasing the expression of IgA dimers, trimers or tetramers comprising decreasing the amount of DNA encoding a joining chain (JC) introduced into a first cell relative to the amount of DNA that encodes the light chain (LC) and the heavy chain (HC), wherein increased expression is relative to the amount of IgA trimers or tetramers produced in a second cell introduced with greater amounts of HC and LC DNA relative to the amount of JC DNA.

P 1. The foregoing method of P, wherein the ratio of the amount of DNA encoding the HC to the amount of DNA encoding the LC to the amount of DNA encoding the JC (HC:LC:JC) that is introduced into the first cell is from about 1:1:0.25 to about 1:1:0.5.

Q. In certain non-limiting embodiments, the presently disclosed subject matter provides for a method of increasing the production of IgA1 or IgA2m1 polymers comprising expressing, in a first cell, an IgA1 or IgA2m1 antibody having a substitution at amino acid V458, wherein increased production is relative to the amount of IgA1 or IgA2m1 polymers produced in a second cell expressing an IgA1 or IgA2m1 antibody that does not have a substitution at amino acid V458.

Q1. The foregoing method of Q, wherein amino acid is substituted with an isoleucine (V4581).

R. In certain non-limiting embodiments, the presently disclosed subject matter provides for a method of increasing the production of IgA2m2 dimers comprising expressing, in a first cell, an IgA2m2 antibody having a substitution at amino acid I458, wherein increased production is relative to the amount of IgA2m2 dimers s produced in a second cell expressing an IgA2m2 antibody that does not have a substitution at amino acid I458.

R1. The foregoing method of R, wherein amino acid is substituted with a valine (I458V).

S. In certain non-limiting embodiments, the presently disclosed subject matter provides for a method of increasing the production of an IgA1 or IgA2m1 polymer comprising expressing, in a first cell, an IgA1 or IgA2m1 antibody having a substitution at amino acid N459 or S461, wherein increased production is relative to the amount of IgA1 or IgA2m1 polymers produced in a second cell expressing an IgA1 or IgA2m1 antibody that does not have a substitution at amino acid N459 or S461.

S1. The foregoing method of S, wherein amino acid N459 is substituted with a N459Q, N459G or a N459A mutation and/or amino acid S461 is substituted with a S461A mutation.

T. A method of decreasing the production of IgA2m2 polymers comprising expressing, in a first cell, an IgA2m2 antibody with a substitution at amino acid C471, wherein decreased production is relative to the amount of IgA2m2 polymers produced in a second cell expressing an IgA2m2 antibody that does not have a substitution at amino acid C471.

T1. The foregoing method of T, wherein amino acid C471 is substituted with a C471S mutation.

U. In certain non-limiting embodiments, the presently disclosed subject matter provides for a method of increasing transient expression of an IgA2m2 antibody comprising expressing, in a first cell, an IgA2m2 antibody that comprises a substitution at an amino acid selected from the group consisting of N166, S212, N263, N337, I338, T339, N459 and a combination thereof, wherein increased transient expression is relative to the amount of transient expression produced in a second cell expressing an IgA2m2 antibody that does not have a substitution at an amino acid selected from the group consisting of N166, S212, N263, N337, I338, T339, N459 and a combination thereof.

V. In certain non-limiting embodiments, the presently disclosed subject matter provides for a method of expressing dimers of IgG-IgA fusion molecules comprising expressing an IgG-IgA fusion molecule comprising a full-length IgG antibody fused at its C-terminus to an Fc region of an IgA antibody, wherein the Fc region of the IgA antibody comprises a sequence comprising P221 or R221 through the C-terminus of the heavy chain of the IgA antibody, and wherein the IgG antibody comprises a deletion of amino acid K447.

W. In certain non-limiting embodiments, the presently disclosed subject matter provides for a method of expressing dimers, trimers or tetramers of IgG-IgA fusion molecules comprising expressing an IgG-IgA fusion molecule comprising a full-length IgG antibody fused at its C-terminus to an Fc region of an IgA antibody, wherein the Fc region of the IgA antibody comprises a sequence comprising C242 through the C-terminus of the heavy chain of the IgA antibody.

W1. The foregoing method of W, wherein the IgG antibody comprises a deletion of amino acid K447.

X. In certain non-limiting embodiments, the presently disclosed subject matter provides for a method for purifying an IgA antibody from a mixture comprising an IgA antibody and at least one host cell protein comprising:

(a) applying the mixture to a column comprising Protein L to bind the IgA antibody;

(b) washing the Protein L column with a wash buffer comprising PBS; and

(c) eluting the IgA antibody from the Protein L column by an elution buffer comprising phosphoric acid.

Y. In certain non-limiting embodiments, the presently disclosed subject matter provides for a method for purifying an oligomeric state of an IgA antibody or an IgG-IgA fusion molecule from a mixture comprising an IgA antibody or an IgG-IgA fusion molecule and at least one host cell protein comprising:

(a) applying the mixture to an affinity purification column comprising Protein L or Protein A to bind the IgA antibody or IgG-IgA fusion molecule;

(b) washing the affinity purification column with a wash buffer;

(c) eluting the IgA antibody or IgG-IgA fusion molecule from the affinity purification column by an elution buffer to form a first eluate; and

(d) applying the first eluate to a size exclusion chromatography column to separate different oligomeric states of the IgA antibody or IgG-IgA fusion molecule and to obtain a flowthrough comprising an oligomeric state of the IgA antibody or IgG-IgA fusion molecule.

The following examples are merely illustrative of the presently disclosed subject matter and should not be considered as limitations in any way.

### EXAMPLE 1

### Assessment of the Role of Glycosylation and FcRn Binding on the Pharmacokinetic Parameters of Polymeric IgA In Mice

IgA antibodies have broad potential as a novel therapeutic platform based on their superior receptor-mediated cytotoxic activity, potent neutralization of pathogens, and ability to transcytose across mucosal barriers via polymeric immunoglobulin receptor (pIgR)-mediated transport, as compared to traditional IgG-based drugs. However, the transition of IgA into clinical development has been challenged by complex expression and characterization, as well as rapid serum clearance that is thought to be mediated by glycan receptor scavenging of recombinantly produced IgA monomer bearing incompletely sialylated N-linked glycans. In the present example, a comprehensive biochemical, biophysical and structural characterization of recombinantly produced monomeric, dimeric and polymeric human IgA is provided. In addition, two strategies to overcome the rapid serum clearance of polymeric IgA are identified: (1) removal of N-linked glycosylation sites creating an aglycosylated or partially aglycosylated polymeric IgA and (2) engineering in of FcRn binding with the generation of a polymeric IgG-IgA Fc fusions.

Methods:

Plasmid cloning and sequence alignments. Antibody variable domain sequences used include a humanized anti-human HER2 antibody (Carter et al., Proc Natl Acad Sci USA 89:4285-9 (1992)) and a murine anti-murine IL-13 antibody (Genentech). Protein sequences of human IgA constant heavy chains IgA1, IgA2m1 and IgA2m2, other IgA species and human J chain were obtained from Uniprot (www.uniprot.org) or NCBI (www.ncbi.nlm.nih.gov/protein). Other species that were obtained include a mutation in IgA2m1, i.e., P221R, that stabilizes the light chain-heavy chain disulfide as previously reported (Chintalacharuvu et al., J Immunol 157:3443-9 (1996)). Genes encoding a fusion of the antibody variable domains to the human light chain and human IgA1, IgA2m1 and IgA2m2 heavy chain constant domains were synthesized and cloned into the mammalian pRK vector (Eaton et al., Biochemistry 25:8343-7 (1986)). Site-directed mutagenesis was used to introduce point mutations. All plasmids were sequence verified. Sequence alignments were done using GSeqWeb (Genentech) and Excel (Microsoft).

Small-Scale Antibody Expression and Purification. Expi293T™ cells were transiently transfected at the 30 mL scale with 15 μg of DNA of both LC and HC for IgA monomers or a total of 30 μg of DNA of varying ratios of LC, HC and JC for IgA oligomers (Bos et al., Journal of Biotechnology 180:10-6 (2014) and Bos et al., Biotechnol Bioeng 112:1832-42 (2015)). IgAs were affinity purified in batch with Protein L (GE Healthcare) as all antibodies contained kappa light chains. Protein L eluate was characterized by analytical SEC-HPLC (Tosoh Bioscience LLC TSKgel SuperSW3000 column, Thermo Scientific Dionex UltiMate 3000 HPLC). A constant volume was loaded on the column and the area under each curve was quantitated using Chromeleon Chromatography Data System software (Thermo Scientific).

Large-Scale Antibody Expression and Purification. IgA, IgG and IgG-IgA Fc fusions were transiently expressed in CHO DP12 cells as previously described (Wong et al., Biotechnol Bioeng 106:751-63 (2010)). For low expressing clones, TI stable cell lines were generated. IgG and IgG-IgA Fc fusions were affinity purified using Mab Select Sure (GE Healthcare) followed by size-exclusion chromatography (SEC) with a HiLoad Superdex 200 pg column (GE Healthcare). IgAs were affinity purified using Capto L (GE Healthcare) followed by SEC. For IgA samples where DNA ratios successfully biased expression to mainly one oligomeric state, a HiLoad Superdex 200 pg column (GE Healthcare) was used for SEC. For IgA samples containing complex mixtures of oligomers, a 3.5 μm, 7.8 mm×300 mm Xbridge Protein BEH 450 Å SEC column (Waters) was used for better separation of dimer and tetramer peaks.

SEC-MALS. Polymeric IgAs were run over a 3.5 μm, 7.8 mm×300 mm Xbridge Protein BEH 200 Å SEC column (Waters) and directly injected onto a DAWN HELEOS/Optilab T-rEX II (Wyatt) multi-angle light scattering detector for molar mass determination and polydispersity measurement.

Differential Scanning Fluorimetry (DSF). DSF was performed as described previously (Lombana et al., Sci Rep 5:17488 (2015)).

In vitro Transcytosis Assay. Madin-Darby canine kidney (MDCKII) cells (European Collection of Authenticated Cell Cultures, Salisbury, U.K.) cells were transduced with retrovirus containing cDNA coding for the human pIgR gene (Retro-X, Takara Bio; OriGene, Rockvile, Md.). Expression of the pIgR gene was confirmed by qRT-PCR and Western Blotting. MDCKII cells expressing pIgR were maintained in DMEM supplemented with 10% FBS, 100 U/ml penicillin and 100 μg/ml streptomycin (Thermo Fisher, Carlsbad, Calif.), and 2 μg/ml Puromycin (Takara Bio, Mountain View, Calif.). For the transcytosis assay, cells were seeded on 0.4 μm Millicell 24-well cell culture insert (Millipore, Burlington, Mass.) and cultured for 4 days. On the day of the experiment, the cells were washed twice with FluoroBrite DMEM (Thermo Fisher) and 6μg of IgA molecules were added to the basolateral compartments. After 24-hour of incubation, media from both apical and basolateral compartments were collected for analysis by ELISA.

Electron Microscopy. Purified anti-IL-13 IgA2m2 dimer and tetramer samples were first crosslinked by incubating in 0.015% glutaraldehyde (Polysciences, Inc.) for 10 minutes at room temperature. Once fixed, the samples were diluted using TBS buffer to achieve a concentration of 10 ng/μL. Then 4 μl of each sample were incubated for 40 s on freshly glow discharged 400 mesh copper grids covered with a thin layer of continuous carbon before being treated with 2% (w/v) uranyl acetate negative stain (Electron Microscopy Sciences). IgA dimers and tetramers were then imaged using a Tecnai Spirit T12 (Thermo Fisher) operating at 120 keV, at a magnification of 25,000× (2.2 Å/pixel). Images were recorded using a Gatan 4096×4096 pixel CCD camera under low dose conditions. About 5000 particles for both IgA dimer and tetramers were then selected and extracted using the e2boxer.py software within the EMAN2 package (Tang et al., J Struct Biol 157:38-46 (2007)) using a 128-pixel particle box size. Reference free 2D classification, within the RELION image software package (Scheres J Struct Biol 180:519-30 (2012)) was used to generate averaged images of both samples.

Global N-linked Glycan Composition Analysis (LC-MS analysis). Ten μg of each IgA sample were denatured with 8 M guanidine HCl at 1:1 volume ratio and reduced with 100 mM dithiothreitol (DTT) for 10 min at 95° C. Samples were diluted with 100 mM Tris HCl, pH 7.5, to a final concentration of 2 M guanidine HCl, followed by overnight N-linked deglycosylation at 37° C. with 2 μl of P0705S PNGase F (New England BioLabs). After deglycosylation, 150 ng of each sample were injected onto an Agilent 1260 Infinity LC system and eluted by an isocratic gradient of 2% to 32% solvent B (solvent A: 99.88% water containing 0.1% formic acid and 0.02% trifluoroacetic acid; solvent B: 90% acetonitrile containing 9.88% water plus 0.1% formic acid and 0.02% trifluoroacetic acid). The HPLC system was coupled via an Agilent G4240A Chip Cube MS system to a G6520B Q-TOF mass spectrometer. The samples were glycan enriched and separated using porous graphitized carbon columns built within a G4240-64025 mAb-Glyco chip in the Chip Cube MS system. Data acquisition: 1.9 kV spray voltage; 325° C. gas temperature; 5 l/min drying gas flow; 160 V fragmentor voltage; 65 V skimmer voltage; 750 V oct 1 RF Vpp voltage; 400 to 3000 m/z scan range; positive polarity; MS1 centroid data acquisition using extended dynamic range (2 GHz) instrument mode; 3 spectra/s; 333.3 ms/spectrum; 3243 transients/spectrum; and a CE setting of 0. Acquired mass spectral data were searched against a glycan library in the Agilent MassHunter Qualitative Analysis software utilizing a combination of accurate mass with a mass tolerance of 10 ppm and expected retention time for glycan identification. N-linked glycans were label-free quantified relative to all identified N-linked glycans within each sample based on the AUC in the extracted compound chromatogram of each glycan.

N-linked Glycopeptide Site Mapping Analysis (LC-MS/MS analysis). Ten μg of IgA was reduced with 10 mM DTT at 37° C. for 1 hr, alkylated with 10 mM iodoacetamide at room temperature for 20 minutes, digested with 0.2 μg of trypsin (Promega) and 0.2 μg of chymotrypsin (Thermo Fisher Scientific) separately at 37° C. overnight, quenched with 0.1% trifluoroacetic acid (TFA) and subjected clean up with C18 (3M Empore C18 extraction disks) stage-tip (50% acetonitrile, 49.9% water, 0.1% TFA). 200 fmol of sample were injected onto a Waters NanoAcquity UPLC system via an autosampler and separated at 45° C. on a Waters Acquity M-Class BEH C18 column (0.1 mm×100 mm, 1.7 μm resin). A gradient of 2% to 40% solvent B was used for elution (solvent A: 99.9% water, 0.1% formic acid; solvent B 99.9% acetonitrile, 0.1% formic acid).

Separated peptides were analyzed on-line via nanospray ionization into an Orbitrap Elite mass spectrometer (Thermo Fisher Scientific) using the following parameters for data acquisition: 60000 resolution; 375-1600 m/z scan range; positive polarity; centroid mode; 1 m/z isolation width with 0.25 activation Q and 10 ms activation time; CID activation; and a CE setting of 35. Data was collected in data dependent mode with the precursor ions being analyzed in the FTMS and the top 15 most abundant ions being selected for fragmentation and analysis in the ITMS. Acquired mass spectral data was searched against the protein sequence using Protein Metrics Byonic software and analyzed in Protein Metrics Byologic software. Peptide identification for each glycosylation site was manually validated based on a combination of MS2 fragmentation spectra, extracted ion chromatogram (XIC), and retention time. N-linked glycopeptides were label-free quantified relative to its unmodified peptide by AUC integration of the XICs.

Mouse Studies. Female Balb/C mice (6-8 weeks old) were obtained by Charles River laboratories. Upon arrival, all mice were maintained in a pathogen-free animal facility under a standard 12 h light/12 h dark cycle at 21° C. room temperature with access to food and water ad libitum. All mice received a single intravenous (IV) injection of respective antibody (IgG or IgA). Blood samples (150-200 μL) were collected via either via retro-orbital sinus or cardiac puncture under isoflurane anesthesia at various times post injection. Samples were collected into serum separator tubes. The blood was allowed to clot at ambient temperature for at least 20 minutes. Clotted samples were maintained at room temperature until centrifuged, commencing within 1 hour of the collection time. Each sample was centrifuged at a relative centrifugal force of 1500-2000×g for 5 minutes at 2-8° C. The serum was separated from the blood sample within 20 minutes after centrifugation and transferred into labeled 2.0-mL polypropylene, conical-bottom microcentrifuge tubes.

Only animals that appeared to be healthy and that were free of obvious abnormalities were used for the study. All animal work performed was reviewed and approved by Genentech' s Institutional Animal Care and Use Committee (IACUC).

IgA ELISA for transcytosis and pharmacokinetic studies. IgA antibody levels were measured by sandwich ELISA. Wells of 384-microtiter plates were coated overnight at 4° C. with 2 μg/ml of goat anti-human Kappa antibody (SouthernBiotech, Cat # 2060-01) in 25 μl of coating buffer (0.05 M sodium carbonate, pH 9.6), followed by blocking with 50 μl of 0.5% BSA in PBS for 2 hours at 37° C. Samples (25 μl) diluted in sample buffer (1× PBS, pH 7.4, 0.5% BSA, 0.35 M NaCl, 0.05% Tween20, 0.25% CHAPS, 5 mM EDTA) were then added to the blocked plates and incubated for 2 hours at room temperature. After incubation, 25 μl of horseradish peroxidase-conjugated goat anti-human IgA (SouthernBiotech, Cat # 2053-05) were added and incubated for 1 hour at room temperature. The plates were then incubated with 25 μl of TMB (Moss, Cat #TMBE-1000) for 15 min and the reaction was stopped with 25 μl 1M H₃PO₄. Absorbance was measured at 450 nm with reduction at 630 nm using a plate reader. In between steps, plates were washed six times with 200 μl of washing buffer (0.05% Tween-20 in PBS). As a reference for quantification, a standard curve was established using serially diluted stock material (20 ng/ml-0.15 ng/ml) for each IgA molecule. The IgA ELISA tolerates biological matrices up to 10% mouse serum and 10% tissue lysates.

Radiochemistry. Iodine-125 [¹²⁵I] was obtained as sodium iodide in 0.1 N sodium hydroxide from Perkin Elmer (Boston, Mass.). 1 mCi of ¹²⁵I (˜3 μL) was used to label randomly through tyrosine residues at a specific activity of ˜10 μCi/μg with ¹²⁵I using the indirect Iodogen method (Pierce Chemical Co., Rockford, Ill.). Radiosynthesis of ¹¹¹In labeled antibodies (˜8 μCi/μg) was achieved through incubation of ¹¹¹In and 1,4,7,10-tetraazacyclododecane-1,4,7,10-tetraacetic acid (DOTA)-conjugated (randomly through lysines) mAb in 0.3 M ammonium acetate pH 7 at 37° C. for 1 hour. Purification of all radioimmunoconjugates was achieved using NAPS columns equilibrated in PBS and confirmed by size-exclusion chromatography.

Antibodies were radioiodinated using an indirect iodogen addition method (Chizzonite et al., J Immunol; 147:1548-56 (1991)). The radiolabeled proteins were purified using NAP5™ columns (GE Healthcare Life Sciences, cat. 17-0853-01) pre-equilibrated in PBS. Following radioiodination, the labeled antibodies were characterized by SEC-HPLC to compare to the unlabeled antibodies. Samples were injected onto an Agilent 1100 series HPLC (Agilent Technology, Santa Clara, Calif.) and a Yarra SEC-3000, 3 μM 300 mm×7.8 mm (Phenomenex, Torrance, Calif., cat. 00H-4513-K0) size exclusion columns connected in series and eluted with Phosphate Buffer Saline (PBS pH 7.0) at a flow rate of 0.8 mL/min for 20 minutes. Elution was monitored by absorption at 280 nm and by measuring the radioactivity of the eluted fractions in an in-line Gabi gamma counter (Elysia-Raytest, Germany).

Tissue Distribution study design and analysis. The protocol, housing, and anesthesia were approved by the Institutional Animal Care and Use Committees of Genentech Laboratory Animal Resources, in compliance with the Association for Assessment and Accreditation of Laboratory Animal Care regulations.

Female BALB-c mice in a 20-30 g body weight range and 6-7 weeks age range were obtained from Jackson/West (CA). Six groups of 12 mice each were used for this study. To prevent thyroid sequestration of ¹²⁵I, 100 μL of 30 mg/mL of sodium iodide was intraperitoneally administered 1 and 24 hours prior to dosing. All mice received a single IV injection consisting of a mixture of ¹²⁵I - and ¹¹¹In-labeled antibodies (5 μCi of each) plus the respective unmodified antibody for a total dose of 5 mg/kg. Cohorts of 4 mice were bled retro-orbitally under Isoflurane (inhalation to effect) at 5 min, 15 min, 30 min, 1 hr, 4 hrs, 12 hrs, 1 day, 2 days, and 3 days after injection. At 1 hour, 1 day, and 3 days; 4 animals were euthanized under anesthesia of ketamine (75-80 mg/kg)/xylene (7.5-15 mg/kg) by thoracotomy. The following tissues collected, rinsed in cold PBS, blotted dry, weighed and frozen: Brain, liver, lung, kidney, spleen, heart, stomach, small intestine, muscle, skin, fat, large intestine. Sample radioactivity was counted for radioactivity using a 1480 WIZARD™ Gamma Counter in the energy windows for ¹¹¹In (245 key; decay t_{1/2}=2.8 days) and ¹²⁵I (35 keV; decay t_{1/2}=59.4 days) with automatic background and decay correction. Data were analyzed and graphed using GraphPad Prism (version 7.00 for Windows, GraphPad Software, San Diego Cali. USA, www.graphpad.com).

Mouse Plasma Stability. Mouse plasma (with anti-coagulant Lithium Heparin) was obtained from BioIVT (Westbury, N.Y.) and a buffer control was made by mixing Bovine Serum Albumin (Sigma-Aldrich; St. Louis, Mo., cat. A2058) with PBS (PBS+0.5% BSA). Radiolabeled antibodies were mixed into mouse plasma or buffer control at 5 μCi of radiolabeled tracer and then was incubated in an incubator set at 37° C. with 5% CO₂. At set time point of 0, 24, and 96 hours of incubation, the samples were removed from the incubator and stored at −80° C. freezer until analysis.

The samples were analyzed by SEC-HPLC method described above with a 1:1 sample dilution in PBS. The result chromatograms were compared between the time points to monitor the changes from the parent peak at time zero.

Antibody Kinetics by Wasatch. A 96×96 array-based SPR imaging system (Carterra USA) was used to analyze the kinetics at 25° C. of purified IgA, IgG-IgA Fc fusions or IgG. Antibodies were diluted at 10 μg/ml in 10 mM sodium acetate buffer pH 4.5 and using amine coupling, were directly immobilized onto a SPR sensorprism CMD 200M chip (XanTec Bioanalytics, Germany) using a Continuous Flow Microspotter. Antigens diluted in running buffer (10 mM HEPES pH 7.4, 150 mM NaCl, 0.05% Tween 20, 1 mM EDTA) were injected at various concentrations for 3 minutes and allowed to dissociate for 10 minutes, with regeneration between cycles using 10 mM glycine pH 2.5. Antigens were from R&D Systems (mIL-13, 413-ML-025/CF; mpIgR, 2800-PG-050; hpIgR, 2717-PG-050; hFcαRT, 3939-FA-050), Sino Biologicals (hHER2, 10004-H08H), or Genentech, co-expressed with species specific βeta-2 microglobulin (m/hFcRn). The data was processed with the Wasatch kinetic software tool.

Antibody Kinetics by Biacore. The binding kinetics of the anti-IL-13 or anti-HER2 IgA2m2 antibodies was measured using surface plasmon resonance on a Biacore T200 instrument (GE Healthcare). All kinetics experiments were performed at a flow rate of 30 μL/min, at 25° C., and with a running buffer of 10 mM HEPES pH 7.4, 150 mM NaCl, 0.05% Tween 20, and 1 mM EDTA. Fab Binder from the Human Fab Capture Kit (GE Healthcare) was immobilized on a CM5 sensor chip via amine-based coupling. IgA antibodies with a concentration of 50-100 ug/mL were captured at 5 uL/min for 210 seconds. Recombinant human FcαRI antigen (R&D Systems, 3939-FA-050) binding to the antibody was measured using concentrations of 1000 nM, 333 nM, and 111 nM. Sensorgrams for binding were recorded using an injection time of 90 seconds followed by 120 seconds of dissociation time and regeneration of the surface between cycles with two 60 second injections of glycine pH 2.1. A 1:1 Langmuir binding model was used to calculate the kinetics and binding constants.

Results:

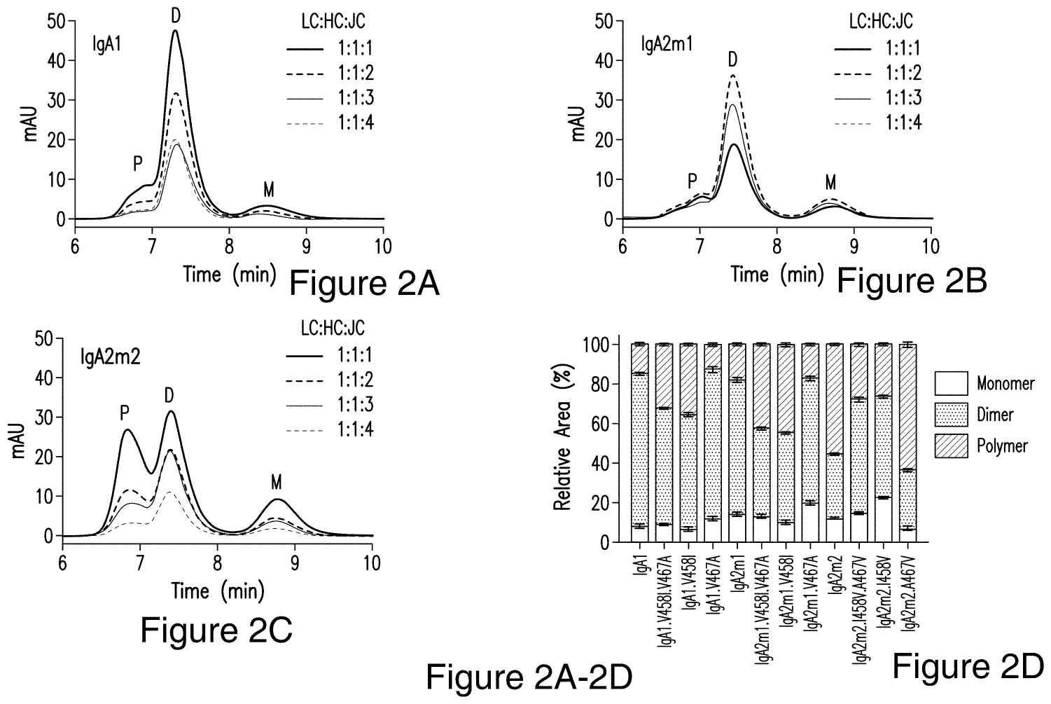

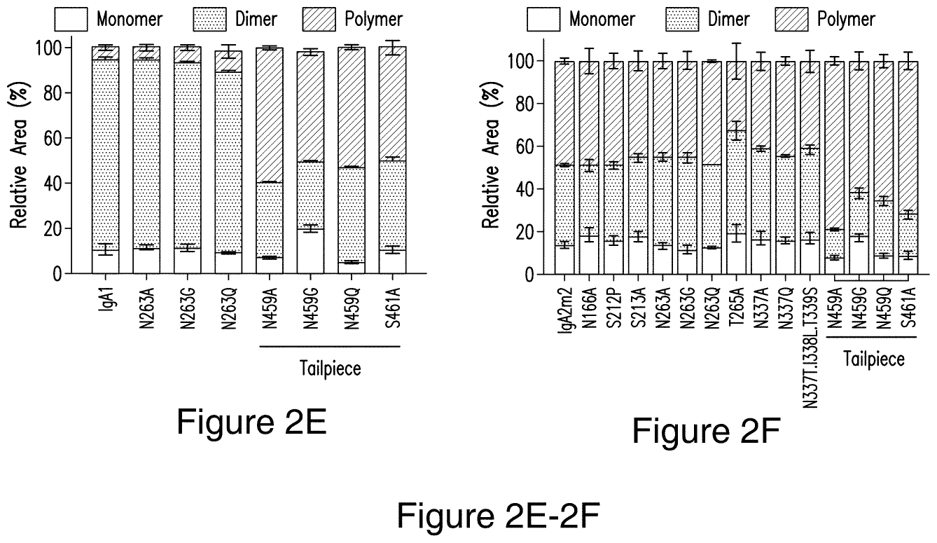

Factors Affecting IgA Oligomer Formation. Recombinant production of monomeric IgA is well understood and can be achieved by coexpression of light chain (LC) and heavy chain (HC), similar to the production of IgG. The assembly of polymeric IgA, in contrast, requires coexpression of LC, HC and joining chain (JC) and the resulting IgA oligomeric states are less well characterized. To gain a better understanding of the assembly process of IgA oligomers, the expression of various human IgA isotypes and allotypes, including IgA1, IgA2m1, IgA2m1.P221R (disulfide stabilized LC-HC pairing) and IgA2m2 (FIG. 1A) were characterized. The murine variable domains of an anti-mouse interleukin-13 (mIL-13) antibody were cloned as chimeras with the human kappa LC and IgA HC constant domains. The chimeric LCs and HCs were then coexpressed in presence and absence of the human JC (FIG. 1B). After affinity purification with Protein L, IgA produced in the absence of cotransfected JC yielded relatively pure monomer from 30 mL Expi293T transient expressions. In these experiments, cells were transfected with equal mass quantities of LC and HC DNA. In contrast, transfection of equal mass quantities of LC, HC and JC DNA produced a variety of oligomeric species, corresponding to IgA monomer, dimer, and polymer that contains three to five IgA monomers (FIG. 1D and FIG. 2A-C). IgA1, IgA2m1 and IgA2m1.P221R were found to produce predominantly dimeric IgA (FIG. 2A-B), while IgA2m2 produced roughly equal amounts of dimer and polymer (FIG. 2C). A similar distribution of oligomers was observed in CHO transient expressions upon scale up to the liter scale.

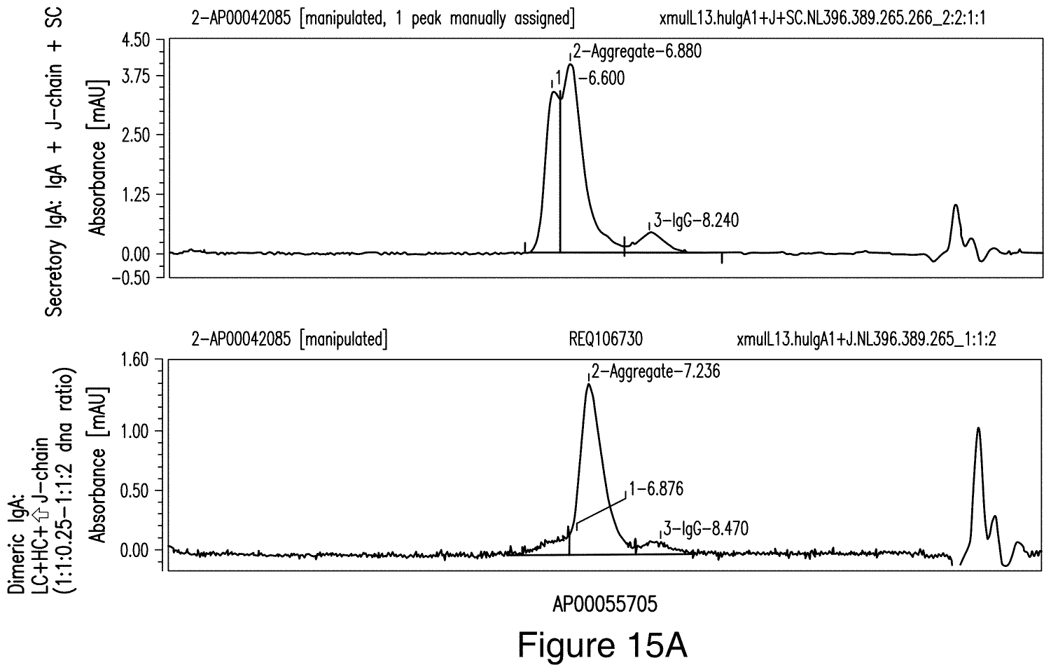

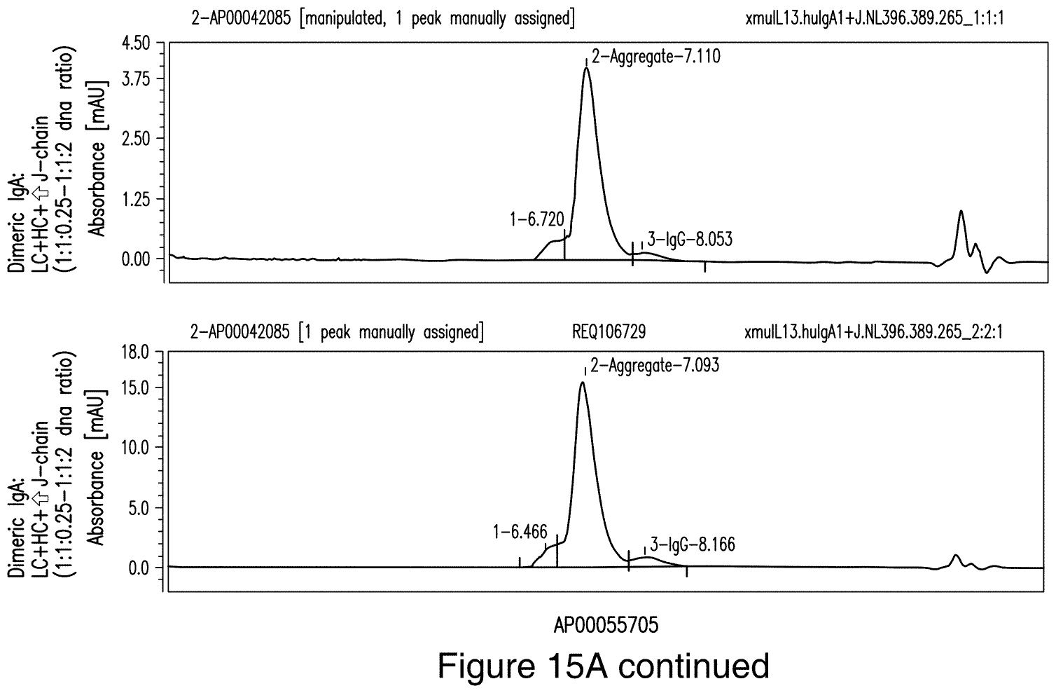

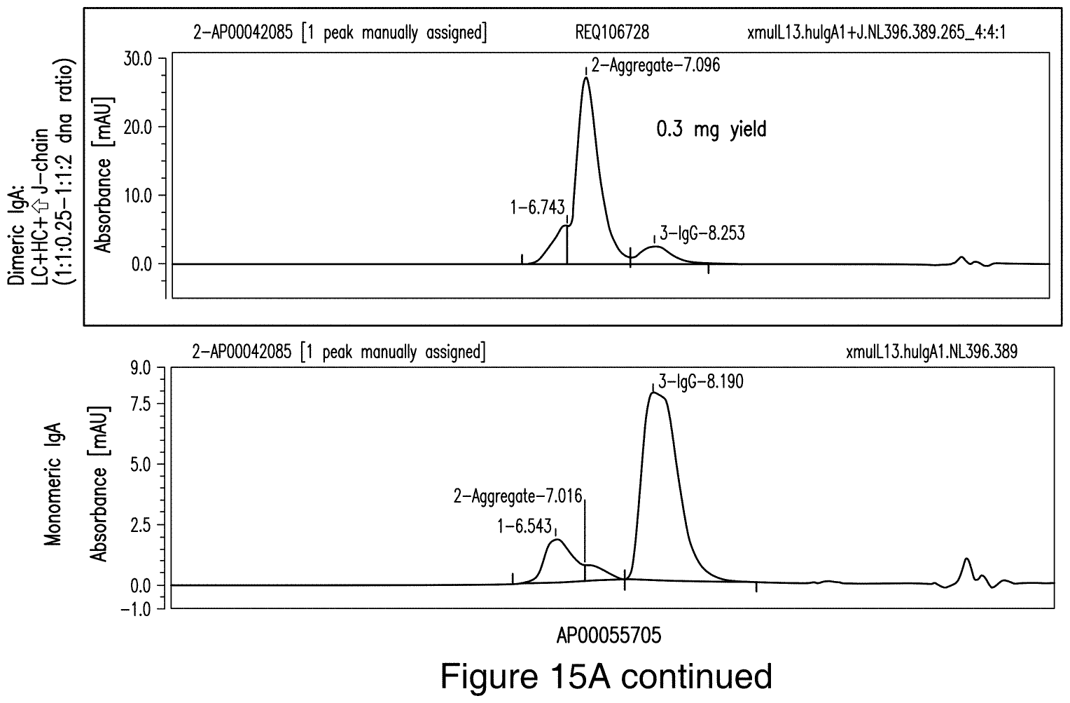

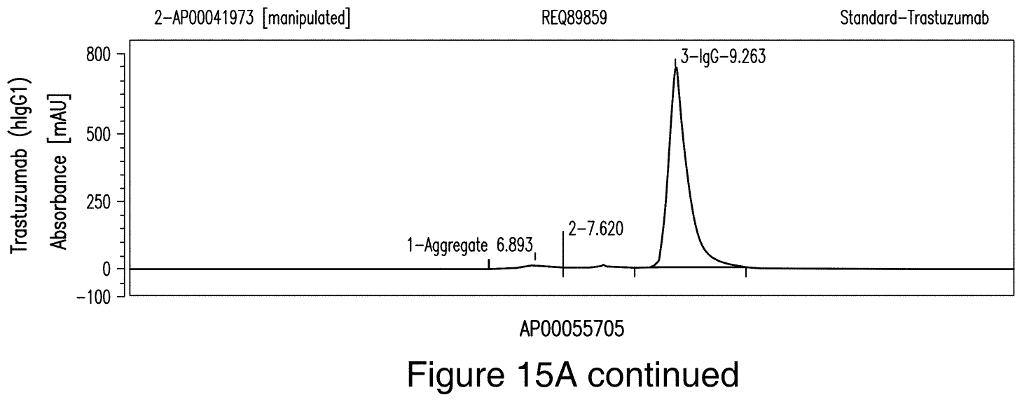

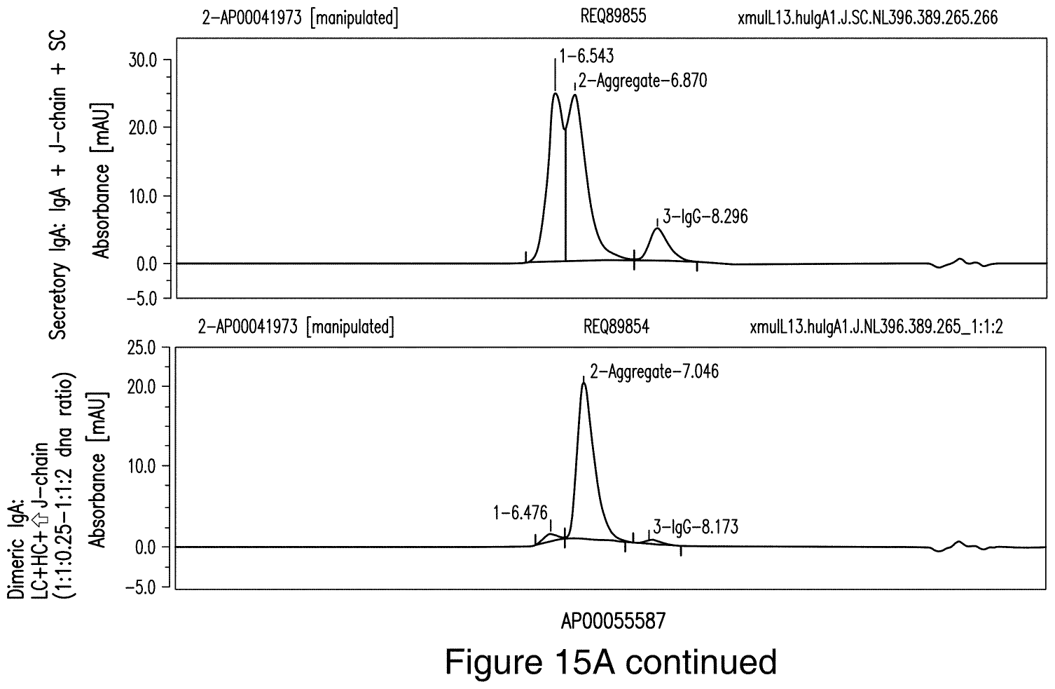

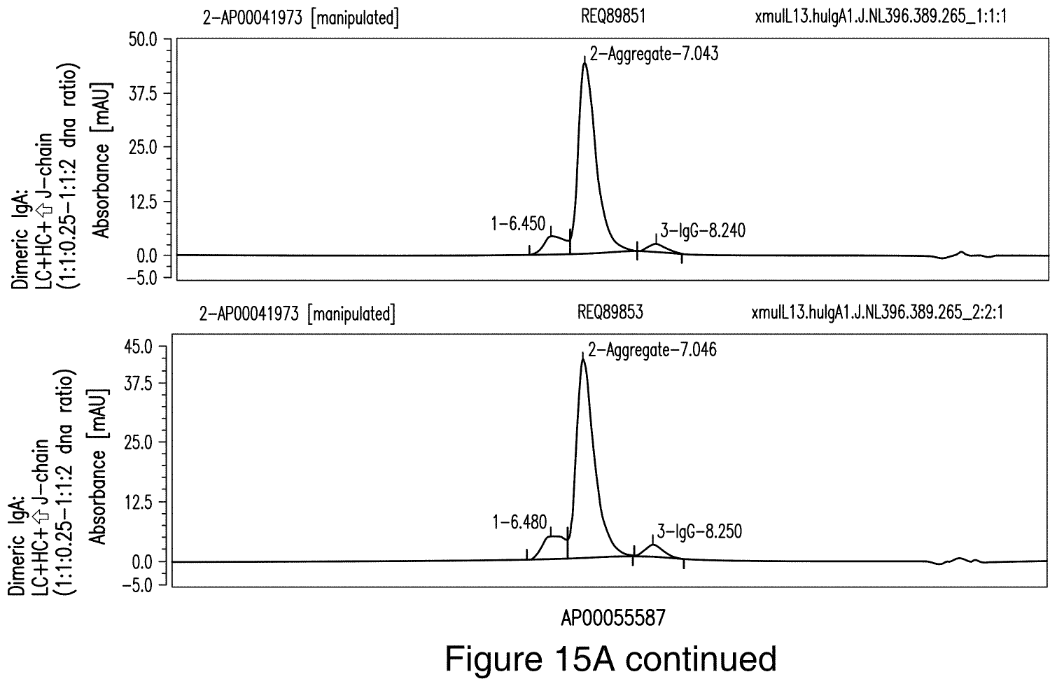

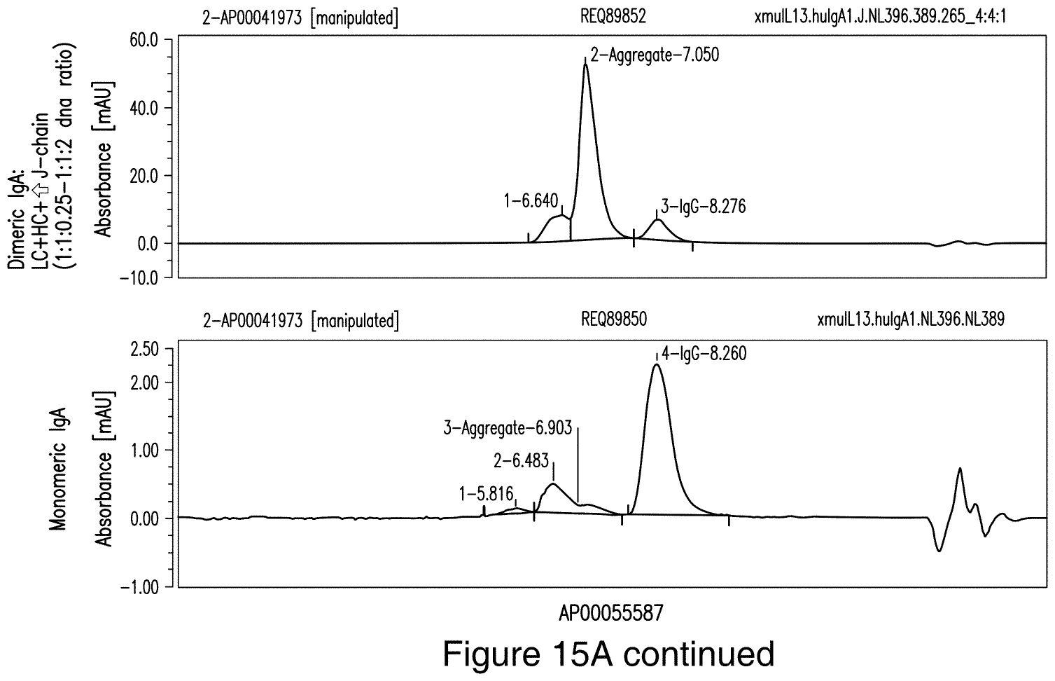

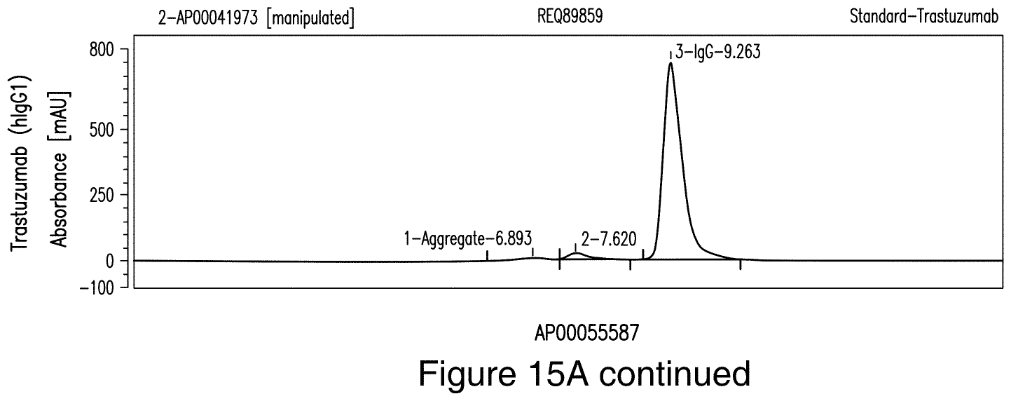

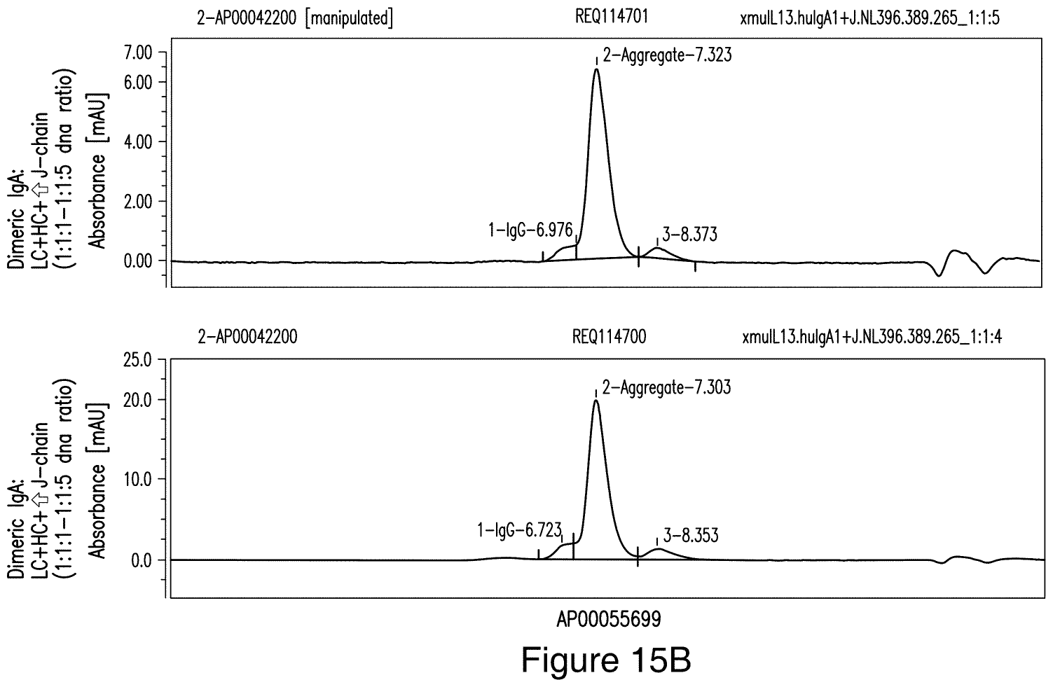

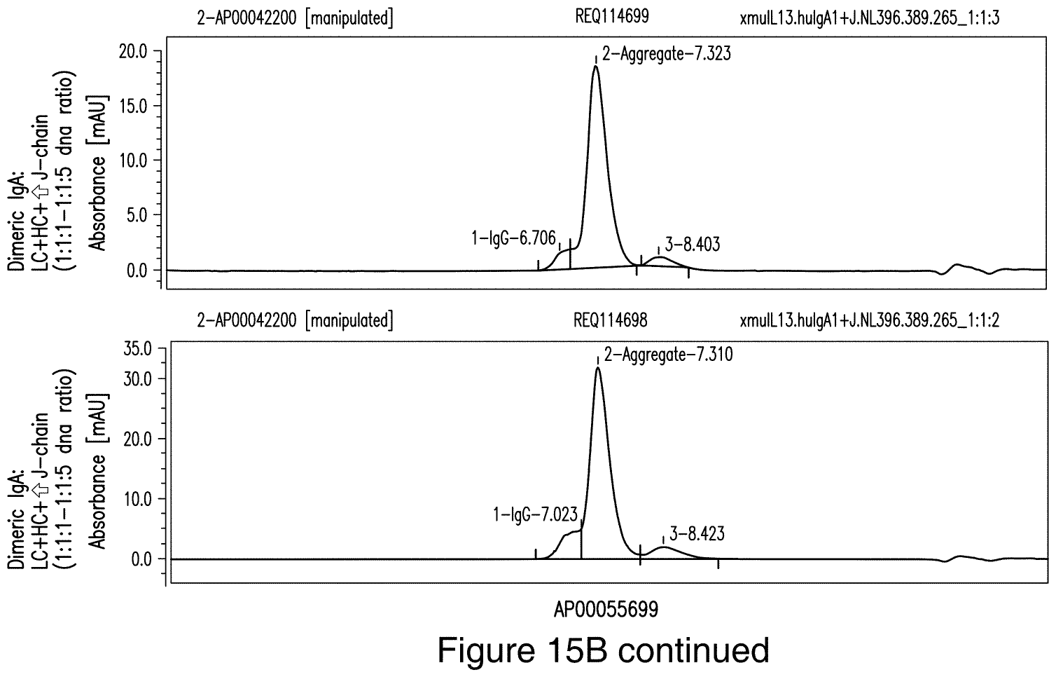

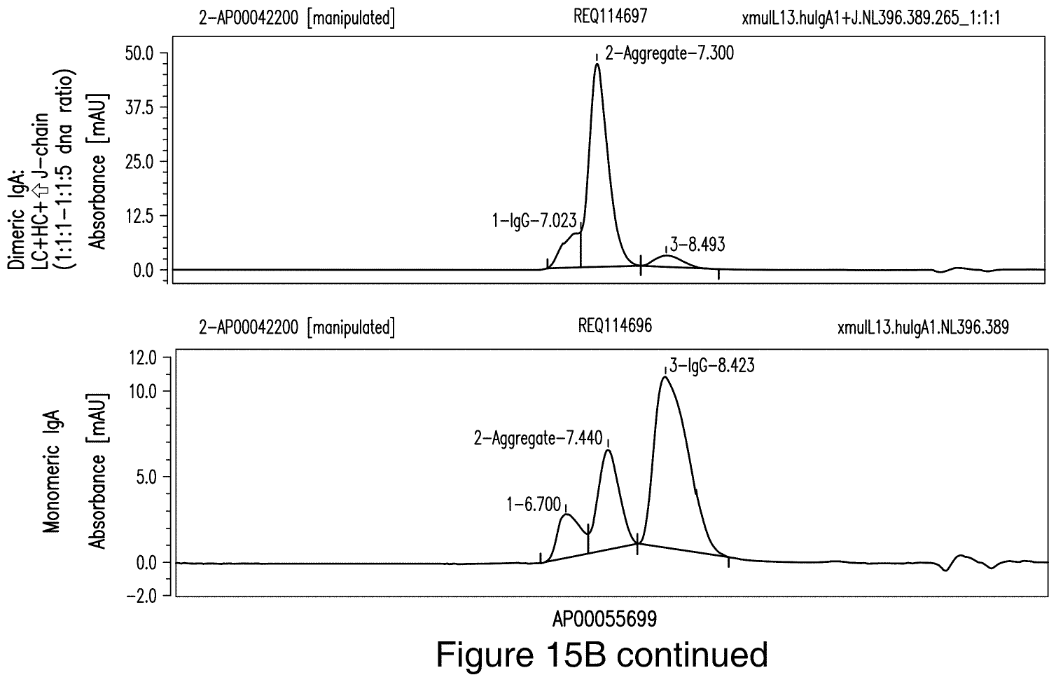

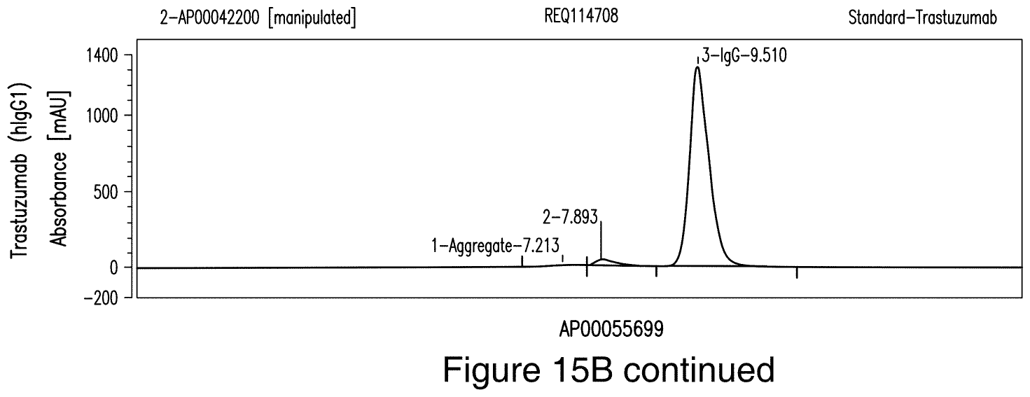

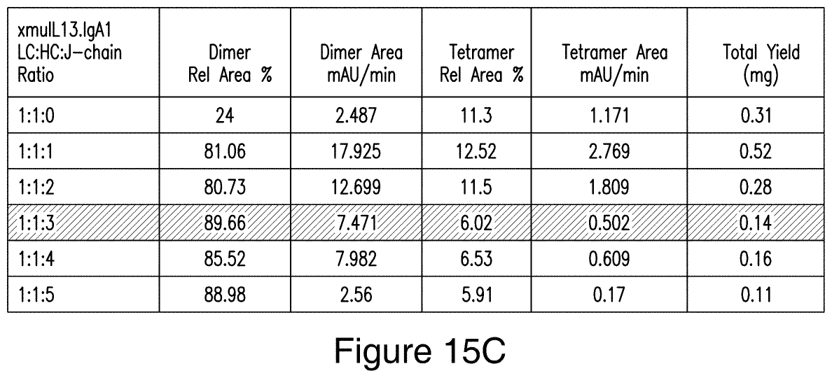

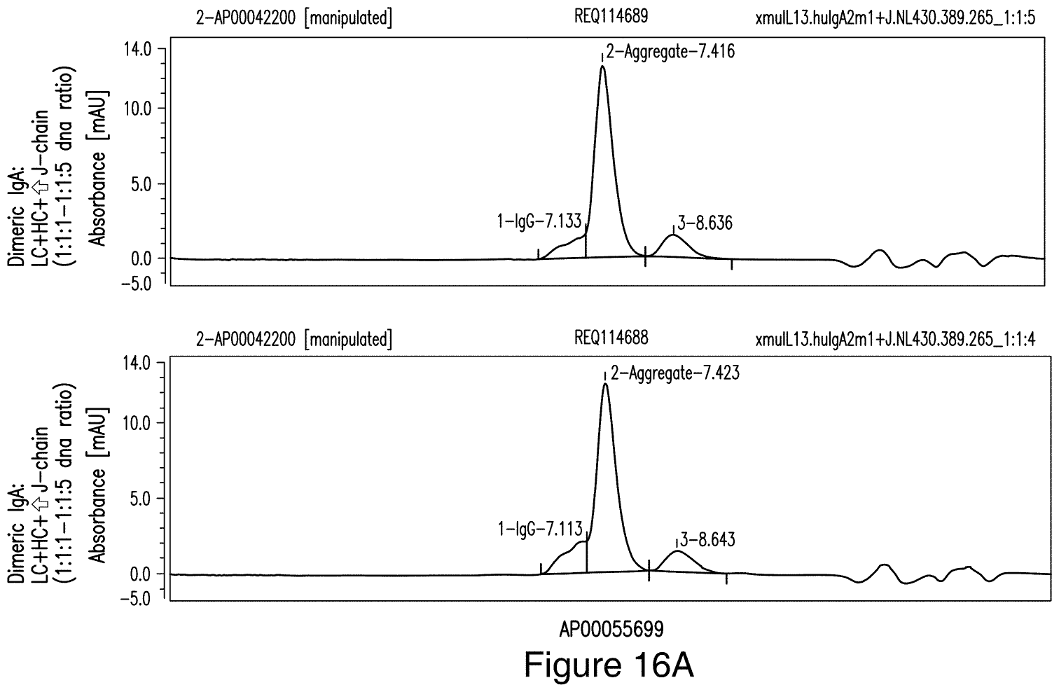

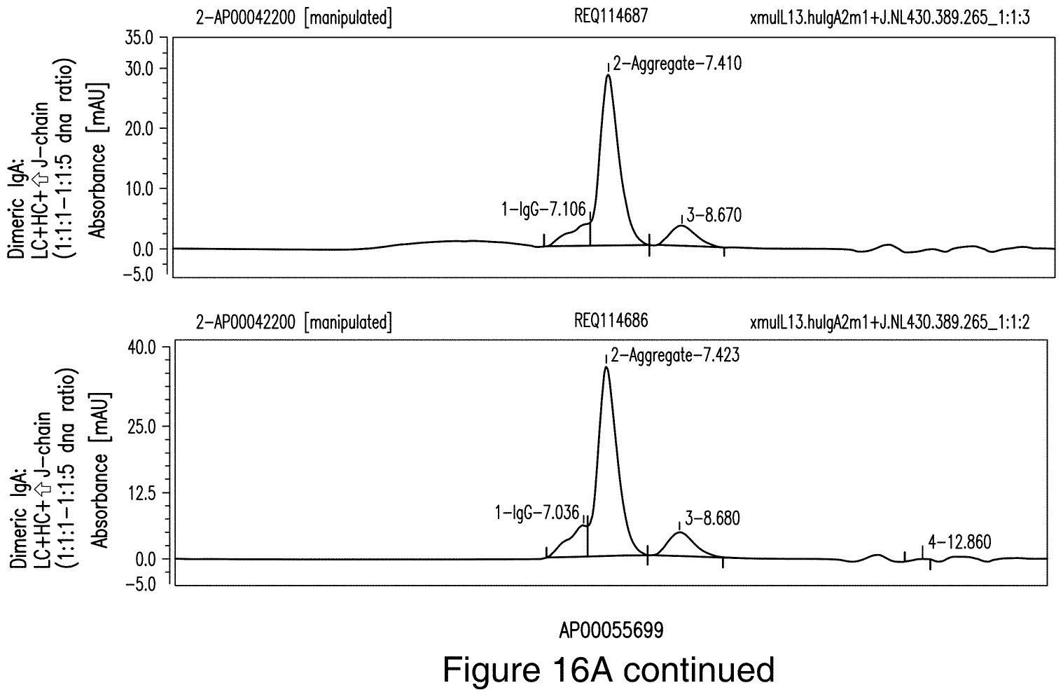

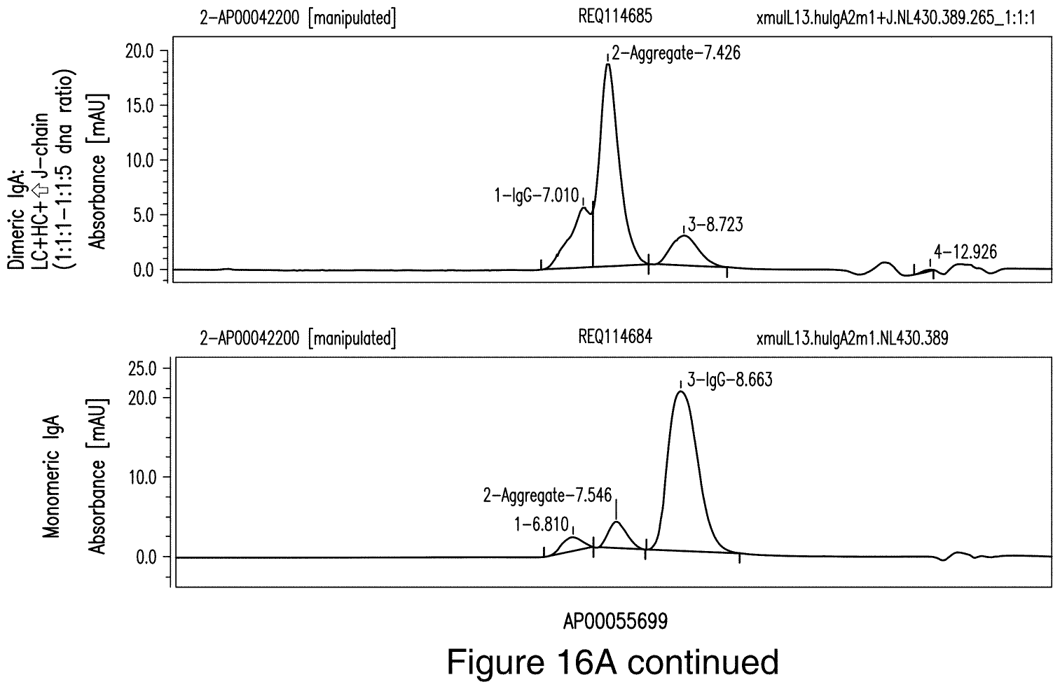

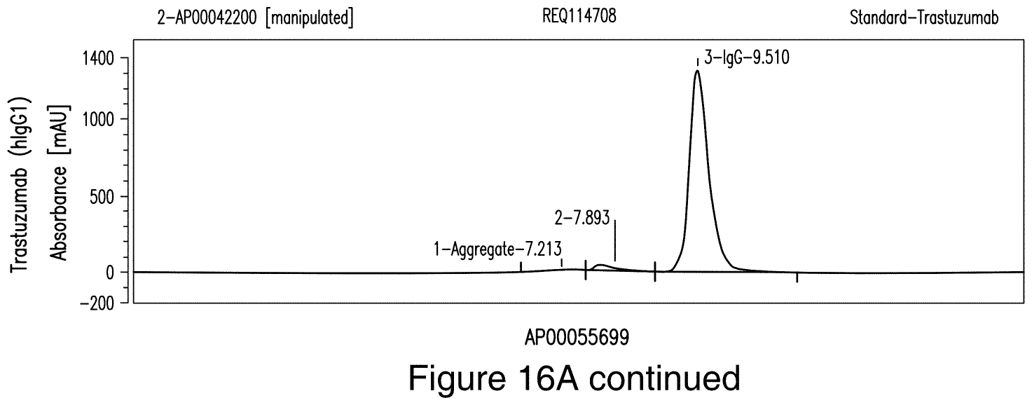

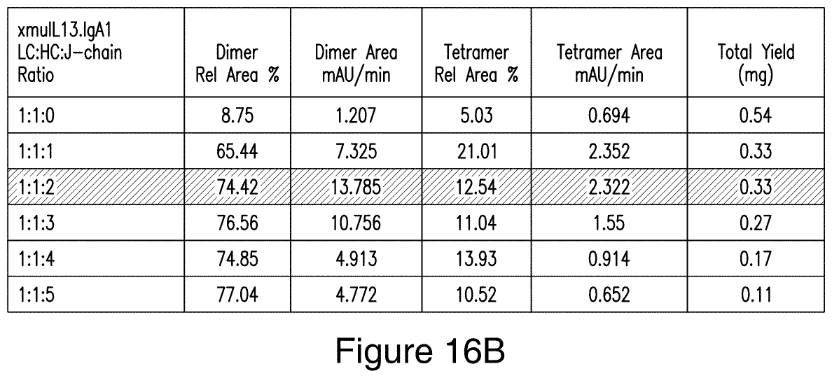

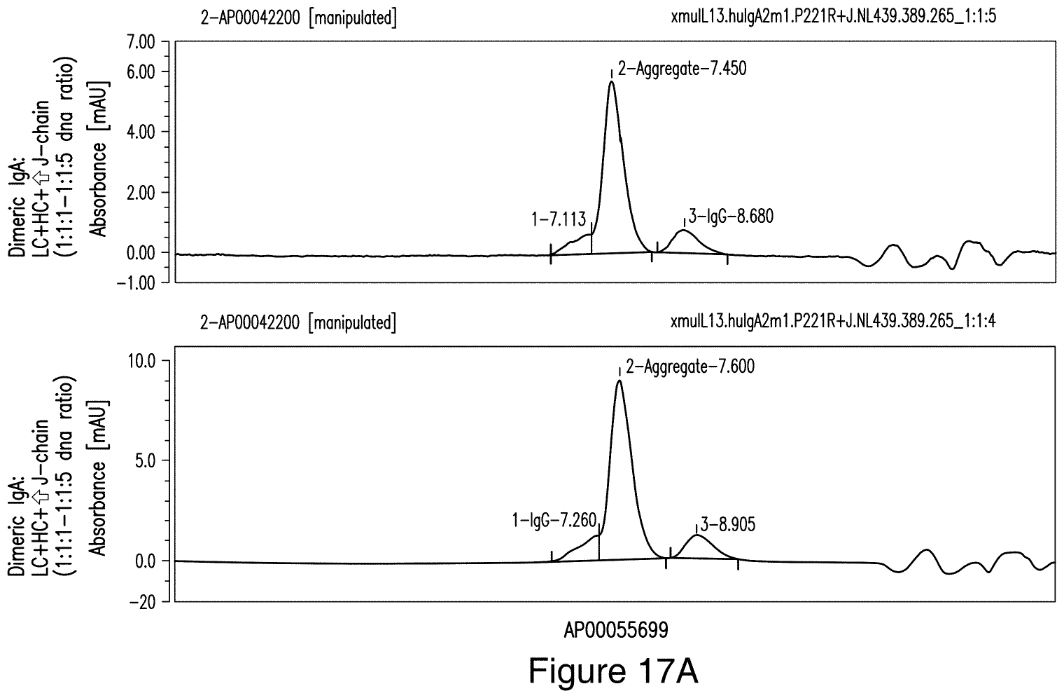

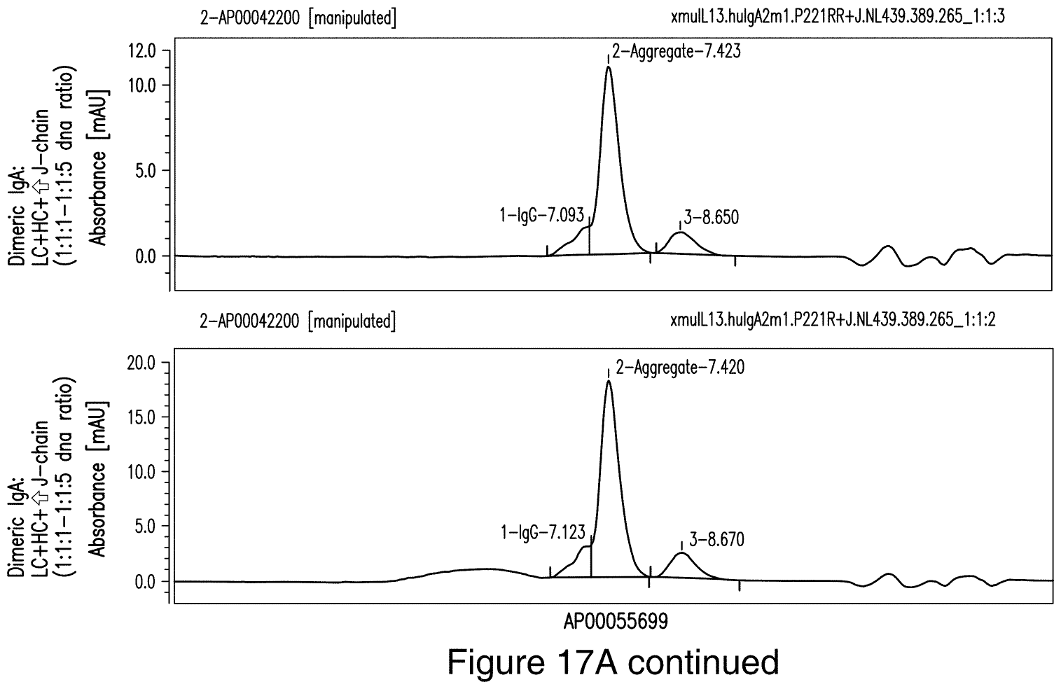

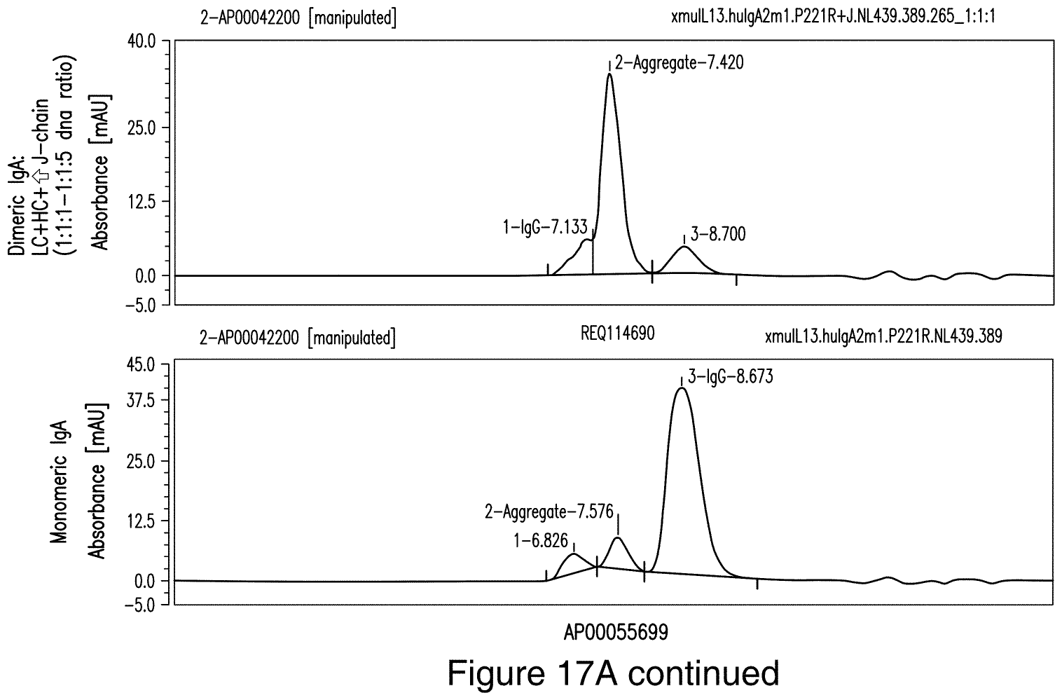

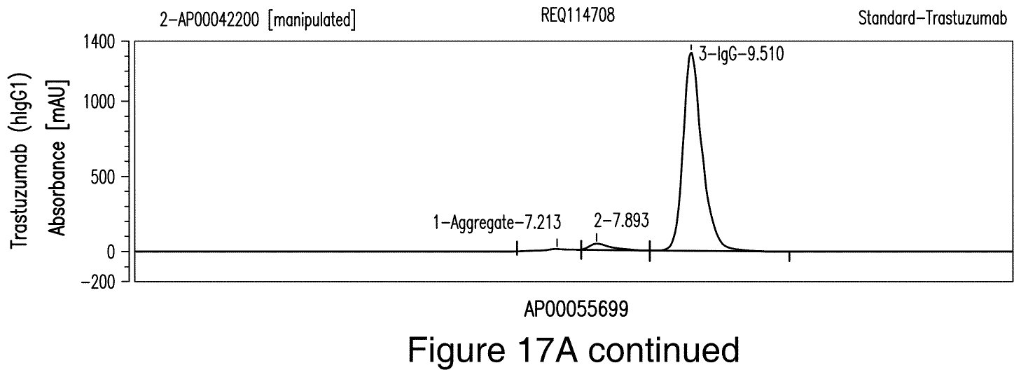

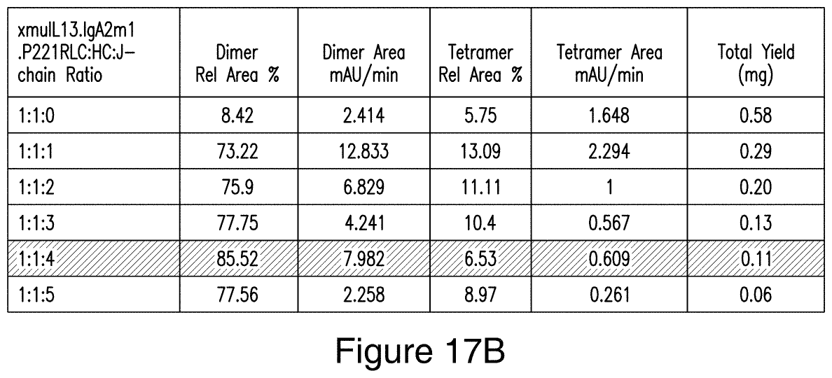

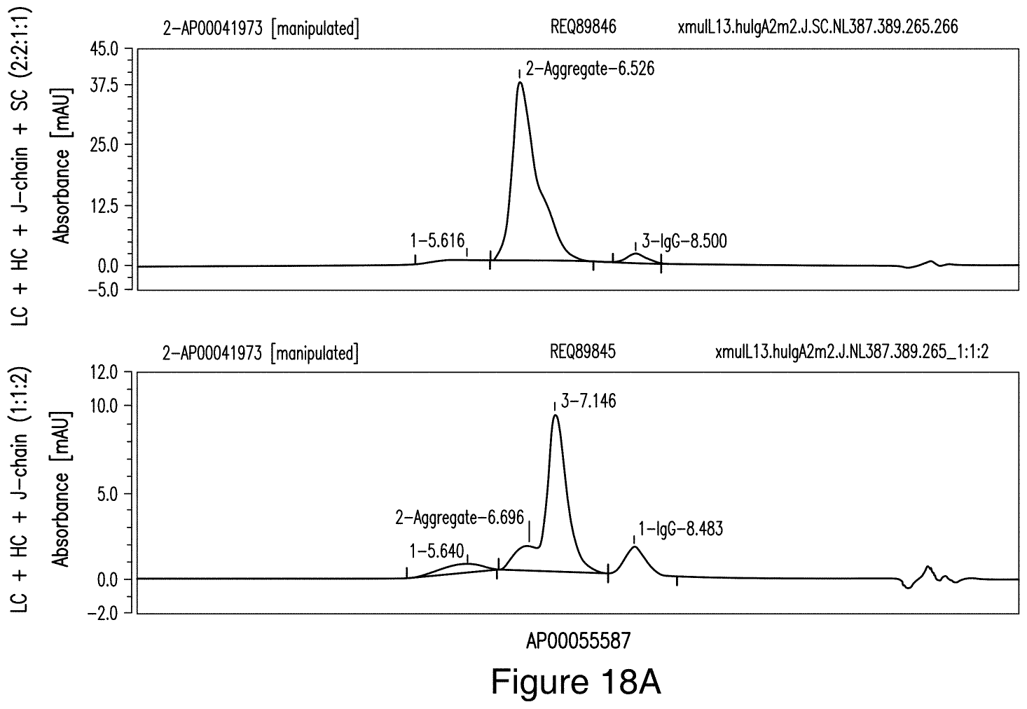

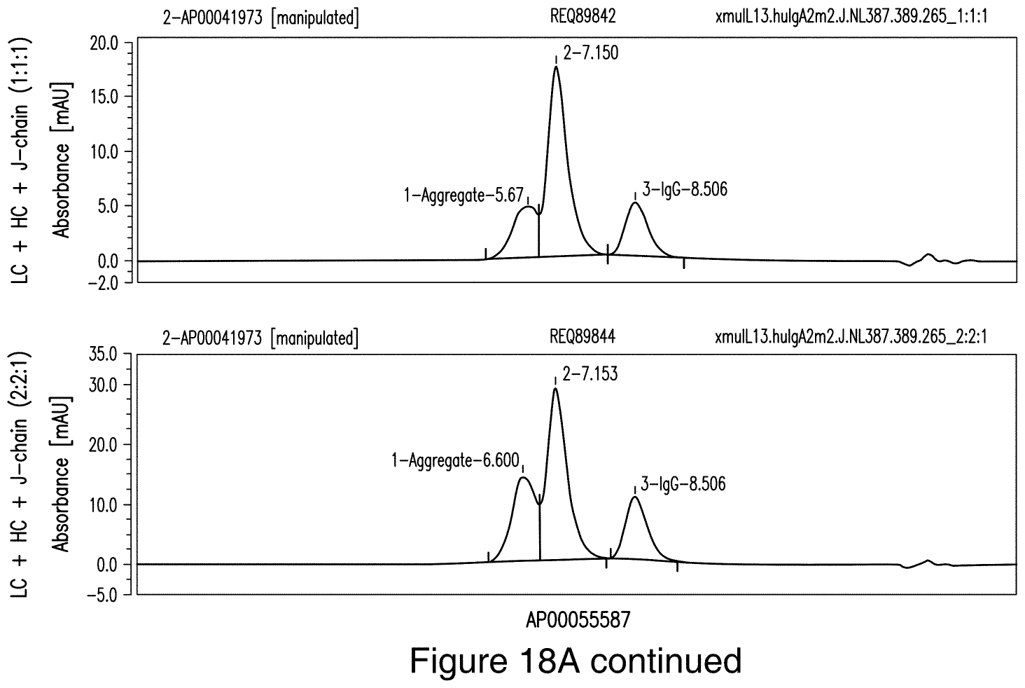

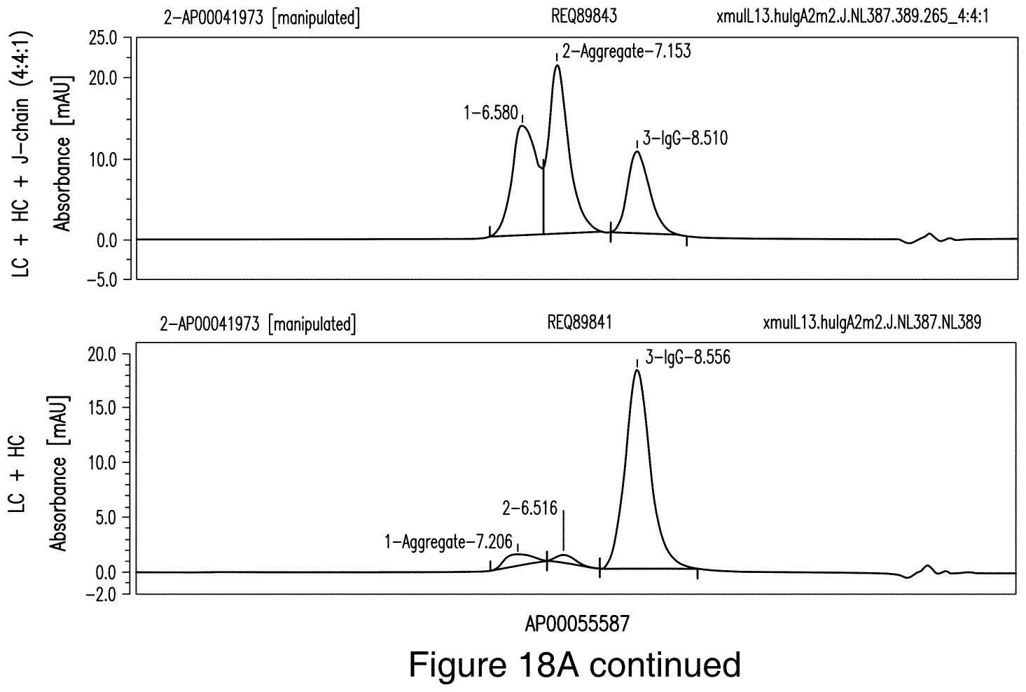

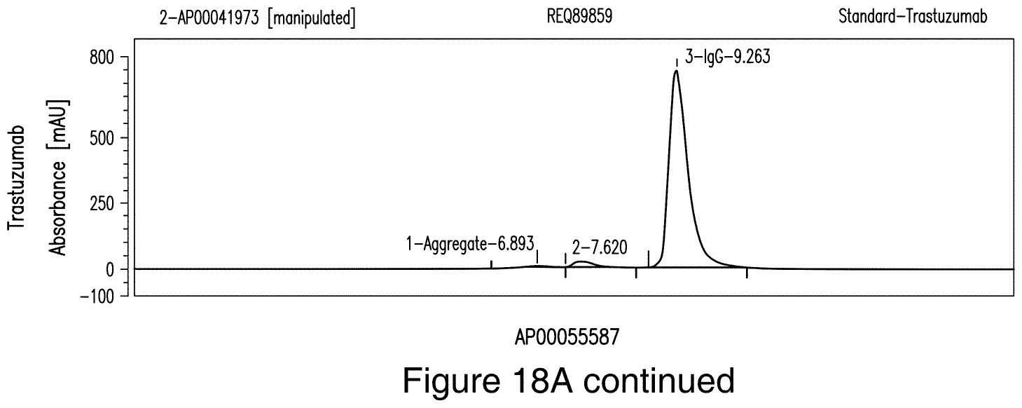

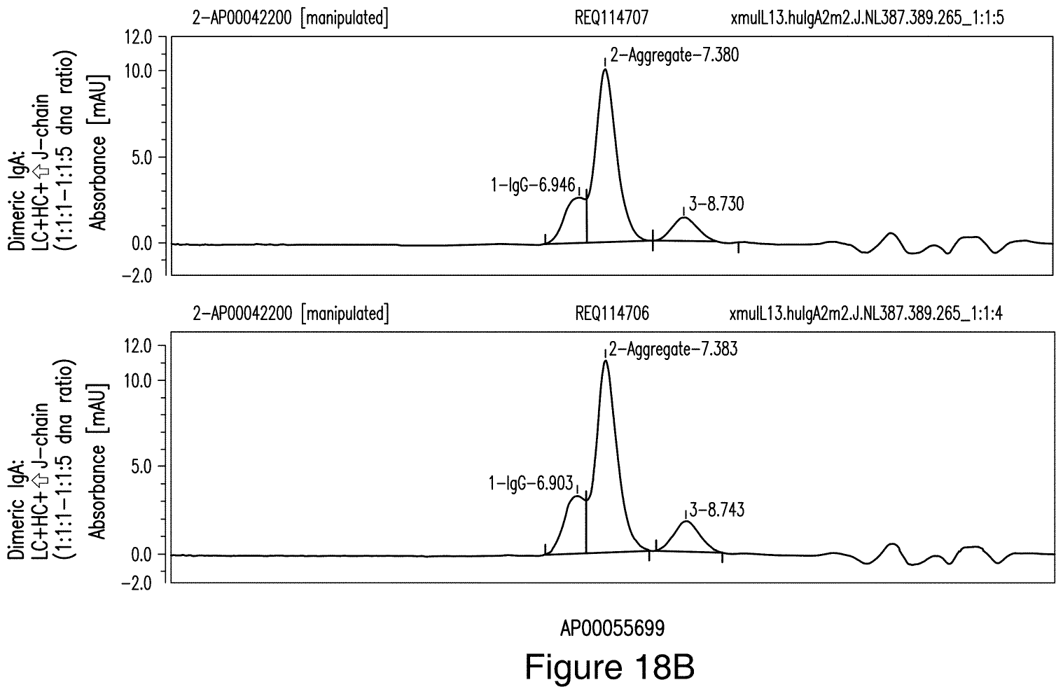

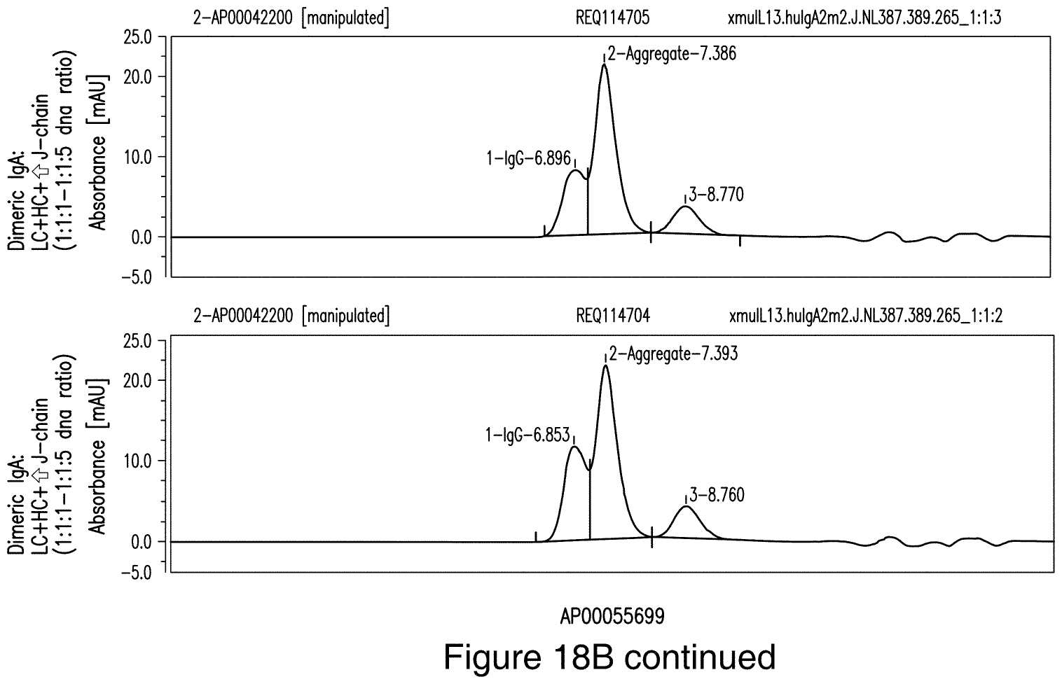

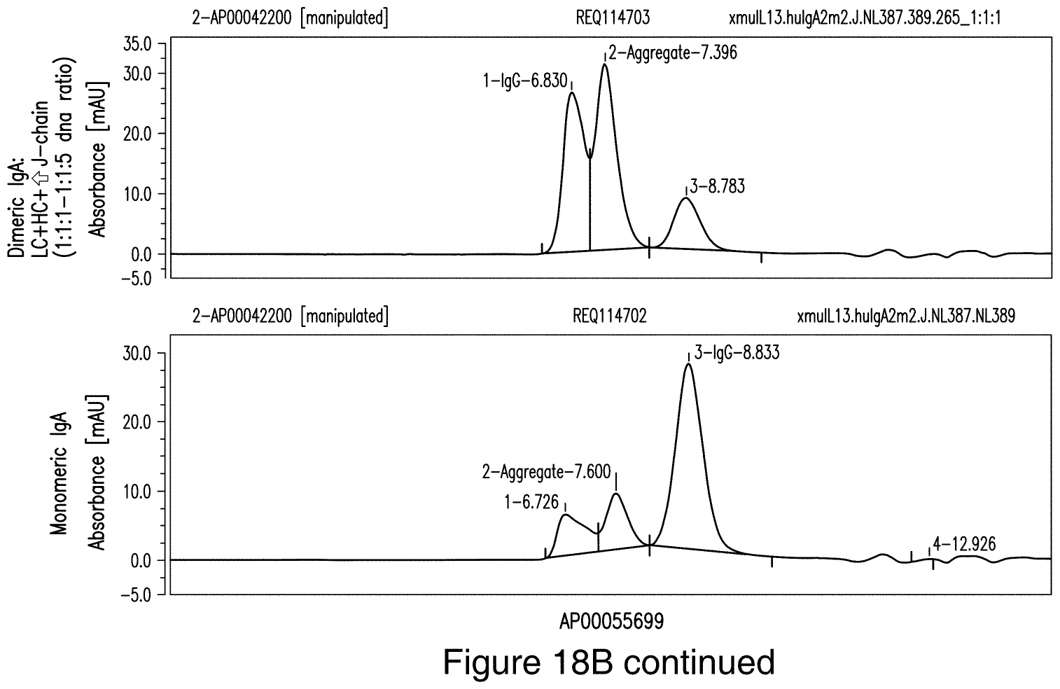

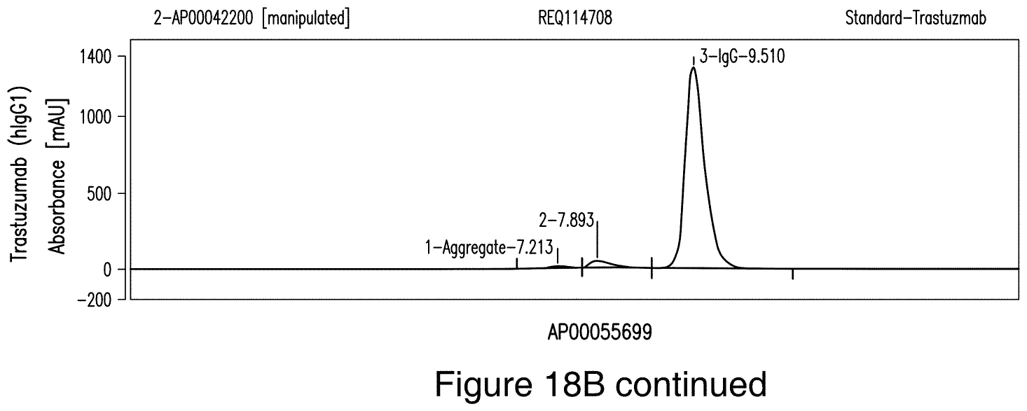

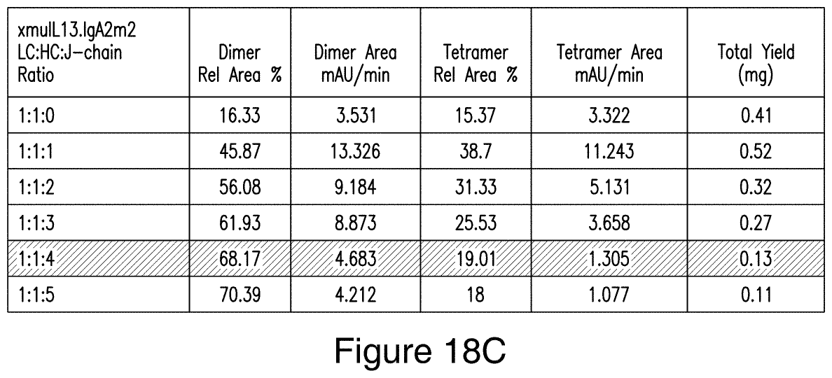

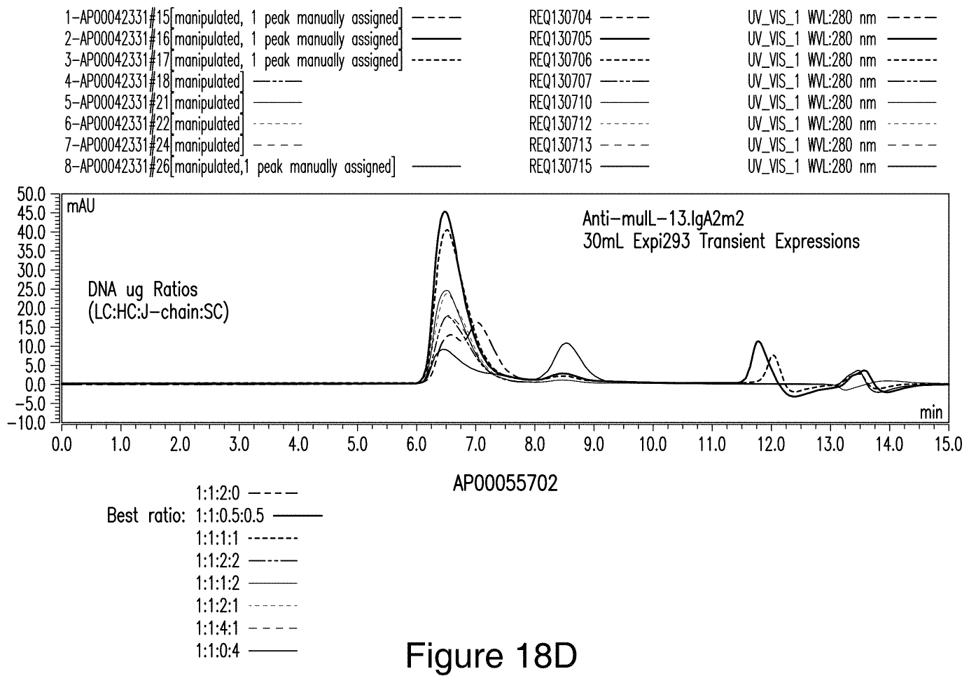

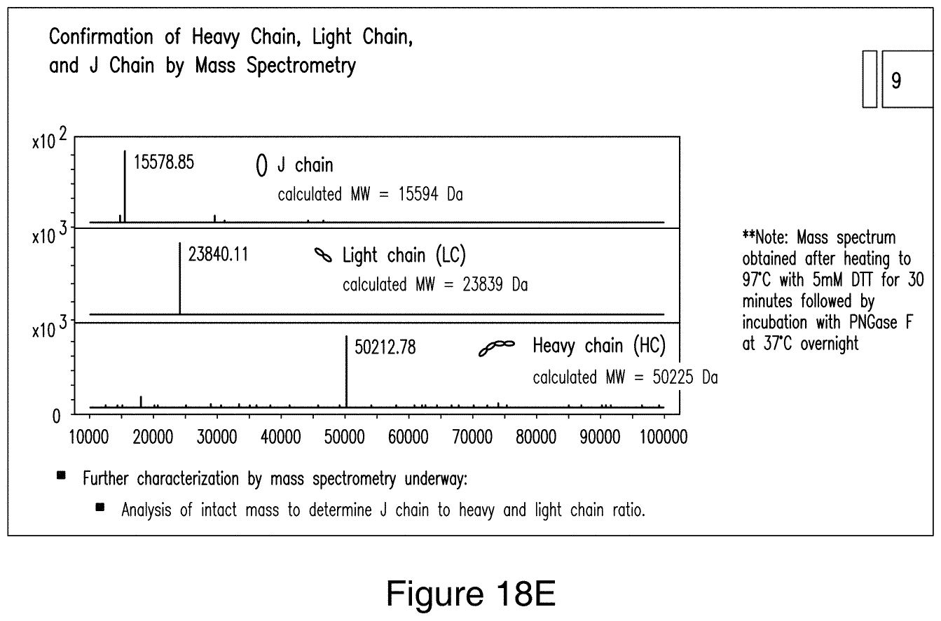

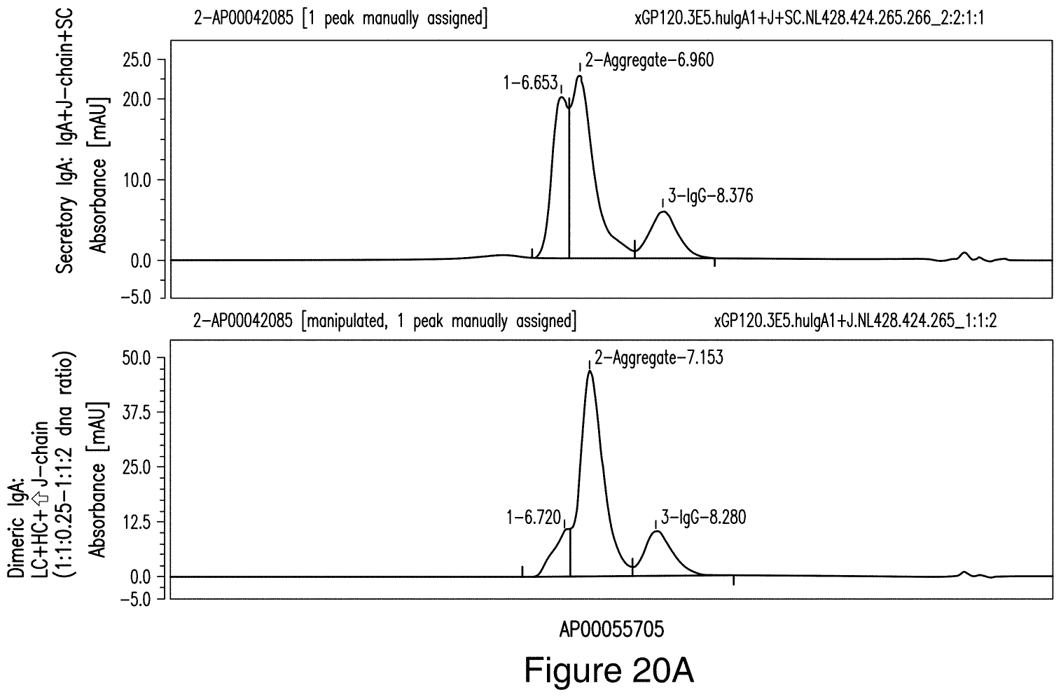

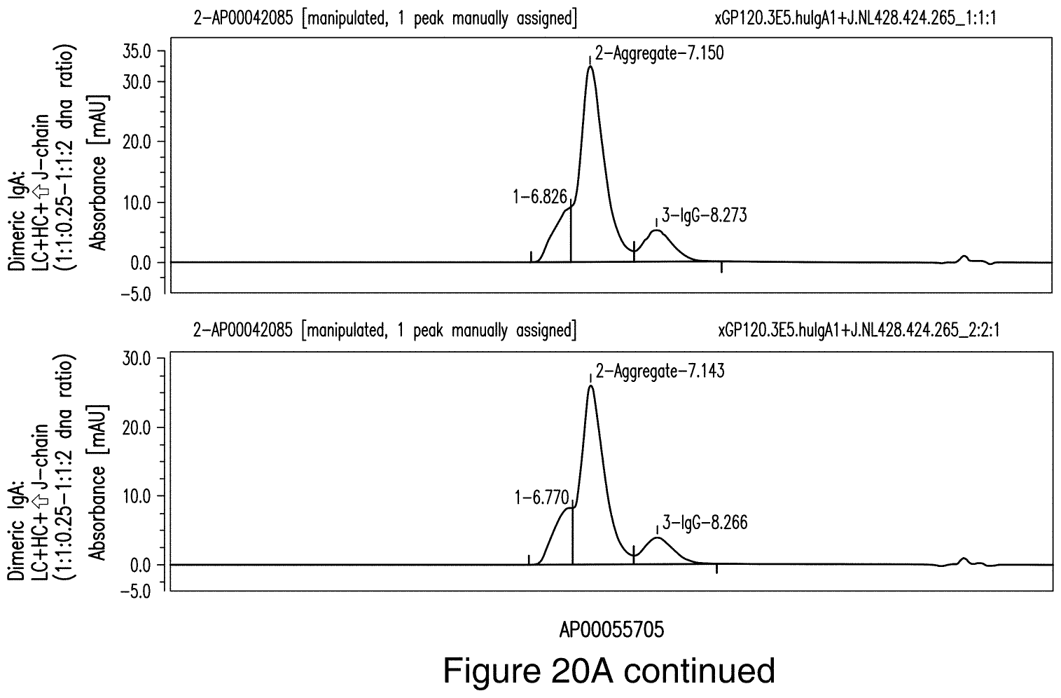

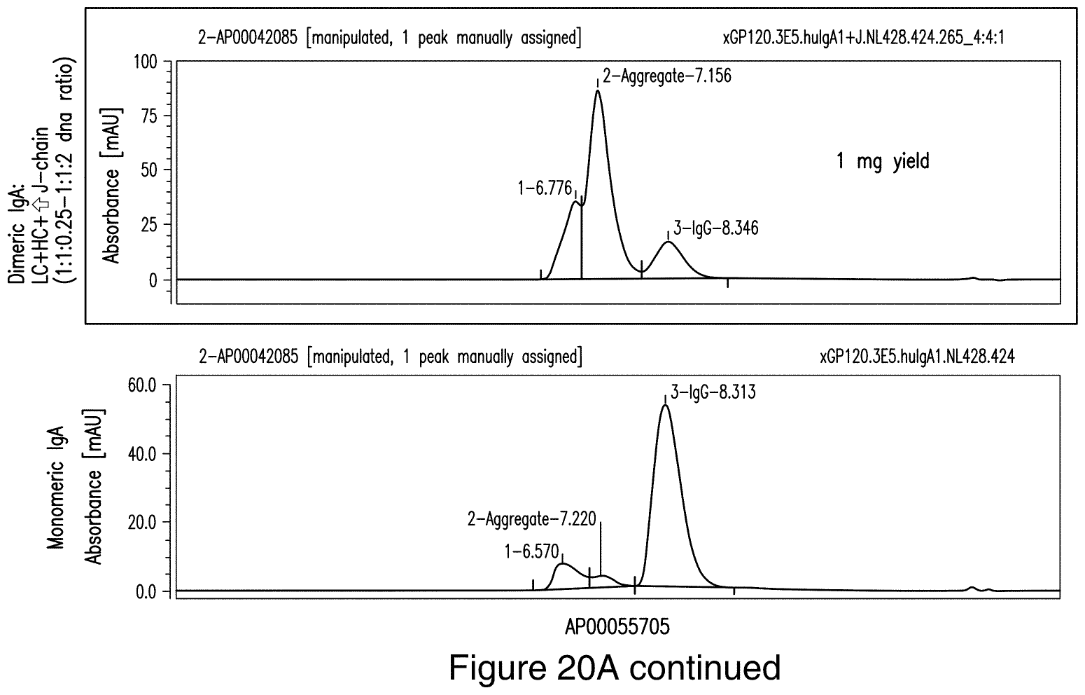

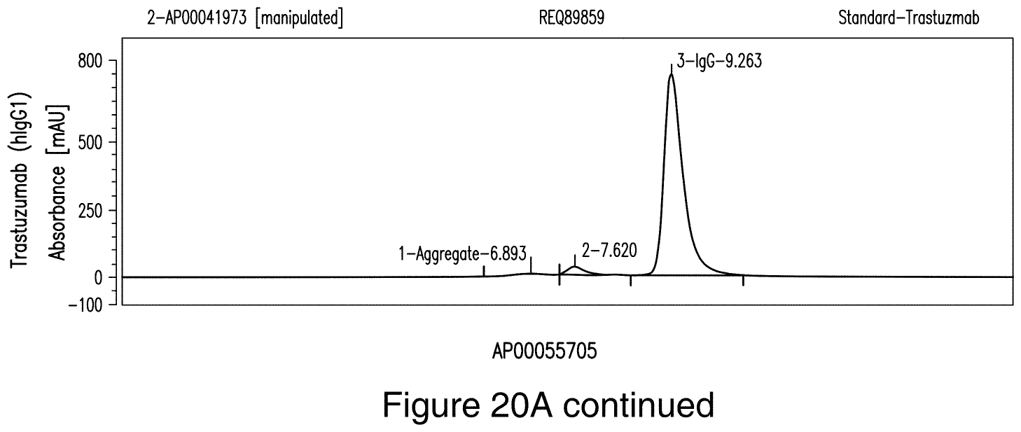

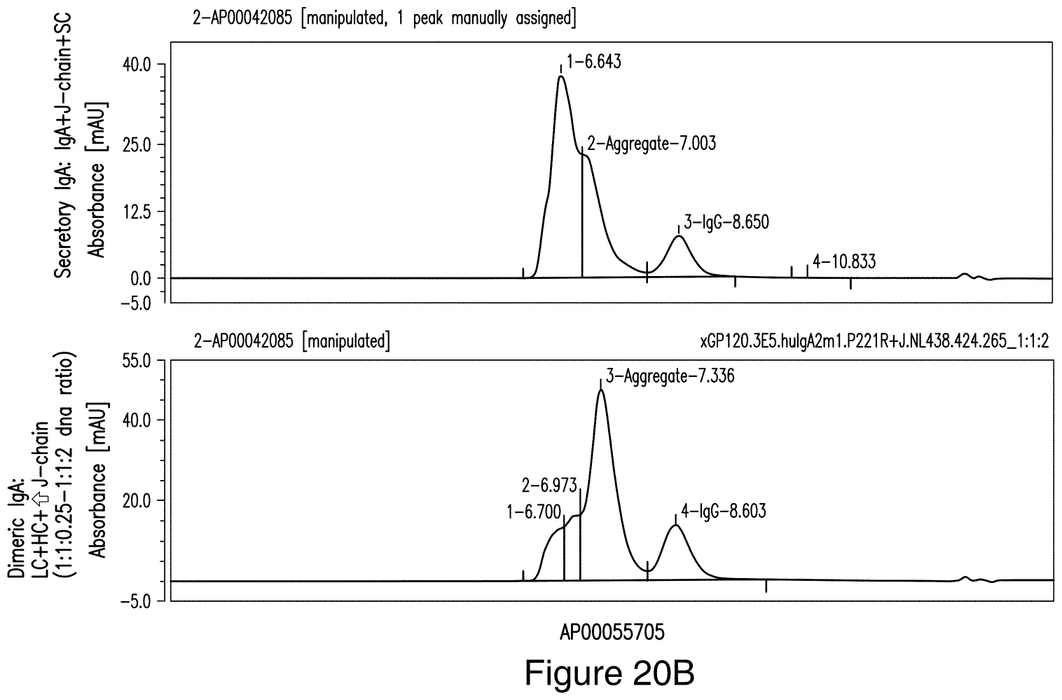

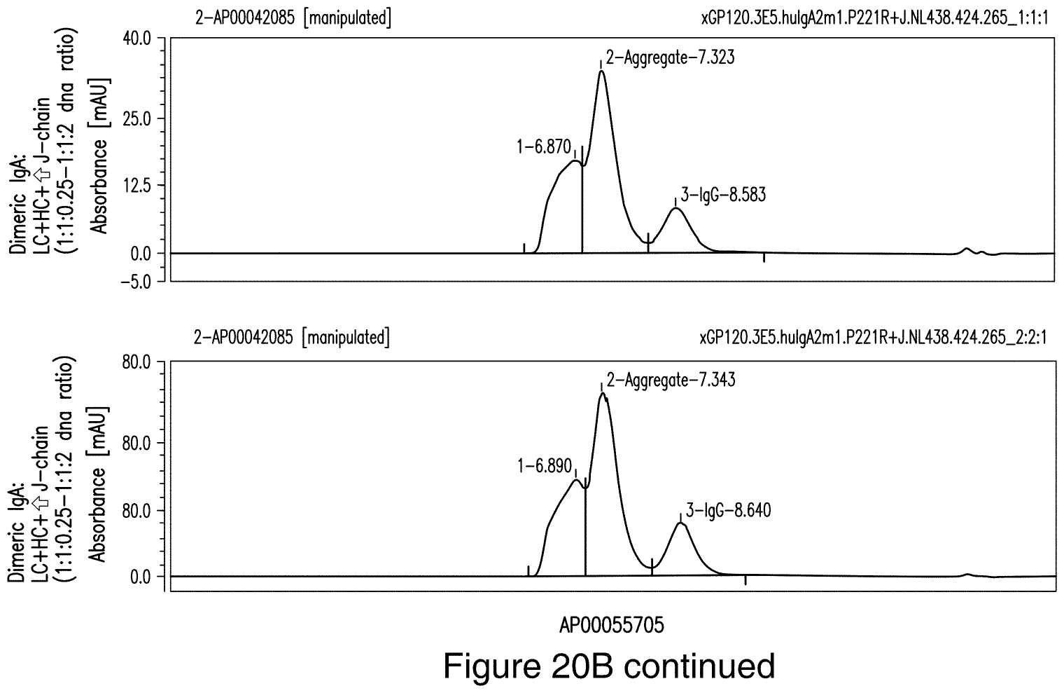

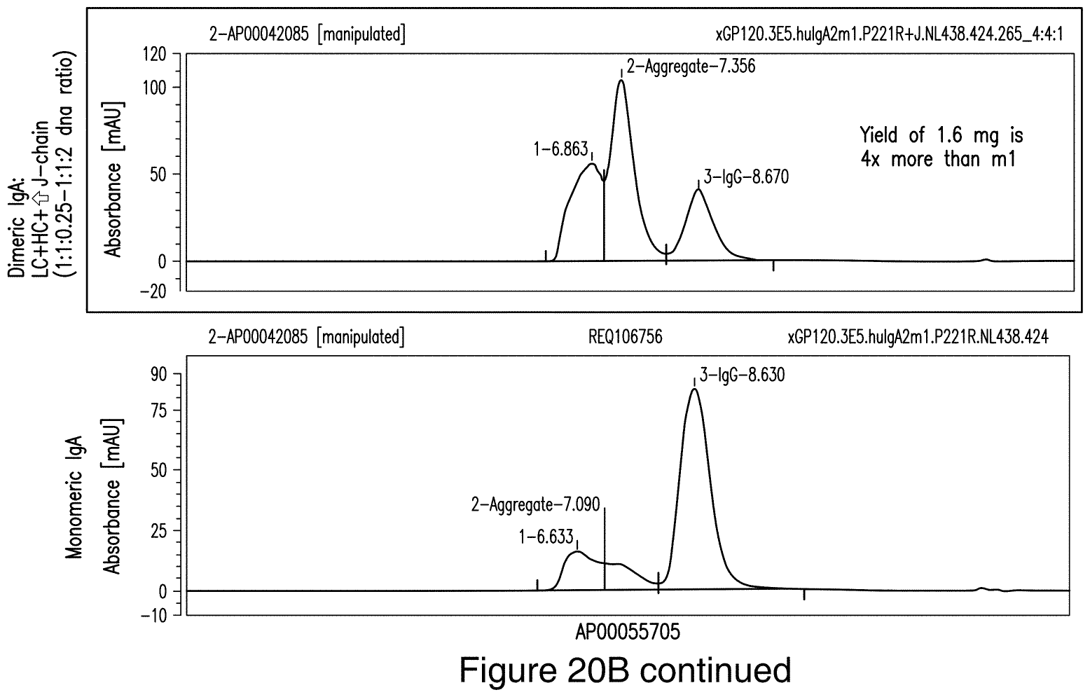

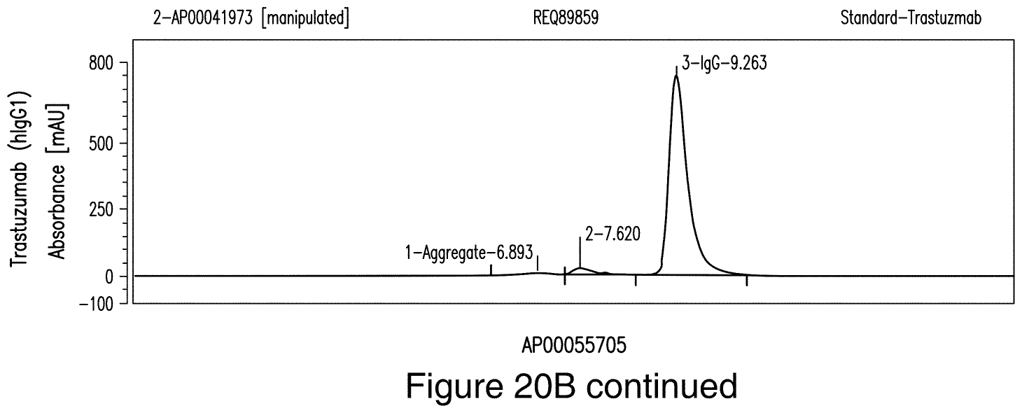

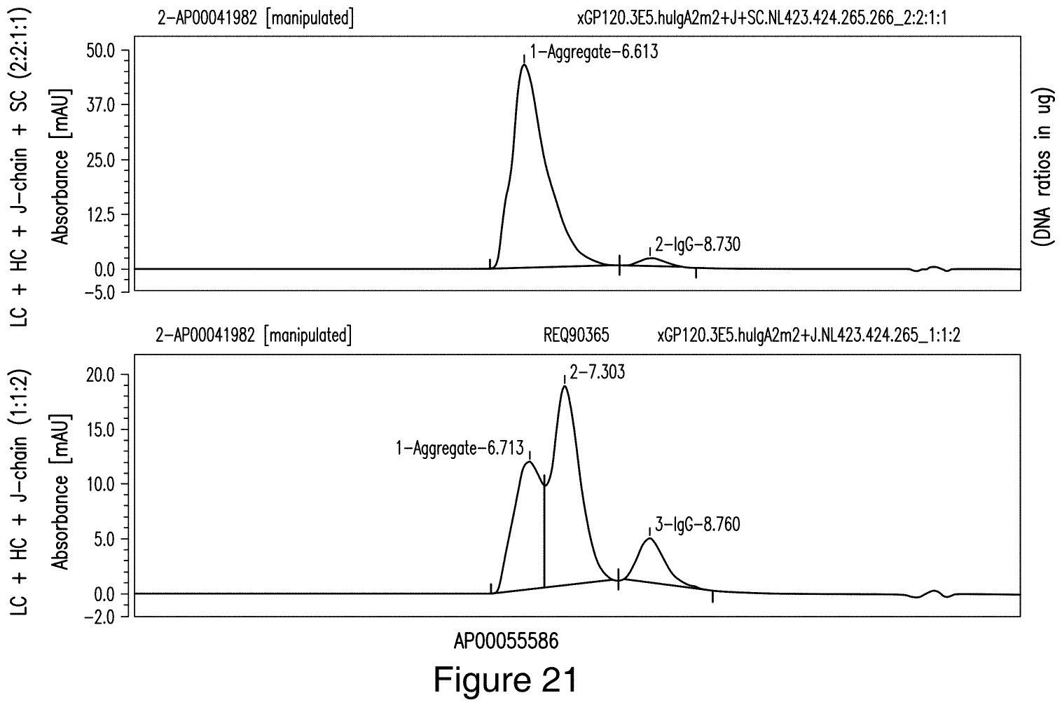

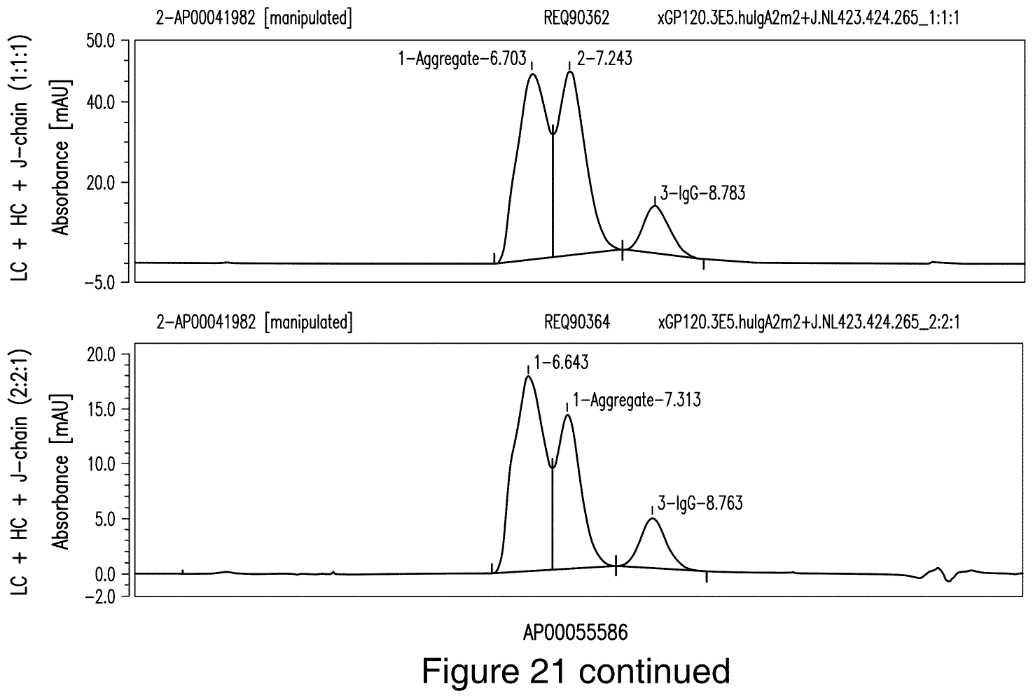

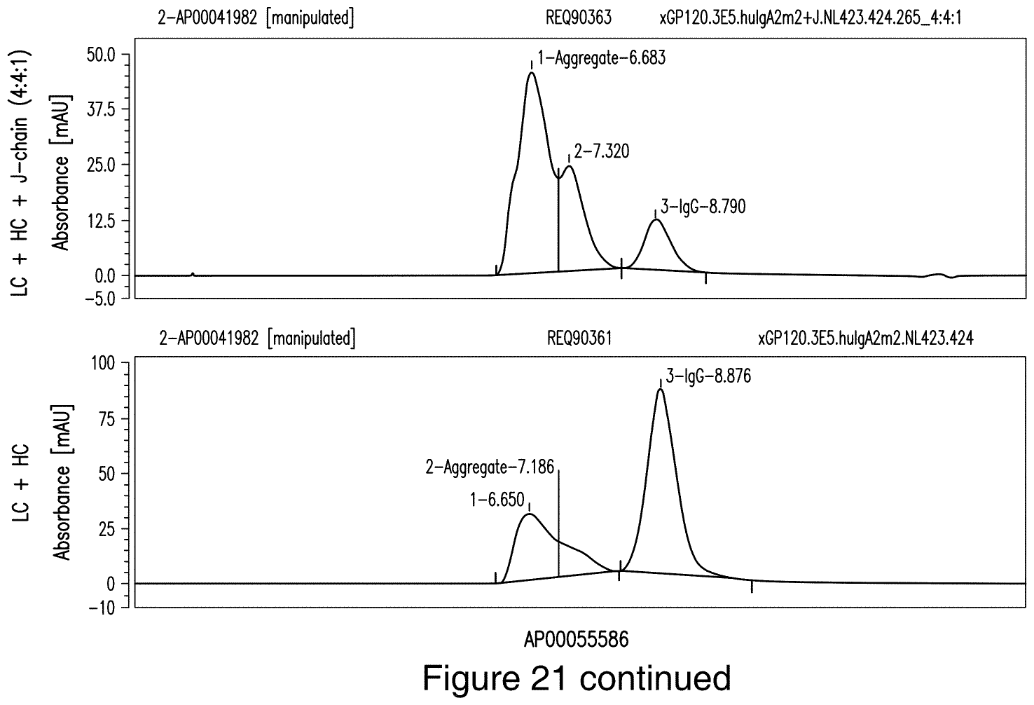

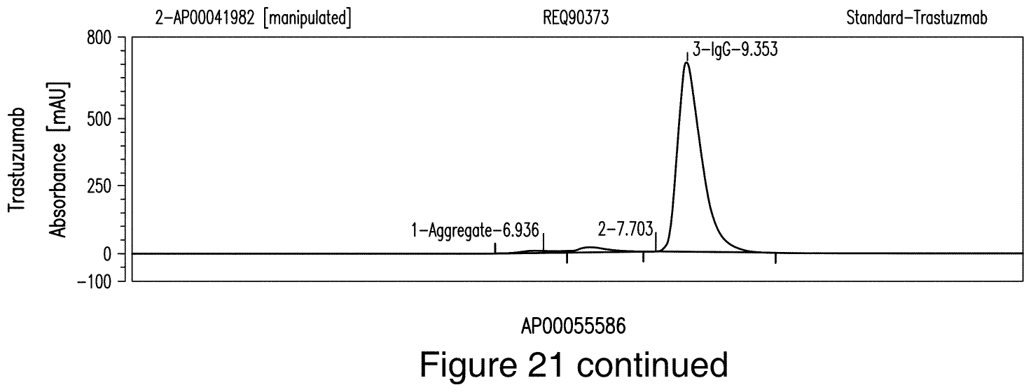

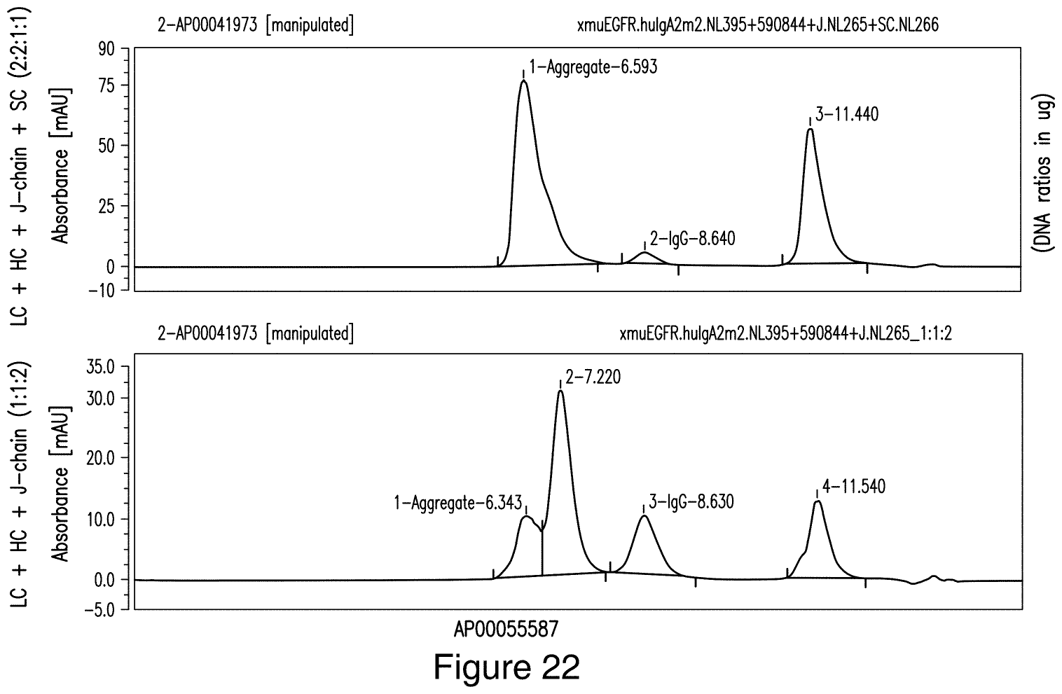

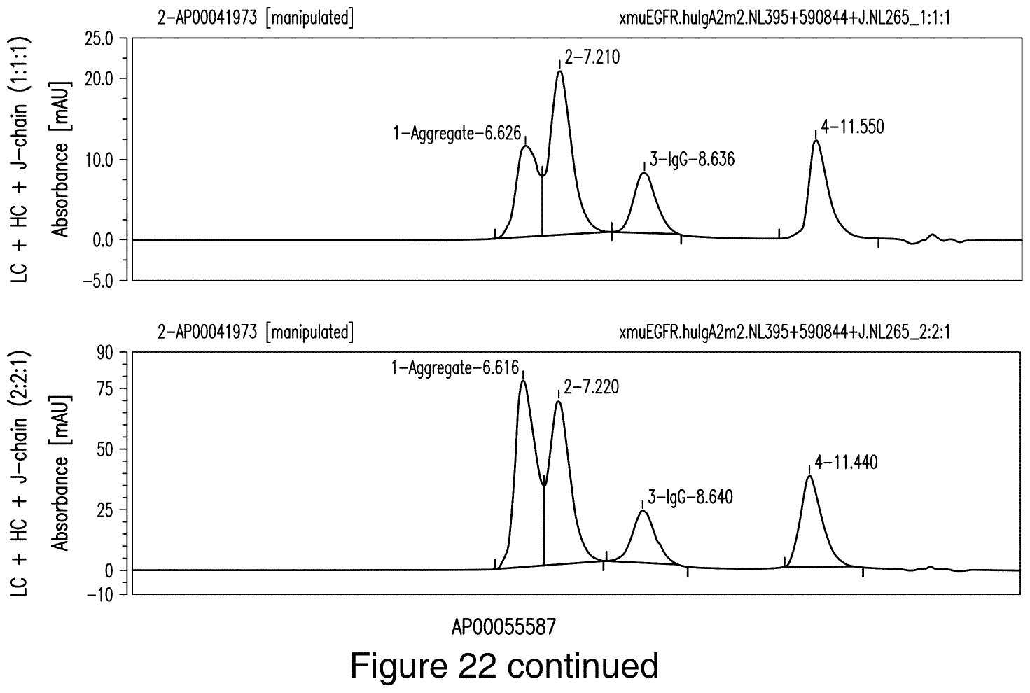

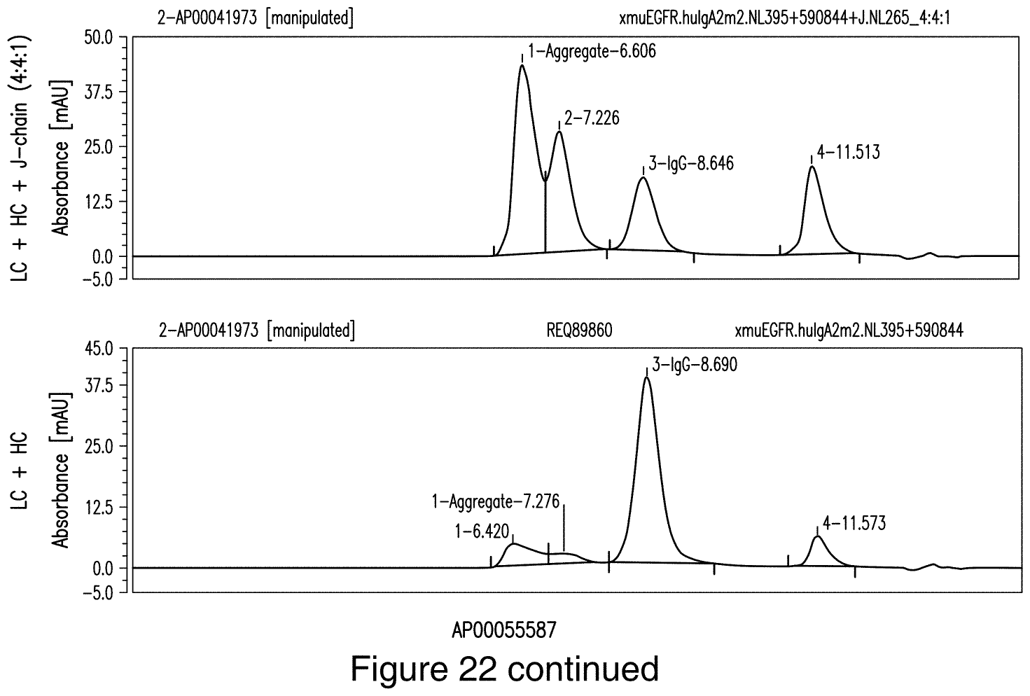

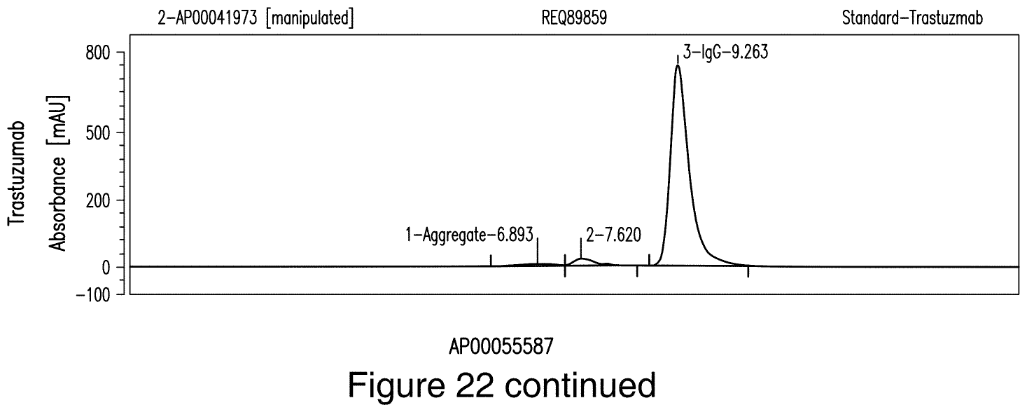

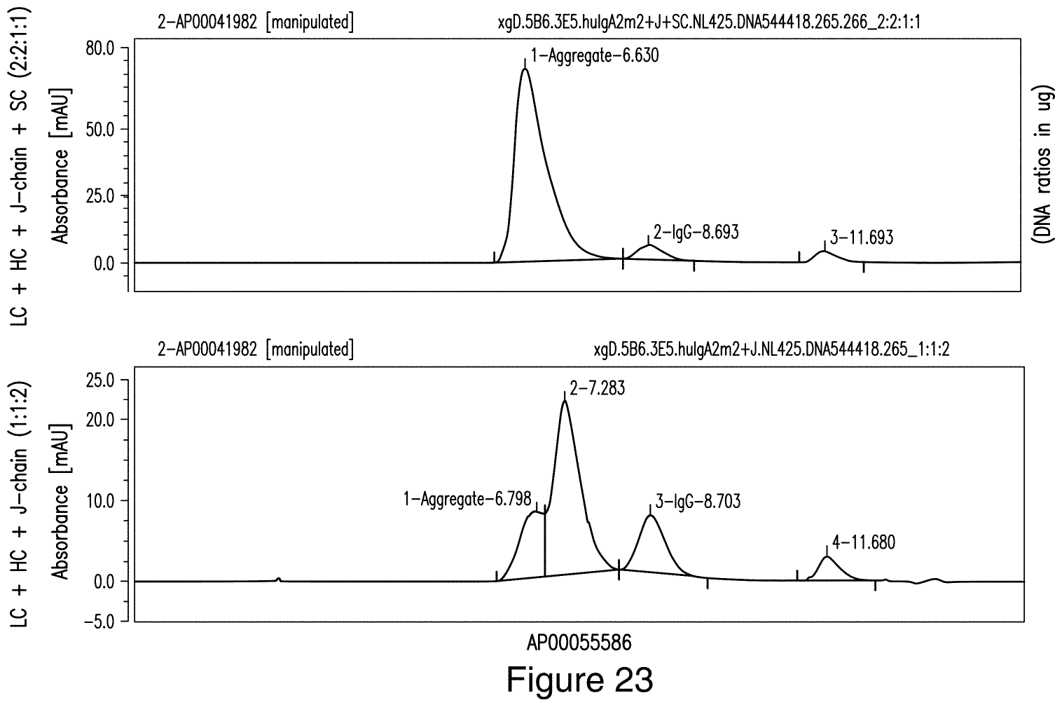

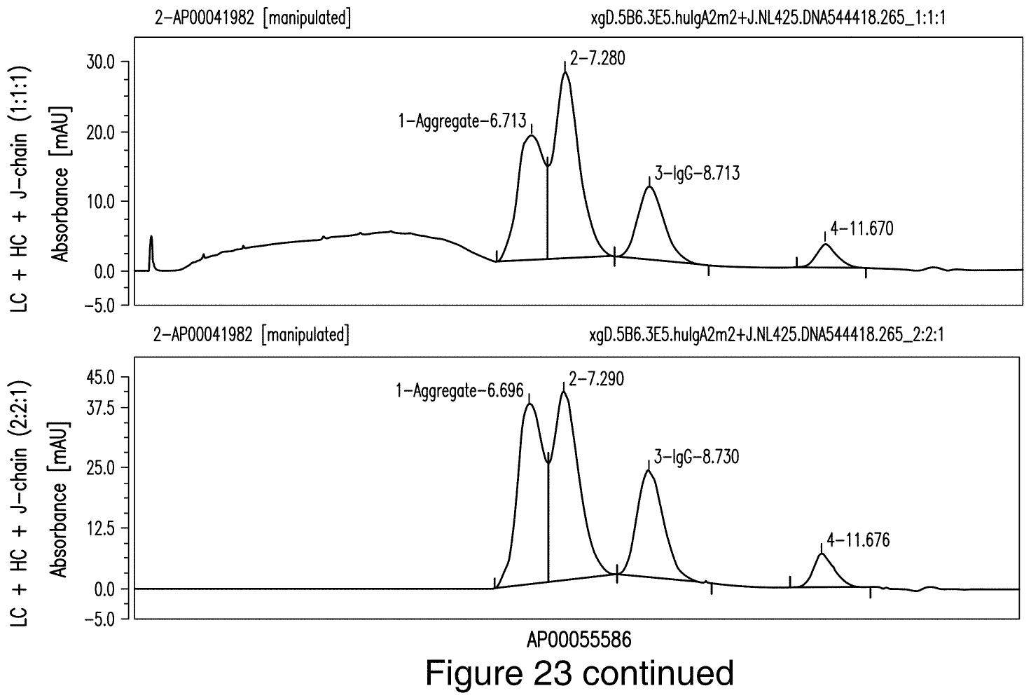

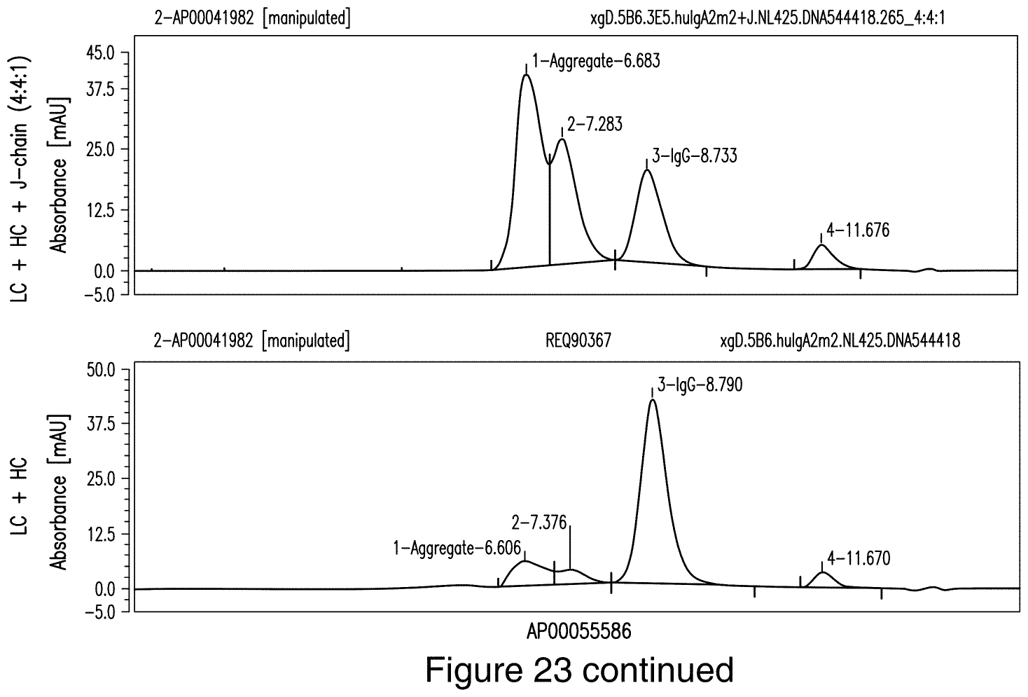

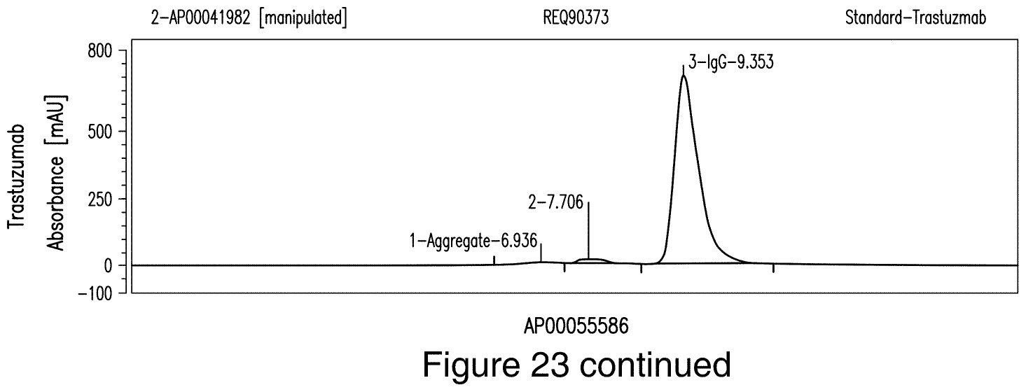

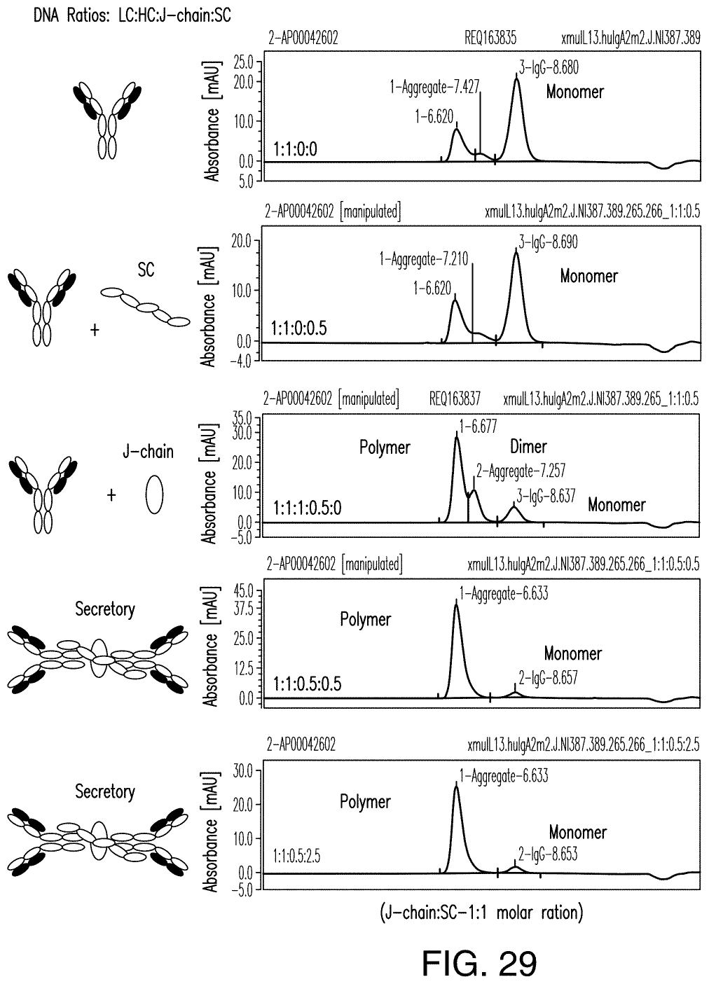

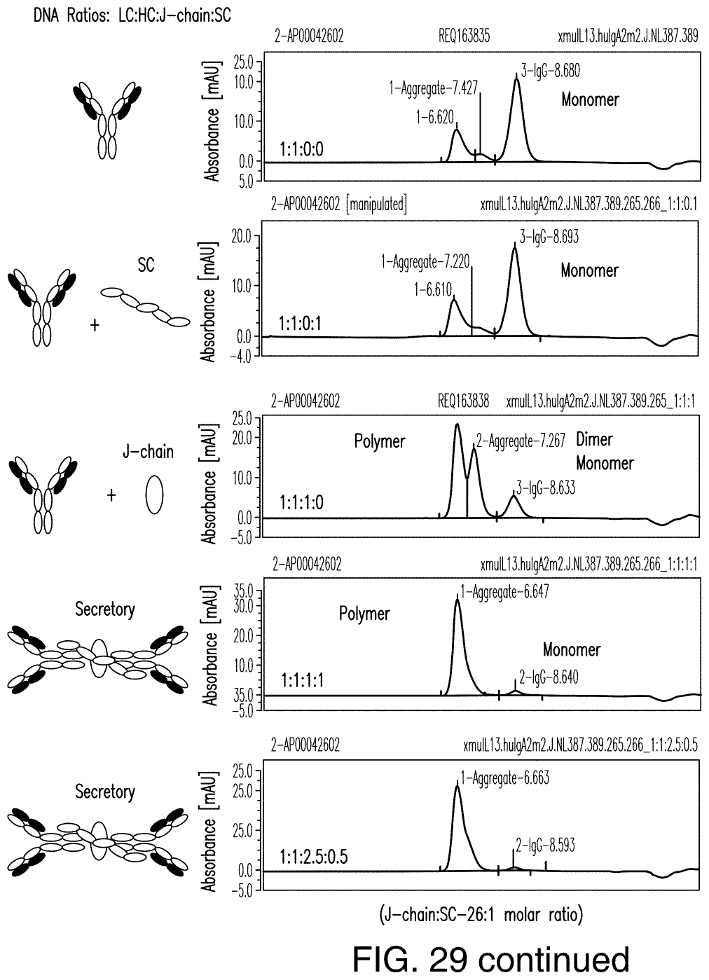

Separating IgA dimer from polymer by secondary purification proved challenging at the larger scale. In an attempt to bias assembly towards dimer formation, the amount of JC DNA relative to both LC and HC DNA amounts were increased to promote increased JC expression levels. This resulted in an increase in the relative percentage of dimer species and a decrease in the relative percentage of polymer species (FIG. 2A-C; see also FIGS. 15-17 and 20). Conversely, decreasing the amount of JC DNA relative to both LC and HC DNA amounts resulted in an increased percentage of higher order polymer (trimer/tetramer/pentamer) (FIGS. 18 and 21-23). The ability to influence oligomeric species based upon the JC DNA amount was most pronounced for the IgA2m2 species. Further, the co-transfection of the secretory component, the joining chain, the light chain and the heavy chain yielded higher order oligomer, as compared to co-transfection of the joining chain, light chain and heavy chain, without the secretory component (FIG. 29).

To understand why IgA2m2 has a higher propensity to form larger oligomers than IgA1 or IgA2m1, the amino acid sequences of the HC tailpieces for the different isotypes/allotypes were compared. While the sequences of the IgA1 and IgA2m1 tailpieces are identical, IgA2m2 differs by two residues. Residues 458 and 467 are both valines in IgA1 and IgA2m1, whereas IgA2m2 has an isoleucine and alanine at these positions, respectively (FIG. 1A, asterisks). Therefore, it was investigated whether these two amino acid differences could explain the unique predisposition of recombinant IgA2m2 to form larger oligomers. Indeed, when isoleucine was substituted for valine at position 458 in IgA1 or IgA2m1, more polymer was produced and this was independent of alanine or valine at residue 467 (FIG. 2D). Conversely, when position 458 in IgA2m2 was changed from isoleucine to valine, the content of polymeric species was reduced in favor of increased dimer content.

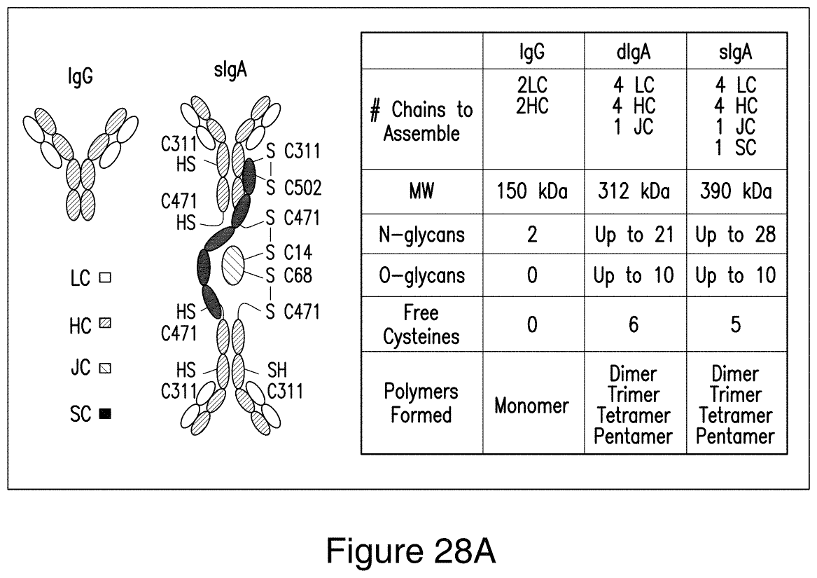

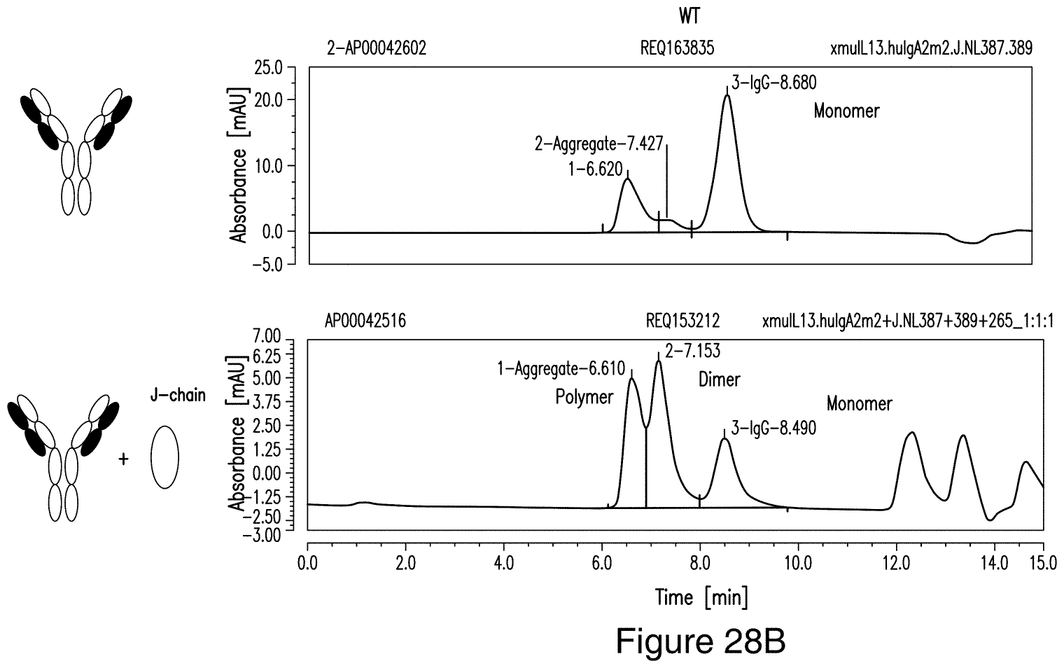

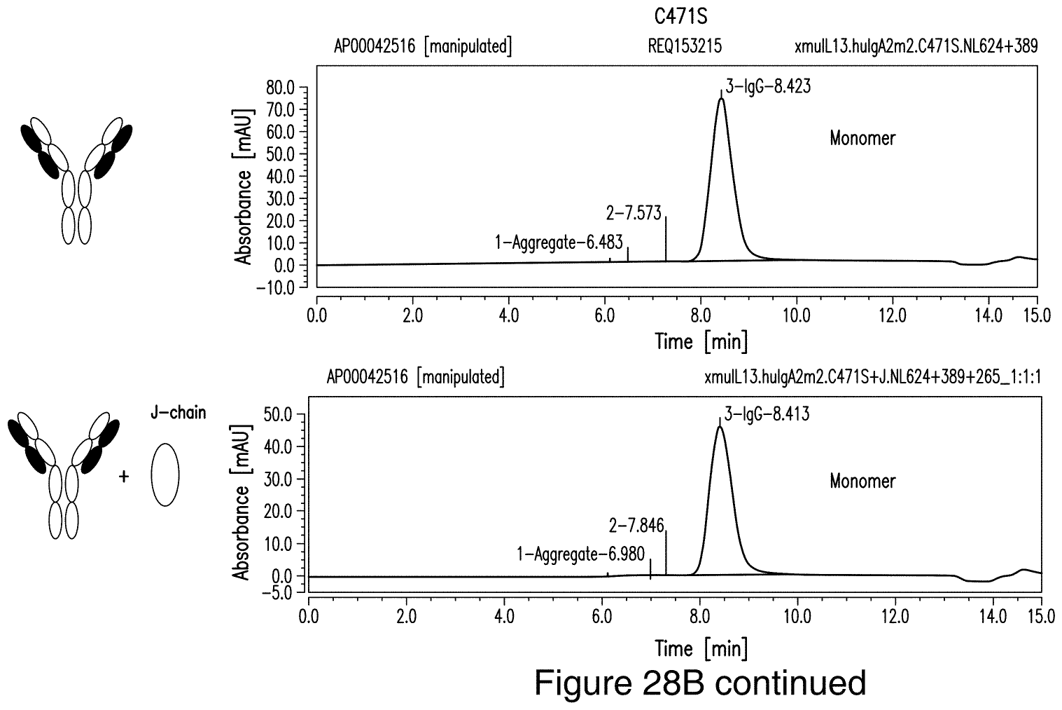

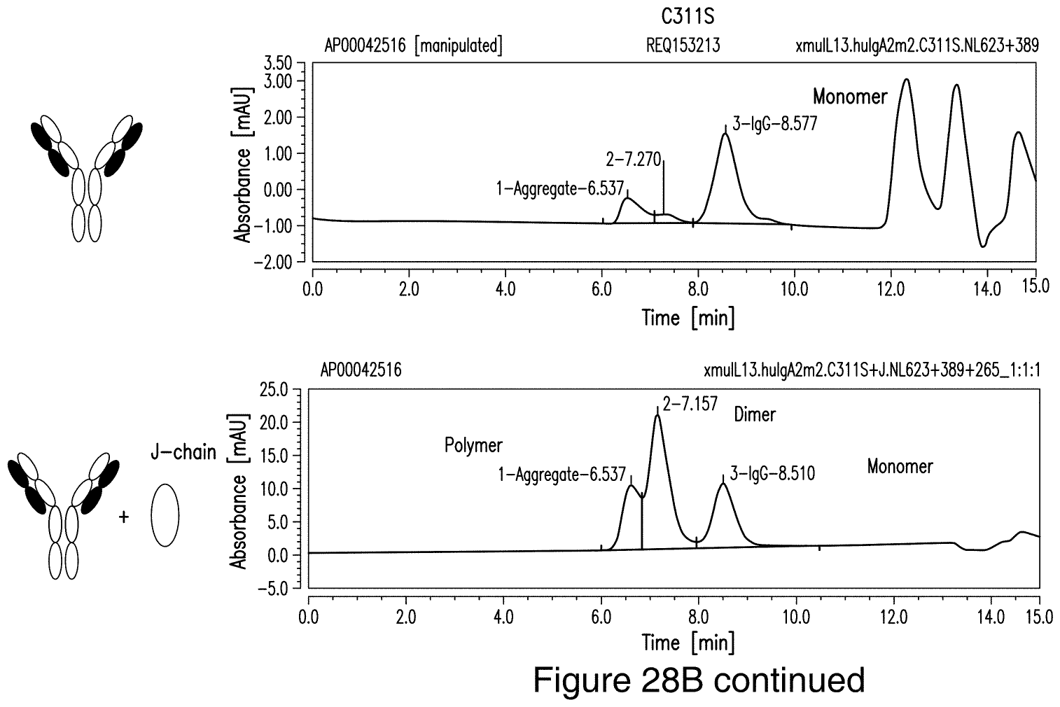

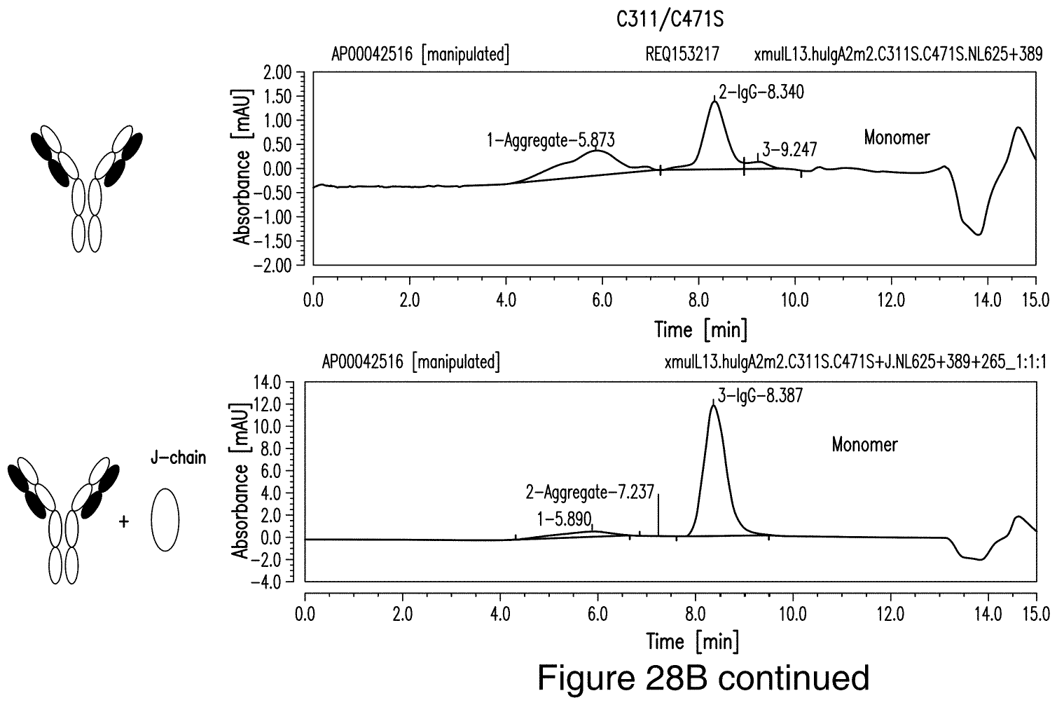

Mutations of certain cysteine residues in the heavy chain of an IgA2m2 antibody were generated to prevent disulfide bonds with the secretory component or the joining chain and analyzed to determine the effect of such mutations on oligomer formation. The mutation of Cys311 to serine prevents disulfide bond with secretory component and the mutation of Cys471 mutation to serine prevents disulfide bond with the joining chain. As shown in FIG. 28B, mutation of C471 but not C311 was required for IgA2m2 dimer and higher order oligomer formation when adding the joining chain to the light chain and heavy chain.

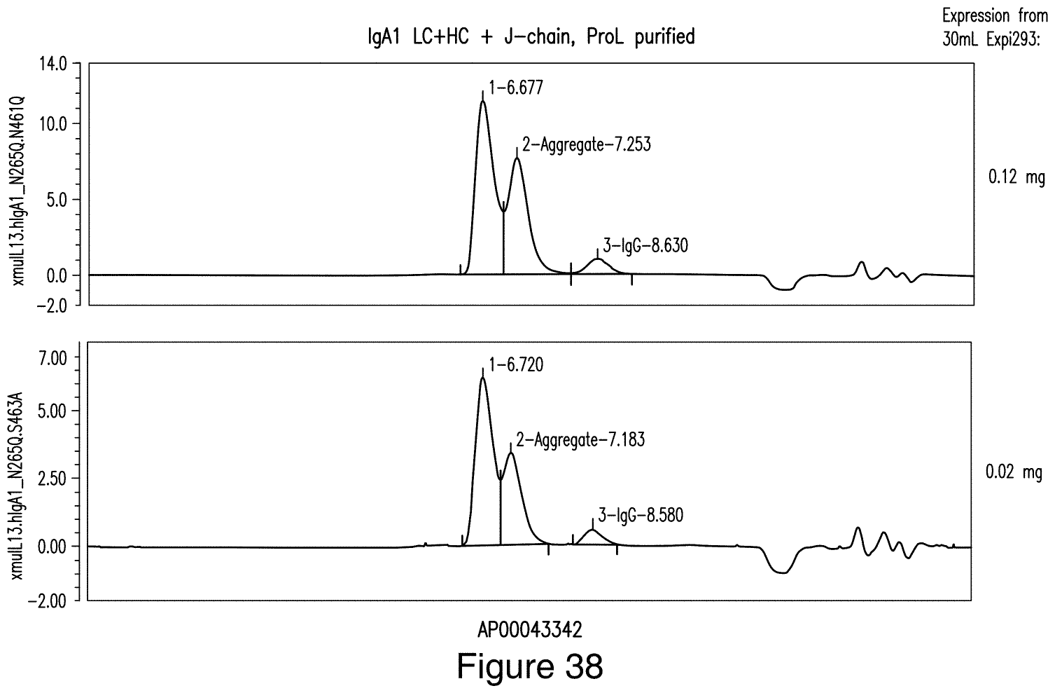

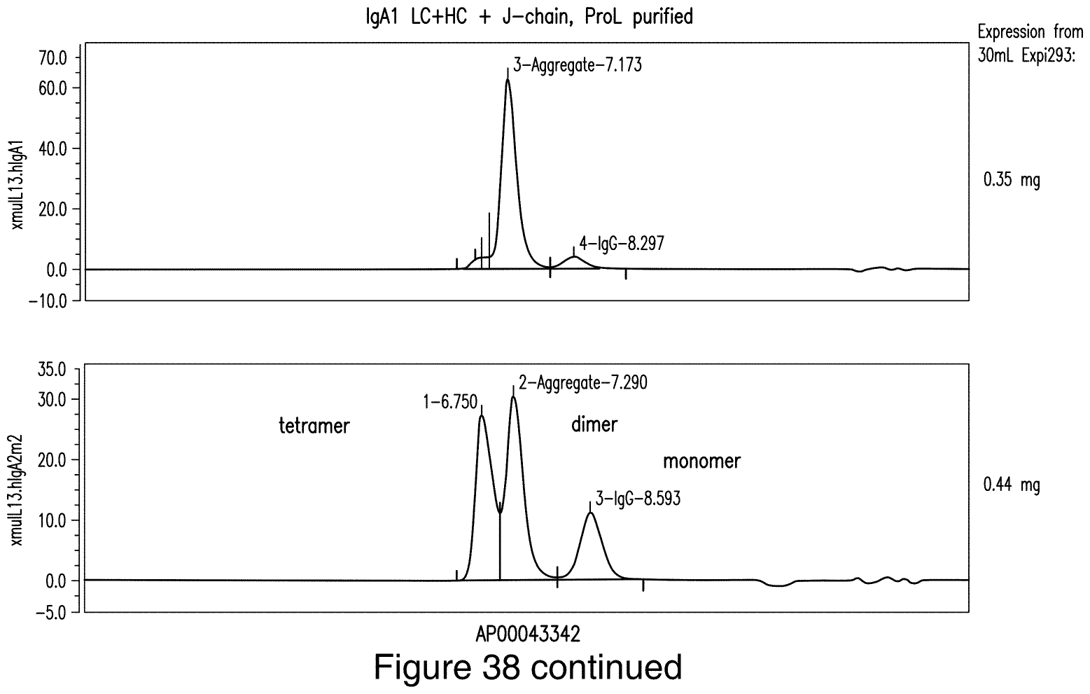

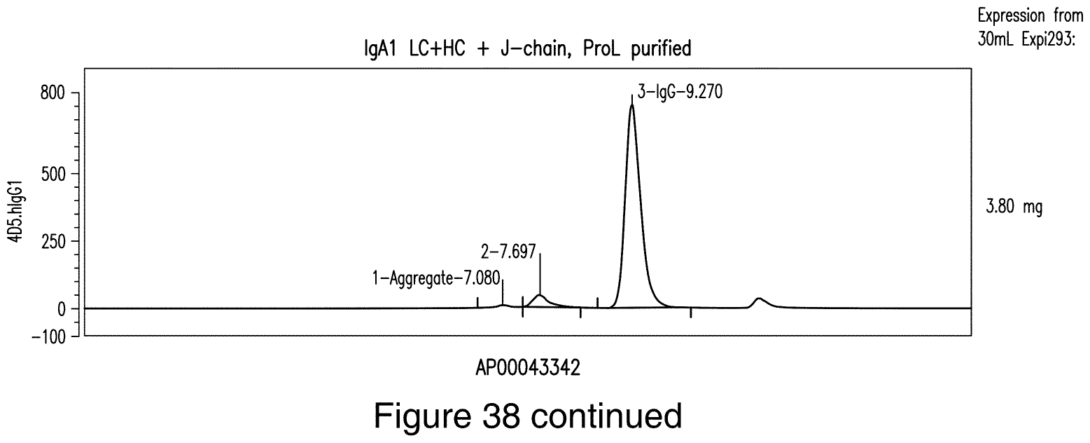

Glycosylation is known to play a role in IgA oligomerization (Chuang et al., J Immunol 158:724-32 (1997)). Accordingly, mutations were made to remove each N-linked glycosylation site in IgA1 and IgA2m2. Four separate mutations (N459A/G/Q or S461A) that removed the N-linked glycosylation site in the tailpiece of IgA1 or IgA2m2 also increased the amount of polymer produced, while mutations to remove glycosylation sites outside the tailpiece did not alter oligomer formation (FIGS. 2E and 2F, respectively; see also FIG. 38). Therefore, in addition to modulating the DNA ratios in transfection, IgA polymer formation can be increased by having isoleucine at tailpiece amino residue 458 or preventing N-linked glycosylation of the IgA tailpiece.

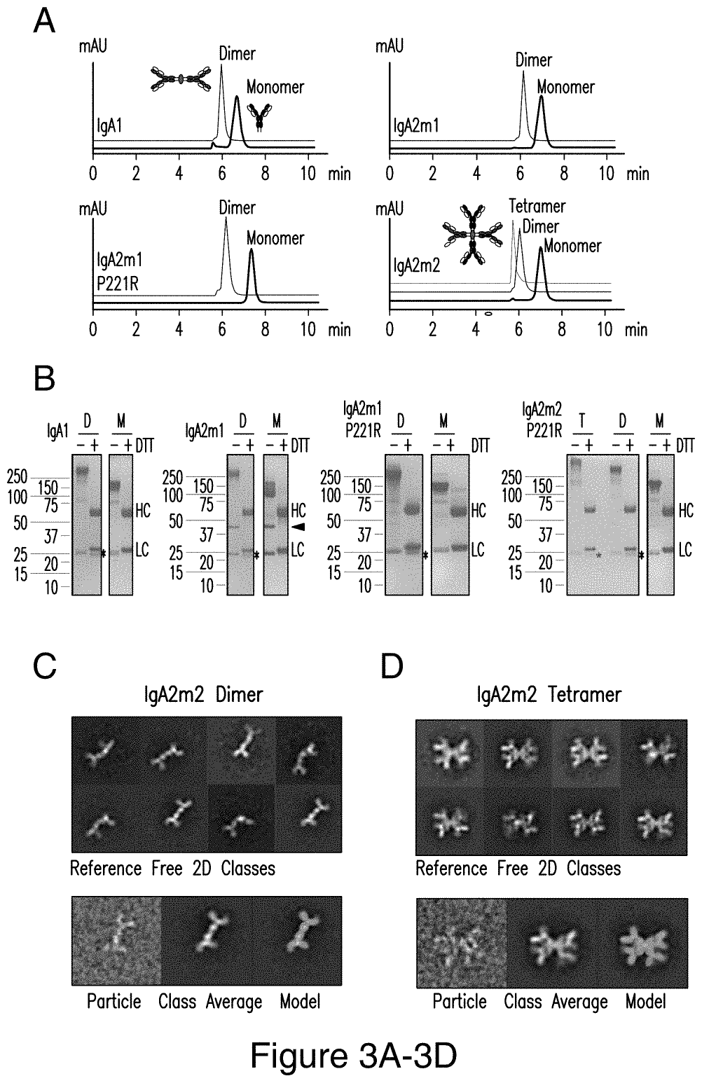

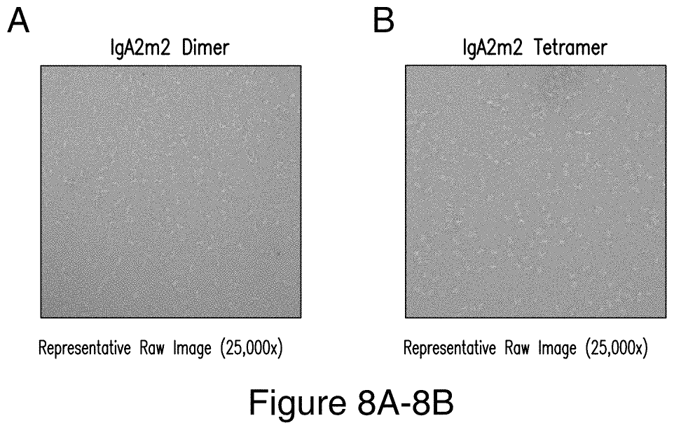

Large Scale Purification and Biophysical Characterization of IgA Monomers and Oligomers. Using insights into IgA oligomer formation gained through small-scale expression, monomeric, dimeric and tetrameric IgA were scaled up using CHO transient expression. The monomeric and dimeric species of IgA1, IgA2m1, IgA2m1.P221R and IgA2m2, as well as the tetrameric species of IgA2m2, were isolated (FIG. 3A). Non-reduced SDS-PAGE analysis of these samples showed predominant bands of molecular weights consistent with the expected masses of ˜150 kDa, ˜310 kDa, and ˜610 kDa for an IgA monomer, dimer and tetramer, respectively (FIG. 3B). These expected masses were based on the amino acid sequence without glycosylation, and assume incorporation of one JC per oligomer. Molar masses of the purified oligomeric species were also measured by SEC-MALS and found to be consistent with the expected masses of dimeric and tetrameric IgA (Table 2). Reduced SDS-PAGE analysis of the purified IgA samples confirms the presence of LC and HC bands for monomers at ˜25 kDa and ˜55 kDa, respectively, whereas in the oligomeric samples a band for JC just below 25 kDa can also be detected (FIG. 3B). The identity of the LC, HC and JC were additionally confirmed by mass spectrometry after reduction and enzymatic deglycosylation (FIG. 18E). Negative stain electron microscopy (EM) was also used to further validate the oligomeric state of the isolated species. Negative stain images of the IgA2m2 dimer (FIG. 3C) and tetramer (FIG. 3D) confirm the presence of two or predominantly four IgA molecules, respectively. In the dimer, two IgA molecules are linked tail-to-tail by their Fc domains into an elongated particle, whereas in the tetramer interactions between four Fc domains give rise to a compact complex of four IgA molecules. Raw images of both samples showed the presence of well-behaved, monodispersed particles (FIG. 8).

TABLE 2
Molar mass of recombinant IgA as measured by SEC-MALS

| | Predicted | SEC-MALS | |
|---|---|---|---|
| | MW | MW | Polydispersity |
| | (Da) | (g/mol) | (Mw/Mn) |
| Anti-mIL-13 | 3.160 × 10⁵ | 3.277 × 10⁵ +/− | 1.001 +/− 1.127% |
| IgA1 dimer | | 0.802% | |
| Anti-mIL-13 | 3.114 × 10⁵ | 3.264 × 10⁵ +/− | 1.001 +/− 0.951% |
| IgA2m1 dimer | | 0.675% | |
| Anti-mIL-13 | 3.117 × 10⁵ | 3.437 × 10⁵ +/− | 1.002 +/− 0.911% |
| IgA2m2 dimer | | 0.646% | |
| Anti-mIL-13 | 6.078 × 10⁵ | 6.580 × 10⁵ +/− | 1.005 +/− 0.720% |
| IgA2m2 tetramer | | 0.510% | |

Proteins were injected over a Waters Xbridge Protein BEH 200 Å analytical size-exclusion chromatography (SEC) column coupled to a Wyatt DAWN HELEOS II/Optilab T-rEX multi-angle light scattering (MALS) detector for molar mass and polydispersity measurement. The predicted molecular weight (MW) of the IgA molecules is based only on amino acid composition, assumes incorporation of one JC per dimer or tetramer, and does not account for any potential N- or O-linked glycans.

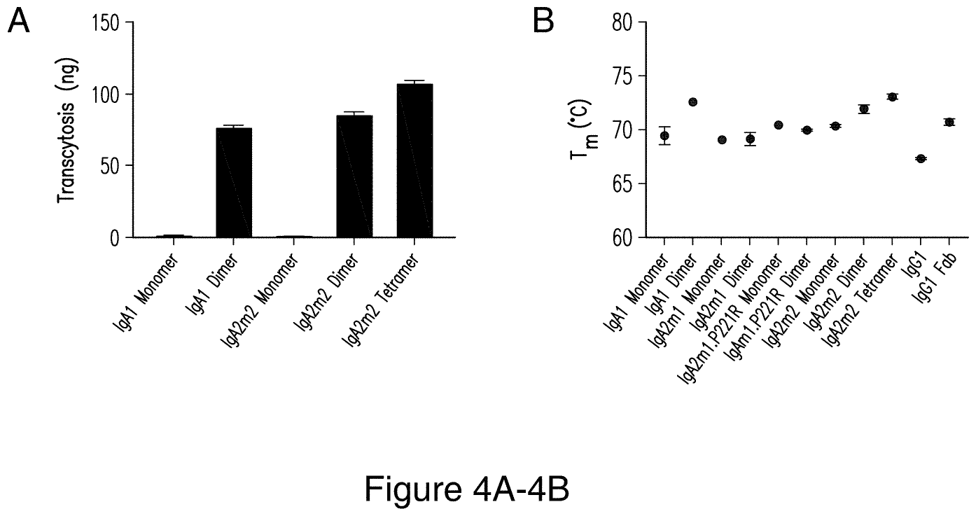

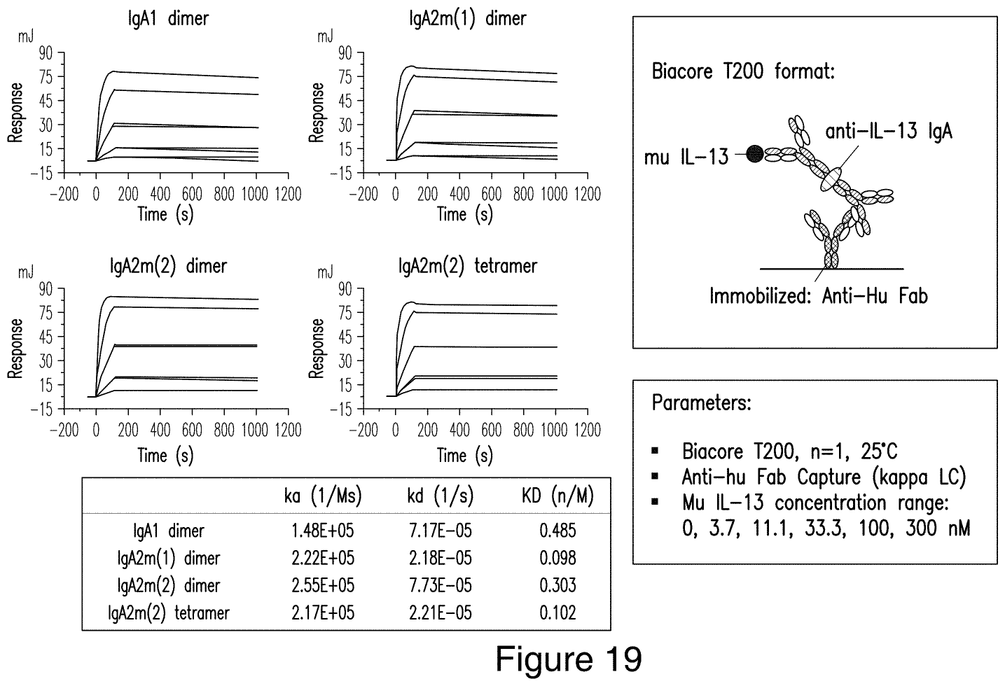

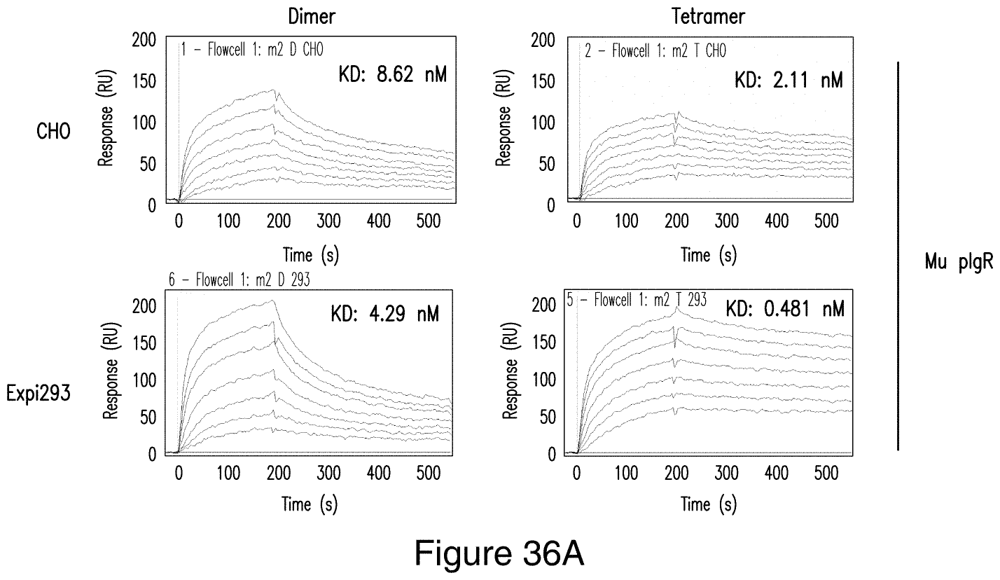

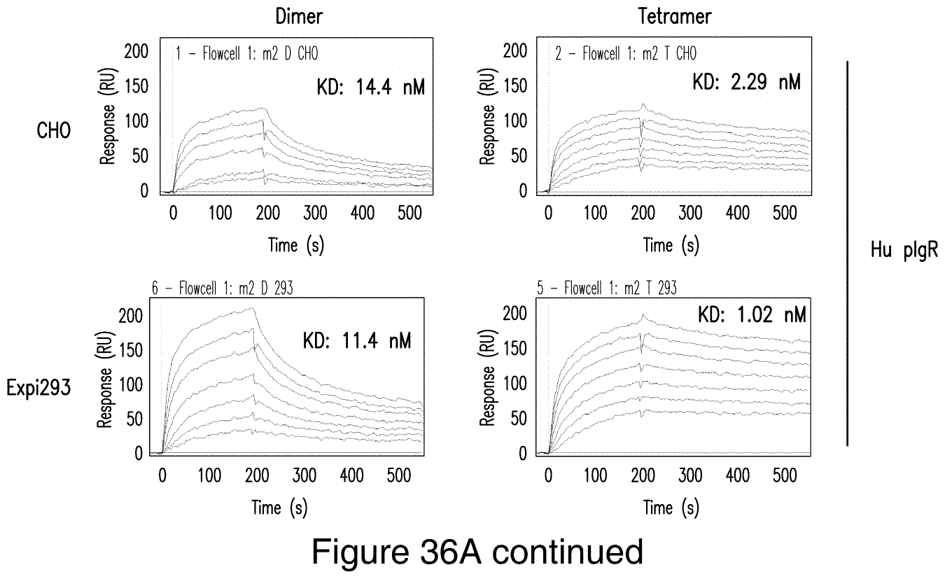

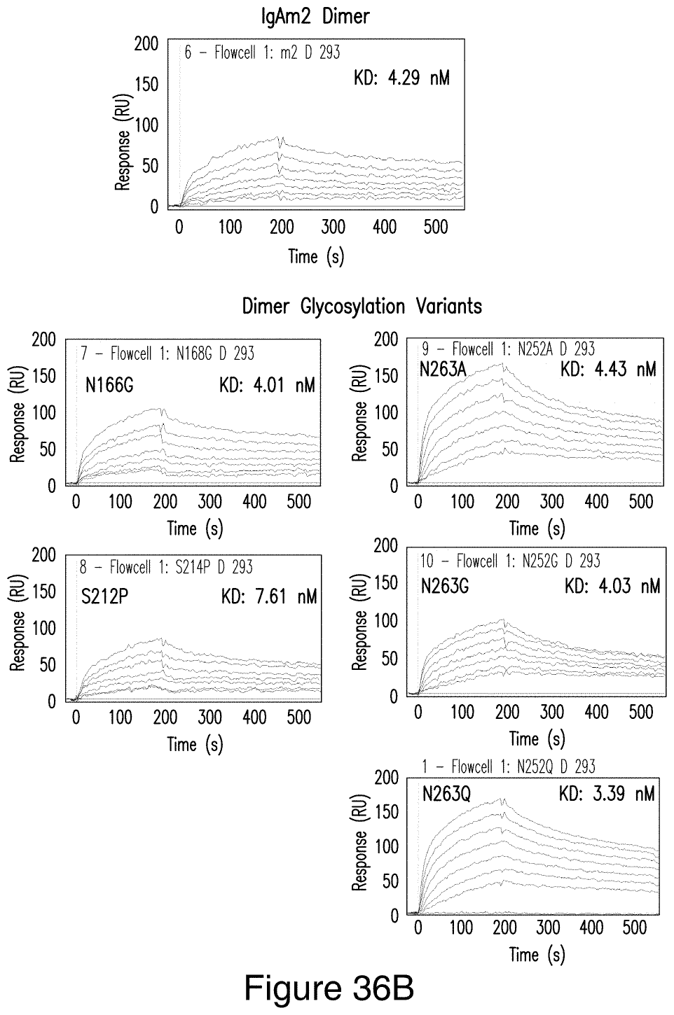

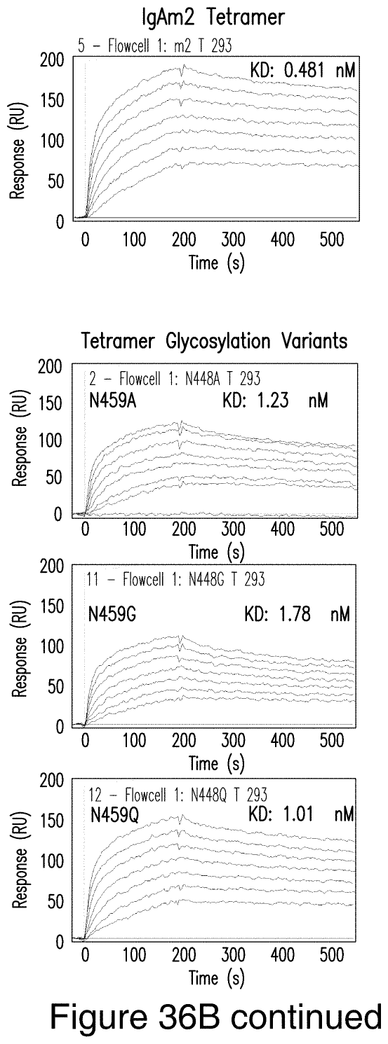

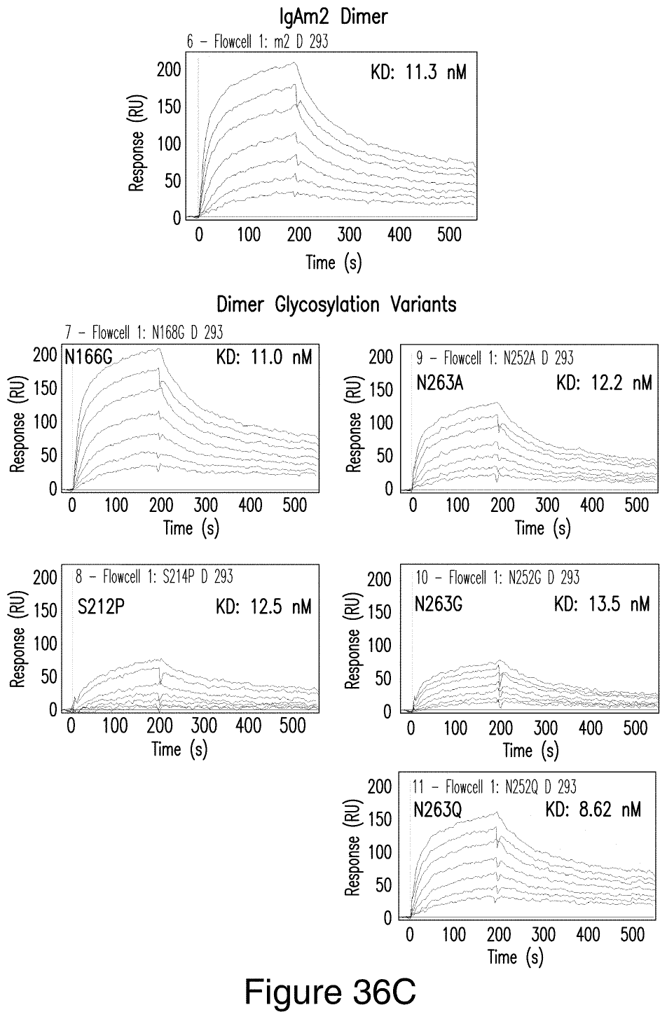

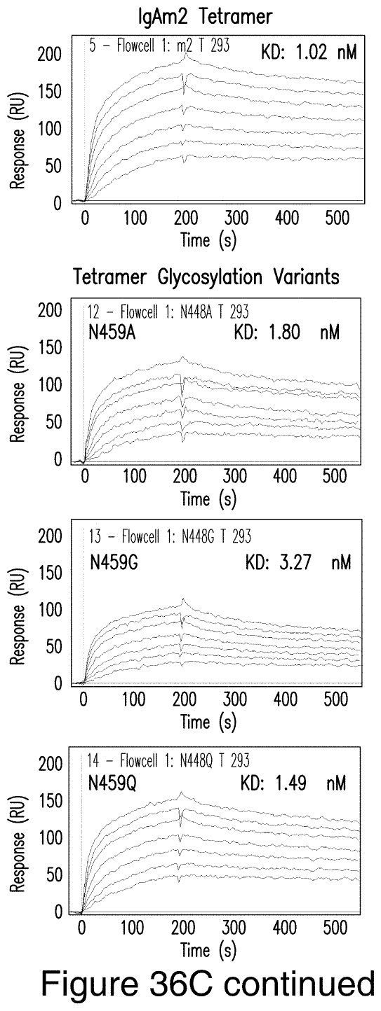

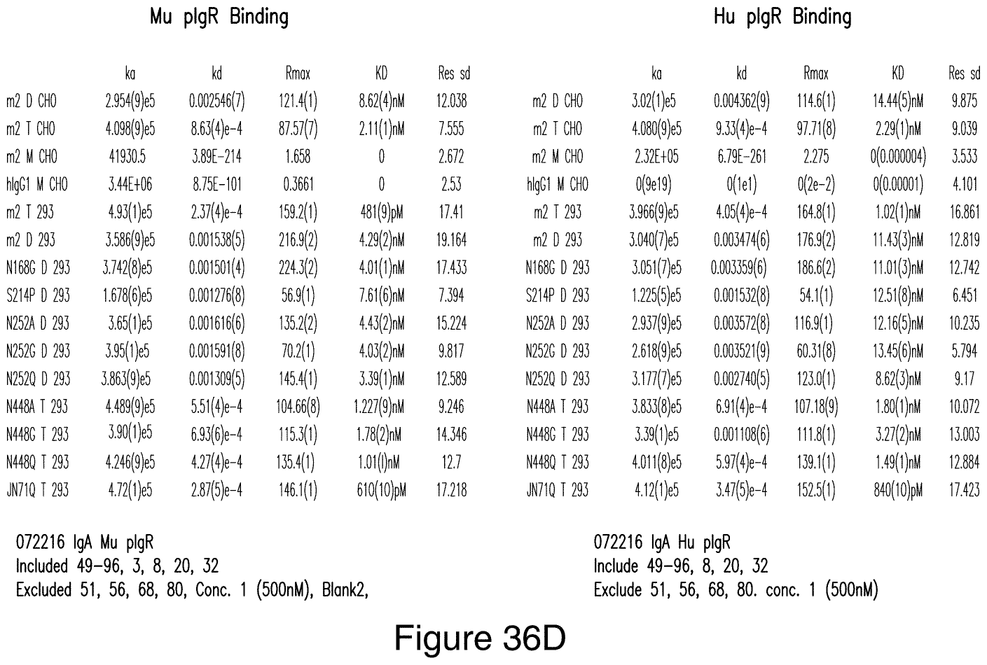

The anti-mIL-13 IgA monomers, dimers, and tetramer bound murine IL-13 with similar affinity as the anti-mIL-13 IgG1 (Table 3 and FIG. 19), indicating that the Fab regions are properly folded and functional in the recombinant IgAs. As expected, mouse and human pIgR binding was only seen for the IgA oligomers, while both monomeric and oligomeric anti-mIL-13 IgA bound with similar affinity to human FcαRI (Table 3). In addition, IgA2m2 purified from transient expression in CHO cells or Expi293 cells exhibited similar binding to mouse and human pIgR (FIG. 36A). Due to pIgR binding capabilities, all IgA oligomers, but not monomers were capable of transcytosis in vitro using an MDCK cell line ectopically expressing human pIgR (FIGS. 4A and 7E). Additionally, the IgA monomers and oligomers all showed increased stability compared to the anti-mIL-13 human IgG1 and similar or increased stability compared to the IgG1 Fab fragment, as measured by differential scanning fluorimetry (DSF) (FIG. 4B).

TABLE 3
Binding affinity of anti-mouse IL-13 IgA and IgG
molecules to antigen and receptors.

| | K_{D} (nM) | | | |
|---|---|---|---|---|
| | Mouse | Mouse | Human | Human |
| | IL-13 | pIgR | pIgR | FcαRI |
| IgAl Monomer | 0.78 ± 0.05 | NB | NB | 425 ± 7 |
| IgA2m1 Monomer | 1.09 ± 0.03 | NB | NB | 429 ± 6 |
| IgA2m1 P221R | 1.16 ± 0.01 | NB | NB | 443 ± 8 |
| Monomer | | | | |
| IgA2m2 Monomer | 0.70 ± 0.01 | NB | NB | 455 ± 5 |
| IgA1 Dimer | 0.34 ± 0.01 | 2.66 ± 1.45 | 8.80 ± 0.55 | 369 ± 3 |
| IgA2m1 Dimer | 0.18 ± 0.01 | 2.54 ± 0.15 | 5.05 ± 0.88 | 499 ± 7 |
| IgA2m1 P221R Dimer | 0.84 ± 0.01 | 5.59 ± 0.03 | 15.5 ± 0.10 | 462 ± 4 |
| IgA2m2 Dimer | 0.81 ± 0.01 | 2.45 ± 0.98 | 13.9 ± 0.10 | 597 ± 5 |
| IgA2m2 Tetramer | 0.97 ± 0.02 | 0.69 ± 0.05 | 1.93 ± 0.02 | 533 ± 5 |
| IgG1 | 0.88 ± 0.05 | NB | NB | NB |

NB: No Binding. All experiments were performed at least n = 3.

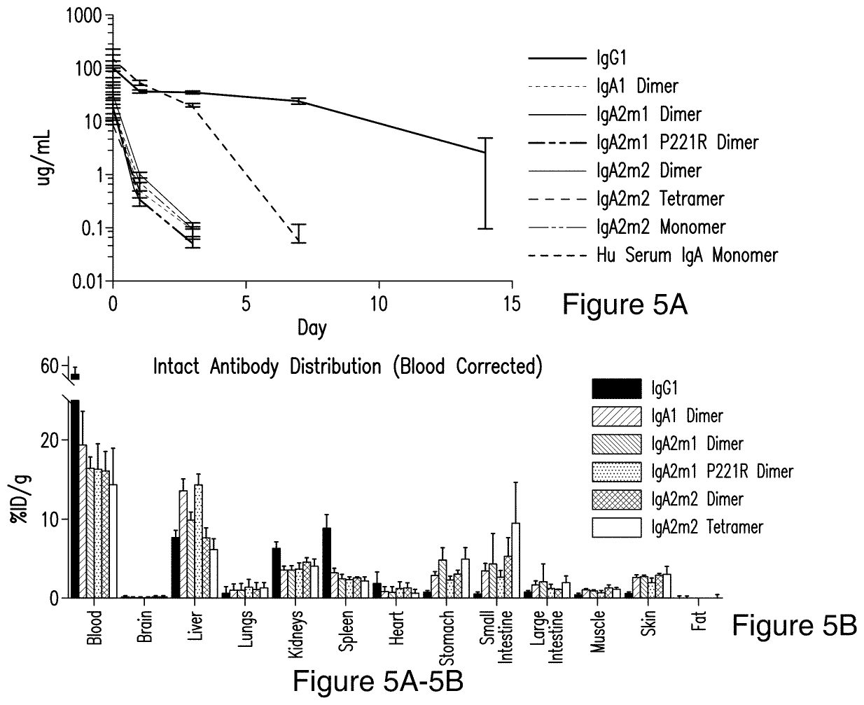

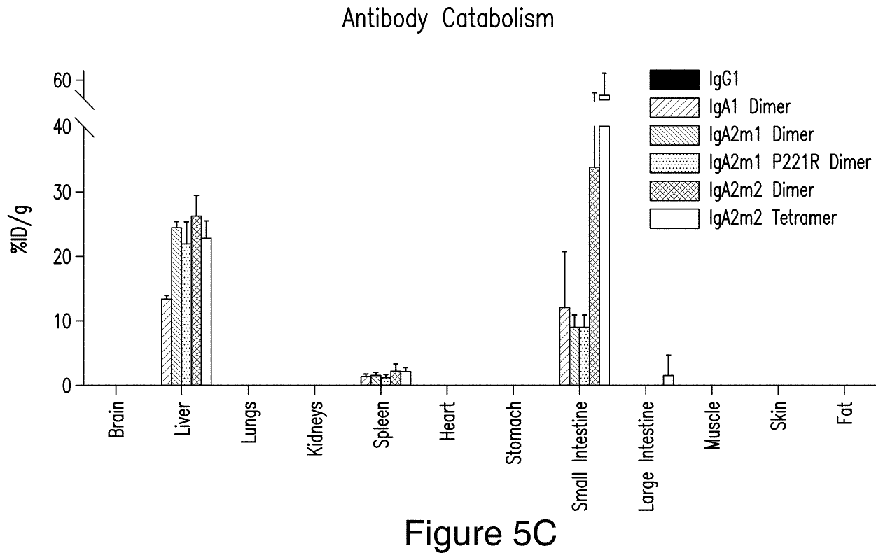

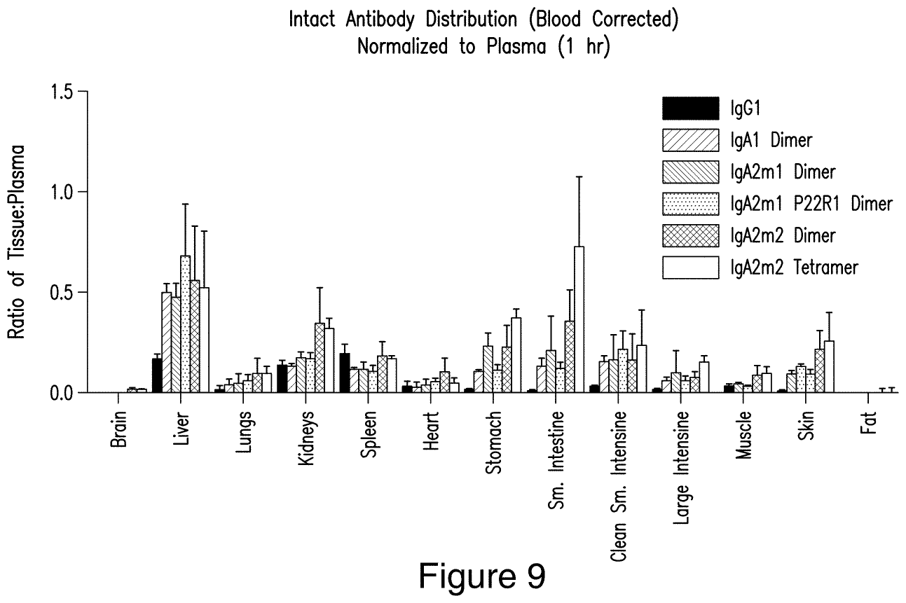

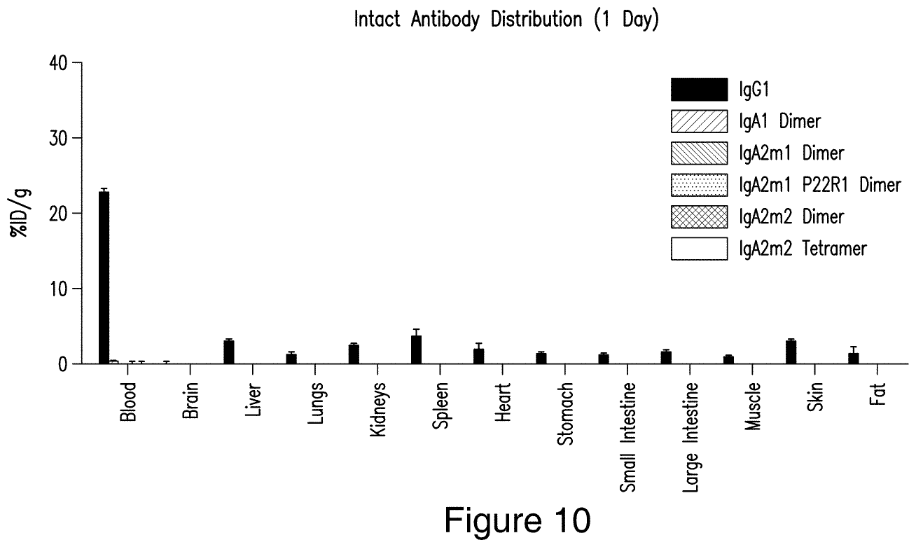

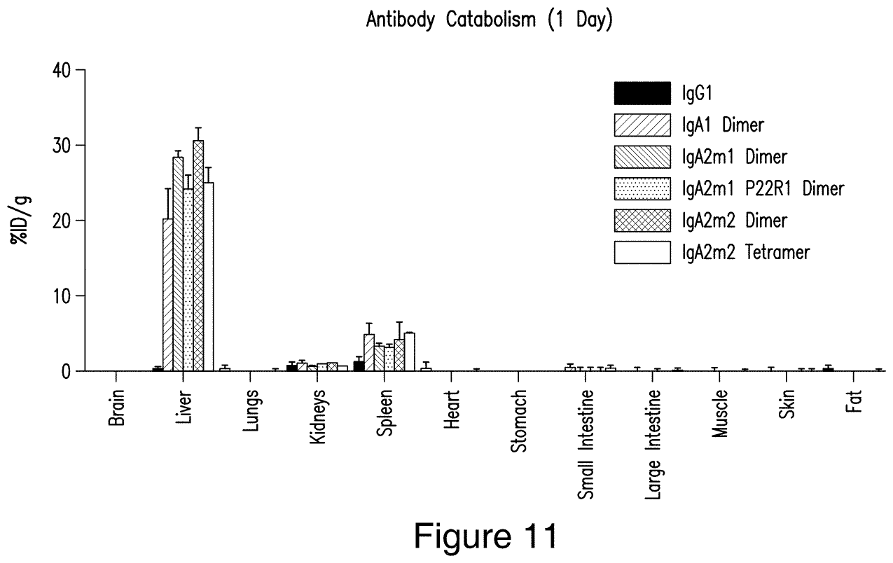

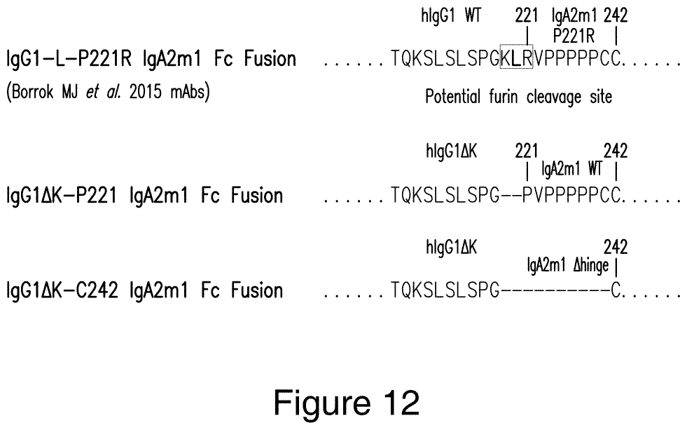

Pharmacokinetic profiles and biodistribution of recombinant IgA. The serum concentration time profiles of the disclosed recombinant IgA oligomers were analyzed, and determined those to be comparable to previously reported data using recombinant monomers (FIG. 5A and Table 4) (Boross et al. (2013), Rouwendal et al. (2016) and Lohse et al., Br J Haematol 112:4170 (2017)). Very rapid serum clearance (>200 mL/day/kg) was observed after a single administration of recombinant IgA oligomers. Serum purified human IgA monomer exhibited slower overall clearance, and a serum PK profile generally in line with that previously reported for a highly sialylated IgA monomer (Rouwendal et al. (2016)). In addition to characterizing the serum concentration time profiles, a radiolabeled biodistribution study in mice with dual I-125 and In-111 labeled antibodies were also performed. The dual tracer approach provided the ability to distinguish between intact antibody prior to lysosomal degradation (I-125) and internalized/catabolized antibody (In-111 minus I-125) as previously described (FIGS. 5B-C) (Boswell et al., Bioconjugate Chem 21:2153-63 (2010), Mandikian et al., Mol Cancer Ther 17:776-85 (2018) and Rajan et al., MAbs 9:1379-88 (2017)). Briefly, iodine rapidly diffuses out of cells and is cleared after iodinated antibodies undergo lysosomal degradation while In-111 labeled antibodies show intercellular accumulation of In-111-adducts following lysosomal degradation. Since the IgA antibodies cleared so rapidly compared to the IgG1, direct comparisons of tissue distribution data are difficult since raw tissue values represent both interstitial and vascular concentrations. Therefore, intact antibody distribution data was blood corrected as previously described to represent only interstitial concentrations in tissues (Boswell et al., Mol Pharmaceutics 11:1591-8 (2014)). Slight enrichment of intact IgA oligomers compared to IgG1 was observed after 1 hour in the liver, stomach, small intestine, large intestine, and skin (all pIgR expressing tissues (Asano et al., Scand J Immunol 60:267-72 (2004) and Wang et al., Scand J Immunol 83:235-43 (2016)), albeit at low levels (FIG. 5B). High levels of IgA degradation were also seen across the formats studied after 1 hour of dosing in the liver and small intestine (FIG. 5C). To account for the difference in total blood concentrations observed between the formats, the ratio of individual tissue to plasma concentrations was also described (FIG. 9). After 1 day, almost no intact IgA antibody was left in the tissues (FIG. 10) and the greatest catabolism in the liver was detected (FIG. 11), although catabolism in the small intestine may not have been detected as In-111 doesn't accumulate very well at later time points in intestinal cells (Boswell et al., British Journal of Pharmacology 168:445-57 (2013)). Without being bound to a particular theory, it was hypothesized that reducing degradation and eventual clearance mechanisms of IgA could further improve uptake of IgA molecules into mucosal tissue compared with IgG.

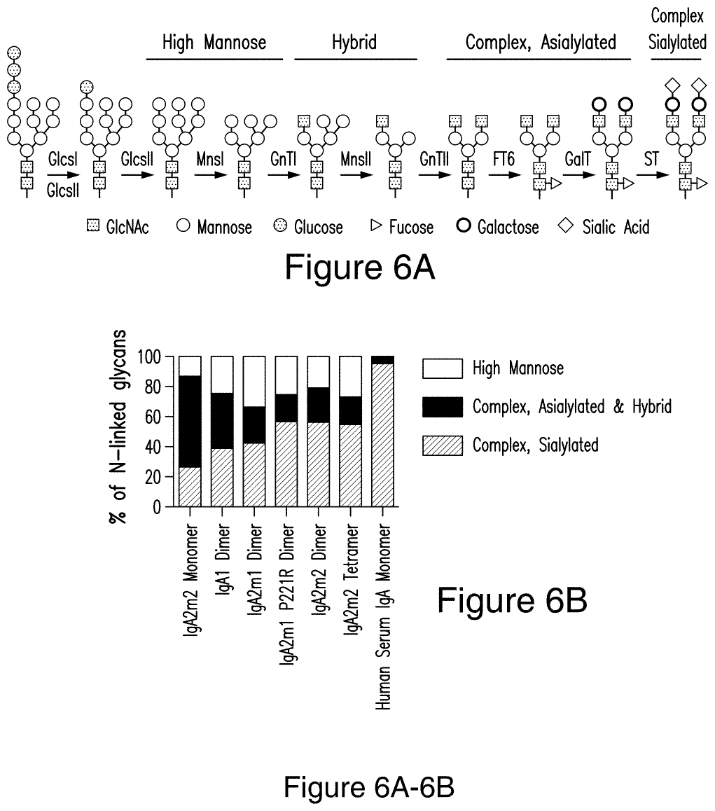

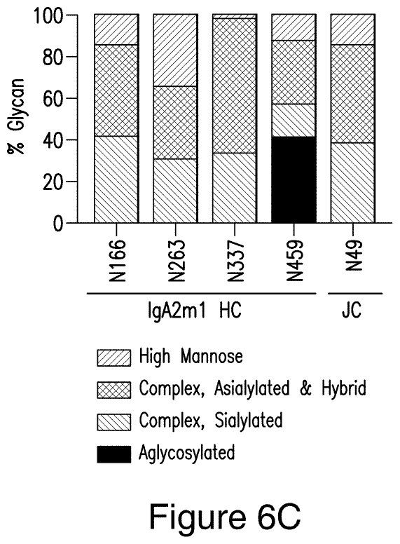

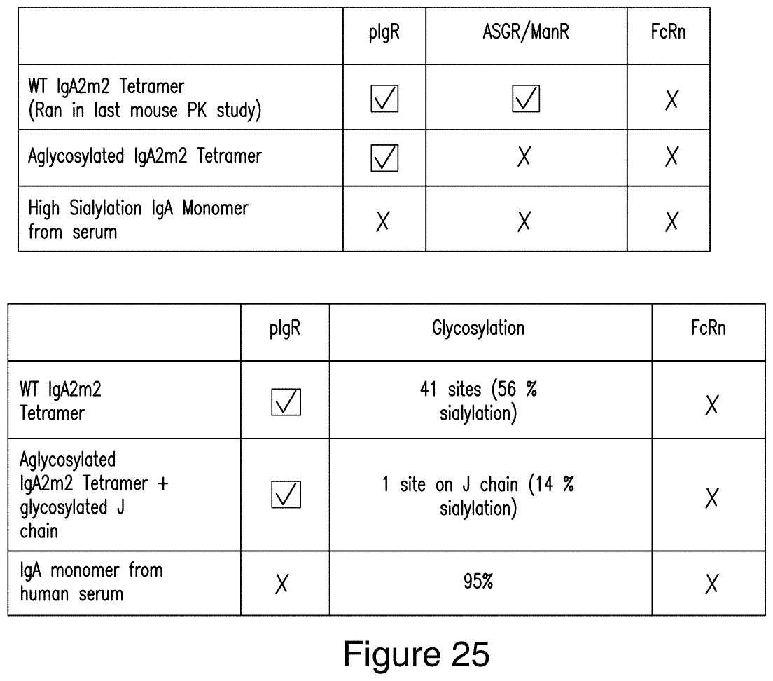

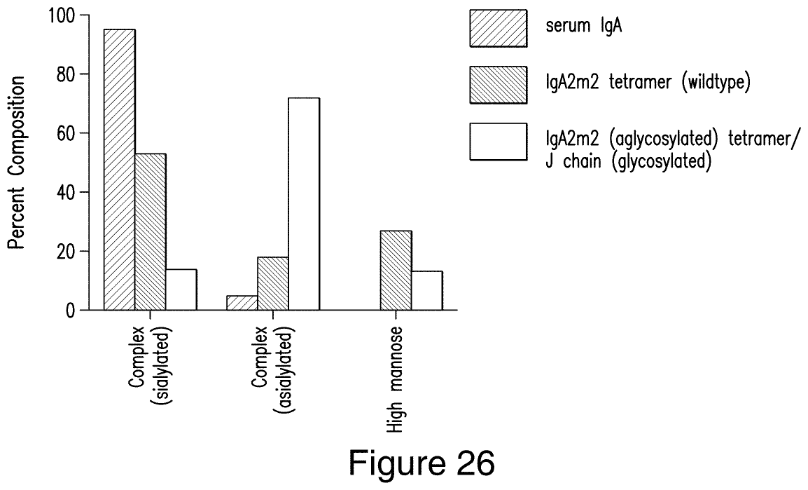

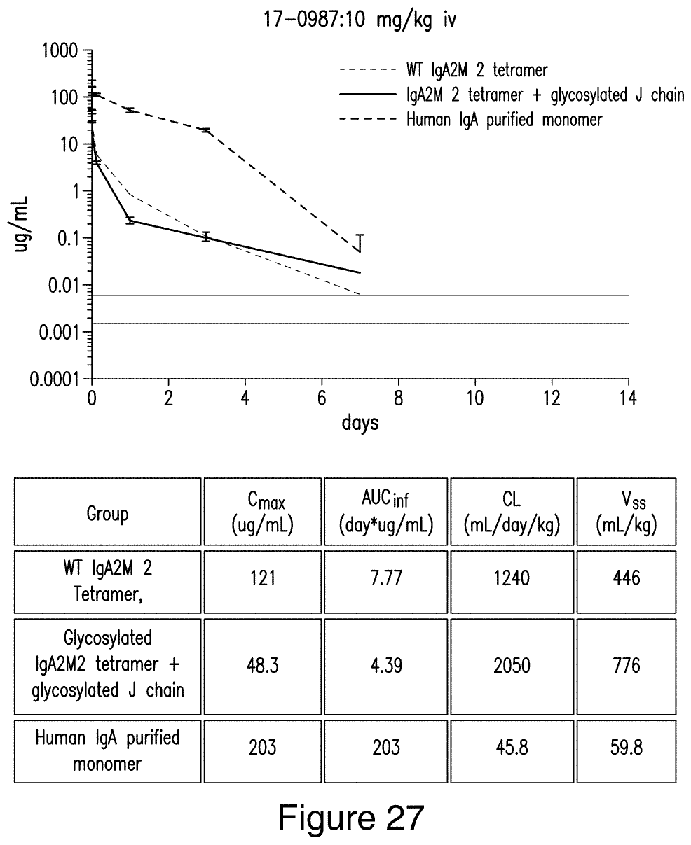

Sialylation content on the N-linked glycans of monomeric IgA molecules has been reported to negatively correlate with antibody clearance via specific glycan receptors (Rouwendal et al. (2016)). Thus, the disclosed IgA molecules were analyzed to determine their overall sialylation content. The glycans were classified into categories based on the level of processing with complex and sialylated being the most desired for the IgA molecules (FIG. 6A). The recombinantly produced dimers of IgA1, IgA2m1, IgA2m1.P221R, IgA2m2 as well as IgA2m2 monomer and tetramer were about 20-50% sialylated (FIG. 6B, FIG. 25 and FIG. 26, and Table 5). This indicates that the IgA molecules contain incompletely processed glycans that can be recognized by glycan receptors. Additionally, the sialylation content at each site on the IgA2m1 dimer were examined and it was found that all sites, including the site on the JC, contained incompletely processed glycans, suggesting the incomplete glycan processing isn't occurring at only one specific site (FIG. 6C and Table 6). In contrast to the disclosed recombinant IgA molecules, IgA purified from human serum has a sialylation content of 95% (FIG. 6B and Table 5), and was monomeric as determined by SEC-MALS. As serum IgA is known to be predominantly monomeric (Kerr (1990)), it may be enriched for highly sialylated molecules since sialylation content positively correlates with the systemic exposure of antibodies. Without being bound by a particular theory, it is thought that this increased sialylation level of the human purified IgA monomer would correlate with decreased serum clearance of the molecule in mice relative to recombinant IgA monomer. Indeed, this was demonstrated to be true, which suggests that binding to specific glycan receptors in the liver may be an important clearance mechanism for IgA monomer (FIG. 5A, FIG. 25 and FIG. 27).

TABLE 4
Pharmacokinetic Parameter Estimates (Mean) after a 5 mg/kg IV
bolus of IgA monomers/oligomers to Balb/C mice

| | | | | Half |
|---|---|---|---|---|
| | Cₘₐₓ | AUCₗₐₛₜ | CL | Life |
| | (μg/mL) | (day · μg/mL) | (mL/day/kg) | (Days) |
| IgA2m2 Monomer | 76.42 | 8.674 | 573.2 | 0.36 |
| IgA1 Dimer | 85.17 | 14.61 | 341.3 | 0.31 |
| IgA2m1 Dimer | 92.53 | 13.40 | 372.9 | 0.26 |
| IgA2m1 P221R Dimer | 107.8 | 17.55 | 284.5 | 0.27 |
| IgA2m2 Dimer | 144.7 | 20.88 | 238.9 | 0.31 |
| IgA2m2 Tetramer | 60.47 | 6.127 | 788.1 | 0.22 |
| Human Serum IgA | 203.3 | 203.0 | 45.74 | 0.97 |
| Monomer^{#} | | | | |
| IgG1 | 100.1 | 339.3 | 14.30 | 2.89 |

AUCₗₐₛₜ = area under the concentration-time curve, last measurable concentration;
CL = clearance;
Cₘₐₓ = maximum concentration observed;
IV = intravenous;
^{#}Dosed 10 mg/kg IV bolus to Balb/c mice.
Note:
As sparse PK analysis was performed for all mouse PK data, data from individual mice per group was pooled and SD was not reported.

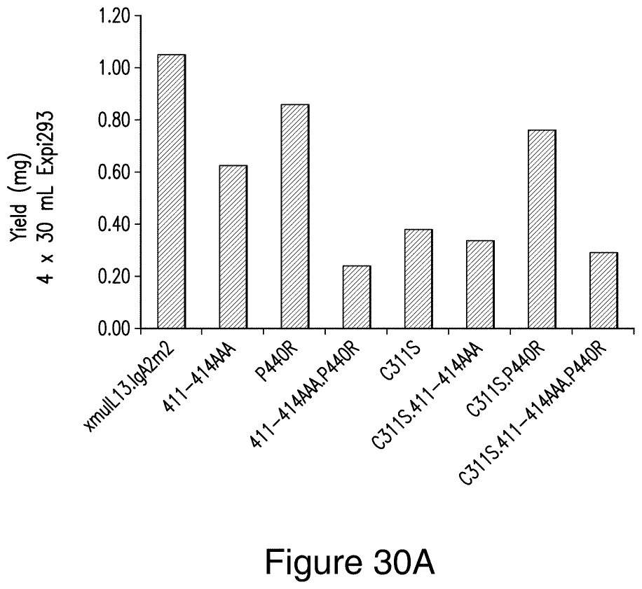

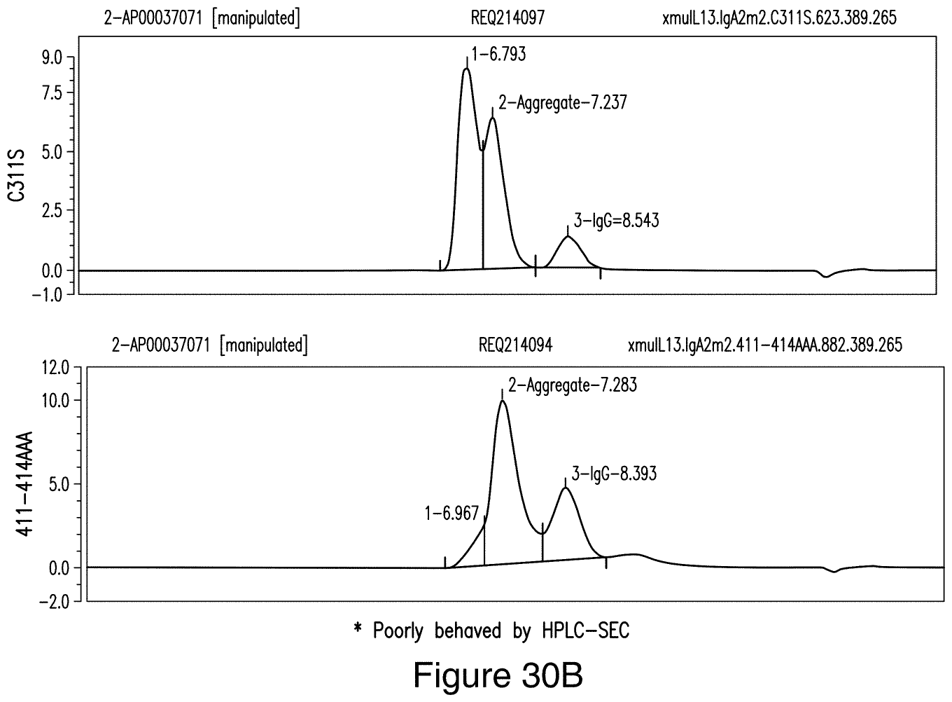

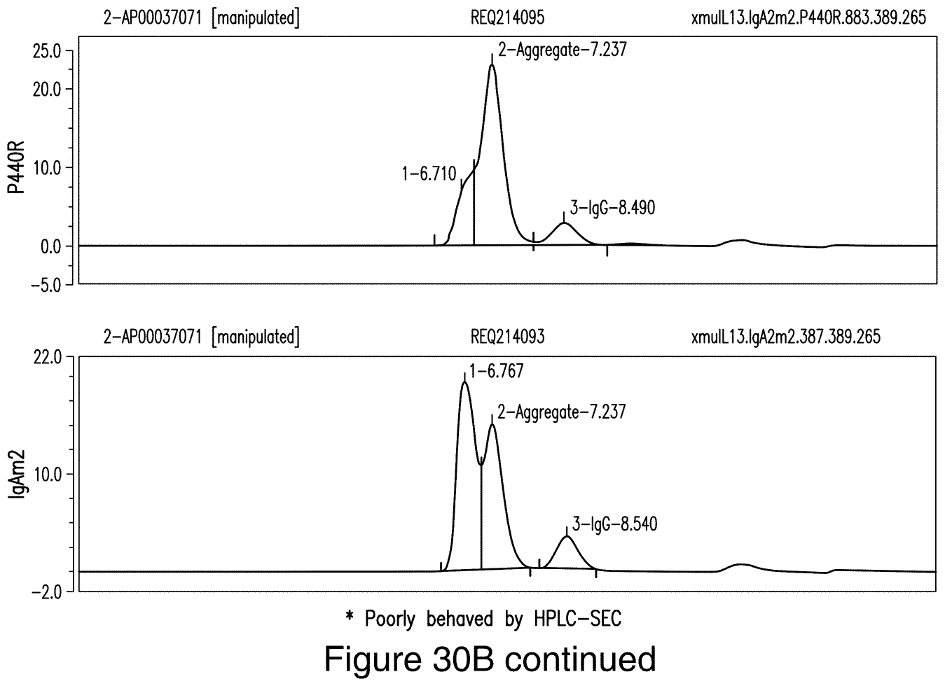

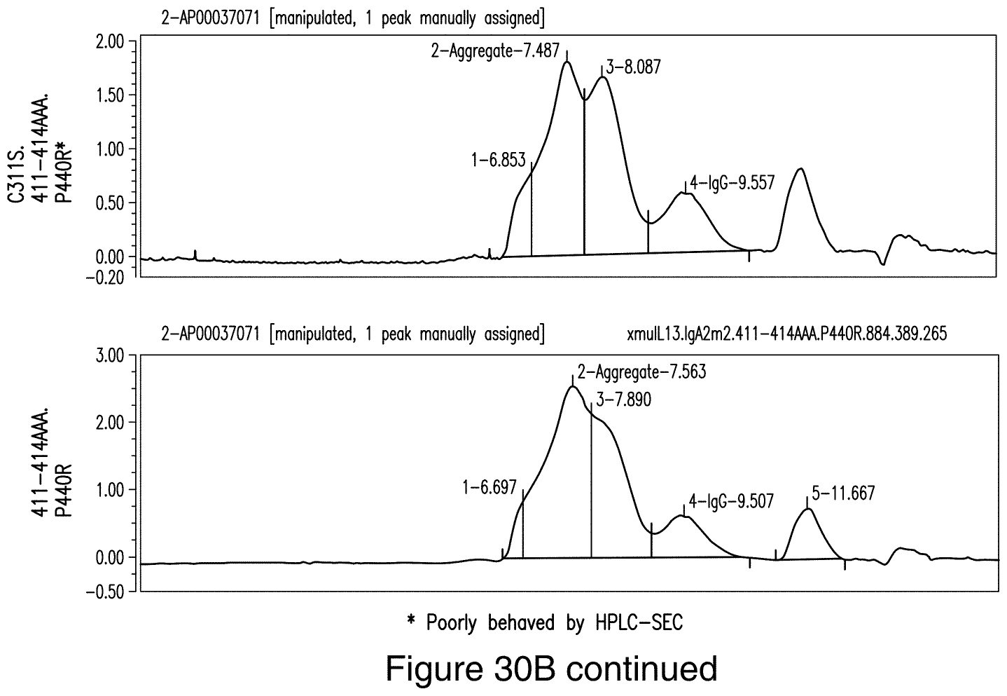

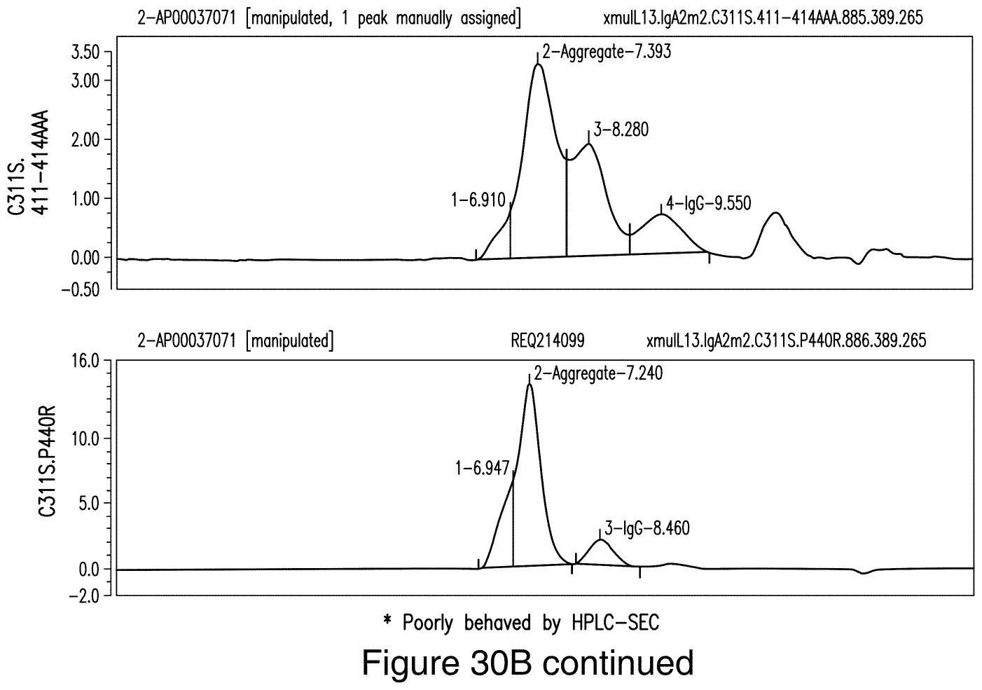

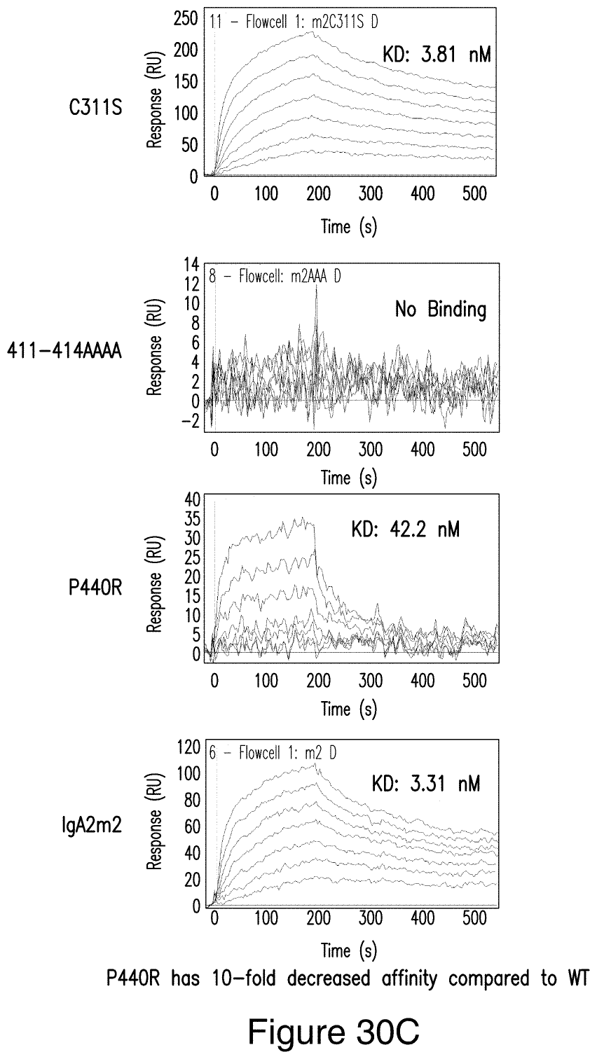

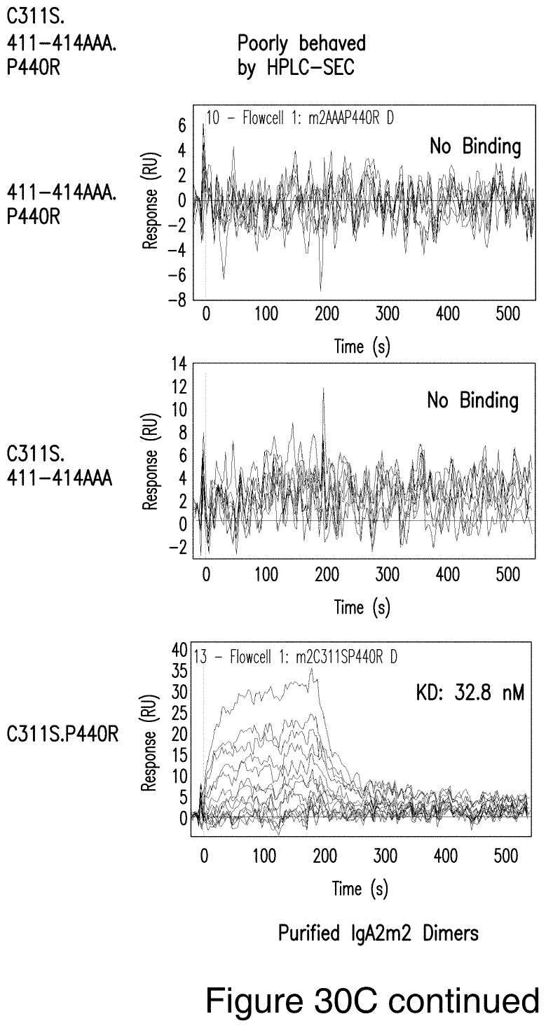

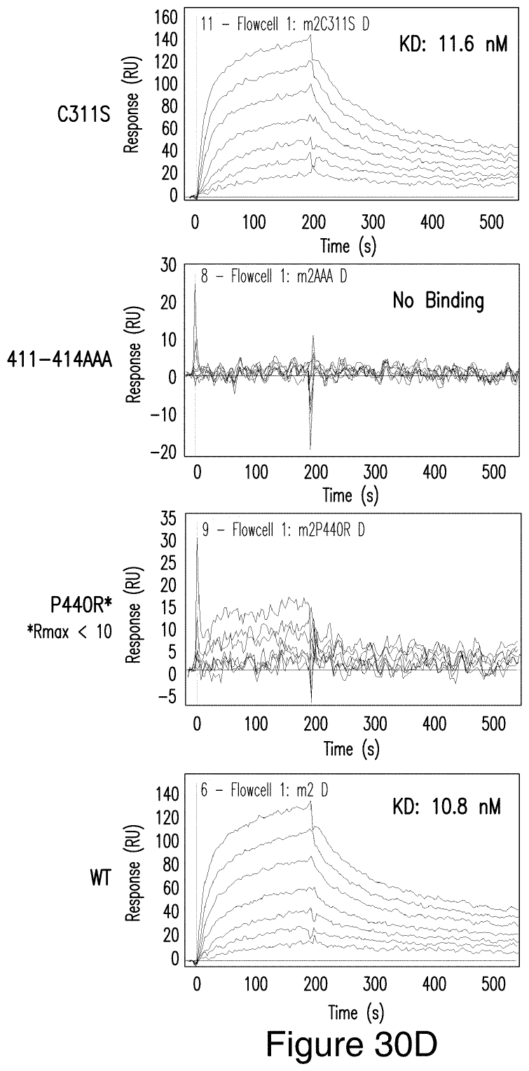

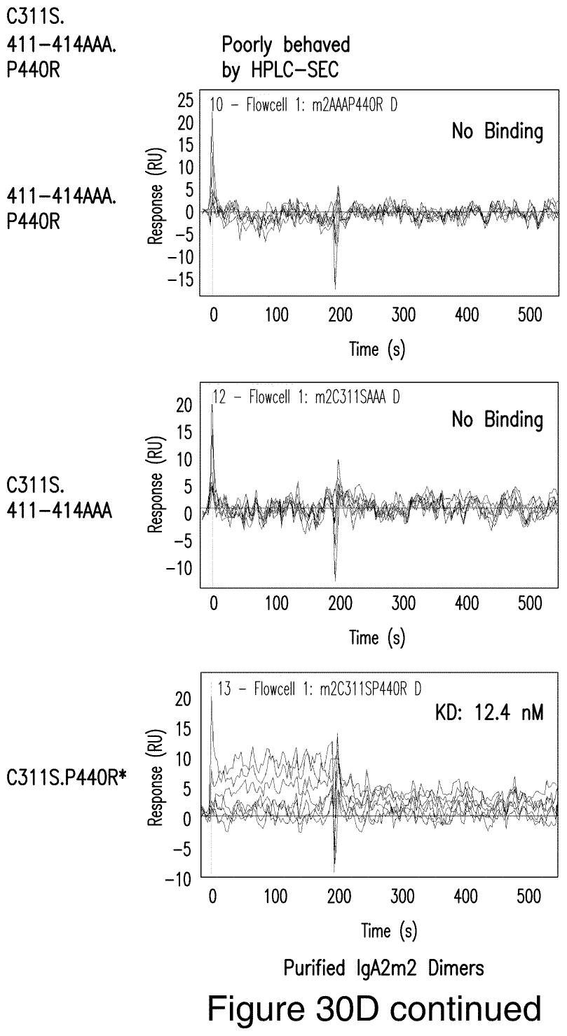

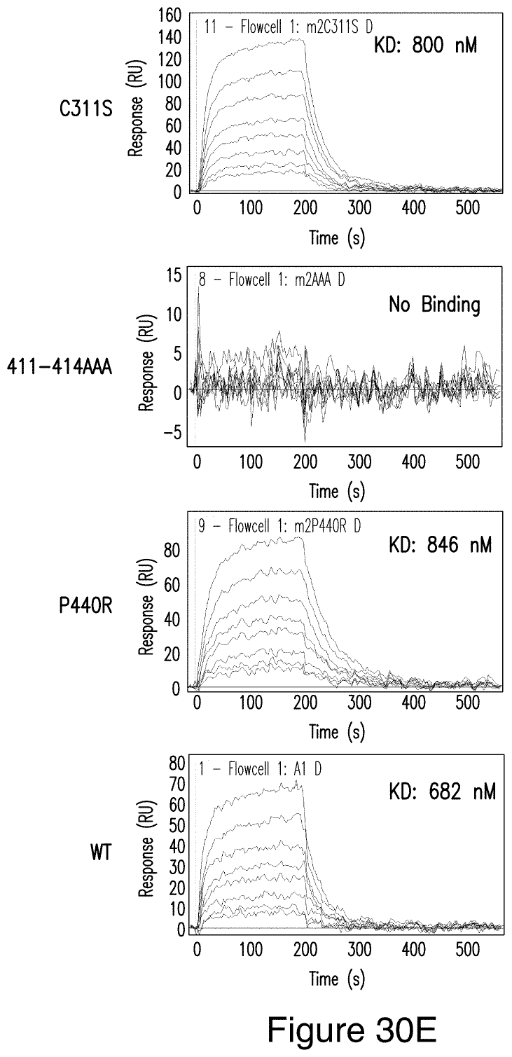

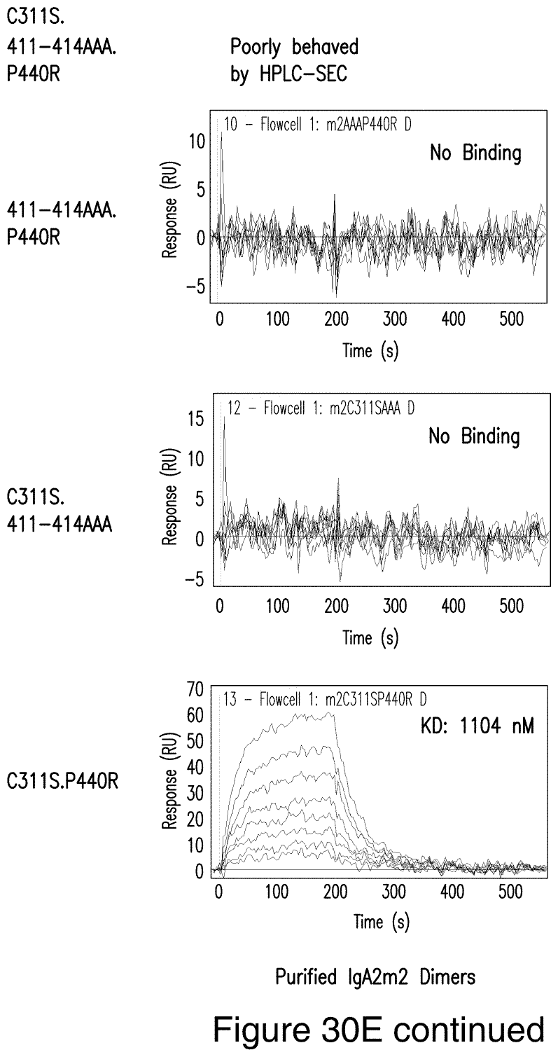

Generation of IgA variants that have reduced pIgR binding. Variants of IgA2m2 were generated to determine the effect of mutations on pIgR binding. IgA2m2 variants that have a mutation of amino acids Y411, V413 and T414 to alanine (referred to herein as “411-414AAA”), a P440R mutation, a C311S mutation or combinations thereof were generated. Expression levels of such variants are provided in FIG. 30A. FIG. 30B provides the SEC characterization of small scale purified anti-IL-13 IgA2m2 variants. As shown in FIG. 30C-D, IgA2m2 variants that have a mutation of amino acids Y411, V413 and T414 do not bind to mouse pIgR or human pIgR while the P440R variant resulted in a 10-fold decreased affinity to murine pIgR and a significant loss in binding capacity to human pIgR. In addition, IgA2m2 variants that have a mutation of amino acids 411, 413 and 414 also do not bind to FcαRI (FIG. 30E).

TABLE 5
Global N-linked glycan analysis of monomeric and polymeric IgA molecules.

| | | | | | | | | Human |
|---|---|---|---|---|---|---|---|---|
| | | | | | IgA2m1 | | | Serum |
| | | IgA2m2 | IgA1 | IgA2m1 | P221R | IgA2m2 | IgA2m2 | IgA |
| | | monomer | Dimer | Dimer | Dimer | Dimer | Tetramer | Monomer |
| High Mannose | [symbol] | 1.4% | 0% | 0% | 0% | 0% | 0% | 0% |
| | [symbol] | 1.7% | 1.1% | 0% | 0.9% | 0% | 2.2% | 0% |
| | [symbol] | 2.7% | 13.6% | 11.8% | 6.4% | 4.7% | 3.9% | 0% |
| | [symbol] | 2.6% | 6.6% | 4.7% | 3.4% | 3.2% | 4.2% | 0% |
| | [symbol] | 2.3% | 0% | 0.8% | 0.5% | 0% | 1.5% | 0% |
| | [symbol] | 1.3% | 0% | 0% | 0% | 0% | 0% | 0% |
| | [symbol] | 1.1% | 0% | 0% | 0% | 0% | 0% | 0% |
| | [symbol] | 0% | 0% | 0% | 0.9% | 0% | 0% | 0% |
| | [symbol] | 0% | 3.9% | 16.8% | 14.0% | 13.2% | 15.1% | 0% |
| Complex, Asialylated | [symbol] | 0.6% | 4.7% | 2.2% | 1.0% | 1.6% | 0.8% | 0% |
| & Hybrid | [symbol] | 0% | 1.3% | 1.0% | 0% | 0% | 0% | 0% |
| | [symbol] | 1.9% | 8.3% | 7.6% | 1.8% | 2.0% | 3.2% | 0% |
| | [symbol] | 1.0% | 2.3% | 0.8% | 0.8% | 0% | 0% | 0% |
| | [symbol] | 0.8% | 0% | 0% | 0% | 0% | 0% | 0% |
| | [symbol] | 0% | 0% | 0.9% | 0% | 0.8% | 0.9% | 2.3% |
| | [symbol] | 0% | 0% | 0% | 0% | 0% | 0% | 1.2% |
| | [symbol] | 1.2% | 2.8% | 1.9% | 2.8% | 3.3% | 0.5% | 0% |
| | [symbol] | 32.8% | 12.7% | 5.8% | 6.7% | 9.3% | 8.3% | 0% |
| | [symbol] | 2.6% | 0% | 0% | 0% | 0% | 0% | 0% |
| | [symbol] | 0% | 0% | 0.9% | 0% | 1.7% | 0% | 0% |
| | [symbol] | 10.5% | 2.6% | 2.9% | 2.8% | 3.4% | 3.6% | 0% |
| | [symbol] | 8.4% | 0% | 0% | 0% | 0% | 0.9% | 0% |
| | [symbol] | 1.2% | 0% | 0% | 0% | 0% | 0% | 0.6% |
| | [symbol] | 0% | 0.8% | 0% | 0% | 0% | 0% | 1.0% |
| | [symbol] | 0% | 0% | 0% | 1.3% | 0.8% | 0% | 0% |
| Complex, Sialylated | [symbol] | 0% | 0% | 0.6% | 0% | 0% | 0% | 0% |
| | [symbol] | 0% | 1.3% | 2.3% | 1.7% | 3.0% | 2.7% | 0% |
| | [symbol] | 0% | 0% | 0% | 0% | 0% | 0% | 1.4% |
| | [symbol] | 0% | 1.8% | 0% | 0% | 0.7% | 0% | 0% |
| | [symbol] | 0% | 0% | 0.9% | 0% | 0% | 0% | 0% |
| | [symbol] | 0% | 14.7% | 0% | 0% | 2.1% | 0% | 6.0% |
| | [symbol] | 0% | 0.7% | 0% | 0% | 0% | 0% | 0% |
| | [symbol] | 0% | 0% | 0% | 0% | 0% | 0% | 6.0% |
| | [symbol] | 0% | 1.2% | 0% | 0.9% | 1.1% | 0.7% | 0% |
| | [symbol] | 0% | 0% | 1.0% | 0% | 0% | 0% | 0% |
| | [symbol] | 1.0% | 0% | 0% | 0% | 0% | 0% | 0% |
| | [symbol] | 0.5% | 0.8% | 3.7% | 0% | 0% | 0% | 33.6% |
| | [symbol] | 10.2% | 0% | 0% | 0% | 0% | 0% | 0% |
| | [symbol] | 0% | 7.0% | 0.5% | 3.4% | 3.8% | 0% | 1.0% |
| | [symbol] | 14.1% | 7.1% | 32.7% | 49.9% | 45.6% | 48.9% | 2.1% |
| | [symbol] | 0% | 1.5% | 0% | 0% | 0% | 0% | 0% |
| | [symbol] | 0% | 0% | 0% | 0% | 0% | 0% | 2.0% |
| | [symbol] | 0% | 0% | 0% | 0% | 0% | 0% | 25.4% |
| | [symbol] | 0% | 0.9% | 0% | 0.8% | 0% | 0% | 0% |
| | [symbol] | 0% | 1.6% | 0% | 0% | 0% | 0% | 0% |
| | [symbol] | 0% | 0.5% | 0% | 0% | 0% | 0.7% | 7.2% |
| | [symbol] | 0% | 0% | 0% | 0% | 0% | 0% | 10.1% |
| | [symbol] | 0% | 0% | 0% | 0% | 0% | 1.7% | 0% |

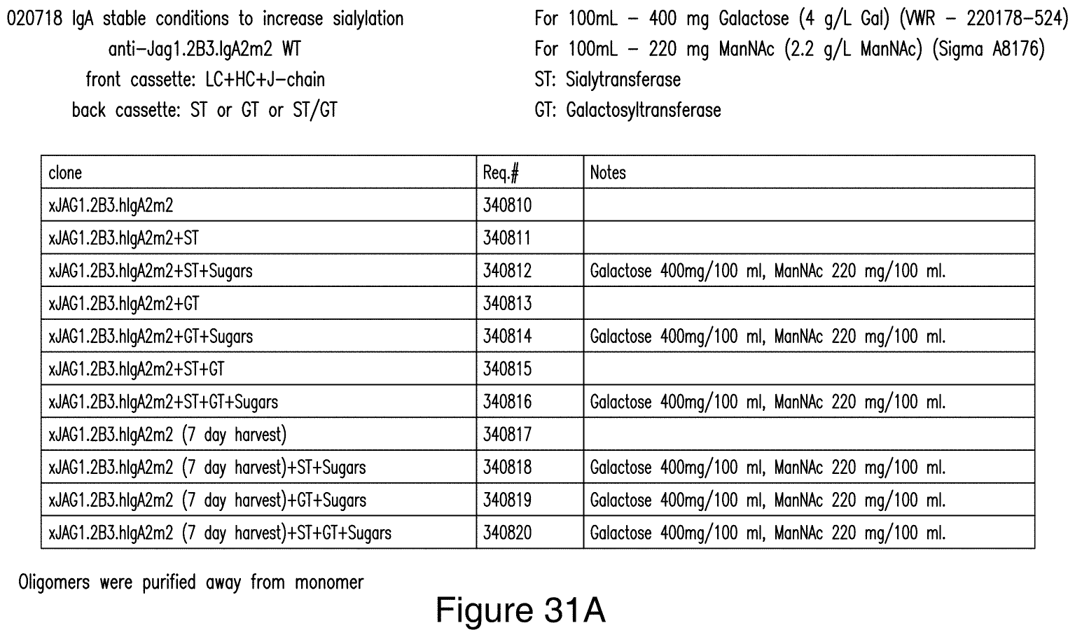

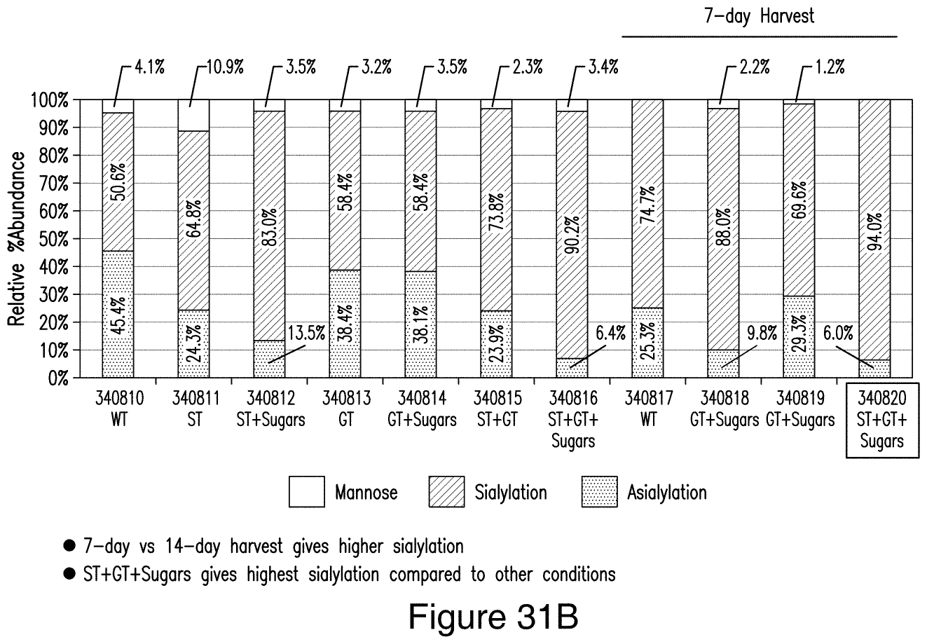

Modifying the cell culture conditions to increase sialylation content of the IgA antibodies. The culturing conditions of cells expressing IgA antibodies were modified to increase the sialylation content of the antibodies. The cell culture conditions that were tested are provided in FIG. 31A. As shown in FIG. 31B, sialylation of the IgA2m2 antibodies increased upon the addition of sialytransferase (ST) and galactosyltransferase (GT) in the presence of galactose and N-Acetylmannosamine (ManNac) with a 7-day harvest.

TABLE 6
Site specific N-linked glycan analysis of IgA2m1 dimer.

| | | HC N166 | HC N263 | HC N337 | HC N459 | JC N49 |
|---|---|---|---|---|---|---|
| Aglycosylated | | 0% | 0% | 0% | 41.4% | 0% |
| High Mannose | [symbol] | 0% | 0.6% | 0% | 0% | 1.7% |
| | [symbol] | 1.8% | 12.5% | 1.7% | 4.2% | 1.6% |
| | [symbol] | 0% | 12.1% | 0% | 5.6% | 2.9% |
| | [symbol] | 0.8% | 6.1% | 0% | 0.7% | 0% |
| | [symbol] | 0% | 3.5% | 0% | 0% | 9.7% |
| | [symbol] | 1.0% | 1.2% | 0% | 0% | 0% |
| | [symbol] | 11.4% | 0% | 0% | 2.6% | 0% |
| Complex, Asialylated & Hybrid | [symbol] | 1.0% | 10.0% | 1.3% | 0.6% | 1.2% |
| | [symbol] | 0% | 4.3% | 0% | 0% | 3.0% |
| | [symbol] | 0.7% | 11.1% | 1.8% | 1.5% | 0.9% |
| | [symbol] | 2.6% | 6.3% | 1.0% | 0.8% | 4.6% |
| | [symbol] | 0.7% | 2.8% | 0.7% | 1.7% | 8.6% |
| | [symbol] | 1.3% | 0% | 0% | 0% | 0% |
| | [symbol] | 0% | 0% | 0% | 0% | 4.8% |
| | [symbol] | 0% | 0% | 0% | 1.0% | 0% |
| | [symbol] | 0% | 0% | 0% | 0% | 1.1% |
| | [symbol] | 0% | 0% | 0% | 0% | 0% |
| | [symbol] | 3.1% | 0% | 2.9% | 0% | 0% |
| | [symbol] | 16.8% | 0% | 24.8% | 1.7% | 10.6% |
| | [symbol] | 0% | 0% | 1.8% | 1.3% | 1.0% |
| | [symbol] | 1.3% | 0% | 1.2% | 0% | 1.6% |
| | [symbol] | 11.6% | 0% | 17.6% | 19.7% | 1.8% |
| | [symbol] | 4.5% | 0% | 9.7% | 0.6% | 3.9% |
| | [symbol] | 0.8% | 0% | 0% | 0% | 0% |
| | [symbol] | 0% | 0% | 1.1% | 0.6% | 0.6% |
| | [symbol] | 0% | 0% | 0.8% | 0% | 0% |
| Compl | [symbol] | 0% | 0% | 0.6% | 0% | 2.7% |
| | [symbol] | 0% | 3.6% | 0% | 0.7% | 0% |
| | [symbol] | 4.2% | 0% | 0% | 0.7% | 1.9% |
| | [symbol] | 0% | 7.8% | 0% | 1.1% | 0% |
| | [symbol] | 0.7% | 0% | 0% | 0% | 0% |
| | [symbol] | 0% | 4.0% | 0% | 2.2% | 0% |
| | [symbol] | 0% | 4.2% | 0% | 0.9% | 2.3% |
| | [symbol] | 1.3% | 10.1% | 0.8% | 1.0% | 1.5% |
| | [symbol] | 6.5% | 0% | 10.6% | 1.2% | 0% |
| | [symbol] | 1.5% | 0% | 0% | 0% | 0% |
| | [symbol] | 11.9% | 0% | 16.9% | 2.0% | 0% |
| | [symbol] | 0.7% | 0% | 0% | 0% | 0% |
| | [symbol] | 1.7% | 0% | 0% | 0% | 0% |
| | [symbol] | 11.4% | 0% | 3.9% | 2.6% | 0.8% |
| | [symbol] | 0% | 0% | 0% | 0.8% | 0% |
| | [symbol] | 0% | 0% | 0% | 0.7% | 0% |
| | [symbol] | 0.7% | 0% | 0.6% | 0.6% | 0.6% |
| | [symbol] | 0% | 0% | 0% | 0% | 0.7% |
| | [symbol] | 0% | 0% | 0% | 0% | 6.0% |
| | [symbol] | 0% | 0% | 0% | 1.2% | 21.1% |

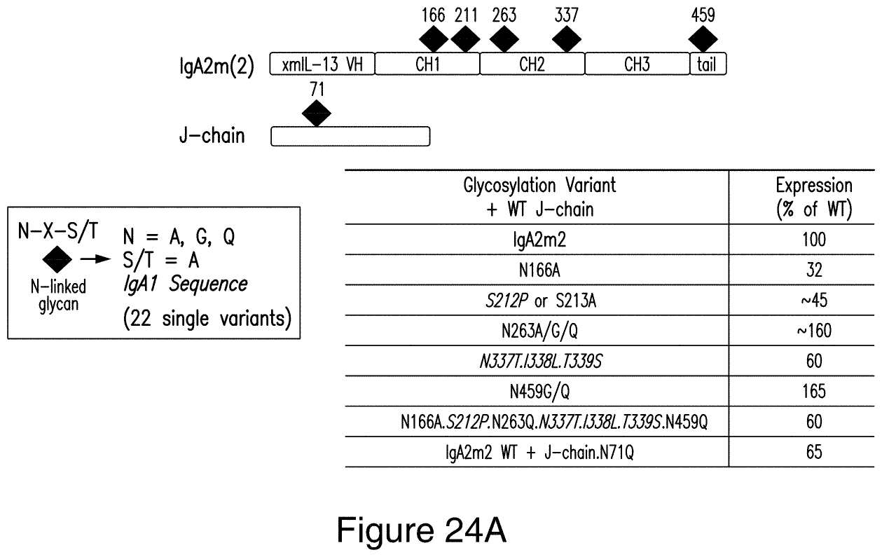

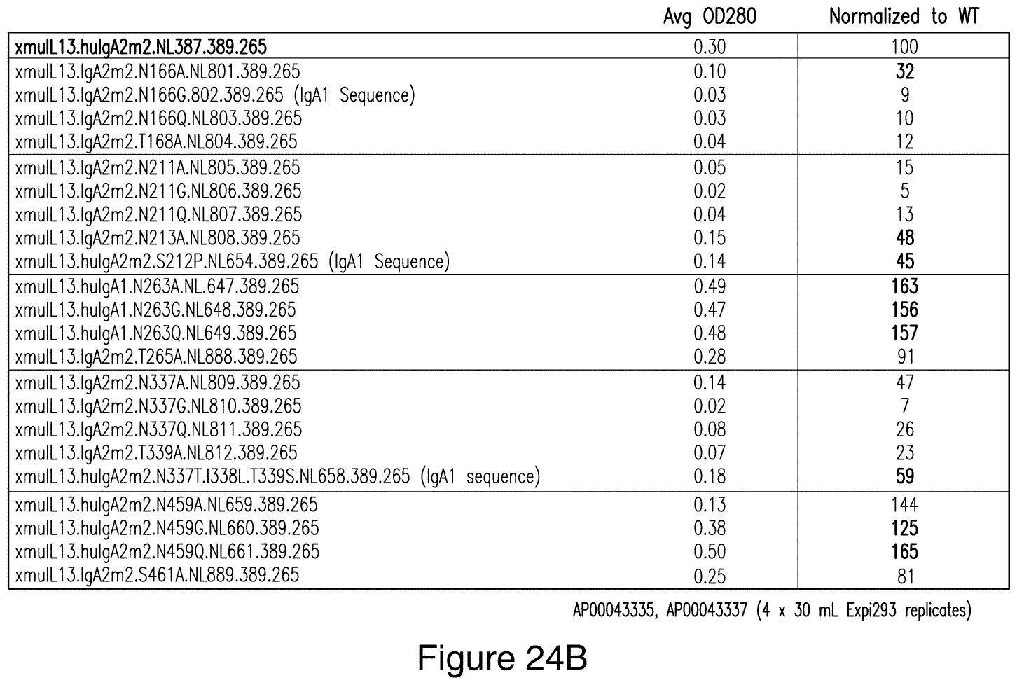

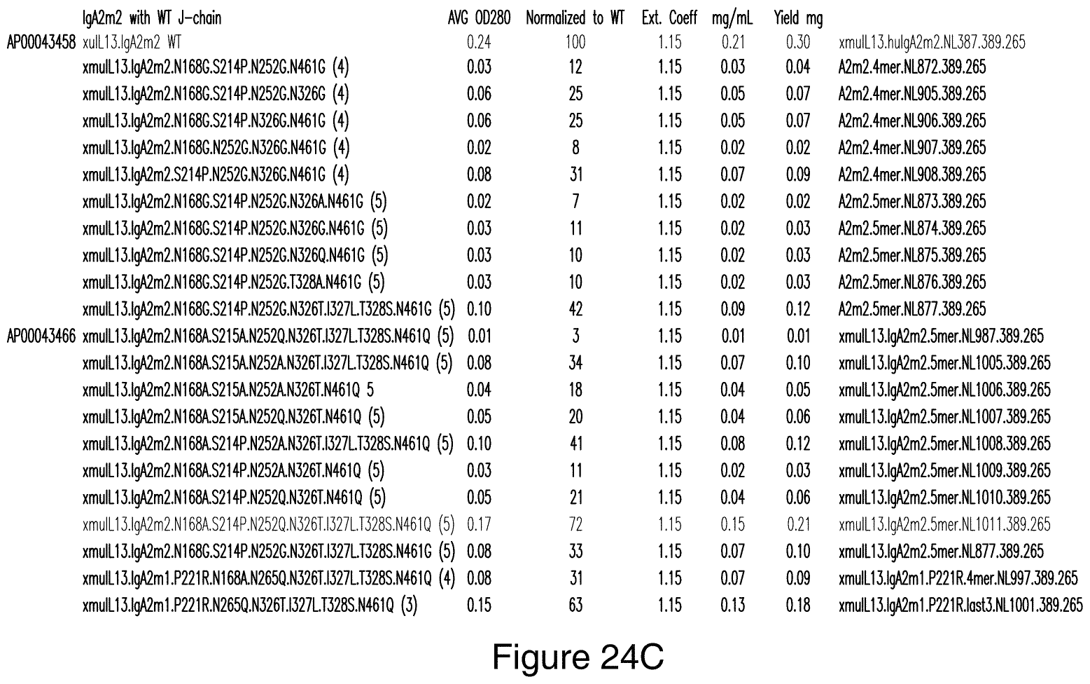

Improving the pharmacokinetic profile of recombinant IgA through glycosylation site and FcRn engineering. Two parallel approaches were taken to reduce the clearance of recombinant polymeric IgA in mice. First, all five N-linked glycosylation motifs in IgA2m2 and the single site in the JC were removed by mutagenesis to produce a molecule without glycans (aglycosylated) (FIG. 7A and FIG. 25). An aglycosylated IgA polymer will not be recognized by glycan receptors and allow the study of pharmacokinetics independent of glycan receptor-mediated clearance mechanisms. As shown in FIG. 36B-D, individual IgA2m2 glycosylation variants have similar binding to mouse pIgR and human pIgR. For removal of the N-linked glycosylation motifs N-X-S/T in IgA2m2, N was mutated to A/G/Q or the S/T was mutated to A or reverted the motif to the non-glycosylated IgA1 sequence in the three instances this occurs (FIG. 1A). The JC residue N49 was mutated to A/G/Q or S51 was mutated to A. It was found that the individual IgA2m2 mutations N166A, S212P, N263Q, N337T.I338L.T339S and N459Q, and the N49Q JC mutation to give the highest levels of transient expression, however the combination of mutations to remove all five glycosylation sites in IgA2m2 resulted in poor expression and the further addition of the J chain N49Q mutation completely abolished it. See also FIG. 24A-C. Chimeric immunoglobulins often show lower expression levels in mammalian cells relative to those from a single species. Therefore, the murine anti-mIL13 variable domains were switched to humanized anti-HER2 to generate humanized IgAs, however this did not improve transient expression levels. Therefore, a CHO targeted integration (TI) stable cell line was produced to increase the expression level of the aglycosylated human anti-HER2 IgA2m2 polymer which was purified as a mixture of oligomeric species.

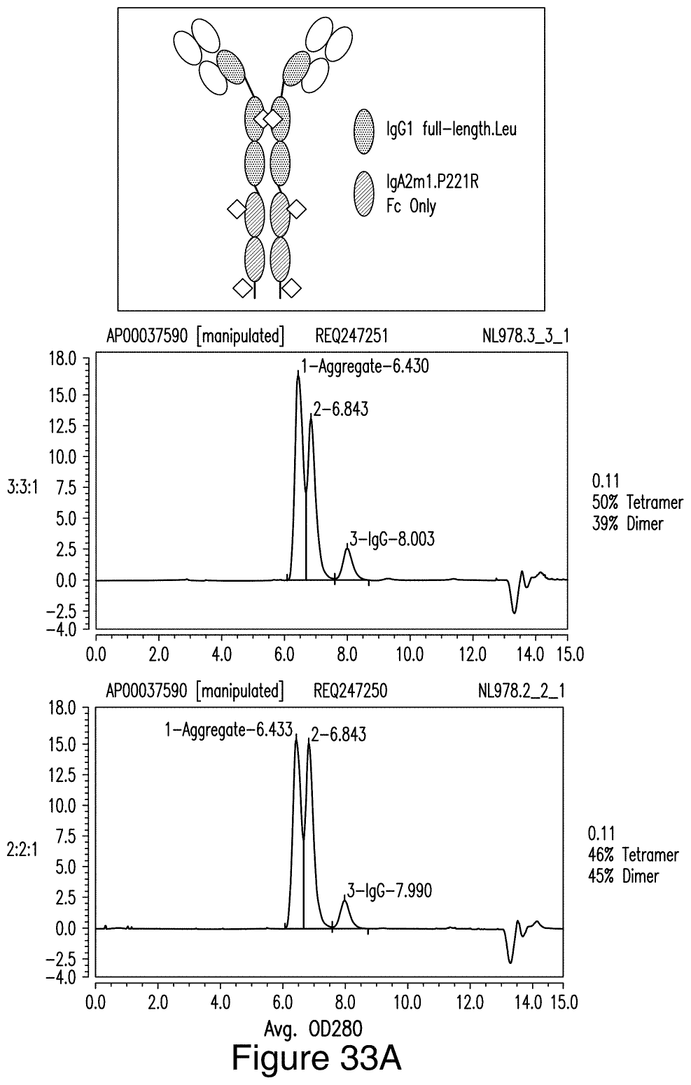

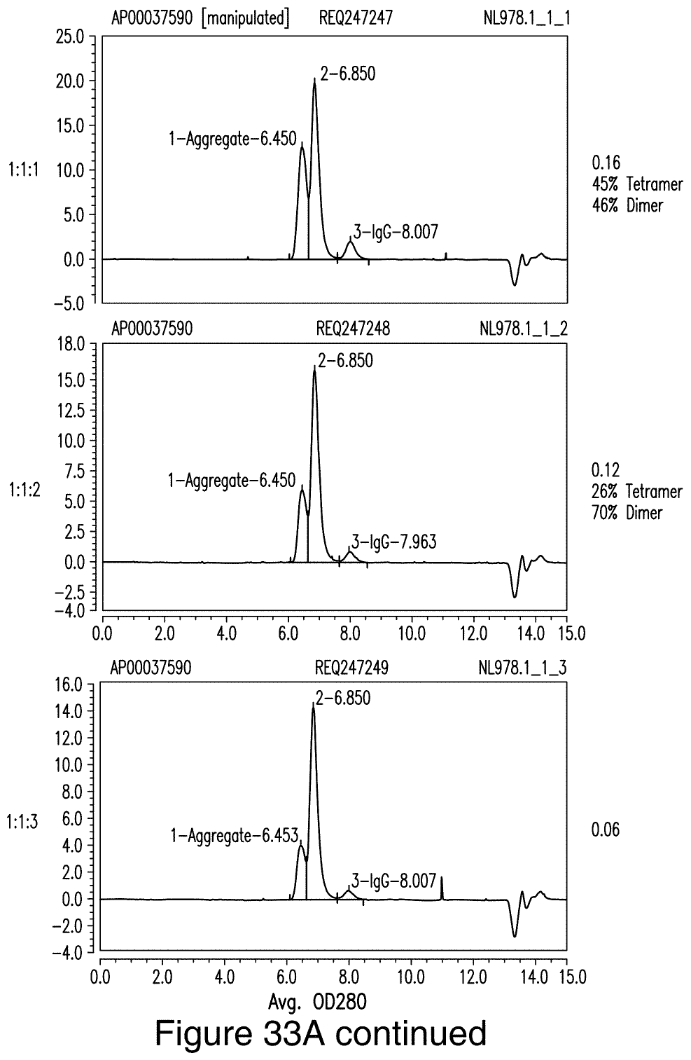

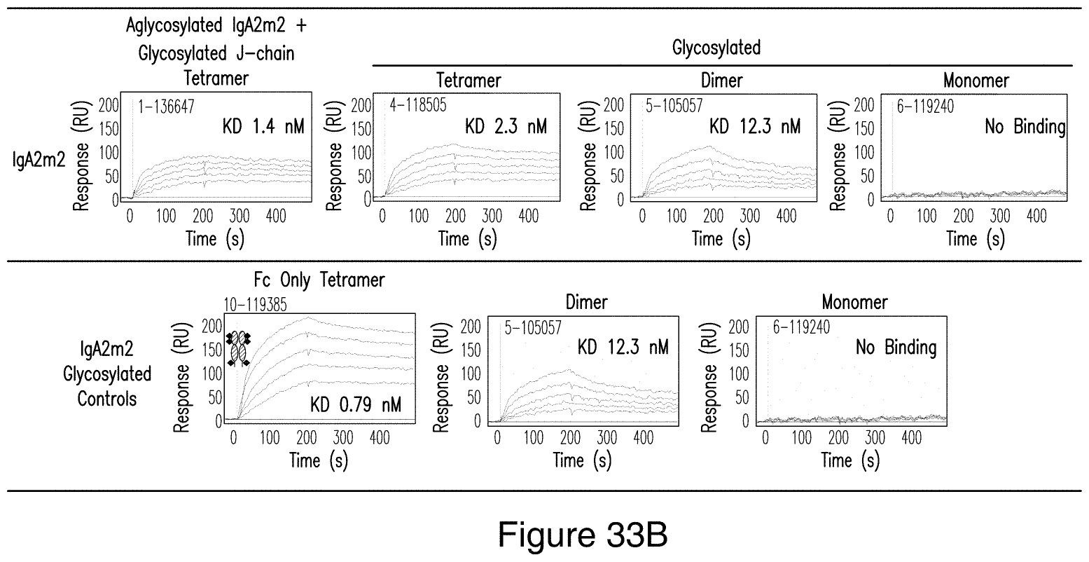

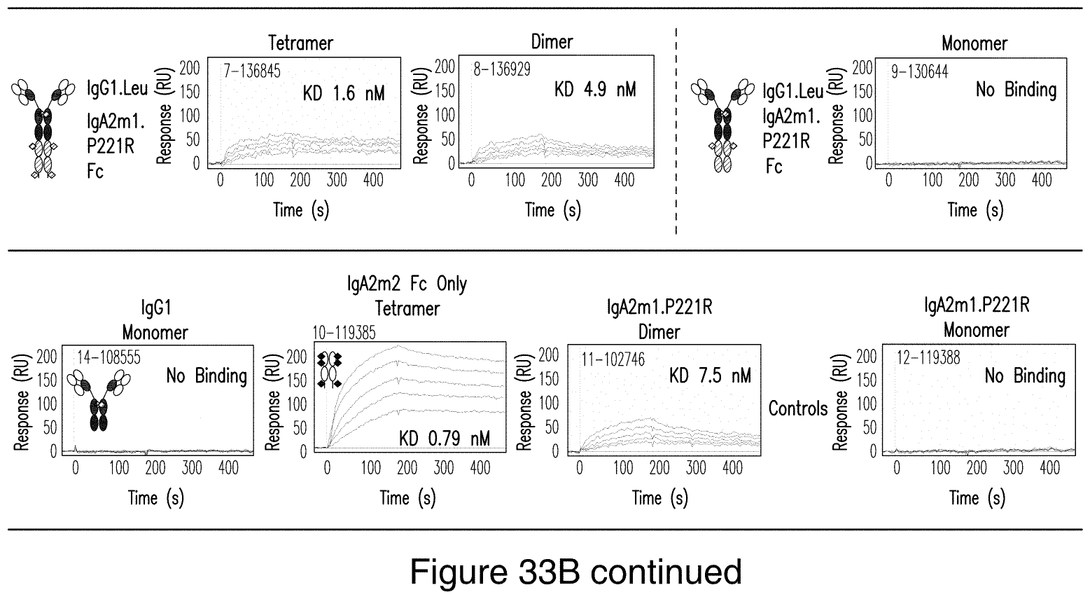

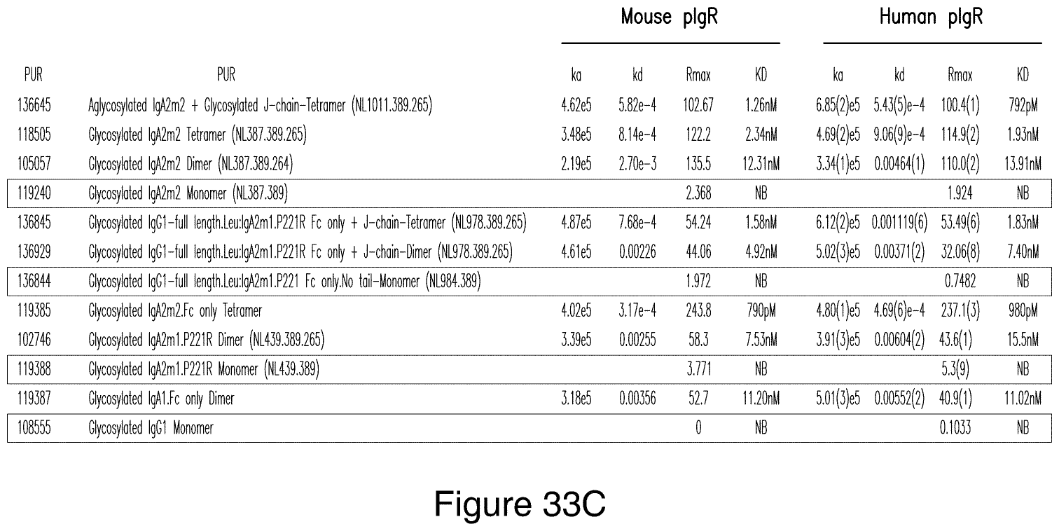

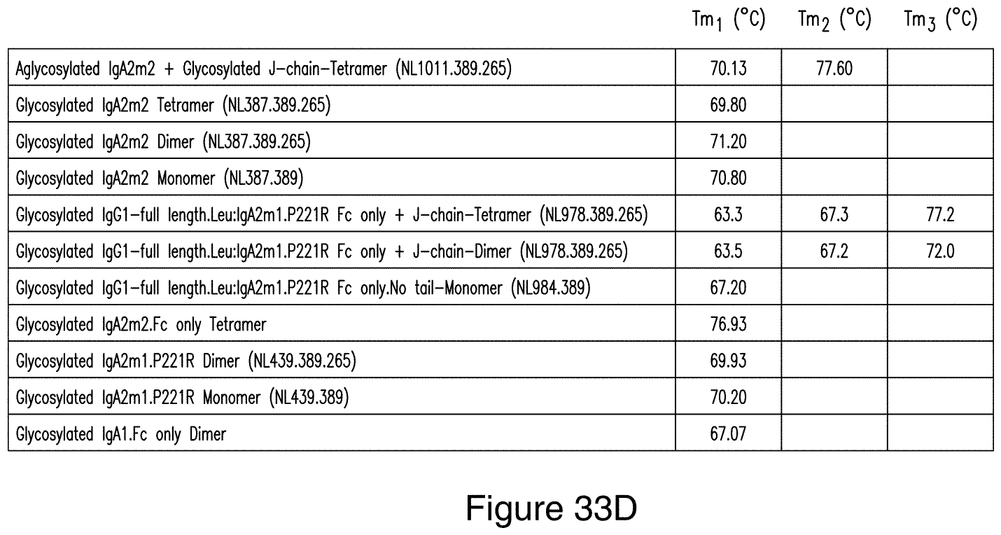

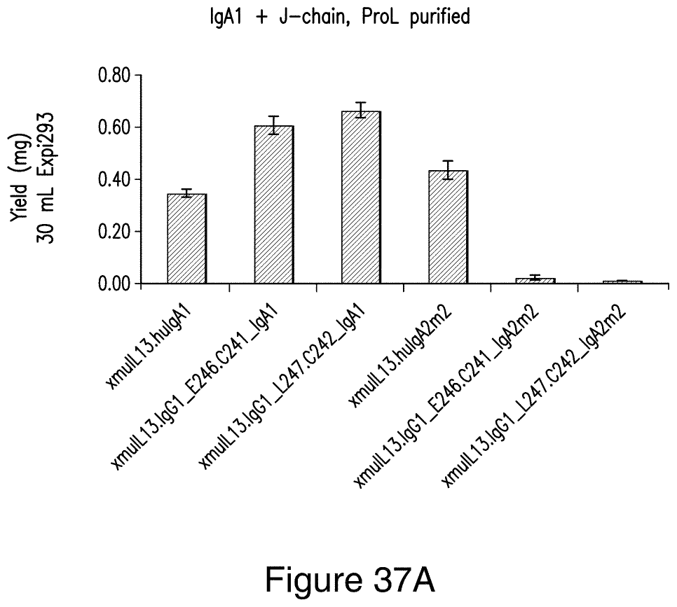

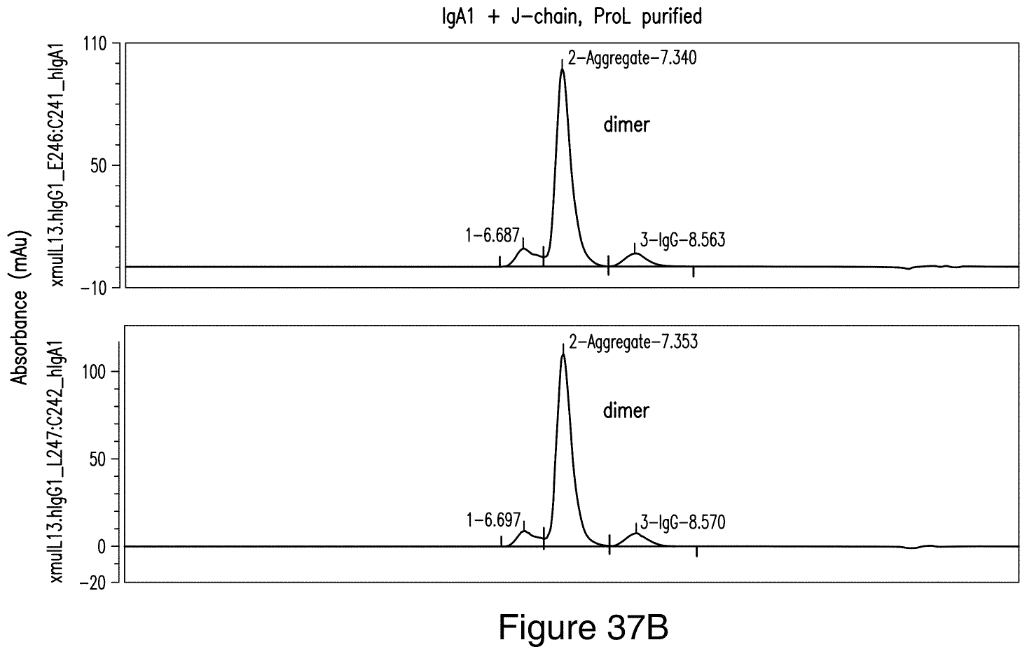

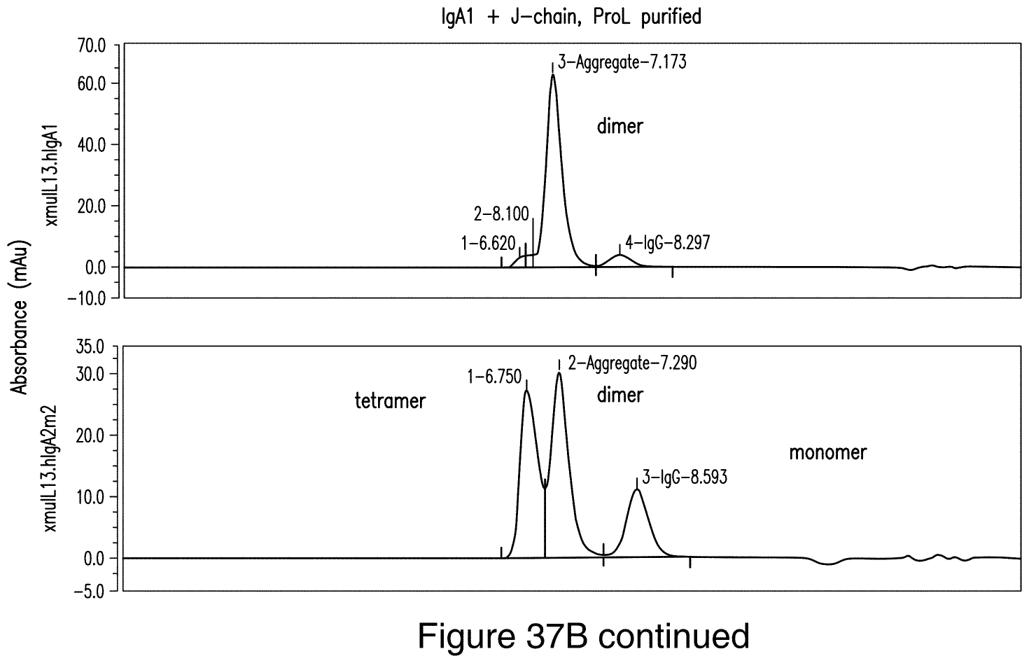

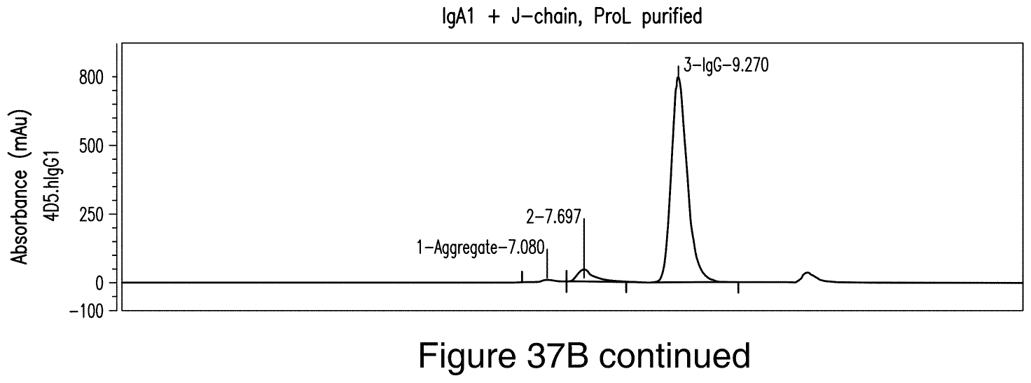

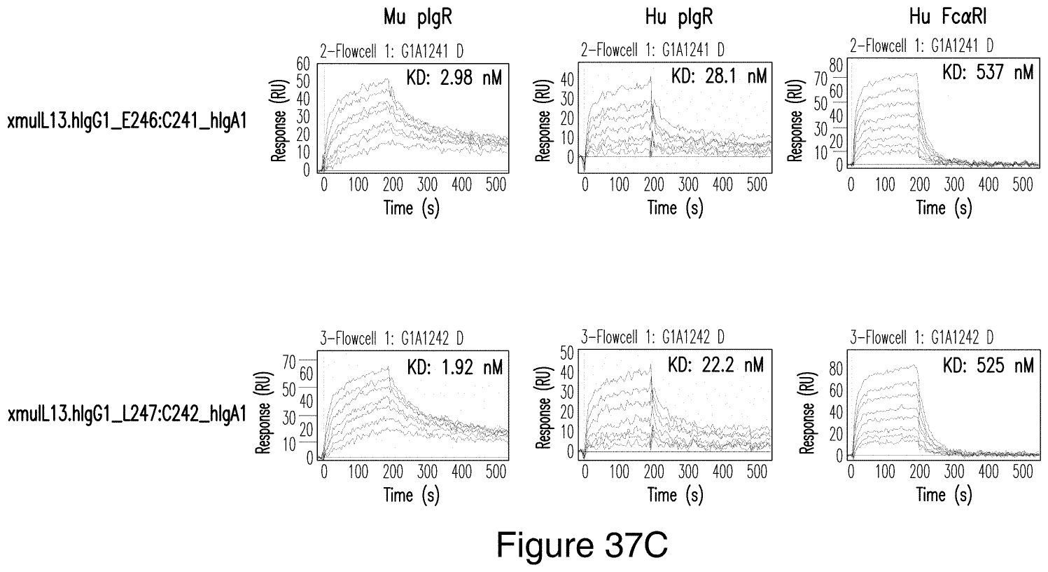

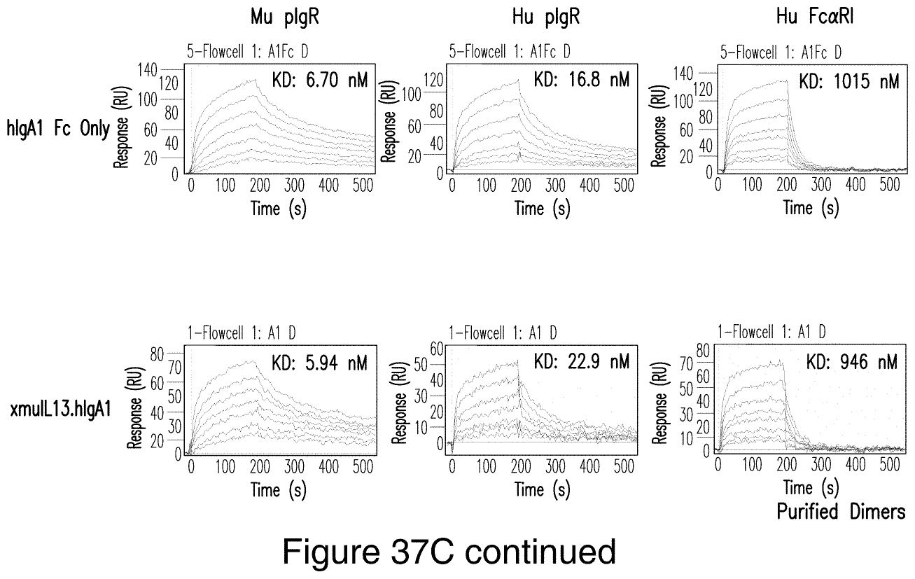

Second, two IgG1-IgA Fc fusions were engineered in order to exploit FcRn binding as a way to reduce lysosomal degradation (FIG. 7B). Previous studies suggested that this approach rescued IgA monomer serum clearance to levels comparable to IgG1 (Li et al. (2017) and Borrok et al. (2015)). Initially, dimeric and tetrameric versions of the previously reported IgG1-IgA2m1 P221R Fc fusion (Borrok et al. (2015)) were made, but observed that these displayed instability in mouse plasma at 4 days (FIG. 7C). The primary truncation product eluted in analytical SEC at a similar time to the full-length anti-HER2 IgG1 (trastuzumab), suggesting that this instability was caused by endoproteases cleaving at the IgG1-IgA2m1 P221R Fc junction. The amino acid sequence of the junction was inspected and a stretch of positively charged residues that resembled a furin-like cleavage site was identified (FIG. 12). To mitigate proteolytic cleavage, these positively charged residues at the junction were eliminated by removal of the C-terminal K447 of the IgG1 heavy chain and started the IgA2m1 Fc with either P221, the native IgA2m1 residue (instead of the P221R mutation), or C242, which deletes the IgA2m1 hinge (FIG. 12 and FIG. 34A). The C242 Fc start was also included as it was the first residue of the IgAl Fc crystal structure construct, so was presumed to be a stable truncated protein (Herr et al., Nature 423:614-20 (2003)). When the reengineered IgG1ΔK-P221 IgA2m1 Fc and IgG1ΔK-C242 IgA2m1 Fc fusions were produced as dimers, both were found to be stable in mouse plasma for up to 4 days (FIG. 7C). FIG. 34B provides the transient expression data for full length anti-IL-13 IgG1-IgA Fc fusions. Some of the engineered fusion molecules exhibit improved expression compared to IgG1 and the original construct (FIG. 34B and FIG. 37A). Further, as shown in FIG. 33A, increasing the amount of JC DNA compared to the amount of LC and HC DNA resulted in the production of more dimer species than higher order oligomeric species.

IgG1-IgA1 fusions were also generated by fusing IgG1 at the lower hinge residue E233 or L234 to the Fc of IgA1 at C241 or C242. As shown in FIG. 37B, the IgG1-IgA1 fusions were predominantly expressed as dimers, similar to IgA1. In addition, the IgG1-IgA1 fusions bound to human and mouse pIgR and human FcαRI in similar manner to IgA1 (FIG. 37C).

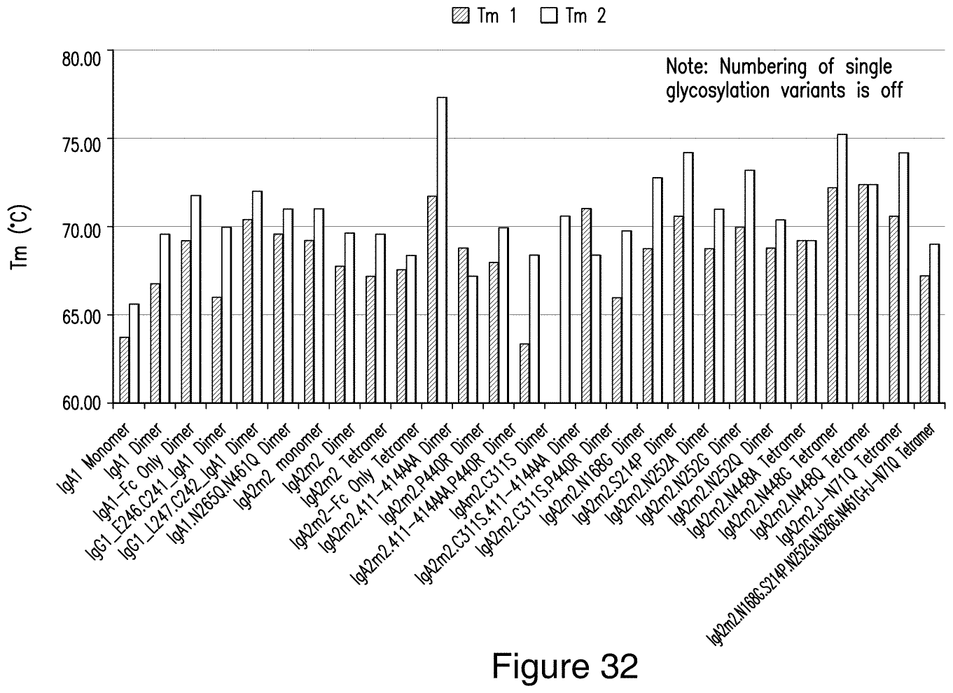

The engineered IgA antibodies and IgG1-IgA fusion molecules were analyzed for stability by differential scanning fluorimetry (DSF) confirming no loss in stability compared to IgA1 dimer (FIG. 32).

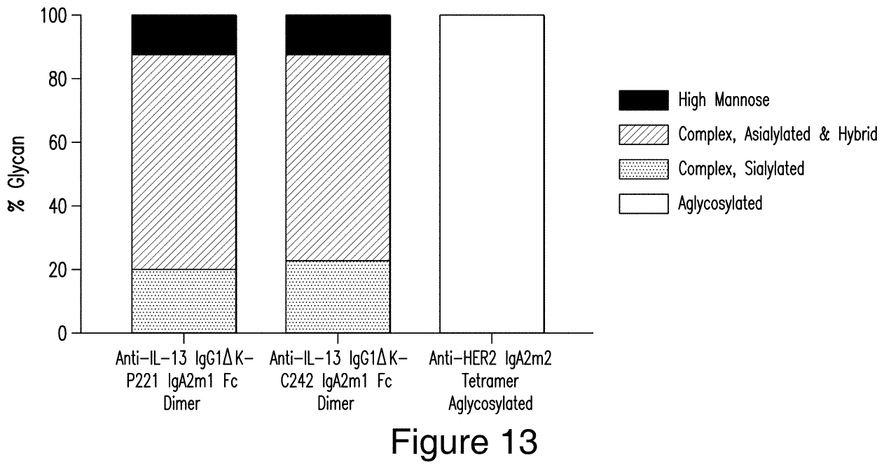

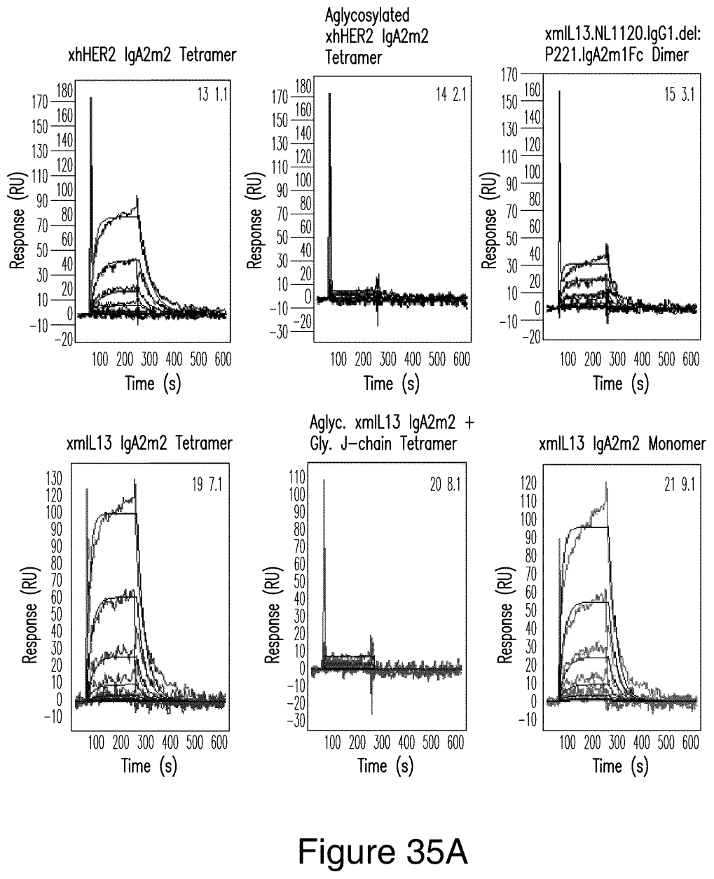

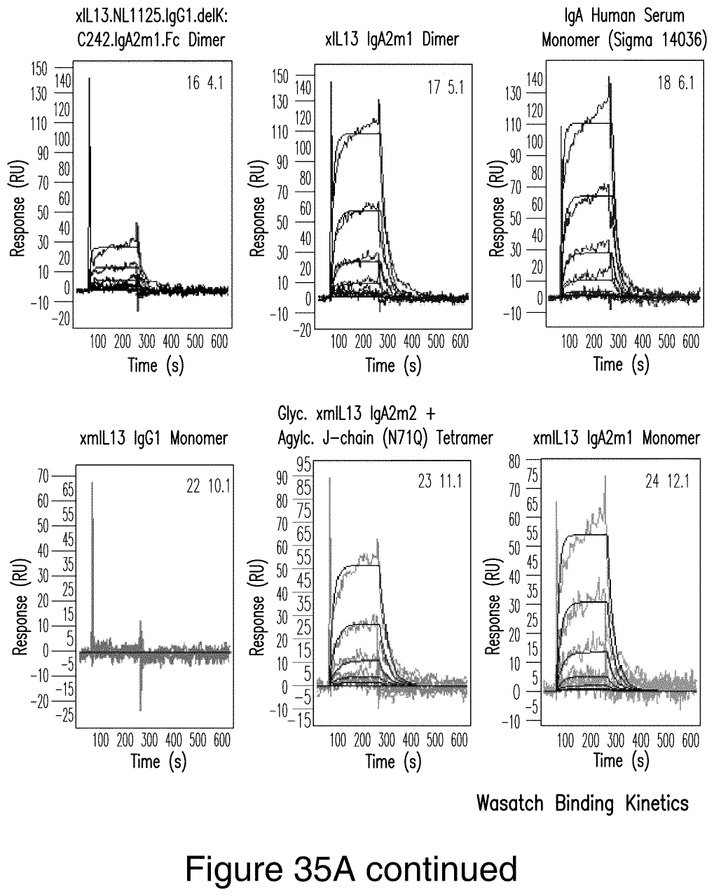

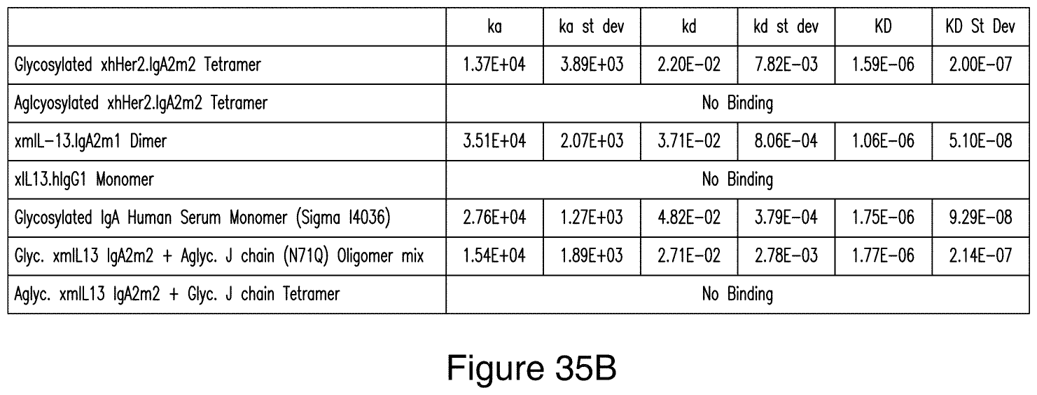

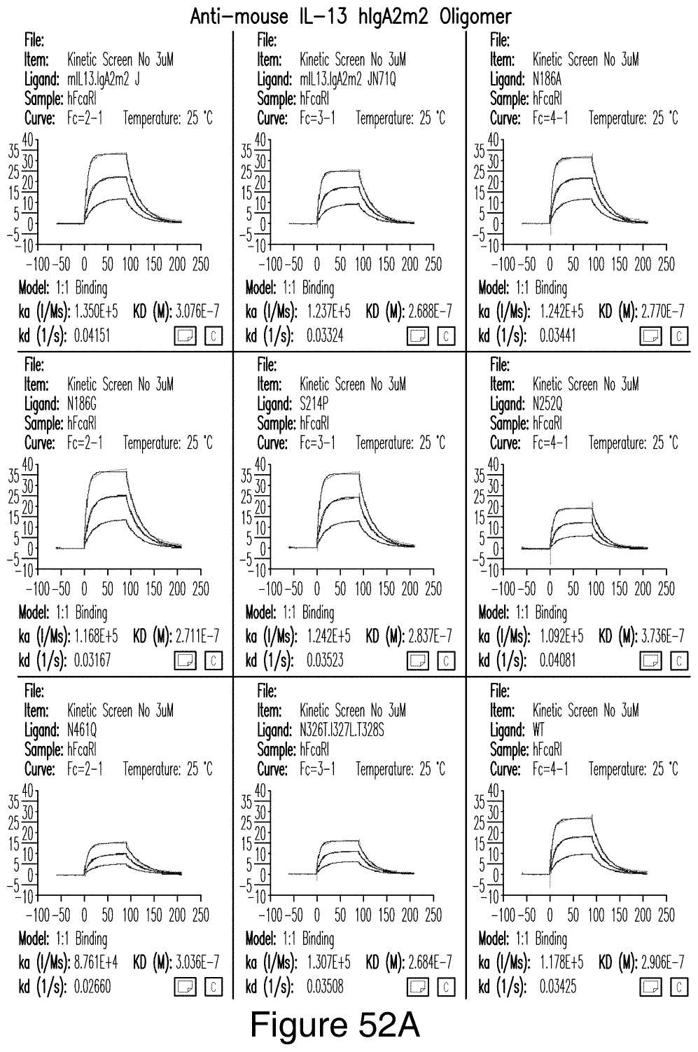

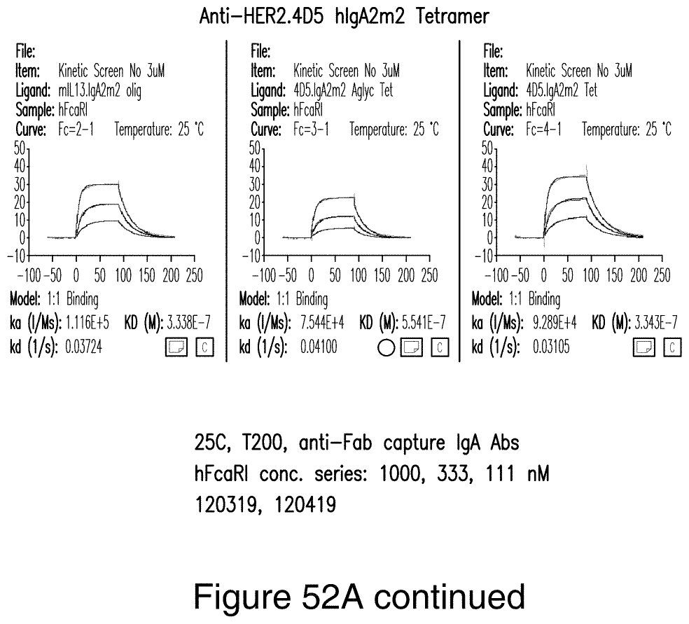

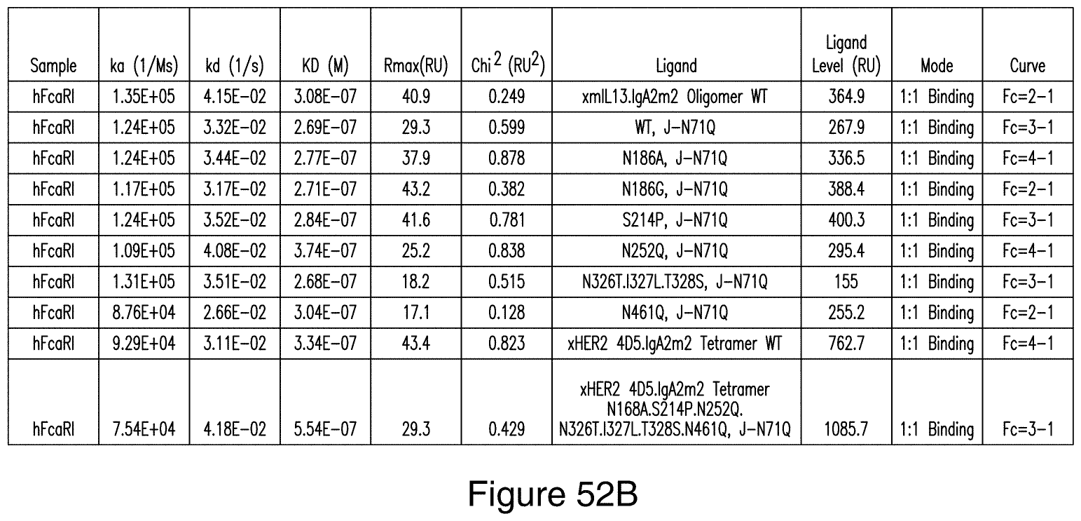

The engineered IgA antibodies and IgG1-IgA fusion molecules were further characterized for global glycan content, antigen binding and receptor binding. The aglycosylated anti-HER2 IgA2m2 polymer indeed had no glycosylation, while the anti-mIL-13 IgG1ΔK-P221 IgA2m1 Fc and IgG1ΔK-C242 IgA2m1 Fc fusions contained only ˜20% complex, sialylated glycans (FIG. 13 and Table 8). The aglycosylated anti-HER2 IgA2m2 polymer was found to have similar binding affinity to human (h)HER2, murine (m)pIgR, and hpIgR as the glycosylated IgA2m2 tetramer, while it did not bind the IgA-specific hFc receptor, hFcαRI, as determined by the Wasatch SPR assay (Table 7; see also FIG. 33B-C). Interestingly, an IgA2m2 tetramer lacking glycosylation on the IgA2m2 HC, but retaining glycosylation on the J-chain, was also unable to bind hFcαRI (FIG. 35A-B) as determined by the Wasatch SPR assay, suggesting that glycosylation of the IgA HC is required for receptor binding. However, as shown using the Biacore SPR system, which is the SPR system commonly used in the pharmaceutical industry, the glycosylation state of the IgA polymers did not affect binding to hFcαRI (FIG. 52A-B). As shown in FIG. 52A-B, aglycosylated anti-HER2 IgA2m2 polymer (referred to as “xHER24D5.IgA2m2 Tetramer N168A.S214P.N252Q.N326T1327L.T328S.N461Q, J-N71Q”) and partially deglycosylated anti-IL-13 IgA2m2 oligomers retained hFcαRI binding as determined by the Biacore SPR assay. Without being bound to a particular theory, the differences in the results obtained from the two SPR systems, i.e., Wasatch and Biacore systems, can be due, in part, to the different strategies used to immobilize the antibodies to the chips used in the SPR systems as disclosed in the methods.

Further, both the anti-mIL-13 IgG1ΔK-P221 IgA2m1 Fc and IgG1K-C242 IgA2m1 Fc dimers had similar binding affinities to mIL-13, mFcRn, and hFcRn as the anti-mIL-13 IgG1 (Table 7), as well as similar binding affinities to mpIgR, hpIgR and hFcαRI as an IgA2m1 dimer (Tables 3 and 7) as determined by the Wasatch SPR assay. Thus, the IgG1-Ig2m1A Fc fusions retain the desired attributes of both IgG and polymeric IgA.

TABLE 7
Binding affinity of IgG1-IgA2m1 Fc fusion dimers and aglycosylated IgA2m2
tetramer to antigens and receptors using the Wasatch binding assay.

| | K_{D} (nM) | | | | | | |
|---|---|---|---|---|---|---|---|
| | | | | | Mouse | Human | |
| | Mouse | Human | Mouse | Human | FcRn* | FcRn* | Human |
| | IL-13 | HER2 | pIgR | pIgR | pH 6.0 | pH 6.0 | FcαRI |
| anti-HER2 IgA2m2 | NB | 0.21 ± 0.08 | 0.35 ± | 0.50 ± | NB | NB | 1,590 ± 200 |
| Tetramer | | | 0.01 | 0.06 | | | |
| anti-HER2 IgA2m2 | NB | 0.27 ± 0.07 | 0.55 ± | 0.72 ± | NB | NB | NB |
| Tetramer | | | 0.10 | 0.06 | | | |
| Aglycosylated | | | | | | | |
| anti-IL-13 | 1.46 ± 0.33 | NB | 3.32 ± | 7.67 ± | 6,800 ± 556 | 8,400 ± | 1,070 ± 69.8 |
| IgG1ΔK- | | | 1.07 | 0.42 | | 294 | |
| P221 IgA2m1 Fc | | | | | | | |
| Dimer | | | | | | | |
| anti-IL-13 | 1.38 ± 0.15 | NB | 3.32 ± | 4.41 ± | 7,400 ± 830 | 9,900 ± | 938 ± 93.9 |
| IgG1ΔK- | | | 1.62 | 1.72 | | 838 | |
| C242 IgA2m1 Fc | | | | | | | |
| Dimer | | | | | | | |
| anti-IL-13 IgG1 | 1.15 ± 0.17 | NB | NB | NB | 7,800 ± 499 | 9,800 ± | |
| | | | | | | 1,081 | NB |
| Human Serum IgA | NB | NB | NB | NB | NB | NB | 1,750 ± 92.9 |
| Monomer | | | | | | | |

NB: No Binding.
*K_{D} was calculated using steady state kinetics.
All experiments were performed at least n = 3.

TABLE 8
Global N-linked glycan analysis of IgG1-IgA2m1
Fc fusion oligomers and aglycosylated IgA2m2 tetramer.

| | | | | Anti-HER2 |
|---|---|---|---|---|
| | | Anti-IL-13 | Anti-IL-13 | IgA2m2 |
| | | IgG1ΔK-P221 | IgG1ΔK-C242 | Tetramer |
| | | IgA2m1 Fc Dimer | IgA2m1 Fc Dimer | Aglycosylated |
| High | [symbol] | 2.7% | 2.2% | 0% |
| Mannose | [symbol] | 1.4% | 2.2% | 0% |
| | [symbol] | 5.0% | 4.0% | 0% |
| | [symbol] | 0.5% | 0.9% | 0% |
| | [symbol] | 2.9% | 3.4% | 0% |
| Complex, | [symbol] | 1.2% | 1.8% | 0% |
| Asialylated | [symbol] | 0% | 0.8% | 0% |
| & Hybrid | [symbol] | 4.4% | 5.3% | 0% |
| | [symbol] | 2.3% | 3.2% | 0% |
| | [symbol] | 1.4% | 2.8% | 0% |
| | [symbol] | 4.1% | 3.2% | 0% |
| | [symbol] | 46.1% | 41.3% | 0% |
| | [symbol] | 1.5% | 1.6% | 0% |
| | [symbol] | 2.5% | 2.5% | 0% |
| | [symbol] | 3.5% | 1.2% | 0% |
| | [symbol] | 0.9% | 1.2% | 0% |
| Complex, | [symbol] | 0.5% | 0.5% | 0% |
| Sialylated | [symbol] | 1.9% | 2.4% | 0% |
| | [symbol] | 0.6% | 1.1% | 0% |
| | [symbol] | 0.7% | 0.8% | 0% |
| | [symbol] | 3.0% | 4.4% | 0% |
| | [symbol] | 4.9% | 4.0% | 0% |
| | [symbol] | 8.0% | 8.2% | 0% |
| | [symbol] | 0% | 0.5% | 0% |
| | [symbol] | 0% | 0.5% | 0% |

The in vitro pIgR mediated transcytosis and serum concentration time profiles were measured in mice of both of these newly generated formats. The IgG1-IgA2m1 Fc fusions showed the most marked improvements in the overall IgA serum-exposures in mice (FIG. 7D, FIG. 34C and Table 9), yet the lowest levels of in vitro transcytosis compared to the aglycosylated IgA2m2 polymer, which showed the highest level of in vitro transcytosis (FIG. 7E).

TABLE 9
Pharmacokinetic Parameter Estimates (Mean) after a
30 mg/kg IV bolus of IgA oligomers to Balb/C mice.

| | | | | Half |
|---|---|---|---|---|
| | Cₘₐₓ | AUCₗₐₛₜ | CL | Life |
| | (μg/mL) | (day · μg/mL) | (mL/day/kg) | (Days) |
| Anti-HER2 IgA2m2 | 48.9 | 617.8 | 612.8 | 0.87 |
| Tetramer | | | | |
| Anti-HER2 IgA2m2 | 159 | 1063.7 | 320.7 | 0.68 |
| Tetramer | | | | |
| Aglycosylated | | | | |
| Anti-IL-13 | 176 | 962.5 | 236.8 | 3.42 |
| IgG1ΔK-P221 | | | | |
| IgA2m1 Fc Dimer | | | | |
| Anti-IL-13 | 177 | 739.9 | 192.3 | 1.94 |
| IgG1ΔK-C242 | | | | |
| IgA2m1 Fc Dimer | | | | |
| Anti-IL-13 IgA2m1 | 115 | 1371.8 | 430.9 | 0.41 |
| Dimer | | | | |

AUCₗₐₛₜ = area under the concentration-time curve, last measurable concentration;
CL = clearance;
Cₘₐₓ = maximum concentration observed;
IV = intravenous;
Note:
As sparse PK analysis was performed for all mouse PK data, data from individual mice per group was pooled and SD was not reported.

Discussion:

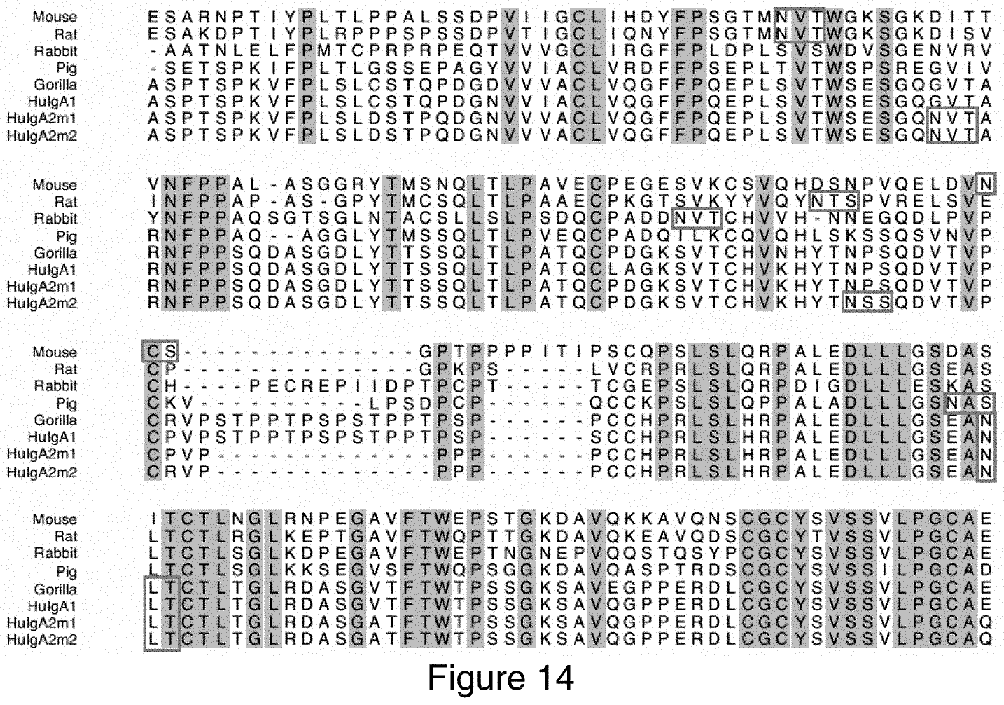

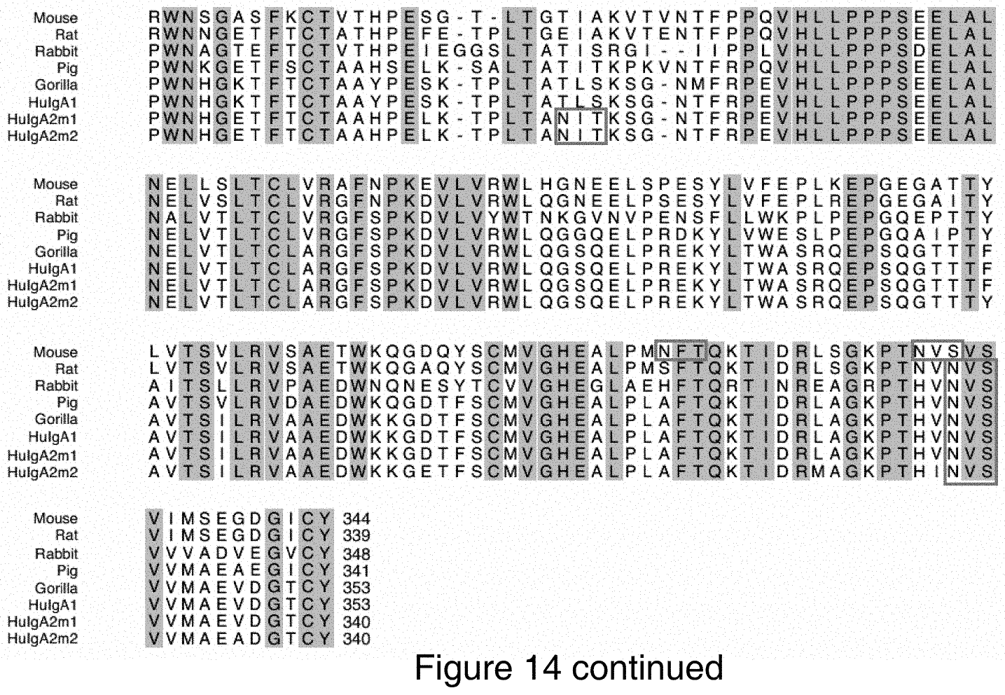

IgA has the potential to extend the therapeutic reach of monoclonal antibodies beyond the current functionalities provided by IgG. In part, this is enabled by the versatility of IgA to form both monomeric and polymeric species. Over the past few years significant progress has been made on the recombinant production of monomeric IgA (Leusen (2015), Dicker et al., Bioengineered (2016), Vasilev et al., Biotechnol Adv (2015) and Virdi et al., Cell Mol Life Sci (2015)), providing a robust path to isolate well-characterized material with increased sialylation content of the N-linked glycans that has resulted in improved serum clearance (Rouwendal et al. (2016)). While the strong cytotoxic properties of monomeric IgA are an attractive feature for oncology indications, polymeric IgA is required to reach targets beyond epithelial barriers via pIgR-mediated transcytosis. Prior to the work described herein, only mixtures of recombinantly made monomer and oligomers were used for in vivo experiments studying transcytosis (Olsan et al. (2015) and Rifai et al. (2000)). The experiments described in this example establishes a robust expression and purification route allowing the enrichment of dimeric and tetrameric IgA. In particular, modulating the amount of JC DNA used in transfection or the glycosylation state of the IgA tail region was able to influence the distribution of oligomeric species. Interestingly, the N-linked glycosylation site on the IgA tail is the only site that is extremely conserved among species (FIG. 14), offering a way to control higher order IgA oligomer formation in vivo. For purification of recombinant IgA, Protein L affinity chromatography was used followed by HPLC-SEC and were readily able to separate dimer from tetramer.

It has been previously demonstrated that increasing the level of sialylation on recombinantly expressed IgA monomer reduces serum clearance by overcoming glycan receptor-mediated catabolism (Rouwendal et al. (2016)). As an alternative strategy, the contribution of glycan to the serum clearance of oligomeric IgA was eliminated and fully aglycosylated IgA2m2 polymer was produced. The lack of N-linked glycans did not impact binding to pIgR, as assessed by surface plasmon resonance. Surprisingly, in the in vitro MDCK transcytosis assay aglycosylated species significantly improved transcytosis compared to glycosylated tetramer or dimer was observed. This improvement was not observed in a previous study with human IgA1 dimer that had N-linked glycans removed from the antibody Fc region, but still had the carbohydrate present on the murine J-chain (Chuang et al., (1997)). Without being bound to a particular theory, one explanation is that the lack of glycan eliminates the binding to glycan receptors, thus providing unperturbed and more efficient transcytosis via pIgR binding. Interestingly, binding of tetrameric IgA2m2 to FcαRI was dependent on glycosylation, which is in contrast to the lack of effect on binding observed for monomeric IgA2m1 engineered to contain a reduced number of glycosylation sites. Although cytotoxic effects of polymeric IgA were not analyzed, an IgA therapeutic that can transcytose without activating FcαRI is desirable for inflammatory diseases, as this would prevent pro-inflammatory responses from neutrophil migration (Aleyd et al., Immunol Rev 268:123-38 (2015)).

Glycosylated IgA oligomers and monomers produced recombinantly in the experiments disclosed herein cleared rapidly from serum similar to what was previously observed for monomeric and polymeric IgA (Rouwendal et al. (2016) and Chuang et al., (1997)). The fast clearance was attributed to potential binding to glycan receptors and subsequent degradation in the lysosome. The biodistribution study with glycosylated IgA supported this conclusion, indicating the most catabolism in liver and small intestine. To better understand the contribution of having glycans, an IgA polymer without glycans (aglycosylated) was generated and its in vivo PK profile was directly compared to the glycosylated version. The aglycosylated IgA2m2 polymer displayed no appreciable difference in overall mouse serum exposure compared to the glycosylated polymer (<2-fold). This suggests that having N-linked glycans play a minimal role in contributing to clearance of IgA polymers in mice. While further studies are necessary to understand why aglycosylated IgA oligomer does not improve serum clearance, it appears that pIgR-mediated transcytosis and/or clearance may play a significant role in determining the overall serum concentrations and the fate of polymeric IgA in mice. Indeed, the equilibrium binding affinity of tetrameric IgA to pIgR is at least in the picomolar range allowing for efficient binding to the abundant pIgR receptor, followed by transcytosis. However, a detailed biodistribution study looking at the tissue distribution profile will be needed to better interpret the serum concentration time profiles of the molecules and the disposition of aglycosylated polymeric IgA. One important caveat to studying pharmacokinetic properties and biodistribution of a polymeric IgA molecule in rodents is that expression patterns of pIgR differ between rodents and humans, potentially confounding eventual clinical translation. In particular, high expression of pIgR in the hepatocytes of rodents and rabbits have been associated with biliary clearance mechanisms of polymeric IgA (Daniels et al. (1989)), not thought to occur in humans, where pIgR expression is found instead in cells of the bile duct (Tomana et al. (1988)). Therefore, the exact role that pIgR plays in the biodistribution and clearance of a polymeric IgA molecule in mice needs to be separately evaluated.

An alternative strategy that was taken to avoid accelerated serum clearance and improve exposure was via engineering IgG1-IgA2m1 Fc fusions. The ability to bind FcRn allows for recycling in the endosome, thus avoiding sorting for lysosomal degradation. Previous reports with monomeric IgG-IgA Fc fusions reported serum clearance comparable to IgG (Li et al. Oncotarget (2017) and Borrok et al. (2015)). In addition, an improvement in pharmacokinetics was also observed for monomeric IgA that was fused to albumin binding peptides (Meyer et al., MAbs 8:87-98 (2016)). Since a large contribution was not observed by removing glycans from the IgA polymers, glycosylated fusion proteins were produced. While the dimeric IgG1-IgA2 Fc fusions showed improved overall serum exposures, it was not comparable to IgG as observed for the monomeric IgG-IgA Fc fusions (Li et al. Oncotarget (2017) and Borrok et al. (2015)). By surface plasmon resonance, it was demonstrated that the dimeric IgG1-IgA2m1 Fc fusions can bind FcRn and pIgR, albeit having reduced in vitro transcytosis in a MDCK model. While the binding to FcRn extended the terminal half-life, IgG1-IgA2m1 Fc dimer interaction with pIgR may have provided a clearance mechanism, particularly in the early phase, which resulted in pIgR-mediated transcytosis and/or clearance, contributing to the reduced serum concentrations compared to IgG.

IgA in serum is constituted predominantly of IgA1 monomer secreted from bone marrow cells, while polymeric IgA2 is secreted from plasma cells in the lamina propria at the location of transcytosis (Yoo et al. 116:3-10 (2005)). The high affinity between polymeric IgA and pIgR may naturally lead to fast scavenging of polymeric IgA from circulation, providing effective clearance of harmful antigens from the circulation as IgA-antigen complexes (Shroff et al., Infect Immun 63:3904-13 (1995)) and in a therapeutic setting can be exploited to restrict drug activity to a defined tissue and short duration, something that may be of particular benefit when agonizing cytokine receptors. The fast clearance of polymeric IgA via pIgR from serum in mice is further supported by the approximately 20-fold increased IgA concentrations in serum of pIgR-deficient NOD mice (Simpfendorfer et al., PLoS ONE 10:e0121979 (2015)).

### EXAMPLE 2

### Purification of Recombinant IgA Antibodies

Recombinant IgAs containing a kappa light chain were expressed in CHO cells as secreted proteins and affinity captured from the cell culture supernatant using a Capto L (GE Healthcare) column. After capture, the column was washed with 5 column volumes (CVs) of Tris buffer (25 mM Tris, pH 7.5, 150 mM NaCl, 5 mM EDTA, 2 mM NaN3), 20 CVs of Triton X-114 buffer (25 mM Tris, pH 7.5, 150 mM NaCl, 5 mM EDTA, 0.1% Triton X-114, 2 mMNaN3) to remove endotoxin, 5 CVs of Tris buffer, 5 CVs of KP buffer (0.4 M potassium phosphate, pH 7.0, 5 mM EDTA, 0.02% Tween20, 2 mM NaN3), and 10 CVs of Tris buffer. IgAs were eluted with 150 mM acetic acid, pH 2.7 and immediately neutralized with ⅕ volume of 1 M arginine, 0.4 M succinate, pH 9.0.

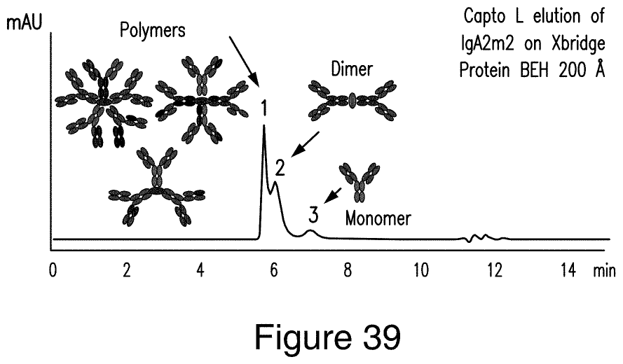

Following affinity purification, recombinant IgAs were purified using size exclusion chromatography (SEC). For recombinant IgA samples where there was mainly one oligomeric state present (≥90% of a single type of oligomer), a HiLoad Superdex 200 pg column (GE Healthcare) was used for SEC followed by peak shaving to avoid contaminants of unwanted oligomeric states. For IgA samples containing complex mixtures of oligomers in near equivalent amounts (e.g., ˜40% dimer and ˜60% higher order polymers), several purification approaches were tested. A human anti-mIL-13 IgA2m2 mixture of oligomers as shown in FIG. 39 was used to test the different types of purifications described as follows.

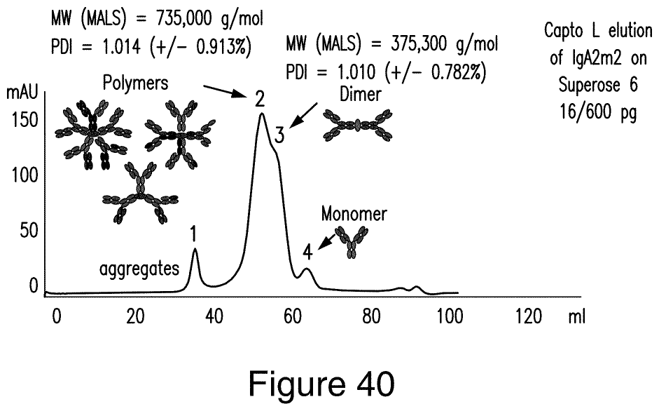

SEC using a HiLoad Superose 6 16/600 pg column (GE Healthcare) gave insufficient resolution in the higher molecular weight range to separate IgA dimers from higher order oligomers as shown in FIG. 40. On the Superose 6 elution profile, peak 1 elutes near ˜35-39 mL which corresponds to the void volume of the column and therefore is likely aggregated protein. Peaks 2 and 3 significantly overlap such that peak 3 appears as a shoulder on the trailing edge of peak 2. Analysis of fractions near the leading edge of peak 2 and the trailing edge of peak 3 by SEC-MALS as described above gave molar masses of 735,000 g/mol and 375,300 g/mol, respectively. The expected molecular weight of the human anti-mIL-13 IgA2m2 monomer is ˜148 kDa, dimer ˜312 kDa, trimer ˜460 kDa, tetramer ˜608 kDa, and pentamer ˜756 kDa. This suggests that peaks 2 and 3 likely contain a mixture of pentamer, tetramer, trimer and dimer. Peak 4 eluting later around ˜60 mL likely corresponds to monomeric IgA.

SEC using either a HiLoad Superdex 200 pg column (GE Healthcare) or a HiLoad Sephacryl 400 pg column (GE Healthcare) also gave insufficient resolution of IgA dimers from higher order oligomers. Attempts to separate oligomers by cation-exchange chromatography using an SP HP column (GE Healthcare), anion-exchange chromatography using a Q FF column (GE Healthcare), and hydrophobic interaction chromatography (HIC) using a 5 μm, 7.8×75 mm ProPac HIC-10 column (Dionex) were also unsuccessful.

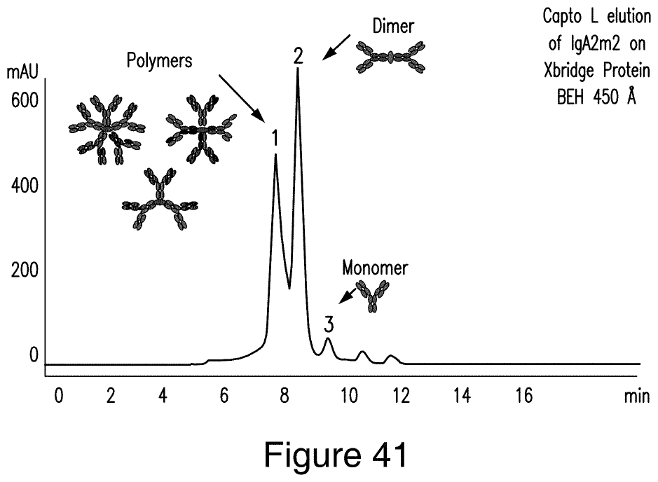

In contrast, carrying out small-scale purifications using a 3.5 μm, 7.8 mm×300 mm)(Bridge Protein BEH 450 A SEC column (Waters) gave the best separation of IgA dimers from higher order oligomers as shown in FIG. 41 for a human anti-mIL-13 IgA2m2. To maximize resolution, less than 1 mg of total protein in an injection volume no larger than 100 μL was run over the column at 1 mL/min using an Agilent 1260 Infinity HPLC with 0.2 M arginine, 0.137 M succinate, pH 5.0 as the mobile phase and 200 μL fractions were collected. Fractions were then selectively pooled to isolate predominantly one oligomeric state. Multiple runs were performed and pooled fractions of a given oligomer from each run were combined.

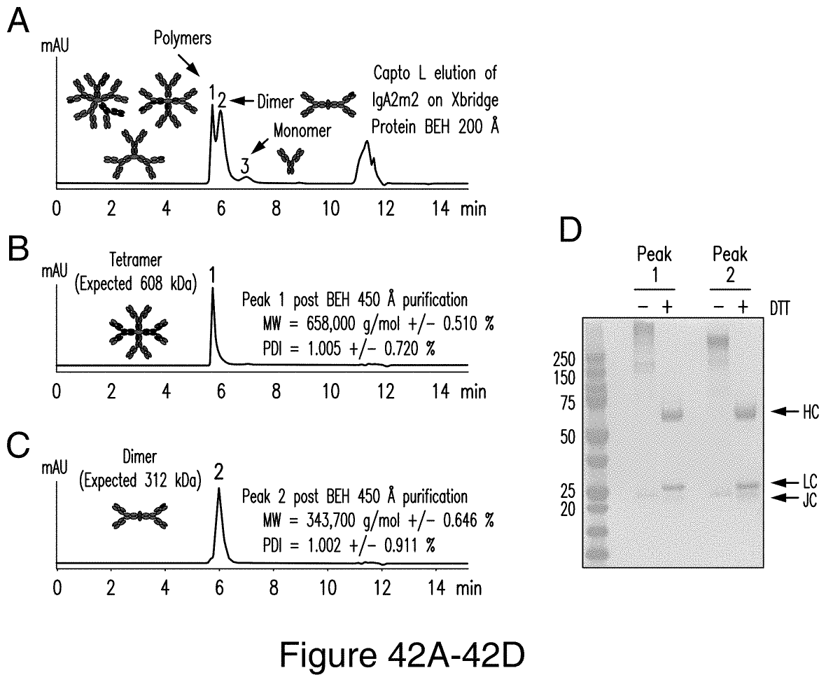

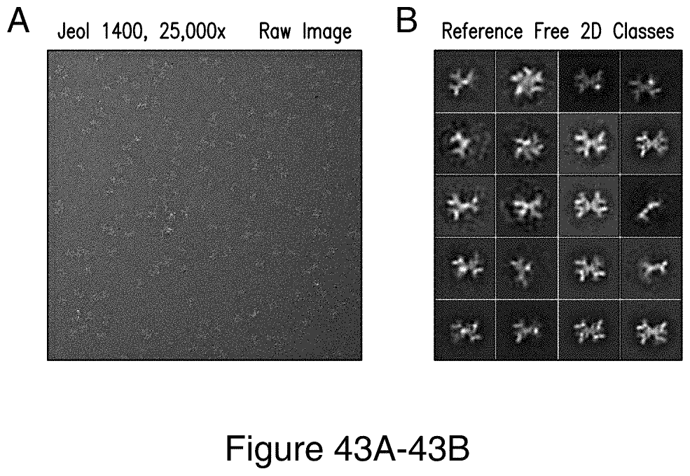

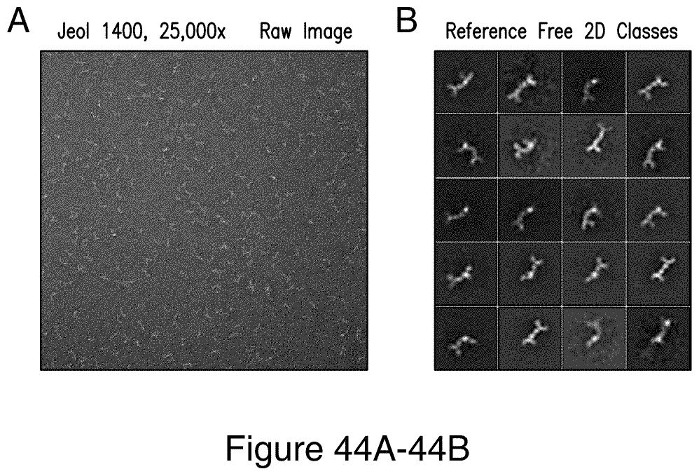

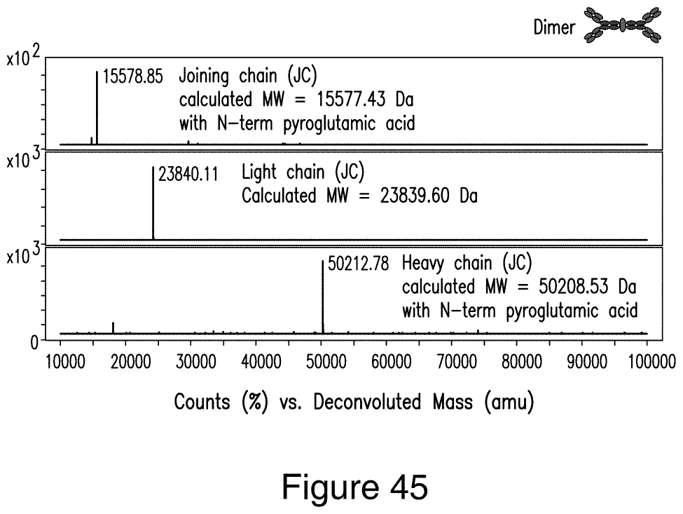

The IgA identity, purity and oligomeric state found in pooled fractions were characterized by SEC-MALS using a 3.5 μm, 7.8 mm×300 mm XBridge Protein BEH 200 Å SEC column (Waters) as described below (FIG. 42), SDS-PAGE as described below (FIG. 42), negative stain electron microscopy as described below (FIGS. 43 and 44) and mass spectrometry as described below (FIG. 45). SEC-MALS was performed by injecting recombinant IgAs onto a 3.5 μm, 7.8 mm×300 mm Waters XBridge Protein BEH 200 Å size-exclusion chromatography (SEC) column at 1 mL/min using an Agilent 1260 Infinity HPLC with 0.2 M arginine, 0.137 M succinate, pH 5.0 as the mobile phase. An example analytical SEC profile of recombinant anti-mIL-13 IgA2m2 from a Capto L affinity purification is shown in FIG. 39. Proteins eluted from the analytical SEC column were directly injected onto a Wyatt DAWN HELEOS II/Optilab T-rEX multi-angle light scattering (MALS) detector to measure the molar mass and polydispersity of the various IgA oligomeric states present in given a sample.

For the anti-mIL-13 IgA2m2 antibody, there were three main peaks identified on analytical SEC using the Waters XBridge Protein BEH 200 Å SEC column (FIG. 42A). The expected molecular weight of the human anti-mIL-13 IgA2m2 monomer is ˜148 kDa, dimer ˜312 kDa, trimer ˜460 kDa, tetramer ˜608 kDa, and pentamer ˜756 kDa. All expected molecular weights are based on amino acid sequence composition and does not factor in potential N-linked or O-linked glycans as the sugar composition is often heterogenous and variable. After separation on the Waters XBridge Protein BEH 450 Å SEC column the molar mass of peak 1 was determined by MALS as 658,000 g/mol +/−0.510% (FIG. 42B). This suggests peak 1 is predominantly tetrameric IgA2m2. The molar mass of peak 2 was determined by MALS as 343,700 g/mol +/−0.646% (FIG. 42C). This suggests peak 2 is predominantly dimeric IgA2m2. Peak 3 eluting later than the dimer is likely monomeric IgA (FIG. 42A).

SDS-PAGE analysis of non-reduced, purified peaks 1 and 2 from FIGS. 42B and 42C, respectively, showed a predominant band migrating near the expected molecular weights for IgA tetramer and dimer, respectively (FIG. 42D). This is consistent with the molar masses identified by MALS (FIGS. 42B and 42C). Upon reduction with DTT, three bands are observed on the gel (FIG. 42D). The expected molecular weight of the heavy chain (HC) is 50.2 kDa, the light chain (LC) is 23.8 kDa, and joining chain (JC) is 15.6 kDa. All expected molecular weights are based on amino acid sequence composition and does not factor in potential N-linked or O-linked glycans as the sugar composition is often heterogenous and variable. The three bands run at roughly the predicted molecular weights of all three chains, with the HC and JC running slightly larger. The HC has five predicted N-linked glycosylation sites and the JC has one predicted N-linked glycosylation site which if occupied would increase the molecular weight and decrease the migration on the gel. SDS-PAGE was performed by mixing recombinant IgA proteins with LDS sample buffer (Thermo Fisher Scientific) with or without 10 mM dithiothreitol (DTT) and heated at 70° C. for 10 minutes. Samples were then run on 4-12% Bolt Bis-Tris Plus gels (Thermo Fisher Scientific) in MES buffer (Thermo Fisher Scientific) and stained with ClearPAGE Instant Blue stain (Expedeon).

The human anti-mIL-13 IgA2m2 purified peaks 1 and 2 from FIGS. 42B and 42C, respectively, were analyzed by negative stain electron microscopy (EM). Purified IgA2m2 samples were first crosslinked by incubating in 0.015% glutaraldehyde (Polysciences, Inc.) for 10 minutes at room temperature. Once fixed, the samples were diluted using TBS buffer to achieve a concentration of 10 ng/μL. Then 4 μL of each sample were incubated for 40 s on freshly glow discharged 400 mesh copper grids covered with a thin layer of continuous carbon before being treated with 2% (w/v) uranyl acetate negative stain (Electron Microscopy Sciences). IgAs were then imaged using a Tecnai Spirit T12 (Thermo Fisher) operating at 120 keV, at a magnification of 25,000×(2.2 Å/pixel). Images were recorded using a Gatan 4096×4096 pixel CCD camera under low dose conditions. About 5000 particles for each IgA sample were then selected and extracted using the e2boxer.py software within the EMAN2 package using a 128-pixel particle box size. Reference free 2D classification, within the RELION image software package was used to generate averaged images of both samples. A raw image file along with reference free 2D classes are shown for the IgAs from purified peaks 1 and 2 (FIGS. 43 and 44). Peak 1 is predominantly tetrameric IgA2m2, with some pentamer, trimer and dimer also present (FIG. 43). Peak 2 is dimeric IgA2m2 (FIG. 44).

Mass spectrometry analysis confirmed the presence of the JC, LC and HC within less than 5 Da of the expected molecular weights with the amino-terminal residues of the JC and HC forming a pyroglutamic acid. Mass spectrometry was performed by heating IgA at 0.5 mg/mL in the presence of 5 mM DTT at 97° C. for 30 minutes to reduce and denature the protein. The sample was then cooled on ice followed by deglycosylation overnight at 37° C. with 1,000 units of PNGaseF (NEB). The reduced, denatured and deglycosylated IgA was then injected onto a 3 μm, 4.6×50 mm reverse-phase chromatography PLRP-S column (Agilent) at 1 mL/min using an Agilent 1290 Infinity UHPLC. A 5%-60% buffer B gradient over 6 minutes was performed with 0.05% trifluoroacetic acid (TFA) in water (buffer A) and 0.05% TFA in acetonitrile (buffer B). Proteins eluted from the reverse-phase column were directly injected onto an Agilent 6230 electrospray ionization time-of-flight mass spectrometer (ESI-TOF) for intact mass measurement.

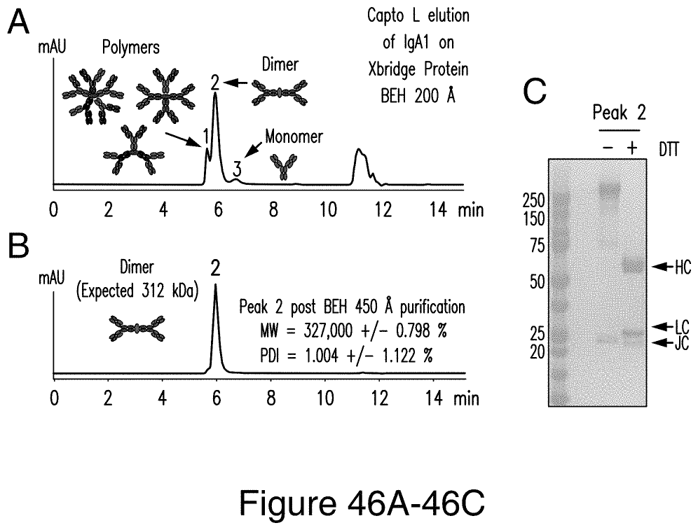

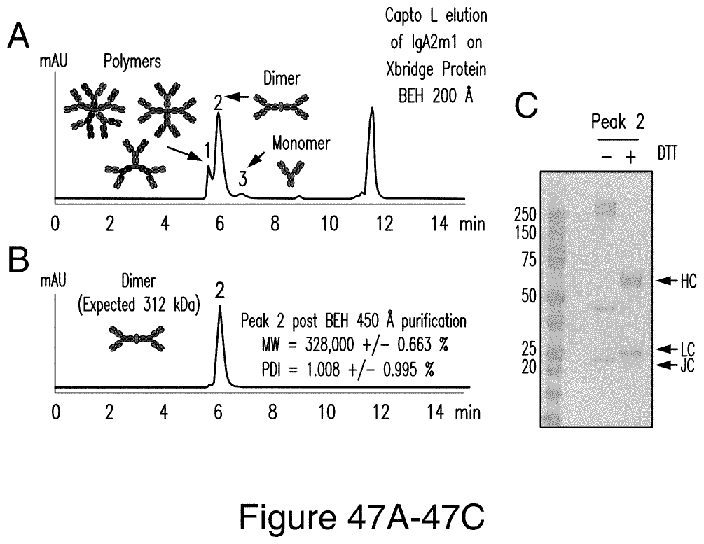

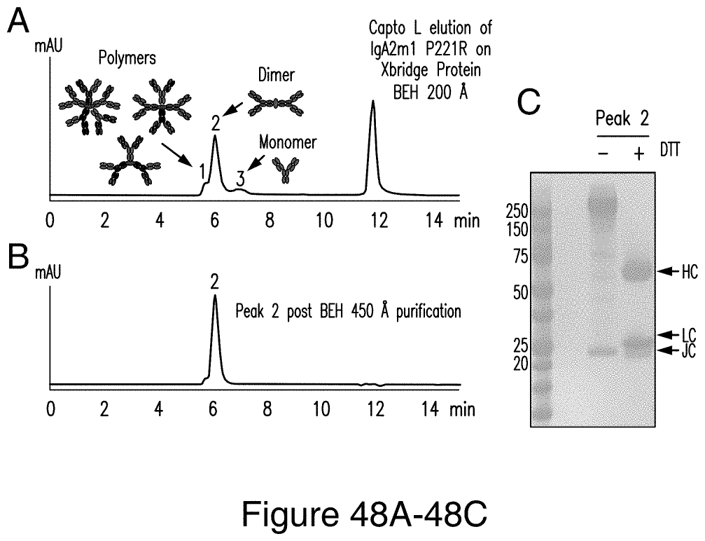

In addition to the success described for the IgA2m2 in FIGS. 41-45, this separation technique was also applicable to all other isotypes and allotypes tested, including IgA1 (FIG. 46), IgA2m1 wild-type (FIG. 47) and IgA2m1 containing the P221R mutation to restore the disulfide bond between the light chain and the heavy chain (FIG. 48).

### EXAMPLE 3

### In Vitro Analysis of Recombinant IgA Antibodies and IgG-IgA Fusion Molecules

The capacity of the IgA antibodies and IgG-IgA fusion molecules for triggering cancer cell death was analyzed in vitro in the HER2+ breast cancer cell lines KPL-4, BT474-M1 and SKBR3 using a CellTiter-Glo luminescent cell viability assay. The assay was performed as follows. Peripheral blood from healthy donors was collected using EDTA as an anticoagulant. Human neutrophils, which were used as effector cells, were isolated from the peripheral blood by using the EasySep™ Direct Human Neutrophil Isolation Kit (STEMCELL Technologies) following manufacture's instruction. Neutrophils and HER2-amplified target cells (SK-BR3; at a density of 10,000 cells per well) were incubated in 20:1 ratio in the presence of testing reagents for 48 hours in black, clear-bottomed 96-well plates (Corning). Target cell viability was measured by luminescence relative light units (RLU) using Cell Titer-Glow Luminescent Cell Viability reagent (Promega cat#G7570). Target cell killing activity was calculated as: ((RLU without treatment—RLU with treatment)/RLU without treatment)×100%.

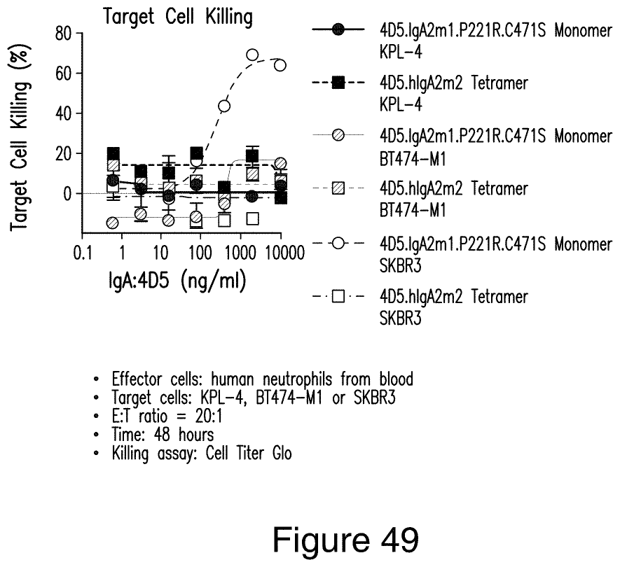

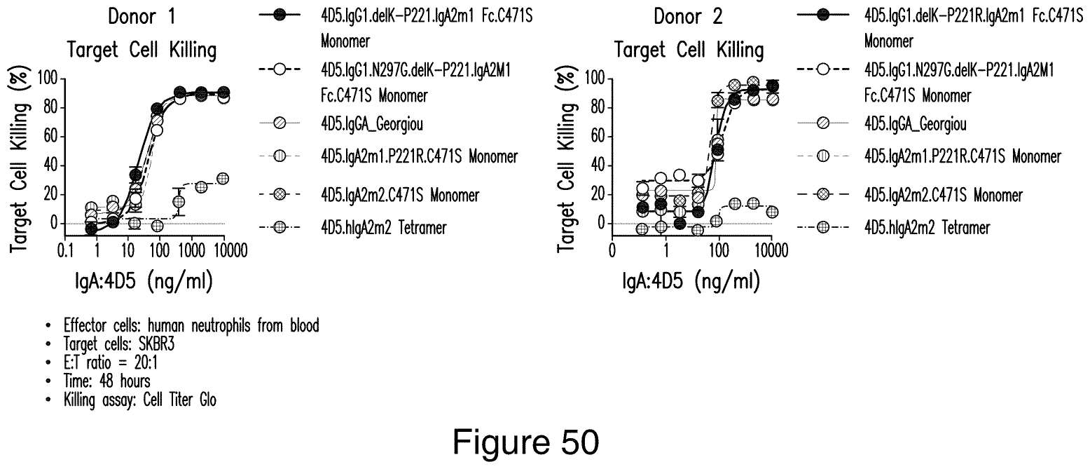

As shown in FIG. 49, the SKBR3 and BT474-M1 cell lines were sensitive to the anti-HER IgA2m1 monomer (referred to as “4D5.IgA2m1.P221R.C471S Monomer” in FIG. 49). In particular, the anti-HER IgA2m1 monomer resulted in significant killing of SKBR3 cells compared to its effect on the viability of the BT474-M1 cells. However, the KPL-4 cell line was not sensitive to the anti-IgA antibodies. To further analyze whether the ability of IgA antibodies to result in the death of cancer cells is specific to the donor of the neutrophils, neutrophils from two separate donors were used in the cell viability assay. As shown in FIG. 50, neutrophils from two different donors were able to mediate the death of SKBR3 cells in the presence of monomeric anti-HER2 IgA antibodies and monomeric IgG-IgA fusion molecules indicating that the efficacy of the antibodies and fusion molecules are not donor specific. Polymeric anti-HER2 IgA antibodies resulted in less cell death as compared to the monomeric anti-HER2 IgA antibodies (FIG. 50).

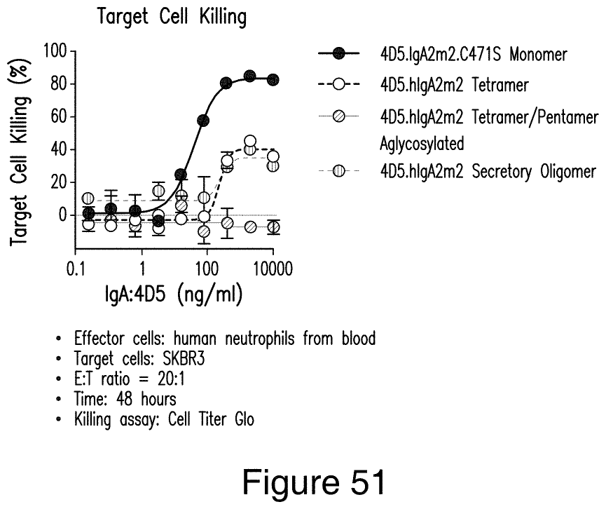

Additional experiments were performed to determine if the glycosylation state of the antibody affects its ability to result in cancer cell death. The glycosylated monomeric and tetrameric anti-HER IgA antibodies resulted in significant killing of SKBR3 cells (FIG. 51). However, the aglycosylated tetrameric anti-HER IgA antibodies did not result in the death of the targeted SKBR3 cells. Without being bound to a particular theory, these results suggest that glycosylation can affect the effectiveness of the IgA antibody.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

What is claimed is:

1. An isolated IgA antibody, or a fragment thereof, wherein the IgA antibody comprises one or more of the following:
(a) a substitution at amino acid V458, N459 and/or S461;
(b) a substitution at amino acid 1458; and
(c) a substitution at an amino acid selected from the group consisting of N166, T168, N211, S212, S213, N263, T265, N337, I338, T339, N459, S461 and a combination thereof.

2. The isolated IgA antibody of claim 1, wherein:
(a) amino acid V458 is substituted with an isoleucine (V4581), amino acid N459 is substituted with a glutamine (N459Q), a glycine (N459G) or an alanine (N459A), and/or amino acid S461 is substituted with an alanine (S461A);
(b) amino acid I458 is substituted with a valine (I458V); and/or
(c) the substitutions at amino acids N166, S212, N263, N337, I338, T339 and N459 are N166A, S212P, N263Q, N337T, I338L, T339S and N459Q.

3. The isolated IgA antibody of claim 1, wherein the IgA antibody is an IgA1, IgA2m1, IgA2m2 or IgA2mn antibody.

4. The isolated IgA antibody of claim 1, wherein the IgA antibody comprises substitutions at amino acids N337, 1338 and T339 and one or more substitutions at T168, N211, S212, S213, N263, T265, N459, S461 and a combination thereof.

5. An isolated nucleic acid encoding the IgA antibody of claim 1.

6. A host cell comprising the nucleic acid of claim 5.

7. A method of producing an IgA antibody culturing the host cell of claim 6 so that the IgA antibody is produced.

8. A pharmaceutical composition comprising one or more IgA antibodies of claim 1 and a pharmaceutically acceptable carrier.

9. A method of treating an individual having a disease, wherein the method comprises administering to the individual an effective amount of one or more IgA antibodies of claim 1.

10. An isolated IgG-IgA fusion molecule comprising a full-length IgG antibody fused at its C-terminus to an Fc region of an IgA antibody, wherein the Fc region of the IgA antibody comprises:
(a) a sequence comprising P221 or R221 through the C-terminus of the heavy chain of the IgA antibody, wherein the IgG antibody further comprises a deletion of amino acid K447; or
(b) a sequence comprising C242 through the C-terminus of the heavy chain of the IgA antibody.

11. The isolated IgG-IgA fusion molecule of claim 10, wherein (a) the IgG antibody is selected from the group consisting of an IgG1 antibody, an IgG2 antibody, an IgG3 antibody and an IgG4 antibody; and/or (b) the IgA antibody is selected from the group consisting of an IgA1 antibody, an IgA2m1 antibody, an IgA2m2 antibody and an IgA2mn antibody.

12. An isolated nucleic acid encoding the IgG-IgA fusion molecule of claim 10.

13. A host cell comprising the nucleic acid of claim 12.

14. A method of producing an IgG-IgA fusion molecule comprising culturing the host cell of claim 13 so that the IgG-IgA fusion molecule is produced.

15. A pharmaceutical composition comprising one or more IgG-IgA fusion molecules of claim 10 and a pharmaceutically acceptable carrier.

16. A method of treating an individual having a disease, wherein the method comprises administering to the individual an effective amount of one or more IgG-IgA fusion molecules of claim 10.

17. A method of increasing the expression of IgA dimers, trimers or tetramers comprising:
(a) decreasing the amount of DNA encoding a joining chain (JC) introduced into a first cell relative to the amount of DNA that encodes the light chain (LC) and the heavy chain (HC) for increasing the expression of IgA dimers, trimers or tetramers, wherein increased expression of IgA dimers, trimers or tetramers is relative to the amount of IgA trimers or tetramers produced in a second cell introduced with greater amounts of HC and LC DNA relative to the amount of JC DNA; or
(b) increasing the amount of DNA encoding a joining chain (JC) that is introduced into a first cell relative to the amount of DNA that encodes the light chain (LC) and the heavy chain (HC) for increasing the expression of IgA dimers, wherein increased expression of IgA dimers is relative to the amount of IgA dimers produced in a second cell introduced with equal amounts of JC, LC and HC DNA.

18. The method of claim 17, wherein:
(a) the ratio of the amount of DNA encoding the HC to the amount of DNA encoding the LC to the amount of DNA encoding the JC (HC:LC:JC) that is introduced into the first cell for increased expression of IgA dimers, trimers or tetramers is from about 1:1:0.25 to about 1:1:0.5; or
(b) the ratio of the amount of DNA encoding the HC to the amount of DNA encoding the LC to the amount of DNA encoding the JC (HC:LC:JC) that is introduced into the first cell for increased expression of IgA dimers is from about 1:1:2 to about 1:1:5.

19. A method of increasing the production of IgA1 or IgA2m1 polymers, increasing production of IgA2m2 dimers or decreasing the production of IgA2m2 polymers comprising:
(a) expressing, in a first cell, an IgA1 or IgA2m1 antibody having a substitution at amino acid V458, wherein increased production of IgA1 or IgA2m1 polymers is relative to the amount of IgA1 or IgA2m1 polymers produced in a second cell expressing an IgA1 or IgA2m1 antibody that does not have a substitution at amino acid V458;
(b) expressing, in a first cell, an IgA1 or IgA2m1 antibody having a substitution at amino acid N459 or S461, wherein increased production of IgA1 or IgA2m1 polymers is relative to the amount of IgA1 or IgA2m1 polymers produced in a second cell expressing an IgA1 or IgA2m1 antibody that does not have a substitution at amino acid N459 or S461;
(c) expressing, in a first cell, an IgA2m2 antibody having a substitution at amino acid I458, wherein increased production of IgA2m2 dimers is relative to the amount of IgA2m2 dimers produced in a second cell expressing an IgA2m2 antibody that does not have a substitution at amino acid 1458; or
(d) expressing, in a first cell, an IgA2m2 antibody with a substitution at amino acid C471, wherein decreased production of IgA2m2 polymers is relative to the amount of IgA2m2 polymers produced in a second cell expressing an IgA2m2 antibody that does not have a substitution at amino acid C471.

20. The method of claim 19, wherein (a) amino acid V458 is substituted with an isoleucine (V4581), (b) amino acid I458 is substituted with a valine (I458V), (c) amino acid N459 is substituted with a N459Q, N459G or a N459A mutation, (d) amino acid S461 is substituted with a S461A mutation and/or (e) amino acid C471 is substituted with a C471S mutation.

21. A method of increasing transient expression of an IgA2m2 antibody comprising expressing, in a first cell, an IgA2m2 antibody that comprises a substitution at an amino acid selected from the group consisting of N166, S212, N263, N337, I338, T339, N459 and a combination thereof, wherein increased transient expression of the IgA2m2 antibody is relative to the amount of transient expression produced in a second cell expressing an IgA2m2 antibody that does not have a substitution at an amino acid selected from the group consisting of N166, S212, N263, N337, I338, T339, N459 and a combination thereof.

22. A method of expressing dimers of IgG-IgA fusion molecules or expressing dimers, trimers or tetramers of IgG-IgA fusion molecules comprising:
(a) expressing an IgG-IgA fusion molecule comprising a full-length IgG antibody fused at its C-terminus to an Fc region of an IgA antibody for producing dimers the IgG-IgA fusion molecule, wherein the Fc region of the IgA antibody comprises a sequence comprising P221 or R221 through the C-terminus of the heavy chain of the IgA antibody, and wherein the IgG antibody comprises a deletion of amino acid K447; or
(b) expressing an IgG-IgA fusion molecule comprising a full-length IgG antibody fused at its C-terminus to an Fc region of an IgA antibody for producing dimers, trimers or tetramers of the IgG-IgA fusion molecule, wherein the Fc region of the IgA antibody comprises a sequence comprising C242 through the C-terminus of the heavy chain of the IgA antibody.

23. A method for purifying an IgA antibody or an oligomeric state of an IgA antibody or an IgG-IgA fusion molecule from a mixture comprising an IgA antibody or an IgG-IgA fusion molecule and at least one host cell protein,
(i) wherein the method for purifying an IgA antibody from a mixture comprising an IgA antibody and at least one host cell protein comprises:
(a) applying the mixture to a column comprising Protein L to bind the IgA antibody;
(b) washing the Protein L column with a wash buffer comprising PBS; and
(c) eluting the IgA antibody from the Protein L column by an elution buffer comprising phosphoric acid; or
(ii) wherein the method for purifying an oligomeric state of an IgA antibody or an IgG-IgA fusion molecule from a mixture comprising an IgA antibody or an IgG-IgA fusion molecule and at least one host cell protein comprises:
(a) applying the mixture to an affinity purification column comprising Protein L or Protein A to bind the IgA antibody or IgG-IgA fusion molecule;
(b) washing the affinity purification column with a wash buffer;
(c) eluting the IgA antibody or IgG-IgA fusion molecule from the affinity purification column by an elution buffer to form a first eluate; and
(d) applying the first eluate to a size exclusion chromatography column to separate different oligomeric states of the IgA antibody or IgG-IgA fusion molecule and to obtain a flowthrough comprising an oligomeric state of the IgA antibody or IgG-IgA fusion molecule.


SEQUENCE LISTING (21 sequences)

SEQ ID NO: 1 - protein, 19 aa, Artificial Sequence
  source: /note=“Description of Artificial Sequence Synthetic peptide”
TQKSLSLSPGPVPPPPPCC

SEQ ID NO: 2 - protein, 11 aa, Artificial Sequence
  source: /note=“Description of Artificial Sequence Synthetic peptide”
TQKSLSLSPGC

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 5 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAGKPTHINVSVVMAEADGTCY

SEQ ID NO: 6 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 21 aa, Artificial Sequence
  source: /note=“Description of Artificial Sequence Synthetic peptide”
TQKSLSLSPGKLRVPPPPPCC

SEQ ID NO: 9 - protein, 344 aa, Mus sp.
ESARNPTIYPLTLPPALSSDPVIIGCLIHDYFPSGTMNVTWGKSGKDITTVNFPPALASGGRYTMSNQLTLPAVECPEGESVKCSVQHDSNPVQELDVNCSGPTPPPPITIPSCQPSLSLQRPALEDLLLGSDASITCTLNGLRNPEGAVFTWEPSTGKDAVQKKAVQNSCGCYSVSSVLPGCAERWNSGASFKCTVTHPESGTLTGTIAKVTVNTFPPQVHLLPPPSEELALNELLSLTCLVRAFNPKEVLVRWLHGNEELSPESYLVFEPLKEPGEGATTYLVTSVLRVSAETWKQGDQYSCMVGHEALPMNFTQKTIDRLSGKPTNVSVSVIMSEGDGICY

SEQ ID NO: 10 - protein, 339 aa, Rattus sp.
ESAKDPTIYPLRPPPSPSSDPVTIGCLIQNYFPSGTMNVTWGKSGKDISVINFPPAPASGPYTMCSQLTLPAAECPKGTSVKYYVQYNTSPVRELSVECPGPKPSLVCRPRLSLQRPALEDLLLGSEASLTCTLRGLKEPTGAVFTWQPTTGKDAVQKEAVQDSCGCYTVSSVLPGCAERWNNGETFTCTATHPEFETPLTGEIAKVTENTFPPQVHLLPPPSEELALNELVSLTCLVRGFNPKDVLVRWLQGNEELPSESYLVFEPLREPGEGAITYLVTSVLRVSAETWKQGAQYSCMVGHEALPMSFTQKTIDRLSGKPTNVNVSVIMSEGDGICY

SEQ ID NO: 11 - protein, 348 aa, Unknown
  source: /note=“Description of Unknown Rabbit IgA sequence”
AATNLELFPMTCPRPRPEQTVVVGCLIRGFFPLDPLSVSWDVSGENVRVYNFPPAQSGTSGLNTACSLLSLPSDQCPADDNVTCHVVHNNEGQDLPVPCHPECREPIIDPTPCPTTCGEPSLSLQRPDIGDLLLESKASLTCTLSGLKDPEGAVFTWEPTNGNEPVQQSTQSYPCGCYSVSSVLPGCAEPWNAGTEFTCTVTHPEIEGGSLTATISRGIIIPPLVHLLPPPSDELALNALVTLTCLVRGFSPKDVLVYWTNKGVNVPENSFLLWKPLPEPGQEPTTYAITSLLRVPAEDWNQNESYTCVVGHEGLAEHFTQRTINREAGRPTHVNVSVVVADVEGVCY

SEQ ID NO: 12 - protein, 341 aa, Sus sp.
SETSPKIFPLTLGSSEPAGYVVIACLVRDFFPSEPLTVTWSPSREGVIVRNFPPAQAGGLYTMSSQLTLPVEQCPADQILKCQVQHLSKSSQSVNVPCKVLPSDPCPQCCKPSLSLQPPALADLLLGSNASLTCTLSGLKKSEGVSFTWQPSGGKDAVQASPTRDSCGCYSVSSILPGCADPWNKGETFSCTAAHSELKSALTATITKPKVNTFRPQVHLLPPPSEELALNELVTLTCLVRGFSPKDVLVRWLQGGQELPRDKYLVWESLPEPGQAIPTYAVTSVLRVDAEDWKQGDTFSCMVGHEALPLAFTQKTIDRLAGKPTHVNVSVVMAEAEGICY

SEQ ID NO: 13 - protein, 353 aa, Gorilla sp.
ASPTSPKVFPLSLCSTQPDGDVVVACLVQGFFPQEPLSVTWSESGQGVTARNFPPSQDASGDLYTTSSQLTLPATQCPDGKSVTCHVNHYTNPSQDVTVPCRVPSTPPTPSPSTPPTPSPPCCHPRLSLHRPALEDLLLGSEANLTCTLTGLRDASGVTFTWTPSSGKSAVEGPPERDLCGCYSVSSVLPGCAEPWNHGKTFTCTAAYPESKTPLTATLSKSGNMFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 14 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 15 - protein, 25 aa, Homo sapiens
KVEPKSCDKTHTCPPCPAPELLGGP

SEQ ID NO: 16 - protein, 24 aa, Homo sapiens
PVPSTPPTPSPSTPPTPSPSCCHP

SEQ ID NO: 17 - protein, 11 aa, Homo sapiens
PVPPPPPCCHP

SEQ ID NO: 18 - protein, 11 aa, Homo sapiens
RVPPPPPCCHP

SEQ ID NO: 19 - protein, 50 aa, Artificial Sequence
  source: /note=“Description of Artificial Sequence Synthetic polypeptide”
MHEALHNHYTQKSLSLSPGKLRVPPPPPCCHPRLSLHRPALEDLLLGSEA

SEQ ID NO: 20 - protein, 40 aa, Artificial Sequence
  source: /note=“Description of Artificial Sequence Synthetic polypeptide”
MHEALHNHYTQKSLSLSPGCHPRLSLHRPALEDLLLGSEA

SEQ ID NO: 21 - protein, 48 aa, Artificial Sequence
  source: /note=“Description of Artificial Sequence Synthetic polypeptide”
MHEALHNHYTQKSLSLSPGPVPPPPPCCHPRLSLHRPALEDLLLGSEA